SEQUENCE LISTING (149 sequences)

SEQ ID NO: 1 - DNA, 843 bp, Amycolatopsis circi
  sig_peptide 1..102
  mat_peptide 103..840
  source 1..843: mol_type = genomic DNA
  CDS 1..840
atgccttcgcttcctggtcggatttctcgcggaaccgtagcggccgccctgctgatcggcagttcggcctgcgcgctcggggccggcggcacggcggaaagcgcgccgcttccctcgccgcacagccaggtcaaagacgttcccctgcccaccccgcacgccgggttcgactatcagatcggcggtccctaccagccgccggcgggcgtccaggtcgtcagccgcgaccacagcgccccggcggccgccgggctgtacaacatctgctacgtcaacgctttccaggctcagcccggcgccgaaaaggagtggggcgacctggtcctgcgcgacggcgacggcaaggtcgtcatggatcccgattggaacgaggcgctgctcgacctgcgcaccgcggacaagcggagccggatcgcggacaaggtcggcacgtggatcgacgagtgcgcgggcaagggctaccaggccgtcgagccggacaactacgacagcttcacccggtcccagggcctgctcagcgcgcggaacgcccaggacctcgtcaagttgctttcccagcgcgcgcacggaaaaggcctcgcgatcggccagaagaacacctccgaactggcctcgtcgcgggcggacaacgggctggacttcgccgtcgccgaggaatgcggcgaccaggacaattgcgccgagtacacccagtatttcggcaaccgcgtgatcgtcatcgaatacaccgaggacggcttgcgcaacgcgtgcgacaagtggggcgccgcgctcagcgtcgtccgccgcgaccgggacgtcacgccgaagggcgattccgcttacgtccgcgaaacctgctga

SEQ ID NO: 2 - protein, 280 aa, Amycolatopsis circi
MPSLPGRISRGTVAAALLIGSSACALGAGGTAESAPLPSPHSQVKDVPLPTPHAGFDYQIGGPYQPPAGVQVVSRDHSAPAAAGLYNICYVNAFQAQPGAEKEWGDLVLRDGDGKVVMDPDWNEALLDLRTADKRSRIADKVGTWIDECAGKGYQAVEPDNYDSFTRSQGLLSARNAQDLVKLLSQRAHGKGLAIGQKNTSELASSRADNGLDFAVAEECGDQDNCAEYTQYFGNRVIVIEYTEDGLRNACDKWGAALSVVRRDRDVTPKGDSAYVRETC

SEQ ID NO: 3 - protein, 246 aa, Amycolatopsis circi
APLPSPHSQVKDVPLPTPHAGFDYQIGGPYQPPAGVQVVSRDHSAPAAAGLYNICYVNAFQAQPGAEKEWGDLVLRDGDGKVVMDPDWNEALLDLRTADKRSRIADKVGTWIDECAGKGYQAVEPDNYDSFTRSQGLLSARNAQDLVKLLSQRAHGKGLAIGQKNTSELASSRADNGLDFAVAEECGDQDNCAEYTQYFGNRVIVIEYTEDGLRNACDKWGAALSVVRRDRDVTPKGDSAYVRETC

SEQ ID NO: 4 - DNA, 834 bp, Amycolatopsis sp.
  sig_peptide 1..102
  mat_peptide 103..831
  source 1..834: mol_type = genomic DNA
  CDS 1..831
atgcattcgcttcctggtcggatttctcgcgccaccgcggcggccgcgctgctggtcggcagttcggcgtgcgcgctcggaaccgccggcccggcggaaagcgcgccgctgccctcggcgcacagccaagtcccgctgcctgctccgcacgccgggttcgactaccagatcggcggcccctacgcgccgccgccgggcgtccaggcggtcagccgcgaccacagcgccccggcggcggccgggctgtacaacatctgctacgtcaacgctttccaggcacagcccgacgccgagaaggaatggggcgacctggtcctgcgcgacgccgacggcaacgtcgtgaaggatcccgactggaacgaggcgctgctcgacctgcgcaccgccgacaaacggggccggatcgccgacaaggtcggcgggtggatcgacgagtgcgcgggcaagggctaccaggccgtcgagccggacaactacgacagcttcacccggtcccaggacctgctcaccgcgcaggacgcacaggatctcgtcaagctgctttccgcgcacgcgcacggaaaaggcctcgcgatcgcgcagaagaacacctcggagctggcctcctcgcgcacggccaacgggctcgacttcgccgtcgccgaggaatgcggcgaacaggacaattgcgcggagtacacccaatacttcgacaaccacgtgatcgtcatcgaatacaccgaagccggtctgcgcaacgcgtgtgccaagtggggcgccactctcagcgtcgtgcgccgcgaccgggacgtcacgccgaagggcgattccgcctacgtccgcgaaacctgctga

SEQ ID NO: 5 - protein, 277 aa, Amycolatopsis sp.
MHSLPGRISRATAAAALLVGSSACALGTAGPAESAPLPSAHSQVPLPAPHAGFDYQIGGPYAPPPGVQAVSRDHSAPAAAGLYNICYVNAFQAQPDAEKEWGDLVLRDADGNVVKDPDWNEALLDLRTADKRGRIADKVGGWIDECAGKGYQAVEPDNYDSFTRSQDLLTAQDAQDLVKLLSAHAHGKGLAIAQKNTSELASSRTANGLDFAVAEECGEQDNCAEYTQYFDNHVIVIEYTEAGLRNACAKWGATLSVVRRDRDVTPKGDSAYVRETC

SEQ ID NO: 6 - protein, 243 aa, Amycolatopsis sp.
APLPSAHSQVPLPAPHAGFDYQIGGPYAPPPGVQAVSRDHSAPAAAGLYNICYVNAFQAQPDAEKEWGDLVLRDADGNVVKDPDWNEALLDLRTADKRGRIADKVGGWIDECAGKGYQAVEPDNYDSFTRSQDLLTAQDAQDLVKLLSAHAHGKGLAIAQKNTSELASSRTANGLDFAVAEECGEQDNCAEYTQYFDNHVIVIEYTEAGLRNACAKWGATLSVVRRDRDVTPKGDSAYVRETC

SEQ ID NO: 7 - DNA, 825 bp, Amycolatopsis sp.
  sig_peptide 1..111
  mat_peptide 112..822
  source 1..825: mol_type = genomic DNA
  CDS 1..822
gtgcccaccatcatccggagttcgatgatgcccttctcccgcgttcggttcctcgccagtgccgcgctcgcggccaccgtcctcggtggcaccgcgtgcacggcttcggcggccccggccgccgtgaccccgccgccggtcaaggcgggcttcgactaccagatcggcggcgcctacaccccgcccgcgggtgtccaggtcgtcagccgcgaccacggcgacgccccggccgccgggctctacaacatctgctacgtcaacgctttccaggcccagcccggctccgagggggagtggggcgacctgctgctgcgcgacgcgaacggcaaggtcgtcgtcgacgaggactgggacgaggcgctgctcgacctgcacaccgcggacaagcgcacgcgggtcgcggccaaggtgaacgcctggatcgacgactgcgccgcgaagggttaccaggcgatcgagccggacaactacgacagcttcacgcgctcccgcggtctgctctccgacagcgacgcgcaggcctacatccgcctgctttccgcccacgcccacgggaagggcctggcgatcgcgcagaagaacacctccgagctggccgggcagcgtcaggccaacgggctcgacttcgccatcgccgaagagtgcgggcagcagaagaactgcgacgagttcacgcccgcgttcggcgaccacgtgatcgtcatcgagtacaccgacggcgggctcaagaccgcctgcagccggtggagctcgctcagcatcgtccgccgcgacctcgacgtcgtccccaagggcgcgtccggctacgtccgcaaaacctgctga

SEQ ID NO: 8 - protein, 274 aa, Amycolatopsis sp.
VPTIIRSSMMPFSRVRFLASAALAATVLGGTACTASAAPAAVTPPPVKAGFDYQIGGAYTPPAGVQVVSRDHGDAPAAGLYNICYVNAFQAQPGSEGEWGDLLLRDANGKVVVDEDWDEALLDLHTADKRTRVAAKVNAWIDDCAAKGYQAIEPDNYDSFTRSRGLLSDSDAQAYIRLLSAHAHGKGLAIAQKNTSELAGQRQANGLDFAIAEECGQQKNCDEFTPAFGDHVIVIEYTDGGLKTACSRWSSLSIVRRDLDVVPKGASGYVRKTC

SEQ ID NO: 9 - protein, 237 aa, Amycolatopsis sp.
APAAVTPPPVKAGFDYQIGGAYTPPAGVQVVSRDHGDAPAAGLYNICYVNAFQAQPGSEGEWGDLLLRDANGKVVVDEDWDEALLDLHTADKRTRVAAKVNAWIDDCAAKGYQAIEPDNYDSFTRSRGLLSDSDAQAYIRLLSAHAHGKGLAIAQKNTSELAGQRQANGLDFAIAEECGQQKNCDEFTPAFGDHVIVIEYTDGGLKTACSRWSSLSIVRRDLDVVPKGASGYVRKTC

SEQ ID NO: 10 - DNA, 831 bp, Streptomyces parvulus
  sig_peptide 1..117
  mat_peptide 118..828
  source 1..831: mol_type = genomic DNA
  CDS 1..828
atgagccgcacgcaggtgcacacctcgaagaagaagccggtgctggcgggcgtctgcgccgccgccgtcctcgccaccgccgccgccctcgtcccgggcagcacccccgccgaggccgcggtaccggccccgcccccggccggcgccggcttcgactaccagatcggcggcgcctacaccccgccttccggcgtacgggtggtcagccgcgaccacacggcctcccccgccgcgggcctctacaacatctgctacgtcaacgccttccaggcccagcggggcgccgagggcgagtgggacgacgacctgctgctgcgcgacggctcggggaaggtcgtctacgacaccgagtggaaggaggccctgctcgacacccgcaccgccgacaagcgccggcgcatcgccgacaaggtcggtacctggatcgacgcctgcgcggacaagggcttccaggccgtcgagccggacaactacgacagctacacccgctcgaagaagctcttggacgccgccgacgcccaggccttcgtcaagctgctcgccgagcgcgcgcacgccggcggactggccatcgggcagaagaacaccgtcgaactcgccgggaaccgcgtcgccaacggtctggacttcgcggtcgccgaggagtgcggcgagtgggacgagtgcggggactacacggccgagttcggcgaccacgtgatcgtcgtcgagtacaccgccgcgggcctgaggaaggcgtgctccgggttcggcgacacgctgagcatcgtacggcgcgacctggacgtctcgccgaagggcagcagctcctacgtccgcgagacctgctga

SEQ ID NO: 11 - protein, 276 aa, Streptomyces parvulus
MSRTQVHTSKKKPVLAGVCAAAVLATAAALVPGSTPAEAAVPAPPPAGAGFDYQIGGAYTPPSGVRVVSRDHTASPAAGLYNICYVNAFQAQRGAEGEWDDDLLLRDGSGKVVYDTEWKEALLDTRTADKRRRIADKVGTWIDACADKGFQAVEPDNYDSYTRSKKLLDAADAQAFVKLLAERAHAGGLAIGQKNTVELAGNRVANGLDFAVAEECGEWDECGDYTAEFGDHVIVVEYTAAGLRKACSGFGDTLSIVRRDLDVSPKGSSSYVRETC

SEQ ID NO: 12 - protein, 237 aa, Streptomyces parvulus
AVPAPPPAGAGFDYQIGGAYTPPSGVRVVSRDHTASPAAGLYNICYVNAFQAQRGAEGEWDDDLLLRDGSGKVVYDTEWKEALLDTRTADKRRRIADKVGTWIDACADKGFQAVEPDNYDSYTRSKKLLDAADAQAFVKLLAERAHAGGLAIGQKNTVELAGNRVANGLDFAVAEECGEWDECGDYTAEFGDHVIVVEYTAAGLRKACSGFGDTLSIVRRDLDVSPKGSSSYVRETC

SEQ ID NO: 13 - DNA, 942 bp, Fusarium solani
  sig_peptide 1..66
  mat_peptide 67..939
  source 1..942: mol_type = genomic DNA
  CDS 1..519
  CDS 571..939
atgaagctgaaccgcctcatcaatagcatcattctccttgtggcatcttctgtgcctgcactcgcggtcccttccaacgcgaacgcccaggtggctgaagcactttctcctcgaggcgttaccactcgcaaagagctatggaagcctaaagttggcactccttggcaaatcgttctttccgaggtcatcaagatcccgaaaggcggcgccaagaatctgaagccaaacgtccccatctacgacgtggacttgtttgagaactcaaaggagacttttgacgctctgcataaagcgggcaagcatgtcatctgttacttcagcgccggatcttgggaaaattggcgcgatgataggaagagcttccagaagaaggatcttggcaagacgttgagtggatggccggacgaaaagtatgtcaacatcaacagcccttcagtacgggccattatggcaaagcgtatcaagttggctgccgagaagggctgtgatgccatcgatcctgacaacttggatggctacgtaagtcatgacccgtggacaacgaggctatcgctaacgtcttttttttagcaagcggataatggcctgggtctaaccgaggctgacacaatctcatatgtcaagttcctctccaaagaagcagccaagtaccgtatgacgacgggcatgaagaacggagggagcattacaaagcaggttctcccatatgtcggtttctgcatcaacgagtcttgcatacaatactcagagtgcgacttatatgcaccctacatcaaagcaggaaaacccgtcttcaatattgagtacccggcgggtgcaccaaaagtgaaggctgccgacaaaaaaaggatctgctcagtcactggtgccgccagcgggtcgaaagggttcagcaaggttatcaagaagatgaatctggatagttgggttatgtattgctag

SEQ ID NO: 14 - protein, 296 aa, Fusarium solani
MKLNRLINSIILLVASSVPALAVPSNANAQVAEALSPRGVTTRKELWKPKVGTPWQIVLSEVIKIPKGGAKNLKPNVPIYDVDLFENSKETFDALHKAGKHVICYFSAGSWENWRDDRKSFQKKDLGKTLSGWPDEKYVNINSPSVRAIMAKRIKLAAEKGCDAIDPDNLDGYQADNGLGLTEADTISYVKFLSKEAAKYRMTTGMKNGGSITKQVLPYVGFCINESCIQYSECDLYAPYIKAGKPVFNIEYPAGAPKVKAADKKRICSVTGAASGSKGFSKVIKKMNLDSWVMYC

SEQ ID NO: 15 - protein, 274 aa, Fusarium solani
VPSNANAQVAEALSPRGVTTRKELWKPKVGTPWQIVLSEVIKIPKGGAKNLKPNVPIYDVDLFENSKETFDALHKAGKHVICYFSAGSWENWRDDRKSFQKKDLGKTLSGWPDEKYVNINSPSVRAIMAKRIKLAAEKGCDAIDPDNLDGYQADNGLGLTEADTISYVKFLSKEAAKYRMTTGMKNGGSITKQVLPYVGFCINESCIQYSECDLYAPYIKAGKPVFNIEYPAGAPKVKAADKKRICSVTGAASGSKGFSKVIKKMNLDSWVMYC

SEQ ID NO: 16 - DNA, 1064 bp, Fusarium solani
  sig_peptide 1..111
  mat_peptide 112..1061
  source 1..1064: mol_type = genomic DNA
  CDS 1..51
  CDS 100..297
  CDS 360..1061
atggtggccttcagccgtattgctgtcaccactctcgcccttggcggttctgtaagcgctcgtcgtagaggagaatgtcctgctgctgacgctggcaagaccgcacaagccaagcctgccacggcggcgagggctactgggctcgccgacttcaagccaggtgtgcagtgggagatttgtattcatcatcccatcaagcatgatagtgccgcagacttgatcccgaccaaggccaaggtctgggatatcgacatgggccacgctcaggagttccctaacatgatccccatgctcaaggtaagcagacttacttcaacattttggtatggataagccatactaaccagattgcctaacagagcgctggcaaattcgttatttgttacttcaacgccggagcccttcaggactgggatgacgacaagagcaagttccccaaggaggtcatcggtcactccctgtcctacccctacgatagcgaagaatggtacctcgacatccgggactcgcgtgtcctcgagttgcaaacggcacgactagacattgctgccaagatcggctgcgatgccgtcgatcctgacaacgttgacgcctggcaacaagatgacgaagaccccacgggcttcaagctcaagtcttccgactacaccaactacctcaagaacctcgccaagtatgcccactcgatcaagaccaaggacggtcagcccctcctcgtcgggcagaagaacgcccccgagatcgccgaggacctcgtctcgaccctcgactttgccgtgctcgagtcctgccgcggcaacagcgaccccaacgaagagagctggcccttttgcgaggacttccagacctacatcgacgccggcaagcccgtcctccagatcgagtaccccccctcggtggagaagacgggcaaggtcagcgcctcggataacaagtactactgcactgccgaggatgaggacaagggcttcagcaagatcatcaagtgggcttctgctcagcttgatggctggggtcagtactgtggcgaggagcccttccgtactcctgcggccaagtattaa

SEQ ID NO: 17 - protein, 317 aa, Fusarium solani
MVAFSRIAVTTLALGGSTAQAKPATAARATGLADFKPGVQWEICIHHPIKHDSAADLIPTKAKVWDIDMGHAQEFPNMIPMLKSAGKFVICYFNAGALQDWDDDKSKFPKEVIGHSLSYPYDSEEWYLDIRDSRVLELQTARLDIAAKIGCDAVDPDNVDAWQQDDEDPTGFKLKSSDYTNYLKNLAKYAHSIKTKDGQPLLVGQKNAPEIAEDLVSTLDFAVLESCRGNSDPNEESWPFCEDFQTYIDAGKPVLQIEYPPSVEKTGKVSASDNKYYCTAEDEDKGFSKIIKWASAQLDGWGQYCGEEPFRTPAAKY

SEQ ID NO: 18 - protein, 296 aa, Fusarium solani
KPATAARATGLADFKPGVQWEICIHHPIKHDSAADLIPTKAKVWDIDMGHAQEFPNMIPMLKSAGKFVICYFNAGALQDWDDDKSKFPKEVIGHSLSYPYDSEEWYLDIRDSRVLELQTARLDIAAKIGCDAVDPDNVDAWQQDDEDPTGFKLKSSDYTNYLKNLAKYAHSIKTKDGQPLLVGQKNAPEIAEDLVSTLDFAVLESCRGNSDPNEESWPFCEDFQTYIDAGKPVLQIEYPPSVEKTGKVSASDNKYYCTAEDEDKGFSKIIKWASAQLDGWGQYCGEEPFRTPAAKY

SEQ ID NO: 19 - DNA, 795 bp, Nonomuraea guangzhouensis
  sig_peptide 1..81
  mat_peptide 82..792
  source 1..795: mol_type = genomic DNA
  CDS 1..792
atgagcctcacccgtagctgcttaggcgttgccgtggcccttgccgccgcgttggccgccacaccgtcgaccgcctcggcggccgcggtcaccctgcccccgacacacgccgggttcgactaccagatcggcggcgcctacacgccgccggccggggtcaccgtggtcaccagggaccgggccgccgcgcccgccgcggggctgtacaacatctgctacgtcaacgccttccaggttcagccgggcgagcagggcgactgggactccgacctgctgctgcgtgacgccaacggtgacgtggtcgtcgacgaggactggggcgaggcactgctggacctgcgtacggatgccaagcggaagcgcgtcgccgcgaaggtgaactcctggatcaccggctgcaagaccaagggctaccaggccatcgagcccgacaactacgacagctacacccggtcgaagaagctgctgacggcgtcgaacgcccaggcgtacatccggctgctcagcagccacgcccacacccagggcctggccatcgcgcagaagaacaccgtggagctggccggctcgcgcaccgcgaacgggctcgacttcgcggtcgcggaggagtgcggccagtacaaggagtgcggcgactacacggcggcgttcggcaacaacgtgatcgtgatcgagtacaacgccaccggtctgtcgagggcgtgctcgggctggggcaccaagctgagcatcgtccggcgcgatgtggacgtctcacccaagggcagcagcggatacgtccgcaagacctgctga

SEQ ID NO: 20 - protein, 264 aa, Nonomuraea guangzhouensis
MSLTRSCLGVAVALAAALAATPSTASAAAVTLPPTHAGFDYQIGGAYTPPAGVTVVTRDRAAAPAAGLYNICYVNAFQVQPGEQGDWDSDLLLRDANGDVVVDEDWGEALLDLRTDAKRKRVAAKVNSWITGCKTKGYQAIEPDNYDSYTRSKKLLTASNAQAYIRLLSSHAHTQGLAIAQKNTVELAGSRTANGLDFAVAEECGQYKECGDYTAAFGNNVIVIEYNATGLSRACSGWGTKLSIVRRDVDVSPKGSSGYVRKTC

SEQ ID NO: 21 - protein, 237 aa, Nonomuraea guangzhouensis
AAVTLPPTHAGFDYQIGGAYTPPAGVTVVTRDRAAAPAAGLYNICYVNAFQVQPGEQGDWDSDLLLRDANGDVVVDEDWGEALLDLRTDAKRKRVAAKVNSWITGCKTKGYQAIEPDNYDSYTRSKKLLTASNAQAYIRLLSSHAHTQGLAIAQKNTVELAGSRTANGLDFAVAEECGQYKECGDYTAAFGNNVIVIEYNATGLSRACSGWGTKLSIVRRDVDVSPKGSSGYVRKTC

SEQ ID NO: 22 - DNA, 819 bp, Streptomyces sp.
  sig_peptide 1..99
  mat_peptide 100..816
  source 1..819: mol_type = genomic DNA
  CDS 1..816
atgcgcatcgaccgcggttccgctctccgactcaccgcgcttccggcgctgttggccgccgcactcgtcccgtcgctcgcgatggaccaggccggcgcggcgtccgaagcggtgtcaccgccgccggcccacgccggtttcgactaccagatcggcggcgcgtaccccccgcccgccggggtgagcgtggtcagccgcgaccacaccgacccgcccgccgccgggctgtacaacatctgctacgtcaacgcgttccaggcacagcccggcgccgagggcgactgggactccgacctcctgctccacgacgccaagggaaaggtcgtctacgacgaggactggggcgaggcgctgctcgacaccagcaccgcgaacaagcggcagcggatcgccgagaaggtcgacggctggatcgacgactgcgcggccaagggctaccaggcgatcgagcccgacaactacgacagctacagccgctccggcaagctgctgacctccgccgaggcggaggcgtacatcaccctgctgtccgcgcacgcgcacgccgacggcctggccatcgcgcagaagaacacctccgagctggcgggcgaccgggcgaagaccggactggacttcgcggtcgcggaggagtgcgggacgtacgacgagtgcggcgactacaccgccgcgttcggcgaccacgtcatcgtcatcgagtacaccgcgaaggggctgcagaaggcgtgctccggctggggcgacgagttgagcgtcgtccgccgtgacctggacgtctctccggccggcagcaagggctacgtccgccagacctgctga

SEQ ID NO: 23 - protein, 272 aa, Streptomyces sp.
MRIDRGSALRLTALPALLAAALVPSLAMDQAGAASEAVSPPPAHAGFDYQIGGAYPPPAGVSVVSRDHTDPPAAGLYNICYVNAFQAQPGAEGDWDSDLLLHDAKGKVVYDEDWGEALLDTSTANKRQRIAEKVDGWIDDCAAKGYQAIEPDNYDSYSRSGKLLTSAEAEAYITLLSAHAHADGLAIAQKNTSELAGDRAKTGLDFAVAEECGTYDECGDYTAAFGDHVIVIEYTAKGLQKACSGWGDELSVVRRDLDVSPAGSKGYVRQTC

SEQ ID NO: 24 - protein, 239 aa, Streptomyces sp.
ASEAVSPPPAHAGFDYQIGGAYPPPAGVSVVSRDHTDPPAAGLYNICYVNAFQAQPGAEGDWDSDLLLHDAKGKVVYDEDWGEALLDTSTANKRQRIAEKVDGWIDDCAAKGYQAIEPDNYDSYSRSGKLLTSAEAEAYITLLSAHAHADGLAIAQKNTSELAGDRAKTGLDFAVAEECGTYDECGDYTAAFGDHVIVIEYTAKGLQKACSGWGDELSVVRRDLDVSPAGSKGYVRQTC

SEQ ID NO: 25 - DNA, 864 bp, Vibrio sp.
  sig_peptide 1..69
  mat_peptide 70..861
  source 1..864: mol_type = genomic DNA
  CDS 1..861
atgccttattatcgtccattaaactacgttatctgcttgctcggtctagcgctgcttgttggctgtgggagcgatagtaaagactctgaaagcgacttcgaaattctcacccccgtaacgggtggcaattggtatcaacccacccgccaaacgacctggcagtggcagctcacaaatacgcttaactcaagctacaatgtgacggtatatgacattgaccttttcgataccagcccgagtgacatccaagcgttacaaaccgagggccacaaggtcatctgctacttttctgctggttcgtttgaagattggcgtgacgacgccgctgaattctcatcaagcgtattaggtgatacgcttgatggctgggaagatgaacgctggttagacattcgacaatcatccgttcgcgatatcatgtacgctcgcttacaactggcacaaactaaaggctgtgatggtgttgaaccggataatgtcgatggctacaccaacaattctggctttaaccttggttattcagaccaactcatgttcaatcgttacctcgccaactcagcccaccaactcggcttgtccatagggctgaaaaacgatttacggcaaattaccgatttagtcgaatattttgacttcgccgtcaacgagcaatgctttgaatacaacgagtgtggctacctagcaccttttattgacgccaacaaagcggtgtttaacgccgagtatgaatcgacttacgtcgataataccagcgcacgtgatgcattgtgtaatgacgccaaccaacgtcaattcagcacgttagtcctgccattgtacttggatgatagctttcgttacagctgtctaagcgaacctttttga

SEQ ID NO: 26 - protein, 287 aa, Vibrio sp.
MPYYRPLNYVICLLGLALLVGCGSDSKDSESDFEILTPVTGGNWYQPTRQTTWQWQLTNTLNSSYNVTVYDIDLFDTSPSDIQALQTEGHKVICYFSAGSFEDWRDDAAEFSSSVLGDTLDGWEDERWLDIRQSSVRDIMYARLQLAQTKGCDGVEPDNVDGYTNNSGFNLGYSDQLMFNRYLANSAHQLGLSIGLKNDLRQITDLVEYFDFAVNEQCFEYNECGYLAPFIDANKAVFNAEYESTYVDNTSARDALCNDANQRQFSTLVLPLYLDDSFRYSCLSEPF

SEQ ID NO: 27 - protein, 264 aa, Vibrio sp.
SDSKDSESDFEILTPVTGGNWYQPTRQTTWQWQLTNTLNSSYNVTVYDIDLFDTSPSDIQALQTEGHKVICYFSAGSFEDWRDDAAEFSSSVLGDTLDGWEDERWLDIRQSSVRDIMYARLQLAQTKGCDGVEPDNVDGYTNNSGFNLGYSDQLMFNRYLANSAHQLGLSIGLKNDLRQITDLVEYFDFAVNEQCFEYNECGYLAPFIDANKAVFNAEYESTYVDNTSARDALCNDANQRQFSTLVLPLYLDDSFRYSCLSEPF

SEQ ID NO: 28 - DNA, 726 bp, Synthetic construct
  misc_feature 1..726: note = Synthetic gene
  source 1..726: mol_type = other DNA
  CDS 1..726
ctgaatggcggctactggactccgccggccgacgtacgcgggacgtttcagaaaagcccgaacggcgttccggtcgatcaggatttgaaactggcgattgttcccttgtttggagcgaacgaggcccagatccagaaactgcagagcgagggcatttcgacaatctgtacgatcaatgtcggctacgtcctgaacaccgatcccgattacgatcagtttcggccctcaatgatcggtaagctcgtttgggaaggcaacgttccgcattggatcgatatccgcaacaccggtgtccgagccttgatgcaagcgcgaatggaacatgccaaggctattggatgtaagggcgtcgttacccagagtatggaactttggtacaacgacaccggtttctatatttcgtacggcgatcagcttgtttataatcggtggttggccgataccgcgcaccaactcgggctctcgatcggcatgcacaacactttttttcaggtgaacgacttagtcagctggtttgacttcgggttggtggaggactgcctcaactctggagattgcgaaacgtacattcccttcctctccgccggaaaaccagtgcttgatattgagtacaactcgatgagtagcgaggtcgatatctgcaaagccggccgcgaactcggcttcaacaccatcatcaagggctacagcgacgacaccaccttcgtcgactgcagggatcgtcga

SEQ ID NO: 29 - protein, 242 aa, Synthetic construct
  REGION 1..242: note = Synthetic Construct
LNGGYWTPPADVRGTFQKSPNGVPVDQDLKLAIVPLFGANEAQIQKLQSEGISTICTINVGYVLNTDPDYDQFRPSMIGKLVWEGNVPHWIDIRNTGVRALMQARMEHAKAIGCKGVVTQSMELWYNDTGFYISYGDQLVYNRWLADTAHQLGLSIGMHNTFFQVNDLVSWFDFGLVEDCLNSGDCETYIPFLSAGKPVLDIEYNSMSSEVDICKAGRELGFNTIIKGYSDDTTFVDCRDRR

SEQ ID NO: 30 - protein, 242 aa, Synthetic construct; identical to SEQ ID NO: 29
  REGION 1..242: note = Synthetic gene

SEQ ID NO: 31 - DNA, 831 bp, Microbacterium saccharophilum
  sig_peptide 1..114
  mat_peptide 115..828
  source 1..831: mol_type = genomic DNA
  CDS 1..828
atgcgcctcctcaccagcggaatcttcggtgcgacggtgctgctcgccctcacggcgtgcgcgagcgccccggctcccgtcccctcggtctggccgacatcgacggcttccgctcagatcgcgcttccgcccgcgggcgcgaaccccgactaccaactcggtggggcatacgacccgcccgacggcgtggccatcgtcgcgcgcgaccgtacggccgaccctgcaccgggtctgtactcgatctgctacgtcaacgccttccagacacagccgggcgagctggccgactggcccggcgagctgatcctgaaggatgcgtccggagagcccgtgcgcgaccccgactggcccgacgaggcgatcgtcgacacccgggatgccgcggcggtggctgccatcgtgacgccctggatcgacgcgtgcgcggcctccggcttcgacgcggtggagttcgacaacctcgacacctacacgcgcaccgatggaaccctcacccgggatgacaacctgaccgtcgccaccctgctggtgcgggcggcacacgacgccggcctggcggcgggacagaagaatgccgccgaggatgcacgcctgctccacgatcgcgccgggttcgacttcgccgtcaccgaagaatgcgccgtgtgggacgagtgcggcgcctacaccgccgtgtacggcgatgcggtgatcgccgtcgagtacaccgacgacctcccccgtccgttcgcggcgatgtgcgagtccgatctgccgccgtccgcggtgctgcgcgaccgcgatctgaccacaccgggcgacccggcctacgtcttcgagagctgtcgctga

SEQ ID NO: 32 - protein, 276 aa, Microbacterium saccharophilum
MRLLTSGIFGATVLLALTACASAPAPVPSVWPTSTASAQIALPPAGANPDYQLGGAYDPPDGVAIVARDRTADPAPGLYSICYVNAFQTQPGELADWPGELILKDASGEPVRDPDWPDEAIVDTRDAAAVAAIVTPWIDACAASGFDAVEFDNLDTYTRTDGTLTRDDNLTVATLLVRAAHDAGLAAGQKNAAEDARLLHDRAGFDFAVTEECAVWDECGAYTAVYGDAVIAVEYTDDLPRPFAAMCESDLPPSAVLRDRDLTTPGDPAYVFESCR

SEQ ID NO: 33 - protein, 238 aa, Microbacterium saccharophilum
QIALPPAGANPDYQLGGAYDPPDGVAIVARDRTADPAPGLYSICYVNAFQTQPGELADWPGELILKDASGEPVRDPDWPDEAIVDTRDAAAVAAIVTPWIDACAASGFDAVEFDNLDTYTRTDGTLTRDDNLTVATLLVRAAHDAGLAAGQKNAAEDARLLHDRAGFDFAVTEECAVWDECGAYTAVYGDAVIAVEYTDDLPRPFAAMCESDLPPSAVLRDRDLTTPGDPAYVFESCR

SEQ ID NO: 34 - DNA, 864 bp, Microbacterium oxydans
  sig_peptide 1..78
  mat_peptide 79..861
  source 1..864: mol_type = genomic DNA
  CDS 1..861
atgctcgaacgagaccgccgcaccgcacgcggcggcatcgctgtactcgccgccctcctcgcgctcaccggctgcgcgtccggcacggcgacggccccgcactccgggcactccgctgacgccccgacagacgcagaccggtcgctgccgcccgccggcgccgtgcccgactaccagctcggcggtgcgtacgagccggcggccgaggtcggcatcgtggggcgcgatcgttcggcggccccggcgcccgacacatactcgatctgctatgtgaacggtttccagacgcagccgggcgagctcgacacctgggacgccgatctgctgctgcgacaggacggcgagaccgtgttcgacccggactggccggacgaggcactgctcgacacgtcgaccgccgaccgccgcgaacgcatcgccgcacggatcatcccctggatcgaaggctgtgccgatgacggcttcgccgcggtcgaattcgacaacctcgattcctacacccggtccggcgacgcgctctcgctcgatgacaacctcgccctcgccgccctcttcgtcgacgccgcacatggcgccggcctcgcggccggccagaagaacgcggcggaagacgccgtggtgctgcacgaacaggcgggtttcgacttcgcggtgaccgaagagtgcgccgtcttcgaggagtgcggcgtctacgccgccgtctacggcacgcacgtgatcgacatcgagtacagcgatgagttgccgcgcagtttcgcggagatgtgcgcggacaccgattcgcccgcctcgatggtgctgcgtgatcgcgacctgctcacccccgacgacccggcgtacgtgttcgagacctgcagcgagtga

SEQ ID NO: 35 - protein, 287 aa, Microbacterium oxydans
MLERDRRTARGGIAVLAALLALTGCASGTATAPHSGHSADAPTDADRSLPPAGAVPDYQLGGAYEPAAEVGIVGRDRSAAPAPDTYSICYVNGFQTQPGELDTWDADLLLRQDGETVFDPDWPDEALLDTSTADRRERIAARIIPWIEGCADDGFAAVEFDNLDSYTRSGDALSLDDNLALAALFVDAAHGAGLAAGQKNAAEDAVVLHEQAGFDFAVTEECAVFEECGVYAAVYGTHVIDIEYSDELPRSFAEMCADTDSPASMVLRDRDLLTPDDPAYVFETCSE

SEQ ID NO: 36 - protein, 261 aa, Microbacterium oxydans
SGTATAPHSGHSADAPTDADRSLPPAGAVPDYQLGGAYEPAAEVGIVGRDRSAAPAPDTYSICYVNGFQTQPGELDTWDADLLLRQDGETVFDPDWPDEALLDTSTADRRERIAARIIPWIEGCADDGFAAVEFDNLDSYTRSGDALSLDDNLALAALFVDAAHGAGLAAGQKNAAEDAVVLHEQAGFDFAVTEECAVFEECGVYAAVYGTHVIDIEYSDELPRSFAEMCADTDSPASMVLRDRDLLTPDDPAYVFETCSE

SEQ ID NO: 37 - DNA, 873 bp, Frigoribacterium faeni
  sig_peptide 1..75
  mat_peptide 76..870
  source 1..873: mol_type = genomic DNA
  CDS 1..870
atgcgcgtacccctcggattcgtcggcctggtcgccgtggccctcgccgtgacgggttgcacgaacgtcgacgccggggtcggtgaacgacccgagcgcgcctcctcgtccgcggtggtggcgtcgggggccgcgacggccgacggcgtcgtgctgccgcccgagggcgcccgcttcgactaccaactgggaggcgcgtcgccggtccccgacggagccaccgtcgtcgcccgcgacagcaccgacgaccccgccgagggcgcctacggcatctgctacgtcaacgggttccagacgcagcccggagtcatgtggccggacgagctgctcgtgcggacggccgacggcgaaccgctggtcgacccgggctggccggacgagcacctgttcgacctgtccaccgaggcgaaccggcaggccgtggccgagcgccaggcctcgacgatcgacgggtgcgccgcctcgggctaccaggcggtcgagttcgacaacctcgactcgtggacgcggtcggacgaggccttcggcgaggacgacgcggtcgccttcgcgacgctgctcgtcgcgcgggcccacgcggccgggctggcgacggcgcagaagaacaccgccgacctcggtgcccggggccgcgacgaggtcggctacgacttcgccgtcaccgaggagtgcgaccgctacgacgagtgcgacgccttcacggacgtctacggcgggctcgtgttcgacgtcgagtacaccgacgacctccgcggctcgatcgccgaggtctgcgcccgcgtcggcgagctcgacccggccccctcgaccatcgtgcgcgaccacgacctggtgccggccgacgacccggcccacgcctacacggcctgctga

SEQ ID NO: 38 - protein, 290 aa, Frigoribacterium faeni
MRVPLGFVGLVAVALAVTGCTNVDAGVGERPERASSSAVVASGAATADGVVLPPEGARFDYQLGGASPVPDGATVVARDSTDDPAEGAYGICYVNGFQTQPGVMWPDELLVRTADGEPLVDPGWPDEHLFDLSTEANRQAVAERQASTIDGCAASGYQAVEFDNLDSWTRSDEAFGEDDAVAFATLLVARAHAAGLATAQKNTADLGARGRDEVGYDFAVTEECDRYDECDAFTDVYGGLVFDVEYTDDLRGSIAEVCARVGELDPAPSTIVRDHDLVPADDPAHAYTAC

SEQ ID NO: 39 - protein, 265 aa, Frigoribacterium faeni
GVGERPERASSSAVVASGAATADGVVLPPEGARFDYQLGGASPVPDGATVVARDSTDDPAEGAYGICYVNGFQTQPGVMWPDELLVRTADGEPLVDPGWPDEHLFDLSTEANRQAVAERQASTIDGCAASGYQAVEFDNLDSWTRSDEAFGEDDAVAFATLLVARAHAAGLATAQKNTADLGARGRDEVGYDFAVTEECDRYDECDAFTDVYGGLVFDVEYTDDLRGSIAEVCARVGELDPAPSTIVRDHDLVPADDPAHAYTAC

SEQ ID NO: 40 - DNA, 762 bp, Serinibacter sp.
  source 1..762: mol_type = genomic DNA
  CDS 1..762
acggacggcggctccgacgtcgtcctcccacccacggccggcgccttcgactaccggctcggcgaggcgtacggggagccgggcgagctcgacgtcgtcgcccgcgacgtcaccgcccagccgctcgcggacgcctacaacgtctgctacctcaacggtttccagacccagcccggcgccctgccggactgggaggacggcgacggcggcgccctgctgcgggacgcgagcggtgccgtcgtgatggatccggagtggccggacgaggccgtgctcgacccctcgacgccccagcagcgcatcgcgatcctcgccgtgatgggaccgcagatccgggactgcgccgacgcgggcttcgacgccgtcgagctcgacaacctcgacacgttcctgaggttcgagggcgtcgaccgcgacggcgcgctcgacctggcgacctcctacgtggaggtcgcgcacgacgcgggcctcgccgtcgggcagaagaacgcggccgacgtcggagccgatgcgcgtgaggtcgcgggcttcgacttcgccgtcgtggaggagtgcgccgtgtacgacgagtgcggcacctaccgcgcggtgtacggcgagcacgtgctgcagatcgagtactccgacacccttcgcgaccgaggcctgacggtcgaggaggtgtgcgcgctgcccgaccgcgcgccgctgaccgtggtgcgcgaccggctgctcgtcggccccgacgacgccgcccacgagcgggagcagtgcccggcgggc

SEQ ID NO: 41 - protein, 254 aa, Serinibacter sp.
TDGGSDVVLPPTAGAFDYRLGEAYGEPGELDVVARDVTAQPLADAYNVCYLNGFQTQPGALPDWEDGDGGALLRDASGAVVMDPEWPDEAVLDPSTPQQRIAILAVMGPQIRDCADAGFDAVELDNLDTFLRFEGVDRDGALDLATSYVEVAHDAGLAVGQKNAADVGADAREVAGFDFAVVEECAVYDECGTYRAVYGEHVLQIEYSDTLRDRGLTVEEVCALPDRAPLTVVRDRLLVGPDDAAHEREQCPAG

SEQ ID NO: 42 - protein, 254 aa, Serinibacter sp.; identical to SEQ ID NO: 41

SEQ ID NO: 43 - DNA, 900 bp, Oerskovia paurometabola
  sig_peptide 1..93
  mat_peptide 94..897
  source 1..900: mol_type = genomic DNA
  CDS 1..897
gtgccctcgctccctctccgcgcgtccgcgcacgcatcgctcatcgtgccggccctggtcctcgtcctccttgccgggtgcgccgtcggcgctgccccgtcgcccacccccggggcgaccgaccgtgagccgtcggcctcgacgagcggcgccgtcgggcaggtcggcgaagcccccgcacccccgcccgccggggccggtctcgactaccagctcggcggggcctacccgccgcccgacggcgtcacggtcgtcgtgcgcgacgtcaccgactcccccgcgggcgcgggctacgacgtctgttacgtcaacgggttccagacccagcccggcgagggcgcgacctggctgcgcgaccaccccgacctcgtcctgcaggacgacggcggtacacctgtcgccgacccaggctggcccgacgagctgctcctcgacacctcgaccgacgccgcgcgccgcgagatcgcgcagatcgtcggcggccaggtggacgcgtgcgcggacgccggcttcgacgcggtcgagcccgacaacctggactcgttcacccgctcggacggcgctctcaccagggacgacaacctcgcgctggccgcgctgctgatcgaacgtgcccacgcccggggcctggcgatcgggcagaagaacacggtcgagctgggcggtcgcggcccggcgctcgggttcgacttcgtggtcaccgaggactgcgcggtccacgacgagtgcggcgactacgccgcggcctacgggacgcgcgtcctggagatcgagtacgacgacgggccggccctcgcggagcgcctcgcctcggcgtgcgctcgcggggcgtcggtcgtgggccgcgaccgcgacctcgcccggccgcgcgagccgggctacgccttcgcgtcctgctga

SEQ ID NO: 44 - protein, 299 aa, Oerskovia paurometabola
VPSLPLRASAHASLIVPALVLVLLAGCAVGAAPSPTPGATDREPSASTSGAVGQVGEAPAPPPAGAGLDYQLGGAYPPPDGVTVVVRDVTDSPAGAGYDVCYVNGFQTQPGEGATWLRDHPDLVLQDDGGTPVADPGWPDELLLDTSTDAARREIAQIVGGQVDACADAGFDAVEPDNLDSFTRSDGALTRDDNLALAALLIERAHARGLAIGQKNTVELGGRGPALGFDFVVTEDCAVHDECGDYAAAYGTRVLEIEYDDGPALAERLASACARGASVVGRDRDLARPREPGYAFASC

SEQ ID NO: 45 - protein, 268 aa, Oerskovia paurometabola
APSPTPGATDREPSASTSGAVGQVGEAPAPPPAGAGLDYQLGGAYPPPDGVTVVVRDVTDSPAGAGYDVCYVNGFQTQPGEGATWLRDHPDLVLQDDGGTPVADPGWPDELLLDTSTDAARREIAQIVGGQVDACADAGFDAVEPDNLDSFTRSDGALTRDDNLALAALLIERAHARGLAIGQKNTVELGGRGPALGFDFVVTEDCAVHDECGDYAAAYGTRVLEIEYDDGPALAERLASACARGASVVGRDRDLARPREPGYAFASC

SEQ ID NO: 46 - DNA, 876 bp, Agreia pratensis
  sig_peptide 1..72
  mat_peptide 73..873
  source 1..876: mol_type = genomic DNA
  CDS 1..873
atgcgtgtgacgagagcggcggtggcgttgatcgcggccgtgctggtggcgttcgcggccgccgggtgcgcgcccgtgaaggtcaacgtcttctctccggtcaccgacggcgacgaagcttccggcgaaacgggacgccagagcatcgtgctgccgccgttcggcgagccgctcgactaccagctcggcggcagctacccggtcgacgagggtgtcggcatcgtgacccgagacagcacctcggagcccgcggccggggtctactcgatctgctacgtcaatggattccagtcgcagcccggcgacgacgagcgctggacggccgacaaccccgatctcgtgctgcgtgacgacgcggggcaggcgatcatcgatccgaactggccagacgagttcattctcgacacctcgactcccgagaagcggcaacgcatcgtcgctatcgtcggcgcctcgatcaccacctgcgcggaggcaggcttcgacgcgatcgagatcgacaacctcgacacctactcccgcagcgacggccggctcgacatcgacgacaacctcgcgctcgcggccctcttcgccgagcgggcgcacggcctggcgctcgcgatcgggcagaagaactctgccgagctggccgcgcgcggccgtgacgaggcgcacttcgacttcgcggtcaccgaggaatgtgtgcgcttcgaggagtgcgcggcgttcagcgacgtctacggcgaggccgtcgtcgacatcgagtacacagacgatctgcccggcagcttcgccgacgtctgcgcaacgaaagaccgcccggcgagcacaacgctgcgagaccgcaacctggtggtcaagggtgacgcggactacgtattcgagcactgctga

SEQ ID NO: 47 - protein, 291 aa, Agreia pratensis
MRVTRAAVALIAAVLVAFAAAGCAPVKVNVFSPVTDGDEASGETGRQSIVLPPFGEPLDYQLGGSYPVDEGVGIVTRDSTSEPAAGVYSICYVNGFQSQPGDDERWTADNPDLVLRDDAGQAIIDPNWPDEFILDTSTPEKRQRIVAIVGASITTCAEAGFDAIEIDNLDTYSRSDGRLDIDDNLALAALFAERAHGLALAIGQKNSAELAARGRDEAHFDFAVTEECVRFEECAAFSDVYGEAVVDIEYTDDLPGSFADVCATKDRPASTTLRDRNLVVKGDADYVFEHC

SEQ ID NO: 48 - protein, 267 aa, Agreia pratensis
PVKVNVFSPVTDGDEASGETGRQSIVLPPFGEPLDYQLGGSYPVDEGVGIVTRDSTSEPAAGVYSICYVNGFQSQPGDDERWTADNPDLVLRDDAGQAIIDPNWPDEFILDTSTPEKRQRIVAIVGASITTCAEAGFDAIEIDNLDTYSRSDGRLDIDDNLALAALFAERAHGLALAIGQKNSAELAARGRDEAHFDFAVTEECVRFEECAAFSDVYGEAVVDIEYTDDLPGSFADVCATKDRPASTTLRDRNLVVKGDADYVFEHC

SEQ ID NO: 49 - DNA, 864 bp, Plantibacter flavus
  sig_peptide 1..108
  mat_peptide 109..861
  source 1..864: mol_type = genomic DNA
  CDS 1..861
atgacccgtctcccccgtcgcgcgcacctgctcaccgcggcgggcgccgccctcctgctcgcggtcaccgccgccggctgctccacgttcccgagcggtgcgagctccgacaccggaccgacccactcagcgggtggattccccacggacgtcgtcttcgactaccagctcggtggcggctacgagccgacggccggcgtgggcggcgtcgcgcgcgacagcaccgactcccccgaccccgaccggtactccatctgctacgtcaacgggttccagagccagccggccgatcgcgacgcctggctggccgagccggacctcgtcctcatcgacgacgccggtgaaccggtcatcgacgagaactggcccgacgaactcatcttcgacctctccaccgacgaccgtcgcgcgcgcatcgccgagcgggtcggtgcgagcatcacccgctgcgcggaggccggcttcgacgccgtcgagatcgacaacctcgactcgtacacccggtccgacggtcggctcaccgttgaggacgcgatcgccctcgccacccggtacgccggcctcgcccacgacgcgggcctcctcatcgggcagaagaacgccgccgagctgggcacgcgagggcgggacgacgtcgggttcgacttcgccgtcgcggaggagtgccaccgcttcgacgagtgcgccacctacaccgaggtgtacggaggcgccgtgctggacatcgagtacaccgacgacctgcgcggcaccttcgacgaggtgtgcgccgatccgcaggtcccgttctccacgatcctgcgcgaccgcgacctccggacggccgacgaccccgcccacgtcttcgacgcctgcgctcggtag

SEQ ID NO: 50 - protein, 287 aa, Plantibacter flavus
MTRLPRRAHLLTAAGAALLLAVTAAGCSTFPSGASSDTGPTHSAGGFPTDVVFDYQLGGGYEPTAGVGGVARDSTDSPDPDRYSICYVNGFQSQPADRDAWLAEPDLVLIDDAGEPVIDENWPDELIFDLSTDDRRARIAERVGASITRCAEAGFDAVEIDNLDSYTRSDGRLTVEDAIALATRYAGLAHDAGLLIGQKNAAELGTRGRDDVGFDFAVAEECHRFDECATYTEVYGGAVLDIEYTDDLRGTFDEVCADPQVPFSTILRDRDLRTADDPAHVFDACAR

SEQ ID NO: 51 - protein, 251 aa, Plantibacter flavus
DTGPTHSAGGFPTDVVFDYQLGGGYEPTAGVGGVARDSTDSPDPDRYSICYVNGFQSQPADRDAWLAEPDLVLIDDAGEPVIDENWPDELIFDLSTDDRRARIAERVGASITRCAEAGFDAVEIDNLDSYTRSDGRLTVEDAIALATRYAGLAHDAGLLIGQKNAAELGTRGRDDVGFDFAVAEECHRFDECATYTEVYGGAVLDIEYTDDLRGTFDEVCADPQVPFSTILRDRDLRTADDPAHVFDACAR

SEQ ID NO: 52 - DNA, 852 bp, Streptomyces miharaensis
  sig_peptide 1..132
  mat_peptide 133..849
  source 1..852: mol_type = genomic DNA
  CDS 1..849
atgcccgccaagggtgtttcccgcgtcggacgtcccgggacgaccgccggcgccgtcgtgtgtgcggtcgcgttggccgtggggctgacgggatgcggatcgagcggctcgccgcagccgtcgcggagtgcctcccctcaggtgcggctcccgccgcggcacgccggtttcgactaccagatcggcggtgcctatcccccgccgaagggcgtgcgcatcgtcagccgcgaccgctcctcgtcccccgcgcccggccgctacaacatctgctacgtcaacgccttccaggcccagccggaggagcgcgccgcctggcccgccgacctgctgctgcgcgatgccgacggcaagctggtcatcgacgaggactggaacgaaccgctgctcgacctccgcaccccggccaagcgcgagcgcgtcgcccggaaggtcgaccgctggatcgacgagtgcgccaccaagggcttcgacgccgtcgagccggacaactacgacagctacacacgctcccaggacctgctcaccgccgacgacgcgaccgcgttcatcgccctgctctcccggcacgcgcacgcccggcacctggccatcggccagaagaacacacccgaactggcgagcgtcaggaagaaggccgggctggacttcgccgtggccgaggagtgcggtgagtacgacgagtgcggcacctatgccgaggcgttcgacgaccgggtggtcgtcgtcgagtactcggacaagggcctgcgcaaggccgtcgcgggcttcggcgacagactgagcatcgtgcgccgggaccggatggtgtcgacaccgggcagtgccggctacgtgcgcaagctacgcccgtga

SEQ ID NO: 53 - protein, 283 aa, Streptomyces miharaensis
MPAKGVSRVGRPGTTAGAVVCAVALAVGLTGCGSSGSPQPSRSASPQVRLPPRHAGFDYQIGGAYPPPKGVRIVSRDRSSSPAPGRYNICYVNAFQAQPEERAAWPADLLLRDADGKLVIDEDWNEPLLDLRTPAKRERVARKVDRWIDECATKGFDAVEPDNYDSYTRSQDLLTADDATAFIALLSRHAHARHLAIGQKNTPELASVRKKAGLDFAVAEECGEYDECGTYAEAFDDRVVVVEYSDKGLRKAVAGFGDRLSIVRRDRMVSTPGSAGYVRKLRP

SEQ ID NO: 54 - protein, 239 aa, Streptomyces miharaensis
SPQVRLPPRHAGFDYQIGGAYPPPKGVRIVSRDRSSSPAPGRYNICYVNAFQAQPEERAAWPADLLLRDADGKLVIDEDWNEPLLDLRTPAKRERVARKVDRWIDECATKGFDAVEPDNYDSYTRSQDLLTADDATAFIALLSRHAHARHLAIGQKNTPELASVRKKAGLDFAVAEECGEYDECGTYAEAFDDRVVVVEYSDKGLRKAVAGFGDRLSIVRRDRMVSTPGSAGYVRKLRP

SEQ ID NO: 55 - DNA, 1056 bp, Urnula craterium
  sig_peptide 1..54
  mat_peptide 55..1053
  source 1..1056: mol_type = genomic DNA
  CDS 1..217
  CDS 299..595
  CDS 677..1053
atgacatcgaaaatactctcgttagcattagcaacgctaaacgtcgcatatgcagcaacaacaatctgtagcaccaaatgcccttacacgaatctcggttcttccgatgcagtaacgaaatccgactggtggaaacctgcgcttggtgctacttggcaggttgtgttgacgcctgctgatgatgatgccgggccgcagttgacggacgattatgaaggtctcctcccctcttctctagatttaatctaatctctctcgaattcccttcagctagctaatatgcccctgaaataaatagtctacgaccttgacgtctttgacgtcgacgctagcgtcttcaaatctctacaagcaaaagacaagaaagtgatatgctacttttcggcagggtcctatgaagactggcgcgaggactgtggatgtttcaagtccagtgacttaggttccgaccttgacgggtgggaaggtgaagcttggttgaatactgatagtcagaatgtgaggaatattatggctgcgagacttgatgtggcggtggaaaaaggatgcaatggcgttgatccagataatgttgatgggtatgataatgataacggtatgccttttccctcattcttctgttcaattttattcgacgctgatagaatctctttttcttttttcttttcgggaataggtctgggtttatccgagtccacggcgaaatcgtatctgcagttcttatcagacgaagcacattcaaggggtttagctattggtctaaaaaacgctggaagtattgcggcggatgtagtggatagtatggaatgggaggtcaacgaagagtgtattgcgcaggagaactgtgaagattataagacctttgttgatgccgggaagccggttttcaacattgagtatccgccggaggatcatatcgatgatgcgtggacggaggcggagattgaggagaggtgtgcgcagaaggcggatgctagcaatggtgctaccaagttctcgacggttttgaagaaccatgagaatgtggatgaatggatcagaacttgcagtgagtag

SEQ ID NO: 56 - protein, 297 aa, Urnula craterium
MTSKILSLALATLNVAYAATTICSTKCPYTNLGSSDAVTKSDWWKPALGATWQVVLTPADDDAGPQLTDDYEVYDLDVFDVDASVFKSLQAKDKKVICYFSAGSYEDWREDCGCFKSSDLGSDLDGWEGEAWLNTDSQNVRNIMAARLDVAVEKGCNGVDPDNVDGYDNDNGLGLSESTAKSYLQFLSDEAHSRGLAIGLKNAGSIAADVVDSMEWEVNEECIAQENCEDYKTFVDAGKPVFNIEYPPEDHIDDAWTEAEIEERCAQKADASNGATKFSTVLKNHENVDEWIRTCSE

SEQ ID NO: 57 - protein, 279 aa, Urnula craterium
ATTICSTKCPYTNLGSSDAVTKSDWWKPALGATWQVVLTPADDDAGPQLTDDYEVYDLDVFDVDASVFKSLQAKDKKVICYFSAGSYEDWREDCGCFKSSDLGSDLDGWEGEAWLNTDSQNVRNIMAARLDVAVEKGCNGVDPDNVDGYDNDNGLGLSESTAKSYLQFLSDEAHSRGLAIGLKNAGSIAADVVDSMEWEVNEECIAQENCEDYKTFVDAGKPVFNIEYPPEDHIDDAWTEAEIEERCAQKADASNGATKFSTVLKNHENVDEWIRTCSE

SEQ ID NO: 58 - protein, 27 aa, Bacillus clausii
MKKPLGKIVASTALLISVAFSSSIASA

SEQ ID NO: 59 - protein, 8 aa, Synthetic construct
  REGION 1..8: note = His-tag
HHHHHHPR

SEQ ID NO: 60-61 - skipped (2 entries)

SEQ ID NO: 62 - protein, 5 aa, Synthetic construct
  VARIANT 4: note = X=L or I
DYQXG

SEQ ID NO: 63 - skipped

SEQ ID NO: 64 - protein, 5 aa, Synthetic construct
AEECG

SEQ ID NO: 65 - protein, 6 aa, Synthetic construct
  VARIANT 5: note = X=A or T
NAFQXQ

SEQ ID NO: 66 - protein, 5 aa, Synthetic construct
WQWQL

SEQ ID NO: 67 - skipped

SEQ ID NO: 68 - protein, 10 aa, Synthetic construct
  VARIANT 2: note = X=any amino acid
  VARIANT 3: note = X=any amino acid
  VARIANT 5: note = X=any amino acid
  VARIANT 6: note = X=N, H, Q, T or S
  VARIANT 10: note = X=P or G
GXXVXXIEYX

SEQ ID NO: 69 - protein, 5 aa, Synthetic construct
VICYF

SEQ ID NO: 70 - protein, 6 aa, Synthetic construct
ICYFSA

SEQ ID NO: 71 - protein, 5 aa, Synthetic construct
DFAVL

SEQ ID NO: 72 - protein, 325 aa, Chaetomium globosum
QVDVPSNFVPGVKWQIIIQNTLDPVAAVNPSDAVVWDLDLYHIARTPEIVSHLRDNNPDAILICYFNAGLAQTSDCDYDSRWNSTASNLLGNPYSPDEPQFADERWVNIKNQTARNWMKDRITLARDVGCDGVDPDNIDGYLNDEDGANGTGWNLSEGDYVSFVTELAEHAHSLTTDRGHTLLIGQKNAPELVEQVGGSLDFAVLEDCKTLNDDEDYVFCGDFQTYIADGKPVFSIEYPSTLGDTETGNCNTNGASDEQYAASCATDQGNSGFSTVLKIQGGAGELNGCTQYCSEAGPGNGVVVSPTDPDLDGRKCPSNAAPPPS

SEQ ID NO: 73 - protein, 276 aa, Stilbella fimetaria
ASNVFDGLEKGTEWNICIHKPIKHDTPDDVVPHKAAVYDIAVDHARDFPGIIPAIKESGTIVLCYFNAGALQDWDADKDAFPEAAIGKTMGGDYNDEWYLDVRSQDVVELMYRRLEEAAALGCDGVDPDNVDAWAQEGEDRTGFGLTQEDYKHYLTKLAKFAHDLGPLMVGQKNAPEMAPALADVLDFAVLESCRKWEFCEDFQVYVKAGKPVFQIEYPESIMDEGELSPEDYEMYCQGDAGDAGFSKVLKRASAQLDGWTQYCGEEPFEQAVIEG

SEQ ID NO: 74 - protein, 333 aa, Volutella ciliata
RRSCPGSGGAVKPPTTTTLLTSTVTAAAQTIETEDPEPSQGGQSGSSAFDKFKPGVKWNIVLHNPIKVDDIAALGPADTEVWDIDLGHATEFENMIPTLKKAGKFIICYFNGGAVQNWDTDKDDFPESIIGKSLGDEYEGEEFYVDVRSDEVVKIMKARLDLAAKVGCDAVDPDNVDAFYDNATGFNLDEADYVTYLTKLADYAHSIKTDSGNPLLVGQKNAPEIAPQLVGVLDFAVLEQCRGSSNPDEESWPFCPDFQPYIAAGKPVLQIEYPPSVEESDGTLSAADKEFYCGAKADDKGFSKVLKWSSPQLDGWTQFCDSDTSFTVPEDDY

SEQ ID NO: 75 - protein, 290 aa, Clonostachys epichloe
GPAALPANYLSTFPLGTKWQIVLHQPIQHNGPNDFIPYDGKIWDIDLGHAESYPNMIPRLRSAKKAVICYINAGAIQSWDIDINSFPAEARGKNMGDEYTDEWWIDVRNPKVIAFMKKRLERATAAGCDGVDLDNIDAWATPEGDRTGFNLTQADYVSYMTQLGNYAHTLPTKNSGGGLAMGQKNAPELVDRLVKVLDFAVLESCLLDNFCADFQPYVKAGKHVFQIEYPESFSRSRTLSTRDNSFYCGTHSTSKTDDNFSMVLKYASWQLDGFGQFCGQKSYTTPTIRE

SEQ ID NO: 76 - protein, 286 aa, Myrothecium sp.
QAGPKVPADFKPNVAWNIILHNPIVHDSEADLIPAEAKVWDIDLGHARDYPIIPLLKSADKLVVCYFNGGAVQAWDADRDAFPEDVIGRSLAYPFEDEEWYVDIRDSRVVDIMKSRFDLAVELGCDAVDPDNVDAWAWSTDPEEDPTGFNLQPEDYAAYLNALADYAHTLTTQSGNNLLVGQKNAPDLIAAVVDNLDFAVLESCIRDDFCGQYQPYIAQGKPVLQIEYPPSVEETGSLSSEDNERFCLNAEGDDNFSKVLKFSTAQVDGWGQYCGGESWETPTFEG

SEQ ID NO: 77 - protein, 321 aa, Botryotrichum piluliferum
QVDVPPNFAIGAKFQIVIQNTLDFEAPLAPTDAVVWDLDLYHIARHSEIVGHLRESNPDVILMCYFNAGLAQTSDCDFESRWNNSGFLGNAYDSDEFSDEFWINIKEQEARDLVKERITLARDIGCDAVDPDNIDGWWNDVDGLNGTGWNLSEADFVNFATELADHAHSLTTEKGHTMLIGQKNAPDIVEQLVGVFDFAVLEDCKTLNDDEDEDWTFCQDFQAYIAQGKPVFSIEYPSTLGDTQTGACNPGGVSPEQYAASCDNTAGNSGFSTVLKIQGDVGELNGCTQYCVEEGEDMEVVTTATDPNKDGETCPDNAVGP

SEQ ID NO: 78 - protein, 288 aa, Myrothecium sp.
QAQPKVPADFKPNVDWNIILHNPMVYQNAEPLVPAEAKVWDIDLGHALEFPEIIPLLKRNNKLVICYFNGGALQDWDDDKEDFPEAAIGKPLAYPFDDEEWYLDIRNADVVAAMKARLSTAVDAGCDAVDPDNVDAWAWGNSDDEDPTGFRLTTDDYAAYLNTLADHAHTLTTQAGNNLLVGQKNAPDLVPAVVQSLDFAVLESCRRDDFCGDFQPYIAGGKPVFQIEYPPSVEETGQLSNADNQFFCLREDDSDDDFSKILKWSTAQVDGWGQYCGGSAWETPTFEG

SEQ ID NO: 79 - protein, 236 aa, Amycolatopsis sp.
AVTLPPTPSAWDYQIGGAYAPPSGVKIVSRDNSDAAASGLYNICYINAFQVNVGDDGQWASDLLLRDANGNKVVDPDWHETLLDLRTADKRSRVAAKINGIVDSCAGKGFQAIEPDNYDSYERSKNLISTANSQDYIKLLSSHAHAKGLAIAQKNTVDLAANRVANGLDFAVAEECGQYTECDVYANAFGNRVVDVEYTSSGLSKACASWKGKISIVRRDEDVVPAGESGYVRKTC

SEQ ID NO: 80 - protein, 564 aa, Methylothermaceae bacteria
EVKDLQAVRWGSEIVFRAQFSPQHSHKQVFIDADGDTSTGYPVGGIGADYLIEDDWFYRYQGRGWRWDFIAEVPMSENNGQSVWRVPAKLIFPSGPLSIIFSGIDDWDSPELSSVIRSGEAVLVSHALTPYQMRTRRGSESDQGLDALATKDQSGTQDKWDDYIEFYPGDKGYAGQFYLRLPKEVKARDIIHLKVVTNYRGERKKYQRWIWKIYDFKESRWQIIGDNGRAPNWHWKSFTFPVNGTPSHYFASNGLAKLRYETSKDVDNSQLDLLKLVVDSISSDTQEDSPQPDPEEGSTPSQPENPGSEGNDSEPPQPPGLPGESSRWIPRPGMSWQWQLSGKIETSVDADIFDVDLFDTPRETIDLLHAKGRKVVCYFSAGTYENWRPDKSKFPDDVIGRRLEDWEGESWLDIRQIDVLRPILAARMDLAKEKHCDGVEPDNVDGYDNKTGFPLKAEDQIRFNRMLAEEAHKRGLPIALKNALGLIEALEGHFDWALNEQCFQYDECEMLQPFLDAGKAVLGVSYVSKGKAGKICRKANQMGFSWLIKRWDLDAWRMDCRNYD

SEQ ID NO: 81 - protein, 252 aa, Amycolatopsis niigatensis
AGPVESAPLPSPHSQVADVPLPAPHAGFDYQIGGPYQPPAGVQVVSRDHSAPAASGLYNICYVNAFQVQPGAEKEWGDLVLRDDDGTVVMDPDWNEALLDSRTADKRSRIADKVGAWIDECAGKGYQAIEPDNYDSFTRSQGLLSARNAQDLVKLLSERAHGKGLAIGQKNTSELASSRAANGLDFAVAEECGQQDNCAEYTRYFGDRVIVIEYTEDGLRTACAKWGNTLSVVRRDRDVTPKGDSAYVRESC

SEQ ID NO: 82 - protein, 249 aa, Streptomyces sp.
AGTAESAPLPAPQSQVALPAPHAGFDYQIGGPYQPTADVQVVSRDHSVPAAAGVYNICYVNAFQAQPGAEGEWGDLVLRDGNGDVVKDPDWNEALLDLRTEDKRNRVAAKVGGWIDDCAGKGYQAVEPDNYDSFTRSHGLLSARNAQDLAKLLSSRAHGKGLAIGQKNTSELSSSRADNGLDFAVAEECGEQDNCAEYTKYFGNHVIVIEYTGDGLRNACAKWGSTLSIVQRDRDVKPKGDPAYVRETC

SEQ ID NO: 83 - protein, 240 aa, Amycolatopsis alba
APAPAAFSPPPVKAGFDYQIGGAYAPPSGVRVVSRDHTAQPAAGLYNICYINAFQAQPGAEGEWGDLVLRDANGEVVMDEDWDEALLDLRTADKRQRVAAKVNAWTDGCAAKGYQAIEPDNYDSFTRSRGLLSEQDAQAYIRLLSTHAHEKGLATGQKNTSELAGNRKANGLDFAVAEECGEQDICGEFTAAFGGNVIVIEYADAGLANACDRWGGSLSIVRRDRDVVPAGASGYVRKTC

SEQ ID NO: 84 - protein, 245 aa, Streptomyces griseoaurantiacus
AETVVLPPVHASFDYQIGGAYEPDKGVQVVTRDVKESPAKDLYNICYVNAFQTQQEGDVSGPQDWDKDLLLTGKNGEPVIDPDWDEVILDISEDGKRERIAQQIDKQIDQCDTKGFDAVELDNYDTYDRDVVDGALTAQDAQAYIRLLSAHAHAKGLAVGQKNTVELAGNHKANGLDFAVAEECGNPKWKECGKYVSAFGDHAIFIEYTEEGLRNACAYGDRVSVVMRDMDVSEPDSEDYVRETC

SEQ ID NO: 85 - protein, 244 aa, Kutzneria albida
ATGTAPPHATVTAPPVTAGFDYQIGGAYTPPSQVRVVSRDHTAQPAAGLYNICYVNAFQAQPGAEGEWGDLLLRDANGNVVMDEDWGEGLLDLRTADKRQRVAAKVNVWVDDCATKGFQAIEPDNYDSFTRSKGLLSDSDAQAYIRVLSAHAHKKGLAVGQKNTSELAGQRQANGLDFAVAEECGDQNKCGEYTPAFGDHVIVIEYTDAGLRTACDRWGGSLSIVRRDHGVAPAGASGYVRKTC

SEQ ID NO: 86 - protein, 244 aa, Microbacterium sp.
TESAGGITLPPSDGLFDYQLGGGYEPGDDVSVVTRDSTDEPADARYSICYVNGFQTQPGAEWPATLILHDSEGAPLADPGWPDEYLLDISTPEKRTEAAAQQAATIADCADRGFQAVEFDNLDSWTRSEGALTEADAVAFAMMLVGAAHHQGMAASQKNSAEMTAAGKNDIGFDFVTVEECDLFDECDEFTSAYGDQVFDIEYTDDLRGTFADVCARDATPARTILRDRDLVTPDNPAYVYEYC

SEQ ID NO: 87 - protein, 249 aa, Microbacterium oleivorans
QSPPPHDPPAGPQLPPAGGHLDYQLGGAFDPPSGVDIVVRDRAAAPVDELYSVCYVNGFQTQPGELDAWPDELLLRDDEGEPVTDPDWPDEVILDTRKADEIAKIVGPWIRDCASDGFDAVEFDNLDTYTRTDGALSRDDALNLANLLVDTAHDLGLAAGQKNAAEDASLLHSRAGFDFAVVEECAAYEECSAYTEVYGDHVLAIEYTDNLPRPWDEVCADPETPESVVLRDRDLVTSDDPAYVFETCG

SEQ ID NO: 88 - protein, 246 aa, Nocardiopsis alba
VELPPEGGFDYQLGGAYPPPEGVTTVVRDSTAEPAPGLYSVCYVNGFQTQPDELDRWLEEDPDLVLRDEDGEPVVDPGWPDERILDTSTGERRERIAEILTESIDRCADAGFDAVEFDNLDSYLRSGEALTVDDNLALASVLVGVAHGRGLAAAQKNTAEEAGRGHDEVGFDLAVTESCAAWHECGAYTGVYGSGNVLAVEYPEELAEAALDFDVVCADPERPDRVLLRDAALVPSSDPEHLFEAC

SEQ ID NO: 89 - protein, 258 aa, Microbacterium oxydans
ETAPHSAHSTDAPAGDDWALPPAGAVPDYQLGGAYEPAAEVGIVGRDRSADPASDTYSICYVNGFQTQPGELDTWDADLLLQHDGDTVFDPDWPDEALLDTSTADRRERIAAQIIPWIEGCADDGFAAVEFDNLDSYTRSGDALSLDDNLALATLFVDAAHGAGLAAGQKNAAEDAVQLHAQAGFDFAVTEECAVFEECDVYATVYGDHVIDIEYSDELPRSFADMCADPDSPASMVLRDRDLVTPDDPAYVFETCGE

SEQ ID NO: 90 - protein, 268 aa, Agreia pratensis
ADSREDDQASPTPTGSTYLIATSDTVTLPPFGEPADYQLGGSYDVDADVKIVTRDSTSEPAEGVYSICYVNGFQSQPGDDERWVVDNPDLVLRDDGGEAIIDPNWPDEFILDTSTPEKRQRISRMNGASIEACADRGFDAVEIDNLDTYSRSEGRLSIDDNLALAKLFADRAHGRAMAIGQKNSAELGDRGRSVANFDFAVTEECFRFEECGAYADVYGSAVIDIEYTDDLPVPFDEVCASDDRPATTILRDRDLVAKGEPDYVFEHC

SEQ ID NO: 91 - protein, 249 aa, Microbacterium lemovicicum
AAPSRDAGFPASLVFDYQLGGGYAPAAGVGGVVRDSTDVAEPGLYSICYVNGFQSQPADRDSWLAEPDLVLIGDDGQPLIDDNWPDELIFDISADDQRERIAARVGERIAVCGRNGFSAVEIDNLDSYTRSDGRLTEDDAIALATLYAQTAHDAGLLIGQKNAAEIGPRGRDEAGFDFAVAEECVAFEECSAYRDVYGDAVVDIEYTDNLPSGFDAACAVDDRPHSTILRDRDLVPADDADHVFQACPA

SEQ ID NO: 92 - protein, 249 aa, Xylanibacterium sp.
APGPVSLPPTDGTFDYQLGGTYTPPGGLDVVVRDATAEPAEGAYNICYVNGFQTQPGDDRWLDHEDLLLHDGEGQVAVDPDWPDEYVLDPSTARQREQILALVGPAVTGCADAGFDAVELDNLDTWTRFDDPATGAIDPAGALALAAAYVELAHSAGLAVGQKNAAEIAATARDDLGFDFAIAEECLAYDECGAYTAAYGEHVLVVEYPDTLGTPFEEACAGSGALPLTILRDRVLAAPGDAGYVRRQC

SEQ ID NO: 93 - protein, 255 aa, Curtobacterium oceanosedimentum
SPHAASSSSSGTSTNLPTAGIPDYQLGGGYTPASGVTIVERDSTDRPAPGKYGICYVNGFQTQPEDADTWLDRHPDAVLRDDDGDPVSDPGWPDEMLLDTSTAAKRAEIVDVLTTSIERCADRGFDAVEFDNLDSWTRSDGALTRAGNLALAASLVRVGHEHGLAVGQKNTPQLGASGRKTTGFDFVVAEECVHYQECAAYTKAYGAHVIDVEYSDDVGRSWSSVCADEDRPAMTILRDRDLVTPSQEGYVFEHC

SEQ ID NO: 94 - protein, 250 aa, Leucobacter tardus
AEQPADPAADGAHAFPAGAEFDYQLGAAYALPAGVTMVTRDRTAEPEPGAYSICYLNGFQTQPGEIDDWPEDAILRIDGEVRTDPDWPDEALLDIATKSGREIVIDRVSTWIDGCRDAGFQAVEFDNLDTFTRSDGAIGREDAAAVATALVAAAHDAGLAAGQKNAAEHAVWLRAEAGFDFAVSEECAAFDECGTYTEVYGDAVMDIEYTDALPRPFDEVCGDAATPQATILRDRALVAPDDPDYVFEVC

SEQ ID NO: 95 - protein, 254 aa, Salinibacterium amurskyense
APPTTEPSAPSTATASPSLFAAGAVADYQLGGAYEPDASVTVVVRDAGQEPLAGAYSICYLNGFQSQPGESWPSELLLRDAEGDPVIDANWPDETLLDIRTASNRAAIAERLAPLIEDCAGKGFVAVEFDNLDSYTRSEGTLEREQAVAMATLFVQLGHSSGLAVAQKNSAELLGEAASAIGFDFAIVEECDQYSECGDFTDFYGDAVIDVEYTDELRRPFTEVCADAATPPITMLRDRDLTPPGSAEYTFEHC

SEQ ID NO: 96 - protein, 244 aa, Synthetic construct
GEPTPVALPPAGGAPDYQLGGAYDPDPRVEIVARDRSDEPAPGLYSICYLNGFQTQPGERDDWPDDLVLRVDGADVIDPDWPDEALLDTRGAQNRAAIAERITGFIEGCAAAGFDAVEFDNLDTYTRSSGMLDAGDNLALAAVLVDAAHAVGLAAAQKNAAEDAPRLRVEAGFDFAVVEECAVFDECGAYTDVYGEHVIAIEYTDAMPRPFADLCADDALPASLVLRDRDLTTPGDPAYAFTLC

SEQ ID NO: 97 - protein, 363 aa, Neonectria candida
APVSARRRGACKPAALYSASSSASFLSTSSPLSETSKGITTVVTAAPTPGQGNGTVTTTTQAVATNTGISTSVAAVSNFKPGVTWDICIHYPIKHDSVDDLIPAEATVWDIDLTHAHEFPNMIPMLKKAGKFVICYFNAGARQTFDDDKDLFPTEVIGKSLSYPYDDEEAYVDIRDDRVLDIMKARLDSAVEVGCDAVDPDNVDAWAQDGTDPTGFSLKSSDYVTYLKNLAAYAHSVKTQEGQPLLVGQKNAPEIAADLVSTLDFAVLEQCRGSSDSSEESYAFCSDFQTYIDAGKPVLQIEYPPSVSESDGTLSSSDESFYCNAEDDDKGFSKVLKWASAQLDGWGQYCGGKSFRTVEAEYE

SEQ ID NO: 98 - protein, 264 aa, Fusarium compactum
FNPRAINTPKRKDLWKPEVGSKWEIILSQVFKLPKGGASKLDPSVPIYDLDLFENSKSTFTAMQKTGKHVICYFSAGSWENWRDDRKDFPAKDLGKVMDGWPDERWVNIRSPEVRAIMAKRIKIAADKGCDAIDPDNMDGYQNDNGLGLTEDDTVSYVKFLSKEAAKYNMVMGMKNGGDVTEQVLPYVAFCINESCIQYSECDLYAPYIEAGKPVFNIEYPKGAPKVKASNKKKICGTTGAAADSDGFSKIIKKMNLDKWTMYC

SEQ ID NO: 99 - protein, 314 aa, Fusarium compactum
RCKGECLVDARAMTAKAKQTTPSASPFELSDFKPGVEWEIVLHQPIKHESAADLIPTTAKVWDIDMGHARDYPEMIPLLKSAGKFIVCYFNAGAVQSWDEDKDQFPEAAVGHSLAYPYDSEEWYLDIRDPTVLRVQRARLDVAAKVGCDAVDPDNVDAWQQNEEDPTGFKLKPSDYATYLKDLAEYTHSIKTKDGNPLLIGQKNAPEIAEDLVSTLDFAVLESCRGTTNPEEESWPFCEDFQIYVDAGKPVLQIEYPPSVEKTGKLSSSDKTFYCTPKDEDQGFSKIIKWASAQLDGWGQYCGGEPFRTPVIKE

SEQ ID NO: 100 - protein, 301 aa, Fusarium avenaceum
EPKPAATASSPSSFGLSDFKPGVQWEIVIHEPIKHDSAANLIPLKAKVWDVDMRHARRYPEMIPLLKSAGKFVICYLNAGAVQAWDDDKDKFPKAVIGYSLAYPYDSEEWYLDIRDSTVLKLQKARLDVAAKIGCDAVDPDNVDAWQQDEEDPTGFNLKSSDYTKYLKSLAQYAHSIKTKDGNPLLVGQKNAPEIADDLVSSLDFAVLESCRGTTDPEEENWPFCEDFQTYIDAGKPVLQIEYPPSVEKTGKLSSSDKTYYCTPKEEDKGFSKIIKWASAQLDGWGQYCGEEPFRTPVIKE

SEQ ID NO: 101 - protein, 377 aa, Preussia aemulans
TFTKGQKWQIILLGTPDVSKMPLPPTDAPVWDIDLFDSAASDIRTLKDAGKIVICYFSAGTREDWRDDAKDFPAGDQGKVLPEWPNEKWIRVGSTKIREIMAKRIKLAADKGCDAIDPDNTDGYQNDNGLNLKNTDAIDYMRWMQKTAEGYGMKIGLKNSIDILDTISSIMDFAVNEQCAQHSECTAYDKFLASGKPVFHIEYPTPLNQNQADGIYCKGPGTNGMSTVLKEMALTGVTIYCDKSQVNTPTRSGNSPPRPSVPPRSSTTRPPISSTSRSSSTLTPSSIATPTTTSIPDTTLISSTTPRPSSTPRPSTTPRPSSSSRTSSSQRPTSSAPGGCRSKHWDQCGGQDWKGCTVCESPYTCKAVSAPWYYQCL

SEQ ID NO: 102 - protein, 298 aa, Fusarium proliferatum
KPAAAASSAFFELSDFKPGVEWEIVLHQPIKHDTVADLIPSKAKVWDIDMGHGRDYPEMIPLLKGAGKFVICYFNAGALQSWDKDIKQFPDAVIGHSLAYPYDSEEWYLDIRDSTVLKLQKARLDVAAKIGCDAVDPDNVDAWQQDGEDPTGFKLKPSDYTKYLKNLAEYAHSIKTKDGNRLLVGQKNAPEVAEDLVSTLDFAVLESCRGTTDPDEENWPFCEDFQTYIDAGKPVLQIEYPPSVEKTGKLSSSDNAYYCTPKGEDKGFSKILKWASAQLDGWGQFCGEDPFHTPVINE

SEQ ID NO: 103 - protein, 299 aa, Fusarium lateritium
EPAATASSPSSFGLSDFKPGVQWEIVIHEPIKHDGTADLIPSKAKVWDIDMGHARDYPKMIPLLKSAGKFVICYFNAGAVQAWDEDKNQFPKAVIGHSLAYPYDSEEWYLDIRDSTVLKLQKARLDVAAKIGCDAVDPDNVDAWQQDEEDPTGFKLKSSDYTKYLKSLAEYAHSIKTKDGNPLLVGQKNAPEIAEDLVSSLDFAVLESCRGTTDPKEENWPFCEDFQTYIDAGKPVLQIEYPPSVEKTGKLSSSDKTYYCTSKEEDKGFSKIIKWASAQLDGWGQYCGEEPFRTPVIKE

SEQ ID NO: 104 - protein, 264 aa, Fusarium proliferatum
FTPHGFTTPKRKELWQPEVGTPWQIILSEVIKVPKAGVSSMTPDVPIWDMDLFENSKSTITAMQKGGKKVICYFSAGSWENWRKDKDSFPKKDLGKVMDGWPDERWVNISSVAVRAIMAQRIKLAAEKGCDAIDPDNMDGYQNDNGLGLTEEDTISYVKFLSAEAAKYNMVMGMKNGGDVAEEVLPYVAFCINESCIQYSECDLYQPYIDAGKPVFNIEYPKGAPKVKAKDKKKICSTSGAAEGSDDFSKVIKKVNLDKWVLYC

SEQ ID NO: 105 - protein, 320 aa, Chaetomium ancistrocladum
QREVPPNFEIGAKFQIVIQHTLDWNAPLAPTDAVVWDLDLYHVARHPEIVEHLRASNPDVILMCYFNAGLAQTSDCDYESRWNNSGFLGNSWGDEFPDEFWINIKEQEARDLVKERITLARDKGCDAVDPDNIDGWWNDADGENGTGWNLSEADYVNFVTELAEHAHGLTTLQGYTMLIGQKNAPDIVDQVHENLDFAVLEDCKTLNDDEDGDWTFCQQFQTYIADGKPVFSIEYPSTLGDTGTGACTPGGVSPEQFEASCDDTAGNSGFSTVLKIQGDVGELNGCTQYCAEQIDDSEVVETATDPLKDGDVCPTDAEGF

SEQ ID NO: 106 - protein, 298 aa, Fusarium verticillioides
KPAAAASSALFELSDFKPGVEWEIVLHQPIKHDTIADLIPSKAKVWDIDMGHGRDYPEMIPLLKGAGKFVICYFNAGALQSWDKDIKQFPKAVIGHSLAYPYDSEEWYLDIRDSTVLKLQKARLDVAAKIGCDAVDPDNVDAWQQDGEDPTGFKLKPSDYTKYLKNLAEYAHSIKTKDGNRLLVGQKNSPEIAEDLVSTLDFAVLESCRGTTDPDEENWPFCEDFQTYIDAGKPVLQIEYPPSVEKTGKLSSSDNAYYCTPKGEDKGFSKILKWASAQLDGWGQFCGDDPFRTPVINE

SEQ ID NO: 107 - protein, 264 aa, Fusarium lateritium
IATRGVSLPKRKELWKPEVGTPWQIILSEVITLPEGGADDLSPDVTIWDMDLFENTKETISAMQESGKRVICYFSAGSWENWRKDKNSFPKKDLGKTLSGWPDEKWVNISSAAVRAIMAKRIKQAADKGCDAIDPDNMDGYQNDNGLGLTQKDTISYVKFLSAEAAKYNMVMGMKNGGDVTKQVLSYVAFCINESCIQYQECDLYTPYIKAGKPVFNIEYPKGAPKVKDSDKKKICATAGDAKGSKGFSKVIKKMNLDSWVMYC

SEQ ID NO: 108 - protein, 317 aa, Chaetomium sp.
QVNPPPNFRPGQDLKWQIAIYSTLDTTVPLQPADADVWDLDLYHVARHPEIVDFLRTNNPNTTLICYFNAGLAQESDCDYQTRWVNSDLLGNPYSPDAPQFSDEFWINIKSQTARDLVKRRITLARDIGCDAVDPDNIDGYLHDEERGDEGTGWNLSQDDYVSFVTELAEHAHSLTTQRGYTMLMGQKNAPEIVGEVSDVLDFAVLEDCRTLNDDEDYTFCPDYQVYIAEGKPVFNIEYPTTLGDEESCIGASAEQYQAACAVTEDNSGFSTVLKNQGDGELNGCTQYCVEGDPSDGVVVTALNSELDGDQCPPEAE

SEQ ID NO: 109 - protein, 331 aa, Scytalidium sp.
QVTVPSSFTVGAKFQIVIHNTIDPDSPLEPADALVWDIDLFHLARTPEVANHIRSTNPSATILCYFNAGLAQTSDCDYPRWDSSGLLGNPYDITDPDLFPDERWINIKNATAREWIKWRISLARDLGCDGVDPDNIDGFLNDYDGDNGTGWSLSEDDYVAFVTELAEHAHGLTTLQGNTMLIGQKNAPELIGRVKDVLDFVVLEDCRTINDEEDYEFCTDFKEAYIDEGKPVFSIEYPSTIGENGECWATGASEDEYHVACEEGAAGFSTVLKIQGEGELNGCTQYCDGGEPGTGVVVTPTDPERDGDPCPNDAIVVGWAGNWPLALEEMR

SEQ ID NO: 110 - protein, 281 aa, Acremonium sp.
KSLDDVNLGAPWQIAIHDPIIHDSADDLVPSQAQVYDIDLGHAQTFPDMIPTLKEAGMVVLCYFNAGAVQDWDNDHDLFPEEVRGTTLGGDYPDEWYLDIRSPIVVDLMKRRLDSAAAVGCAGVDPDNVDGWVQSNGDDRTGFGLTQDDYATYLEELAAYAHTVPVADGDGTLLVGQKNAPALAERLVSSLDFAVLESCREWDFCGDFQVYPSAGKHVFQIEYPDSILEQGQLSAADNEFHCEGEGHGDEGFSKILKRASAQLDGWGQYCGGASFEQELGW

SEQ ID NO: 111 - protein, 321 aa, Chaetomium strumarium
QVDVPPNFTPGVPWQIIIQNTLDVTKPLQPNNVTVWDLDLYHIARNPDIVGYLRTNNPGTILICYFNAGLAQESDCDYDTKWRNSGLLGNVYDPQDPEFSDERWVNIRNQTARDWMKQRITLANQLGCDGVDPDNIDGYLNDQDGNNGTGWNLAQDDYVSFVTDLATHAHSLTTSQRHTLLIGQKNARELVGPLGGVLDFAVLEDCKQLNEAEEPAPFCADFQPYVAAGKPVFSIEYPSTLGDPETGACNAGGADETQYNDSCNPTPDTAGFSTVLKIRGEVGELNNCTQYCTGMAPGTGVVVTATDPELDDIECPPEAAD

SEQ ID NO: 112 - protein, 298 aa, Fusarium oxysporum
KPAAAASSTLFELSDFKPGVEWEIVLHQPIKHDTVADLIPSKAKVWDIDMGHGRDYPEMIPLLKGAGKFVICYFNAGALQSWDKDIKQFPKAVIGHSLAYPYDSEEWYLDIRDSTVLKLQKARLDVAAKIGCDAVDPDNVDAWQQDGEDPTGFKLKPSDYTKYLKNLAEYAHSIKTKDGNRLLVGQKNAPEISEDLVSTLDFAVLESCRGTTDPDEENWPFCEDFQTYIDAGKPVLQIEYPPSVEKTGKLSSSDNTYYCTPKDEDKGFSKILKWASAQLDGWGQFCGEDPFRTPVINE

SEQ ID NO: 113 - protein, 298 aa, Fusarium temperatum
KPAAAVSSALFELSDFKPGVEWEIVLHQPIKHDTVADLIPSKAKVWDIDMGHGRDYPEMIPLLKGAGKFVICYFNAGALQSWDQDIKQFPKAVIGHSLAYPYDSEEWYLDIRDSTVLELQKARLDVAAKIGCDAVDPDNVDAWQQDGEDPTGFELKPSDYTKYLKNLADYAHSIKTMDGNRLLVGQKNAPEIAEALVSALDFAVLESCRGTTDPDEENWPFCEDFQTYVDAGKPVLQIEYPPSVEKTGKLSPSDNTYYCTPKGEDKGFSKILKWASAQLDGWGQFCGDDPFRTPVINE

SEQ ID NO: 114 - protein, 250 aa, Thermoascus taitungiacus
TTAKWQPPVGTTWQIELLYPLNDTSVDADVYDIDLFNNDASTIKKLQDQGRRVICYFSAGSYENWRPDKDKFKDSDLGNTMKGWEDERWLDLTSTNVRNIMLSRLDLAQEKGCDGVDPDNVDGYEKDNDSGLDLTMEDAADFVNFLANETHARNMSIGLKNAGDIIPSVIGNMQWSVNEQCAHYDECDTYAAFIEAGKPVFHIEYPKGDDTNDNSPVTTAQKVSACVFSGPRNFSTIIKNMDLDNWIETC

SEQ ID NO: 115 - protein, 263 aa, Plectosphaerella alismatis
TSDKNRWIPAVGTSWQIVLSAPLQIDSTKPVVTPDVQVYDFDLFDNTPETIAALHKIDKKAICYFSAGTYEDWRPDASRFQSGDFGSPMADWPGEKWLNLKSSNVRAIMADRIKLAADKGCDAVDPDNVDAYSNENGLGLTKDDSADFVKFLTTEAHNRGMSIGLKNAGEIIAQVINDVDFQVNEECVKYGEGRTFEAFIKAGKPVFHIEYPSGAPGAVSPATVSDIATAQGTDKFSTLIKTLSLDGWVQFPDGHTEDTQTKY

SEQ ID NO: 116 - protein, 328 aa, Thermothelomyces hinnuleus
QVRVPPNFEVGVKWQIVIQSTIDVNTPLEPTDAVVWDLDLYHVARTPEIVSYLRENNPNTILICYFNAGLAQKSDCDYETRWEKSGLLGNVYDPEEPQFDDERWVNIKNQTARDWIKQRITLARDVGCDGVDPDNIDGYHNDEDGNNGTGWDLSRDDYVSFVRELAEHAHGLTTKRNYTLLIGQKNAPDLVEDVGDLLDFAVLEDCKSLNGGGGDDDDAPFCSEFQHYIERGRPVFSIEYPSTLGDAETGECKSGGASKAQYEASCDASTASGNLDFSTVLKIQGGVGELNGCTQYCDGLQPGTGIVVTATDSELDGNECPPEATGSS

SEQ ID NO: 117 - protein, 313 aa, Ovatospora brasiliensis
QIKFPPEWKVGVKWQIEIQHPVDGKAPLVPADAVVWDVDLYHLASNPGLIDHLRTGNGPNTTVICYFNAGLVQESDCDWDTVWNTPAYRGLLGQRHPDFPDEAWINIRNQTARDLIKRRIDLAREIGCDGVDPDNIDGYGLDEPGSDHMTGWNLTQQDDIDFILDLATYAHRQTTLRGNTLLIGQKNAPEITRNVSASLDFAVLEDCRGRRTEPPDTPNPFCTVYQDPYIRAGKPVFSIEYPASLGDPESGECSASGTNDDDYTGSCQNGVEGFSEVLKIQGGEGELNGCTEYCDVKGLVVTAVDEADGGECP

SEQ ID NO: 118 - protein, 301 aa, Fusarium acuminatum
EPKPAAKASSPSSFGLSDFKPGVQWEIVIHEPIKHDSAADLIPSKAKVWDIDMGHARRYPEIVPLLKSAGKFVICYFNAGAVQAWDDDKDKFPKAVIGHSLAYPYDSEEWYLDIRDSTVLKLQKARLDVASKIGCDAVDPDNVDAWQQDEEDPTGFKLKSSDYTKYLKSLAQYAHSIKTKDGNPLLVGQKNAPEIADDLVSSLDFAVLESCRGTTDPEEENWPFCEDFQTYIDAGKPVLQIEYPPSVEKTGKLSSSDKTYYCTPKEEDKGFSKIIKWASAQLDGWGQYCGEEPFRTPVIKE

SEQ ID NO: 119 - protein, 296 aa, Fusarium euwallaceae
KPTTTVRATGLADFKPGVQWEICIHHPIKHDSAADLIPTKAQVWDIDMGHAREFPNMIPMLKSAGKFVICYFNAGALQDWDDDKSKFPQEVIGHSLSYPYDDEEWYLDIRDSRVLELQKARLDVAAQIGCDAVDPDNVDAWQQDDEDPTGFKLKSSDYTNYLKNLAKYAHSIKTKDGQPLLVGQKNAPEIAEDLVSTLDFAVLESCRGNSDPNEESWPFCEDFQTYIDAGKPVLQIEYPPSVEKTGKLSASDEKYYCTAEDEDKGFSKIIKWASAQLDGWGQYCGEDPFRTPAAKY

SEQ ID NO: 120 - protein, 274 aa, Fusarium neocosmosporiellum
APSNANTETPEALSPRAVISRKELWKPKVGTAWQIVLSQVIKIPKGGAKNLKPNVPIYDLDLFENSKETFDALHKAGKHVICYFSAGSWENWRDDRKSFQKKDLGKTLSGWPDEKYININSPSVRAIMAKRIKLAAEKGCDAIDPDNLDGYQADNGLGLTEADTIAYVKFLSKEAAKYRMTTGMKNGGSITKQVLPHVGFCINESCIQYSECDLYAPYIKAGKPVFNIEYPAGAPNVKAADKKRICSVTGAAKGSNGFSKVIKKMNLDSWVTYC

SEQ ID NO: 121 - protein, 327 aa, Thielavia antarctica
QVKVNPPAGFDVGVKWQIEIQNTVDITKPLNPADALVWDVDLYHVARNPGIIDYLRSKNPDTIIICYFNAGLAQLSDCDYDSTWKNSGFLGNSYGEEFPDEFWINIKSQAARDLVKRRITLANQLGCDGVDPDNIDGYNMDEDGSDKSSHTGWSLTPQDDITFILDLATHAHALTTARNKTLLIGQKNAPEITAAIAAHLDFAVLEDCKGLTQAASDDDDDDPFSFCGDYTTPYIGAGKPVLSIEYPRSLAGRNTGKCNAGGVSADEYARVCAEAEAEGGGGFSTVLKTRGSAGELNGCTQYCGLGVGKGVVVTAVDEELDGDDCPA

SEQ ID NO: 122 - protein, 265 aa, Acremonium thermophilum
SPKWQPSVNATWQIVLDHPLALADGADATVQPSDADVFDIDLFMHQNATTVAALHRLGKRVVCYFSAGSYEPDRPDSGRFARGDLGAELDGWPGEYWLNVSSPTVRTIMARRIGIAADMGCDAVDPDNVDGYQNTNGLGLTRAAAVDFVRFLAAESAERGLAVGLKNAGDLVDDVLDVVHFSVNEQCAQYDECATFAPFVRAGKPVFHIEYPDGAQKGSVADAAREAACDASGSSGFSTVLKRMELDGWVEYCDGTTATTAVEDE

SEQ ID NO: 123 - protein, 323 aa, Crassicarpon hotsonii
QVRAPPDFKPGVKWQIVIQSTIDINAPLEPTDAVVWDLDLYHVARTPGIVNYLRRNNPDAILICYFNAGLAQKSDCDYKTRWESSGLLGNVYDPEEPQFDDERWVNIKNQTARDWIKERITLARDVGCDGVDPDNIDGWYNDEDGNNGTGWDLSQDDYVSFVRELAEHAHGLTTERGYTLLIGQKNAPDLVEDVGDILDFAVLEDCGQLNSEDDSFCGEFQHYIERGRPVFSIEYPSTLGDPETGECRNGGVSKPQYEASCDTSAAKGNANFSTVLKIQGGVGELNGCTQYCDGHQPGTEIFVTATDPELDGNKCPPEATGSD

SEQ ID NO: 124 - protein, 260 aa, Helicosporium sp.
AFTVGERFQIILSSVPDISVTSMVPDAPVFDIDAFDSNATVISALKKMGKTVICYFSAGTYEPWRPDSNLFKKEDYGLALDGWPDEHWLKLDSTNVRSIMTTRIKMAADKGCDAIDPDNTDGFTNQNGIGLTAKGAVDFITFLHKEATANNMKIGLKNSQGILSQVSSMMSFAVNEECAINGECNGYDAFLSAGKPVYHIEYPSIIPTVAVNDRNKYCVSTASNTNKFSTVLKNMTLDGWVLYCDGSSFITKTTENATSG

SEQ ID NO: 125 - protein, 245 aa, Pyrenochaetopsis sp.
SPLHRRAVAKFTVGAVWDILLDKTNVNLAAMEKAAGTVIDIDLFDNTADGQTTVKDLAKTKQVICYFSAGSREDWRDDADQFTSADYGQALGDWPGENWVDVKSTNVRAIMKARIEAAAKAGCTAVDPDNVDGFDSNQDGYGYPQSAYSDYVNYLAGIAHANNLAIGLKNALDIIPAVLPNIQFAVNEQCHVYNECTKYKAVTAAGLPVFNIEYGNNYCTDPSGVNLSTVIKPADQDLDTLGGQC

SEQ ID NO: 126 - protein, 298 aa, Acremonium fusifioides
ALPGSCNEARAPAGFEPGVDWQICIHQPIKHDSADDFIPKAAQVFDVDLAHAQDYPEMIPNLHNAGKTVICYFNGGAMQNWDDDKDDFPEDVIGKSLDYPYVGDEDGEFYVDIRDSRVLEIMKKRLDNAVSAGCDAVDPDNVDAWAEGTSDDDDPTGFGLKPQDYADYLTKLAEYAHSQETKLGDKLLVGQKNAPDIAESLVSTLDFAVLESCRTWSFCGEFQTYIEAGKPVFQIEYPPSIEETGSLSDEDEEFYCKESEEEHGDKGFSKLLKFASARLDGWTQLCDGSIFEQPMLDW

SEQ ID NO: 127 - protein, 250 aa, Rasamsonia byssochlamydoides
PPNTTTWQPQVRTSWQIELLYALNDTSVDAEVYDIDLFSNNASIIAQLQDAGRRVICYFSAGSYENWRPDANEFQPSDLGNTLDGWPDERWLNINSANVHRIMKARLDMASDKGCDGVDPDNVDGYDNDNGLDLTEADSINYVNFLANEAHARNLSIGLKNAGAIIPSVIDNMQWSVNEQCVQYDECDTYAVFIAAGKPVFHIEYPKGDDTNNNQLVTAAEKAAACNSTNAANFSTILKNMDLDNWIETC

SEQ ID NO: 128 - protein, 276 aa, Acremonium dichromosporum
ATNVFGGLEKGAKWNICIHNPIKHDTPDAVLPREASVYDVAVDHARDFPDIIPAIKESGKTVLCYFNAGALQDWDADKGDFPKAAIGRTMGGDYDDEWYLDIRRQDVVELMYKRLEEAAALGCDGVDPDNVDAWAEAGEDRSGFGLTQQDYTDYLIKLANFAHGLGPLMVGQKNAPDMAADLVGALDFAVLESCREWDFCKDFQVYVEAGKPVFQIEYPESIVEQGELSPDDYKKHCEGDAGDAGFSKVLKRASAQLDGWTQYCNEEPFEQAVIEW

SEQ ID NO: 129 - protein, 248 aa, Thermomyces dupontii
GTVSWKPRVGTTWQIELLYPLNDTSHDVDVYDIDLFINGEKIISKLHGDGRKVICYFSAGSYENWRPDVHKFRPEDLGNTLEGWEDERWLDIRSNNVRDIMKDRLDMARDKGCDGVDPDNVDGYDNDNGLSLTQDDSISFVTLLAREAHARGLSIGLKNAGDIIDSVIDLMQWGVNEQCAEFDECDTYAVFTEQGKPVFHIEYPKGDEINDNKPVTSAQKYSACNAVGSGNFSTIIKNMNLDNWVQIC

SEQ ID NO: 130 - protein, 316 aa, Ovatospora medusarum
QIKLPPGWKVGMKWQIEIQHTVDVNTPLVPSDALVWDVDLYHIARKPEVINHLRSTNPETIVICYFNAGLVQESDCDWETTWQSPEYRDLLGTRHPDFPDEAWVNIRNQTGRDLIKRRIDLARDLGCDGVDPDNIDGYTLDEPGSDHPTGWNLTQQDDITFILDLAAHAHSLTTLRGNTLLIGQKNAPEITDSVAASLDFAVLEDCKSLTTPDGEERFIFCEEYQEPYIRGGKPVFSIEYPKTLGDSNTGVCSSTGTERAEWERSCEVAEYGSEGFSEVLKIQGGEGELNGCTEYCEVKGLVETAVDTEGGSCPGE

SEQ ID NO: 131 - protein, 257 aa, Marasmius oreades
AVTNAKRAITPLPANGKFDYQIGGAYTPDSDVKVVTRDRTESPAQGKYNICYVNAFQTQPDEKAFWQSSERDHLLLRNANGEYFIDPEWPDEFLLDTSKDQNRQEIAAVINGWISDCKQKGFNAIEADNLDTFSRSKGLLTVDNNLQFAKILTDHAHSLDMAFGQKNAGEVAARAKKEAGFDFAVVEQCQEFEECDTYTDVYGNQMLEIEYYNEDLPENGLENFKDACQARGNQISIIFRDVEVRPPGSEDRVYQEC

SEQ ID NO: 132 - protein, 252 aa, Amycolatopsis niigatensis; identical to SEQ ID NO: 81

SEQ ID NO: 133 - protein, 248 aa, Byssochlamys spectabilis
TTAKWQPPVGTTWQIELLYPLNDTSFDVDVYDIDLFDNNATMISTLHDKGHRVICYFSAGTYENWRPDKSRFKDSDLGKALGDWPGEKWLNINSTNVRDIMVSRLDLARQKGCDGVDPDNVDGYDNDNGLGLTEADSVNYVNFLANETHARNMSIGLKNAGAIIPWVIGNMQWSVNEQCAQYHECDTYLPFIEAGKPVFHIEYPKGDSTNNGNLVSTSQKNNACLFNDSGNFSTIIKNMDLDNWVQRC

SEQ ID NO: 134 - protein, 253 aa, Coprinopsis sp.
VAVLEKRAVTLPPANGKFDYQLGGAYTPPTGTTIVTRDRTASPVAGLYNICYINAFQTQASEAAWWKINHDTLLLRKANNQYFEDPDWAGEIFLDTRTAAKRTAIATILNGWIDGCATAGFKAIEPDNLDSYTRSNSLLTKANNLALAKLIADYAHTKNLAIAQKNTAELGSEGKTTAGFDFAVAEECQQWDECDEYTDVYGTGVLEIEYTDNSNAQSVYQAACTARGATLSVILRDRDLVALGQSGYHYEQC

SEQ ID NO: 135 - protein, 301 aa, Fusarium sambucinum
EPEPAAAASSPSSFSLSDFKPGVQWEIVIHEPIKHDGTADLIPSKAKVWDIDMGHARDYPKMIPLLKSAGKFVICYFNAGAVQAWDEDKNQFPKAVIGHSLAYPYDSEEWYLDIRDSTVLKLQKARLDVAAKIGCDAVDPDNVDAWQQDEEDPTGFKLKSSDYTKYLKSLAEYAHSIKTKDGNPLLVGQKNAPEIAEDLVSSLDFAVLESCRGTTDPKEENWPFCEDFQTYIDAGKPVLQIEYPPSVEKTGKLSSSDKTYYCTPKEEDKGFSKVIKWASAQLDGWGQYCGEEPFRTPVIKE

SEQ ID NO: 136 - protein, 242 aa, Amycolatopsis orientalis
APLPAPHSVTLPAPHAGFDYQIGGPYTPPSGVQVVSRDHSAPAAAGVYNICYVNAFQAQPGAEGEWGDLVLRDGNGDVVMDPDWNEALLDLRTADKRSRIADKVGGWIDECAGKGYQAIEPDNYDSFTRSQGLLSARNAQGLVKLLSSRAHGKGLAIAQKNTSELSSSRADNGLDFAVAEECGEQDNCAEYTQYFGDHVIVIEYTEDGLRNACATWGSSLSVVRRDRDVTPKGESGYVRETC

SEQ ID NO: 137 - protein, 291 aa, Synthetic construct
GGPAVGGTSPVPDAARTTTTDATTAGVVELPPTSGILDYQLGGAYDQVDAGSGPVAPDVVVRDATAAPLPGAYNVCYVNGFQTQPDDADLWLDHEELLLHDAGGELVIDPDWPDEFVLDPSTAAQRDGILDLLGPVVTGCADDGFDAVEIDNLDTWTRFDQIDEAGAHALATAYVGLAHDAGLAIAQKNAAEITQVAHDDLGFDFAVTEECAVWDECSAYTDVYGDHVLQVEYPGPLAGEGLTFADVCALGDRAPLTVLRDLYLVTPDTDVSGFDPVEETGEPYSYVFETC

SEQ ID NO: 138 - protein, 238 aa, Pilimelia columellifera
ASSPALPPVHAGFDYQIGGAYTPPSGVKVVSRDHEASPAAGLYNICYVNAFQAQPGAEKGWDSDLLLRKANGDVVYDTNWGEALLDLRTADKRRRVAEKVDGWIDGCADKGFQAVEPDNYDSYTRSSKLLSAADAEAYITLLSQHAHDRGLAIAQKNTSQLSGDRAKTGLDFAIAEECGTWDECGDYTDAFGSNVIVIEYTDKGLSKACSGWGDRLSVVERDLDVSPSGSKGYVRKTC

SEQ ID NO: 139 - protein, 322 aa, Cellulomonas cellasea
GPVQARPTGSAPTTVAPAPDATTTPSADAGTTTGPTPSASATPSPTPSPTATATAPASAGPTAAPAPTPTSATPAPVPAGLGRIVVDYQLGGAYALGPGVGGVVRDSTSEPAPGAYSICYVNGFQTQPDERTTWLREHPVLLLRDAAGRVVADPGWPDEVLLDTSTDDKRARLAAVVGASLQRCADAGFDAVELDNLDSYTRSGGLLTADDALATAARYVAVAHGLGLAVGQKNAADLTRRGHDEVGFDFAVAEECHRWDECGAYTDVYGDQVIDVEYADDLRGTFAEVCADPQTPFSTVLRDHDLTTPASRAHVLEVCPRG

SEQ ID NO: 140 - protein, 260 aa, Synthetic construct
SSVPTTSPAAVTLPPTDGIFDYQLGGAYDTVDAGEGPTAIDVVVRDATAEPLPGAYSVCYVNGFQTQPDQADLWEGREDLLLHDADGGLVVDPEWPDEHVLDPSTAAQRDGILEVLGPVVTGCADAGFDAVELDNLDTWTRFDAIDEAGAYALAQAYVDLAHGAGLAVAQKNAAEISRTAHDELGFDLAVTEECTAWDECAAYTDVYGDHVLQVEYPDTLDDAGLTFADVCALDDRAPLTILRDRDLVAAGEDGHVYDAC

SEQ ID NO: 141 - protein, 252 aa, Microbacterium sp.
SGPPSTGGAVRLPPQGAGFDYQLGGAYPPSDGVDIVVRDRTAPPAGAGYDVCYVNGFQTQPEDSAAVARDRPELLVAGVDGPLVDAGWPDEYLFDTSSEASRSQLVDLVGEQIRGCAADGYAAVEIDNLDSYTRSDGALTWGDNRSLAEAYVRIAHDAGLAVAQKNTAEHAEELAAAGFDFAVAESCAAFAECDDYSAVYPAVLDVEYVDETSRDDFLAACGDADPRVSMIRRDLQLVAPPASGHVFEACAS

SEQ ID NO: 142 - protein, 238 aa, Amycolatopsis bullii
ATTAAVTPPPVKAGFDYQIGGAYTPASDVKVVSRDHTAQPAKGLYNICYVNAFQAQPGAEGEWGDLLLRDANGNVVIDEDWDEALLDLRTADKRQRVAAKVDAWVDDCAAKGYQGVEPDNFDSYTRSRGLLSDSDAQAYIRLLSAHAHAKGLAIAQKNTSELSDQRQANGLDFAIAEECGQQKNCGEFTPAFGDHVIVIEYTDGGLKTACSRWSSLSIVRRDHDVVPKGEAGYVRKTC

SEQ ID NO: 143 - protein, 246 aa, Microbacterium oxydans
HSPSSGVTLPPSGAVPDYQLGGAYAPGPEVGIVGRDRSAEPVPGVYSICYVNGFQTQPGELADWPRDLLLQRGGEVVFDPDWPDEALLDTSTAEHRDRISETVKPWIDECADAGFDAVEFDNLDSYSRSDGSLSFDDNRALASLLVDAAHTAGLAAGQKNAAEDAAPLRARADFDFAVTEECAAYEECEAYTSVYGDHVIDIEYADELPRSFAEMCADDASPAAMVLRDRDLVTPDDGAYVFETCG

SEQ ID NO: 144 - protein, 259 aa, Microbacterium phyllosphaerae
PSETSPTSGGGEATETANAFALPPAGATPDYQLGGAYQPADGVGIVGRDRSDDPAEGLYSICYVNGFQTQPGELDTWPDDLLLQRDGAPVFDPDWPDEALLDTSSDDRRSRIADIVGPWIEGCADAGFDAVEFDNLDSYTRSDGALALDDNLALATLLVDVAHEAGLAAGQKNAAEDAAALHQRAGFDFAVVEECGAYEECPAFTDVYGDAVVDIEYSDELPRAFAEMCADDESPASMVLRDRDLLTPDSDGYMFETCP

SEQ ID NO: 145 - protein, 264 aa, Phycicoccus dokdonensis
ATTPATQQTSPSTSAHRPASGTTTSRSVAAFPAGEPFDYQIGGAFPAAAGVRVVSHDRAERPDPNRYSVCYVNGYQAQPQELAWWQAQHPELLLRDATGRLVIDEDWDEALLDLRTAARRSALVTVIGAWVAGCAGDGFDAVELDNLDSYLRSDGLLTPSDALALATALAEEAHRHGLAVGQKNAPELAARLSSAGYDFAVAEECAEYAECDVYTSAYGAAVVDIEYTRAAFRKACRDPSHPPTTLRDHDVSPPGRPGFVDERC

SEQ ID NO: 146 - protein, 244 aa, Synthetic construct
ADDWYRPPVSVTWQWQLDGVVNEDYDVDLYDIDLFDSSVELIQRLQASGKKVICYFSAGSYEDWRPDAVDFAAKDLGKTLSGWDNERWLEIRSSQVRDVMIRRLDLAVSKGCDGVEPDNVDSYRTNTGFSFRANDQLDYNRFLAAQAHARGLAIGLKNDPDQARALSSDFDFAITEQCFEFSECRSYSSFIKAGKPVLNAEYRRIYVDDEVQRNAMCKQSLALEFSTLVLPVTLDDKFRLSCLP

SEQ ID NO: 147 - DNA, 25 bp, Synthetic construct
  source 1..25: mol_type = other DNA
gacagccgcatccgtggtctccgca

SEQ ID NO: 148 - DNA, 26 bp, Synthetic construct
  source 1..26: mol_type = other DNA
ctaccgccagcagtgtctgcgattta

SEQ ID NO: 149 - protein, 19 aa, Synthetic construct
MKLSWLVAAALTAASVVSA

(19) United States
(12) Patent Application Publication
(10) Pub. No.: US 2024/0425833 A1
(43) Pub. Date: Dec. 26, 2024

(54) POLYPEPTIDES

(71) Applicant: Novozymes A/S, Bagsvaerd (DK)

(72) Inventors: Ming Li, Beijing (CN); Jesper Salomon, Holte (DK); Dorotea Raventos Segura, Rungsted (DK); Mary Ann Stringer, Soborg (DK); Rebecca Munk Vejborg, Allerod (DK); Dorte Marie Koefoed Klitgaard, Birkerod (DK); Dorota Nissen, Bagsvaerd (DK); Wei Peng, Bagsvaerd (DK); Tianqi Sun, Beijing (CN)

(73) Assignee: Novozymes A/S, Bagsvaerd (DK)

(21) Appl. No.: 18/822,802

(22) Filed: Sep. 3, 2024

Related U.S. Application Data
(62) Division of application No. 17/051,454, filed on Oct. 29, 2020, now Pat. No. 12,110,518.

(30) Foreign Application Priority Data
Jun. 1, 2018 (EP) 18175572.9

(51) Int. Cl. C12N 9/24; C11D 3/00; C11D 3/386

(52) U.S. Cl. CPC C12N 9/2402; C11D 3/0036; C11D 3/38636; C11D 3/38672; C11D 2111/12; C12Y 302/01109

(57) ABSTRACT

The invention relates to polypeptides having hydrolytic activity and polynucleotides encoding the polypeptides. The invention also relates to nucleic acid constructs, vectors, and host cells comprising the polynucleotides as well as methods of producing and using the polypeptides.

### CROSS-REFERENCE TO RELATED APPLICATIONS

This application is a division of U.S. application Ser. No. 17/051,454 filed Oct. 29, 2020, now pending, which is a 35 U.S.C. 371 national application of international application no. PCT/CN2019/089237 filed May 30, 2019, which claims priority or the benefit under 35 U.S.C. 119 of European application no. 18175572.9 filed Jun. 1, 2018. The disclosure of each application is fully incorporated herein by reference.

### REFERENCE TO A SEQUENCE LISTING

This application contains a Sequence Listing in computer readable form, which is incorporated herein by reference. The electronic sequence listing was created on Jul. 8, 2024, is named SQ.xml, and is 690 kb in size.

### BACKGROUND OF THE INVENTION

### Field of the Invention

The present invention relates to polypeptides comprising a GH114 domain having hydrolytic activity and polynucleotides encoding the polypeptides. The invention also relates to nucleic acid constructs, vectors, and host cells comprising the polynucleotides as well as methods of producing and using the polypeptides.

### Description of the Related Art

Enzymes have been used in detergents for decades. Usually, a cocktail of various enzymes is added to detergent compositions. The enzyme cocktail often comprises various enzymes, wherein each enzyme targets it specific substrate, e.g., amylases are active towards starch stains, proteases on protein stains and so forth. Textiles and surfaces such as laundry and dishes become soiled with many different types of soiling. The soiling may compose of proteins, grease, starch etc. Biofilm is an example of soiling and the presence of biofilm provides several disadvantages. Biofilm comprises an extracellular polymeric matrix, composed of, e.g., polysaccharides, extracellular DNA (eDNA), and proteins. The extracellular polymeric matrix may be sticky or gluing, which when present on textile, give rise to redeposition or back staining of soil resulting in a greying of the textile. Another drawback is that malodor may be trapped within the organic structure. Biofilm is therefore not desirable in textiles and surfaces associated with cleaning such as washing machines etc. As biofilm is a complex mixture of polysaccharides, proteins, DNA etc. there is a need for enzymes which effectively prevent, remove or reduce components of such soiling, e.g., polysaccharides of components hereof on items such of fabrics. There is a need for enzymes which effectively remove or reduce components of organic soiling such as polysaccharides in, e.g., the EPS in cleaning processes such as laundry and hard surface cleaning. The object of the present invention is to provide enzymes, which are compatible with cleaning compositions, e.g., detergents and which effectively reduce polysaccharides associated, e.g., with EPS.

### SUMMARY OF THE INVENTION

The present invention provides polypeptides with hydrolase activity, wherein the polypeptides have a GH114 domain (CAZy database, cazy.org, Lombard et al., 2014, Nucleic Acids Res. 42: D490-D495). The GH114 domain is a functional domain providing hydrolytic activity to the polypeptide. The invention further provides detergent compositions comprising polypeptides comprising the GH114 domain and the use of such polypeptides for cleaning, e.g., deep cleaning in cleaning processes. The polypeptides of the present invention comprising the GH114 domain have beneficial properties such as cleaning, e.g., deep cleaning in cleaning processes. Cleaning processes include laundry and dish wash.

In a first aspect the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) [VLI]XE[EDSQ]C (SEQ ID NO: 60) or CY[FLIV][SDN][ATVG] (SEQ ID NO: 61); and
(b) one or more cleaning composition components, preferably at least one surfactant, such as an anionic surfactant, wherein the anionic surfactant preferably includes sulfates and sulfonates, such as linear alkylbenzenesulfonates (LAS), alpha-olefinsulfonates (AOS) and alkyl alcohol ethersulfates (AES or AEOS or FES).

In a second aspect the invention relates to a method for laundering an item comprising the steps of:
(a) exposing an item to a wash liquor comprising a composition according to the invention;
(b) completing at least one wash cycle; and
(c) optionally rinsing the item, wherein the item is a textile.

In a third aspect the invention relates to the use of a GH114 glycosyl hydrolase enzyme, preferably having alpha-1,4-polygalactosaminidase activity, wherein the GH114 glycosyl hydrolase enzyme comprises one or more motif(s) selected from the group consisting of [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70) and DFAVL (SEQ ID NO: 71), in a cleaning process, such as laundry and/or dish wash.

In a third aspect the invention relates to use of a GH114 glycosyl hydrolase enzyme, preferably having alpha-1,4-polygalactosaminidase activity, wherein the GH114 glycosyl hydrolase enzyme comprises one or more motif(s) selected from the group consisting of [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70) and DFAVL (SEQ ID NO: 71),
i. for preventing, reducing or removing stickiness of the item;
ii. for preventing, reducing or removing biofilm or biofilm components
iii. for reducing or removing pel stains on the item;
iv. for preventing, reducing or removing redeposition of soil during a wash cycle;
V. for preventing, reducing or removing adherence of soil to the item;
vi. for maintaining or improving whiteness of the item;
vii. for preventing, reducing or removing malodor from the item, wherein the item is a textile.

In one aspect, the present invention relates to a GH114 glycosyl hydrolase comprising the motif [VLI]XE[EDSQ]C (SEQ ID NO: 60) and/or CY[FLIV][SDN][ATVG] (SEQ ID NO: 61), wherein the GH114 glycosyl hydrolase has hydrolytic activity, and wherein the GH114 glycosyl hydrolase comprises or consist of a polypeptide selected from the group consisting of:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(h) a polypeptide having, at least 50% at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(i) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(j) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 36;
(k) a polypeptide having at least 99% or 100% sequence identity to the polypeptide shown in SEQ ID NO: 39;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(m) a polypeptide having at least 85%, at least 90%, at least 95%, at least 96%, at least 97%, at least 98%, at least 99% or 100% sequence identity to the polypeptide shown in SEQ ID NO: 45;
(n) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(o) a polypeptide having at least 98%, at least 99% or 100% sequence identity to the polypeptide shown in SEQ ID NO: 51;
(p) a polypeptide having at least 80%, at least 85%, at least 90%, at least 95%, at least 96%, at least 97%, at least 98%, at least 99% or 100% sequence identity to the polypeptide shown in SEQ ID NO: 54; and
(q) a polypeptide having at least 85%, at least 90%, at least 95%, at least 96%, at least 97%, at least 98%, at least 99% or 100% sequence identity to the polypeptide shown in SEQ ID NO: 57.

The present invention also relates to GH114 glycosyl hydrolases. In particular, the invention relates to polypeptides selected from the group consisting of:
(a) a polypeptide having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54;
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57;
(t) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30 and SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54 and SEQ ID NO: 57, wherein the variant has hydrolytic activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions;
(u) a polypeptide comprising the polypeptide shown in (a) to (t) and a N-terminal and/or C-terminal His-tag and/or HQ-tag;
(v) a polypeptide comprising the polypeptide shown in (a) to (t) and a N-terminal and/or C-terminal extension of between 1 and 10 amino acids; and
(w) a fragment of the polypeptide shown in (a) to (t) having hydrolytic activity and having at least 90% of the length of the mature polypeptide.

The invention further relates to a cleaning composition, e.g., a detergent composition, a ADW composition, a laundry composition, comprising a polypeptide according to the invention. One aspect relates to a cleaning composition comprising:
a) at least 0.001 ppm of at least one GH114 glycosyl hydrolase according to the invention; and
b) one or more cleaning composition components, preferably selected from surfactants, builders, bleach components, polymers, dispersing agents and additional enzymes.

The invention further relates to a method for laundering an item comprising the steps of:
a. exposing an item to a wash liquor comprising a GH114 glycosyl hydrolase according to the invention or a composition according to the invention;
b. completing at least one wash cycle; and
c. optionally rinsing the item, wherein the item is a textile.

The invention further relates to use of a polypeptide according to the invention for cleaning, e.g., deep cleaning of an item, such as textile, e.g., fabric. The invention further relates to the use of a polypeptide according to the invention,
(i) for preventing, reducing or removing stickiness of the item;
(ii) for pretreating stains on the item;
(iii) for preventing, reducing or removing redeposition of soil during a wash cycle;
(iv) for preventing, reducing or removing adherence of soil to the item;
(v) for maintaining or improving whiteness of the item;
(vi) for preventing, reducing or removing malodor from the item, wherein the item is a textile.

The invention further relates to the use of a GH114 glycosyl hydrolase in a cleaning process, such as laundry and/or dish wash. The invention also relates to the use of a GH114 glycosyl hydrolase,
i. for preventing, reducing or removing stickiness of the item;
ii. for preventing, reducing or removing biofilm or biofilm components
iii. for reducing or removing pel stains on the item;
iv. for preventing, reducing or removing redeposition of soil during a wash cycle;
v. for preventing, reducing or removing adherence of soil to the item;
vi. for maintaining or improving whiteness of the item;
vii. for preventing, reducing or removing malodor from the item, wherein the item is a textile.

In one aspect, the invention relates to a granule comprising:
(a) a core comprising a GH114 glycosyl hydrolase according to the invention, and
(b) optionally a coating consisting of one or more layer(s) surrounding the core.

The invention further relates to a polynucleotide encoding the polypeptide of the invention. A nucleic acid construct or expression vector comprising a polynucleotide encoding a polypeptide of the invention, which is operably linked to one or more control sequences that direct the production of the polypeptide in an expression host. The invention further relates to a recombinant host cell comprising a polynucleotide encoding a polypeptide of the invention, which is operably linked to one or more control sequences that direct the production of the polypeptide, wherein the method may further comprise cultivating a cell, which in its wild-type form produces the polypeptide, under conditions conducive for production of the polypeptide and optionally recovering the polypeptide.

### OVERVIEW OF FIGURES

FIG. 1 shows the phylogenetic tree as well as the clades of some of the GH114 glycosyl hydrolase polypeptides.

FIG. 2 shows the phylogenetic of some of the GH114 glycosyl hydrolase polypeptides.

FIG. 3 shows multiple alignment of some of the GH114 glycosyl hydrolase polypeptides.

FIG. 4 shows the clade hierarchy.

### OVERVIEW OF SEQUENCES

SEQ ID NO: 1 DNA encoding full length polypeptide from Amycolatopsis circi

SEQ ID NO: 2 polypeptide derived from SEQ ID NO: 1

SEQ ID NO: 3 mature polypeptide obtained from Amycolatopsis circi

SEQ ID NO: 4 DNA encoding full length polypeptide from Amycolatopsis sp.

SEQ ID NO: 5 polypeptide derived from SEQ ID NO: 4

SEQ ID NO: 6 mature polypeptide obtained from Amycolatopsis sp.

SEQ ID NO: 7 DNA encoding full length polypeptide from Amycolatopsis sp.

SEQ ID NO: 8 polypeptide derived from SEQ ID NO: 7

SEQ ID NO: 9 mature polypeptide obtained from Amycolatopsis sp.

SEQ ID NO: 10 DNA encoding full length polypeptide from Streptomyces parvulus

SEQ ID NO: 11 polypeptide derived from SEQ ID NO: 10

SEQ ID NO: 12 mature polypeptide obtained from Streptomyces parvulus

SEQ ID NO: 13 DNA encoding full length polypeptide from Fusarium solani

SEQ ID NO: 14 polypeptide derived from SEQ ID NO: 13

SEQ ID NO: 15 mature polypeptide obtained from Fusarium solani

SEQ ID NO: 16 DNA encoding full length polypeptide from Fusarium solani

SEQ ID NO: 17 polypeptide derived from SEQ ID NO: 16

SEQ ID NO: 18 mature polypeptide obtained from Fusarium solani

SEQ ID NO: 19 DNA encoding full length polypeptide from Nonomuraea guangzhouensis

SEQ ID NO: 20 polypeptide derived from SEQ ID NO: 19

SEQ ID NO: 21 mature polypeptide obtained from Nonomuraea guangzhouensis

SEQ ID NO: 22 DNA encoding full length polypeptide from Streptomyces sp

SEQ ID NO: 23 polypeptide derived from SEQ ID NO: 22

SEQ ID NO: 24 mature polypeptide obtained from Streptomyces sp

SEQ ID NO: 25 DNA encoding full length polypeptide from Vibrio sp

SEQ ID NO: 26 polypeptide derived from SEQ ID NO: 25

SEQ ID NO: 27 mature polypeptide obtained from Vibrio sp

SEQ ID NO: 28 DNA encoding full length polypeptide from Synthetic gene

SEQ ID NO: 29 polypeptide derived from SEQ ID NO: 28

SEQ ID NO: 30 mature polypeptide obtained from Synthetic gene

SEQ ID NO: 31 DNA encoding full length polypeptide from Microbacterium saccharophilum

SEQ ID NO: 32 polypeptide derived from SEQ ID NO: 31

SEQ ID NO: 33 mature polypeptide obtained from Microbacterium saccharophilum

SEQ ID NO: 34 DNA encoding full length polypeptide from Microbacterium oxydans

SEQ ID NO: 35 polypeptide derived from SEQ ID NO: 34

SEQ ID NO: 36 mature polypeptide obtained from Microbacterium oxydans

SEQ ID NO: 37 DNA encoding full length polypeptide from Frigoribacterium faeni

SEQ ID NO: 38 polypeptide derived from SEQ ID NO: 37

SEQ ID NO: 39 mature polypeptide obtained from Frigoribacterium faeni

SEQ ID NO: 40 DNA encoding full length polypeptide from Serinibacter sp.

SEQ ID NO: 41 polypeptide derived from SEQ ID NO: 40

SEQ ID NO: 42 mature polypeptide obtained from Serinibacter sp.

SEQ ID NO: 43 DNA encoding full length polypeptide from Oerskovia paurometabola

SEQ ID NO: 44 polypeptide derived from SEQ ID NO: 43

SEQ ID NO: 45 mature polypeptide obtained from Oerskovia paurometabola

SEQ ID NO: 46 DNA encoding full length polypeptide from Agreia pratensis

SEQ ID NO: 47 polypeptide derived from SEQ ID NO: 46

SEQ ID NO: 48 mature polypeptide obtained from Agreia pratensis

SEQ ID NO: 49 DNA encoding full length polypeptide from Plantibacter flavus

SEQ ID NO: 50 polypeptide derived from SEQ ID NO: 49

SEQ ID NO: 51 mature polypeptide obtained from Plantibacter flavus

SEQ ID NO: 52 DNA encoding full length polypeptide from Streptomyces miharaensis

SEQ ID NO: 53 polypeptide derived from SEQ ID NO: 52

SEQ ID NO: 54 mature polypeptide obtained from Streptomyces miharaensis

SEQ ID NO: 55 DNA encoding full length polypeptide from Urnula craterium

SEQ ID NO: 56 polypeptide derived from SEQ ID NO: 55

SEQ ID NO: 57 mature polypeptide obtained from Urnula craterium

SEQ ID NO: 58 signal peptide MKKPLGKIVASTALLISVAFSSSIASA

SEQ ID NO: 59 HHHHHHPR His-tag

SEQ ID NO: 60 [VLI]XE[EDSQ]C

SEQ ID NO: 61 CY[FLIV][SDN][ATVG]

SEQ ID NO: 62 DYQ[LI]G

SEQ ID NO: 63 FQ[TAV]Q[PSD]

SEQ ID NO: 64 AEECG

SEQ ID NO: 65 NAFQ[AT]Q

SEQ ID NO: 66 WQWQL

SEQ ID NO: 67 [VLI][GASD]LKN[DGS][VLIP]

SEQ ID NO: 68 GXXVX[NHQTS]IEY[PG]

SEQ ID NO: 69 VICYF

SEQ ID NO: 70 ICYFSA

SEQ ID NO: 71 DFAVL

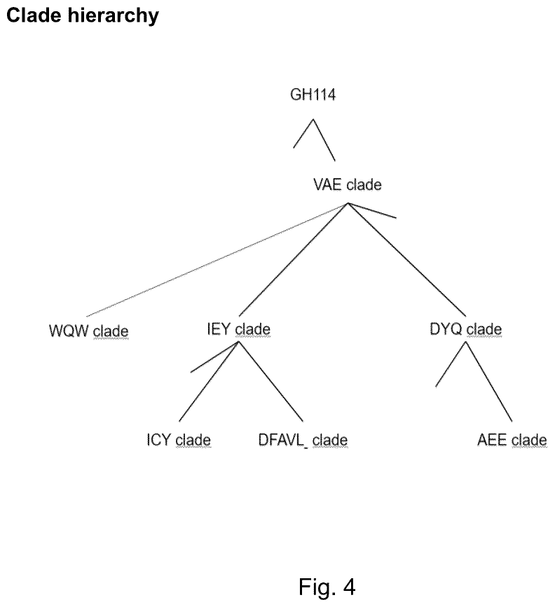

Homologue sequences according to clades as shown in FIG. 4.

VAE clade: SEQ ID NO: 72-SEQ ID NO: 146
DYQ clade: SEQ ID NO: 79, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 90, SEQ ID NO: 91, SEQ ID NO: 92, SEQ ID NO: 93, SEQ ID NO: 94, SEQ ID NO: 96, SEQ ID NO: 131, SEQ ID NO: 132, SEQ ID NO: 134, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144 and SEQ ID NO: 145 AEE clade: SEQ ID NO: 79, SEQ ID NO: 82, SEQ ID NO: 83, SEQ ID NO: 84, SEQ ID NO: 85, SEQ ID NO: 132, SEQ ID NO: 136, SEQ ID NO: 138, and SEQ ID NO: 142.
IEY clade: SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 133 and SEQ ID NO: 135
ICY clade: SEQ ID NO: 98, SEQ ID NO: 101, SEQ ID NO: 104, SEQ ID NO: 107, SEQ ID NO: 114, SEQ ID NO: 115, SEQ ID NO: 120, SEQ ID NO: 124, SEQ ID NO: 125, SEQ ID NO: 127, SEQ ID NO: 129 and SEQ ID NO: 133
DFAVL clade: SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 110, SEQ ID NO: 111, SEQ ID NO: 112, SEQ ID NO: 113, SEQ ID NO: 116, SEQ ID NO: 117, SEQ ID NO: 118, SEQ ID NO: 119, SEQ ID NO: 121, SEQ ID NO: 123, SEQ ID NO: 126, SEQ ID NO: 128, SEQ ID NO: 130 and SEQ ID NO: 135
WQW clade: SEQ ID NO: 80, SEQ ID NO: 81, SEQ ID NO: 89, SEQ ID NO: 95 and SEQ ID NO:

### Definitions

Activity: The present invention relates to glycosyl hydrolases (EC 3.2.1.-), which are a widespread group of enzymes that hydrolyze the glyosidic bond between two or more carbohydrates or between a carbohydrate and a non-carbohydrate moiety. A classification of glycoside hydrolases in families based on amino acid sequence similarities has been proposed. The polypeptides of the invention comprise at least one glycosyl hydrolase domain and are in the present context defined as glycosyl hydrolases. Thus, polypeptides of the invention hydrolyse glyosidic bonds and the polypeptides of the invention have hydrolytic activity. The glycosyl hydrolase domain comprised in the polypeptides of the invention may be classified as a GH114 (GH114, Glycoside Hydrolase Family 114, CAZy database, cazy.org, Lombard et al., 2014, Nucleic Acids Res. 42: D490-D495). A single enzyme of GH114 has been characterized; an endo-alpha-1,4-polygalactosaminidase (EC 3.2.1.109) from Pseudomonas sp. (Tamura et al., 1995, Molecular Cloning and Sequence Analysis of the Gene Encoding an Endo-α-1,4 polygalactosaminidase of Pseudomonas sp. 881. J. Fermentation Bioengineer. 80:305). This enzyme hydrolyzes alpha-1,4-polygalactosamine to oligosaccharides in an endo-acting manner. Alpha-1,4-Polygalactosamine, also known as galactosaminoglycan, is a polymer consisting of alpha-1,4-linked galactosamine residues, which is only partially N-acetylated, and may also contain N-formyl residues. A GH114 glycosyl hydrolase is in the context of the present invention a glycosyl hydrolase comprising glycosyl hydrolase domain (DUF297), which here is termed GH114 (GH114, Glycoside Hydrolase Family 114, CAZy database, cazy.org, Lombard et al., 2014, Nucleic Acids Res. 42: D490-D495). The GH114 glycosyl hydrolase domain is located at position 13 to 246 in SEQ ID NO: 3. The polypeptides of the invention are glycosyl hydrolases, preferably GH114 glycosyl hydrolases. In one embodiment, the polypeptides of the invention are polygalactosaminidases, some GH114 of the invention may comprise α-1,4 polygalactosaminidase activity. The polypeptides of the invention have hydrolytic activity to glyosidic bond. In one or in the context of the present invention the GH114 glycosyl hydrolase is a PelA enzyme, which is active towards the polysaccharide PEL, present in many biofilms. The pellicle (PEL) polysaccharide is synthesized, e.g., by Pseudomonas aeruginosa and is an important biofilm constituent critical for bacterial virulence and persistence. The GH114 of the invention hydrolyse Pel a positively charged exopolysaccharide composed of partially acetylated 1,4 glycosidic linkages of N-acetylgalactosamine and N-acetyl-glucosamine (Jennings et al., September 2015, PNAS 112 (36): 11353-11358; Marmont et al., 2017, J. Biol. Chem. 292 (47): 19411-19422.

Allelic variant: The term “allelic variant” means any of two or more alternative forms of a gene occupying the same chromosomal locus. Allelic variation arises naturally through mutation and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Biofilm: A biofilm is organic matter produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium. Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community. On laundry biofilm or EPS producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp. In one aspect, the biofilm producing strain is Pseudomonas. In one aspect, the EPS producing strain is Pseudomonas aeruginosa, Pseudomonas alcaliphila or Pseudomonas fluorescens, Pseudomonas composti, Pseudomonas lundensis and Pseudomonas corrugate. In one embodiment, the biofilm is caused by microorganisms or group of microorganisms which produce Pel. In another embodiment, the biofilm produces a polysaccharide that is degradable by the GH114 glycosyl hydrolases of the invention. The biofilm that may be formed on the surface, e.g., such as textiles may be caused by any microorganism or group of microorganisms that forms PelA-dependent biofilm including but not limited to Acinetobacter sp., Aeromicrobium sp., Brevundimonas sp., Microbacterium sp., Micrococcus luteus, Staphylococcus epidermidis, Staphylococcus aureus, Pseudomonas sp., Pseudomonas aeruginosa, Pseudomonas alcaliphila, Pseudomonas fluorescens, Stenotrophomonas sp., Paraburkholderia, Burkolderia sp., Candida sp., Bordetella pertussis, Yersinia pestis, Escherichia coli and Aspergillus sp.

Catalytic domain: The term “catalytic domain” means the region of an enzyme containing the catalytic machinery of the enzyme.

Clade: A clade is a group of polypeptides clustered together based on homologous features traced to a common ancestor. Polypeptide clades can be visualized as phylogenetic trees and a clade is a group of polypeptides that consists of a common ancestor and all its lineal descendants. Example 10 describes generation of phylogenetic trees.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Deep cleaning: The term “deep cleaning” means disruption, reduction or removal of organic components such as polysaccharides, e.g., pel, proteins, DNA, soil or other components present in organic matter such as biofilm. The term includes cleaning, i.e., removal of organic stains such as dead cell material, skin debris, sebum, sweat, grease and other stains derived from, e.g., humans (body soils) or microbes.

Cleaning component: The cleaning component, e.g., a detergent adjunct ingredient is different to the polypeptides of this invention. The precise nature of these additional cleaning or adjunct components, and levels of incorporation thereof, will depend on the physical form of the composition and the nature of the operation for which it is to be used. Suitable cleaning components include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

Cleaning Composition: The term cleaning composition includes “detergent composition” and refers to compositions that find use in the removal of undesired compounds from items to be cleaned, such as textiles. The detergent composition may be used to, e.g., clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzyme of the invention, the detergent formulation may contain one or more additional enzymes such as proteases, amylases, lipases, cutinases, cellulases, endoglucanases, xyloglucanases, pectinases, pectin lyases, xanthanases, peroxidases, haloperoxygenases, catalases, mannanases, DNases or any mixture thereof, and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxidoreductases, bluing agents and fluorescent dyes, antioxidants, and solubilizers.

Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of enzyme detergency benefit includes deep cleaning. Examples of enzyme detergency benefit includes biofilm reduction activity, e.g., as measured in example 8. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, color clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide or a catalytic domain having one or more (e.g., several) amino acids absent from the amino and/or carboxyl terminus of a mature polypeptide or domain; wherein the fragment has activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g., a host cell may be genetically modified to express the polypeptide of the invention. The fermentation broth from that host cell will comprise the isolated polypeptide.

Improved wash performance: The term “improved wash performance” is defined herein as an enzyme displaying an increased wash performance in a detergent composition relative to the wash performance of same detergent composition without the enzyme, e.g., by increased stain removal or less re-deposition. The term “improved wash performance” includes wash performance in laundry. The wash performance may be the same as “enzyme detergency benefit”. The term “wash performance” is used as an enzyme's ability to remove stains present on the object to be cleaned during, e.g., wash or hard surface cleaning. The improvement in the wash performance may be quantified by calculating the so-called intensity value (Int) defined in Example 18, herein or as (ΔL) as described in example 5 and 7 herein. See also the wash performance test in Example 5, 6, 18 and 19 herein. The term “wash performance” and “dish wash performance” maybe used interchangeably.

Laundering: The term “laundering” relates to both household laundering and industrial laundering and means the process of treating textiles with a solution containing a cleaning or detergent composition of the present invention. The laundering process can for example be carried out using, e.g., a household or an industrial washing machine or can be carried out by hand.

Malodor: The term “malodor” means an unpleasant odor, which is not desired on clean items. The cleaned item should smell fresh and clean without malodors adhered to the item. One example of malodor is compounds with an unpleasant smell, which may be produced by microorganisms. Another example is unpleasant smells can be sweat or body odor adhered to an item which has been in contact with human or animal. Another example of malodor can be the odor from spices, which sticks to items for example curry or other exotic spices which smells strongly.

Mature polypeptide: The term “mature polypeptide” means a polypeptide in its final form following translation and any post-translational modifications, such as N-terminal processing, C-terminal truncation, glycosylation, phosphorylation, etc. In some aspects, the mature polypeptide is amino acids 1 to 246 of SEQ ID NO: 2 and amino acids −34 to −1 of SEQ ID NO: 2 is a signal peptide. In one aspect, the mature polypeptide is shown in SEQ ID NO: 3. In some aspects, the mature polypeptide is amino acids 1 to 243 of SEQ ID NO: 5 and amino acids −34 to −1 of SEQ ID NO: 5 is a signal peptide. In one aspect, the mature polypeptide is shown in SEQ ID NO: 6. In one aspect, the mature polypeptide is shown in SEQ ID NO: 6. In some aspects, the mature polypeptide is amino acids 1 to 237 of SEQ ID NO: 8 and amino acids −37 to −1 of SEQ ID NO: 8 is a signal peptide. In one aspect, the mature polypeptide is shown in SEQ ID NO: 9. In some aspects, the mature polypeptide is amino acids 1 to 237 of SEQ ID NO: 11 and amino acids −39 to −1 of SEQ ID NO: 11 is a signal peptide. In one aspect, the mature polypeptide is shown in SEQ ID NO: 12. In some aspects, the mature polypeptide is amino acids 1 to 274 of SEQ ID NO: 14 and amino acids −22 to −1 of SEQ ID NO: 14 is a signal peptide. In one aspect, the mature polypeptide is shown in SEQ ID NO: 15. In some aspects, the mature polypeptide is amino acids 1 to 296 of SEQ ID NO: 17 and amino acids −21 to −1 of SEQ ID NO: 17 is a signal peptide. In one aspect, the mature polypeptide is shown in SEQ ID NO: 18. In some aspects, the mature polypeptide is amino acids 1 to 237 of SEQ ID NO: 20 and amino acids −27 to −1 of SEQ ID NO: 20 is a signal peptide. In one aspect, the mature polypeptide is shown in SEQ ID NO: 21. In some aspects, the mature polypeptide is amino acids 1 to 239 of SEQ ID NO: 23 and amino acids −33 to −1 of SEQ ID NO: 23 is a signal peptide. In one aspect, the mature polypeptide is shown in SEQ ID NO: 24. In some aspects, the mature polypeptide is amino acids 1 to 264 of SEQ ID NO: 26 and amino acids −23 to −1 of SEQ ID NO: 26 is a signal peptide. In one aspect, the mature polypeptide is shown in SEQ ID NO: 27. In some aspects, the mature polypeptide is amino acids 1 to 242 of SEQ ID NO: 29 and amino acids −19 to −1 of SEQ ID NO: 29 is a signal peptide. In one aspect, the mature polypeptide is shown in SEQ ID NO: 30. In some aspects, the mature polypeptide is amino acids 1 to 238 of SEQ ID NO: 32 and amino acids −38 to −1 of SEQ ID NO: 32 is a signal peptide. In one aspect, the mature polypeptide is shown in SEQ ID NO: 33. In some aspects, the mature polypeptide is amino acids 1 to 261 of SEQ ID NO: 35 and amino acids −26 to −1 of SEQ ID NO: 35 is a signal peptide. In one aspect, the mature polypeptide is shown in SEQ ID NO: 36. In some aspects, the mature polypeptide is amino acids 1 to 265 of SEQ ID NO: 38 and amino acids −25 to −1 of SEQ ID NO: 38 is a signal peptide. In one aspect, the mature polypeptide is shown in SEQ ID NO: 39. In some aspects, the mature polypeptide is amino acids 1 to 254 of SEQ ID NO: 41 and amino acids −30 to −1 of SEQ ID NO: 41 is a signal peptide. In one aspect, the mature polypeptide is shown in SEQ ID NO: 42. In some aspects, the mature polypeptide is amino acids 1 to 268 of SEQ ID NO: 44 and amino acids −31 to −1 of SEQ ID NO: 44 is a signal peptide. In one aspect, the mature polypeptide is shown in SEQ ID NO: 45. In some aspects, the mature polypeptide is amino acids 1 to 267 of SEQ ID NO: 47 and amino acids −24 to −1 of SEQ ID NO: 47 is a signal peptide. In one aspect, the mature polypeptide is shown in SEQ ID NO: 48. In some aspects, the mature polypeptide is amino acids 1 to 251 of SEQ ID NO: 50 and amino acids −36 to −1 of SEQ ID NO: 50 is a signal peptide. In one aspect, the mature polypeptide is shown in SEQ ID NO: 51. In some aspects, the mature polypeptide is amino acids 1 to 239 of SEQ ID NO: 53 and amino acids −44 to −1 of SEQ ID NO: 53 is a signal peptide. In one aspect, the mature polypeptide is shown in SEQ ID NO: 54. In some aspects, the mature polypeptide is amino acids 1 to 279 of SEQ ID NO: 56 and amino acids −18 to −1 of SEQ ID NO: 56 is a signal peptide. In one aspect, the mature polypeptide is shown in SEQ ID NO: 57.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having activity. In one aspect, the mature polypeptide coding sequence is nucleotides 103 to 840 of SEQ ID NO: 1 and nucleotides 1 to 102 of SEQ ID NO: 1 encodes a signal peptide. In one aspect, the mature polypeptide coding sequence is nucleotides 103 to 831 of SEQ ID NO: 4 and nucleotides 1 to 102 of SEQ ID NO: 4 encodes a signal peptide. In one aspect, the mature polypeptide coding sequence is nucleotides 112 to 822 of SEQ ID NO: 7 and nucleotides 1 to 111 of SEQ ID NO: 7 encodes a signal peptide. In one aspect, the mature polypeptide coding sequence is nucleotides 118 to 828 of SEQ ID NO: 10 and nucleotides 1 to 117 of SEQ ID NO: 10 encodes a signal peptide. In one aspect, the mature polypeptide coding sequence is nucleotides 67 to 939 of SEQ ID NO: 13 and nucleotides 1 to 66 of SEQ ID NO: 13 encodes a signal peptide. In one aspect, the mature polypeptide coding sequence is nucleotides 112 to 1061 of SEQ ID NO: 16 and nucleotides 1 to 111 of SEQ ID NO: 16 encodes a signal peptide. In one aspect, the mature polypeptide coding sequence is nucleotides 82 to 792 of SEQ ID NO: 19 and nucleotides 1 to 81 of SEQ ID NO: 19 encodes a signal peptide. In one aspect, the mature polypeptide coding sequence is nucleotides 100 to 816 of SEQ ID NO: 22 and nucleotides 1 to 99 of SEQ ID NO: 22 encodes a signal peptide. In one aspect, the mature polypeptide coding sequence is nucleotides 70 to 861 of SEQ ID NO: 25 and nucleotides 1 to 69 of SEQ ID NO: 25 encodes a signal peptide. In one aspect, the mature polypeptide coding sequence is nucleotides 58 to 783 of SEQ ID NO: 28 and nucleotides 1 to 57 of SEQ ID NO: 28 encodes a signal peptide. In one aspect, the mature polypeptide coding sequence is nucleotides 115 to 828 of SEQ ID NO: 31 and nucleotides 1 to 114 of SEQ ID NO: 31 encodes a signal peptide. In one aspect, the mature polypeptide coding sequence is nucleotides 79 to 861 of SEQ ID NO: 34 and nucleotides 1 to 78 of SEQ ID NO: 34 encodes a signal peptide. In one aspect, the mature polypeptide coding sequence is nucleotides 76 to 870 of SEQ ID NO: 37 and nucleotides 1 to 75 of SEQ ID NO: 37 encodes a signal peptide. In one aspect, the mature polypeptide coding sequence is nucleotides 91 to 852 of SEQ ID NO: 40 and nucleotides 1 to 90 of SEQ ID NO: 40 encodes a signal peptide. In one aspect, the mature polypeptide coding sequence is nucleotides 94 to 897 of SEQ ID NO: 43 and nucleotides 1 to 93 of SEQ ID NO: 43 encodes a signal peptide. In one aspect, the mature polypeptide coding sequence is nucleotides 73 to 873 of SEQ ID NO: 46 and nucleotides 1 to 72 of SEQ ID NO: 46 encodes a signal peptide. In one aspect, the mature polypeptide coding sequence is nucleotides 109 to 861 of SEQ ID NO: 49 and nucleotides 1 to 108 of SEQ ID NO: 49 encodes a signal peptide. In one aspect, the mature polypeptide coding sequence is nucleotides 133 to 849 of SEQ ID NO: 52 and nucleotides 1 to 132 of SEQ ID NO: 52 encodes a signal peptide. In one aspect, the mature polypeptide coding sequence is nucleotides 55 to 1053 of SEQ ID NO: 55 and nucleotides 1 to 54 of SEQ ID NO: 55 encodes a signal peptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Nomenclature: For purposes of the present invention, the nomenclature [EQ] means that the amino acid at this position may be a glutamic acid (Glu, E) or a glutamine (Gln, Q). Likewise, the nomenclature [VGAI] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the −no brief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labelled “longest identity” (obtained using the −no brief option) is used as the percent identity and is calculated as follows:

(Identical Deoxyribonucleotides×100)/(Length of Alignment−Total Number of Gaps in Alignment)

Variant: The term “variant” means a polypeptide having hydrolase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

### Conventions for Designation of Homologues, i.e., Variants of GH Glycosyl Hydrolases

For purposes of the present invention, the polypeptide disclosed in SEQ ID NO: 18 is used to determine the corresponding amino acid residue in another GH114 glycosyl hydrolase. The amino acid sequence of another GH114 glycosyl hydrolase is aligned with the polypeptide disclosed in SEQ ID NO: 18, and based on the alignment, the amino acid position number corresponding to any amino acid residue in the polypeptide disclosed in SEQ ID NO: 18 is determined using, e.g., the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another GH114 glycosyl hydrolase can be determined by an alignment of multiple polypeptide sequences using several computer programs including, but not limited to, MUSCLE (multiple sequence comparison by log-expectation; version 3.5 or later; Edgar, 2004, Nucleic Acids Research 32:1792-1797), MAFFT (version 6.857 or later; Katoh and Kuma, 2002, Nucleic Acids Research 30:3059-3066; Katoh et al., 2005, Nucleic Acids Research 33:511-518; Katoh and Toh, 2007, Bioinformatics 23:372-374; Katoh et al., 2009, Methods in Molecular Biology 537:39-64; Katoh and Toh, 2010, Bioinformatics 26:1899-1900), and EMBOSS EMMA employing ClustalW (1.83 or later; Thompson et al., 1994, Nucleic Acids Research 22:4673-4680), using their respective default parameters.

When another enzyme has diverged from the polypeptide of SEQ ID NO: 18 such that traditional sequence-based comparison fails to detect their relationship (Lindahl and Elofsson, 2000, J. Mol. Biol. 295:613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25:3389-3402). Even greater sensitivity can be achieved if the family or superfamily for the polypeptide has one or more representatives in the protein structure databases. Programs such as GenTHREADER (Jones, 1999, J. Mol. Biol. 287:797-815; McGuffin and Jones, 2003, Bioinformatics 19:874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313:903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP super families of proteins have been structurally aligned, and those alignments are accessible and downloadable. Two or more protein structures can be aligned using a variety of algorithms such as the distance alignment matrix (Holm and Sander, 1998, Proteins 33:88-96) or combinatorial extension (Shindyalov and Bourne, 1998, Protein Engineering 11:739-747), and implementation of these algorithms can additionally be utilized to query structure databases with a structure of interest in order to discover possible structural homologs (e.g., Holm and Park, 2000, Bioinformatics 16:566-567).

As different amino acids may be present at a given position depending on the selected parent for the variants the amino acid positions are indicated with #₁, #₂, etc. in the definitions below. In describing the variants of the present invention, the nomenclature described below is adapted for ease of reference. The accepted IUPAC single letter or three letters amino acid abbreviations are employed.

Substitutions: For an amino acid substitution, the following nomenclature is used: Original amino acid, position, substituted amino acid. Accordingly, the substitution of valine at position #₁ with alanine is designated as “Val #₁Ala” or “V #₁A”. Multiple mutations are separated by addition marks (“+”) or by commas (,), e.g., “Val #₁Ala+ “Pro #₂Gly” or V #₁A, P #₂G, representing substitutions at positions #₁ and #₂ of valine (V) and proline (P) with alanine (A) and glycine (G), respectively. If more than one amino acid may be substituted in a given position these are listed in brackets, such as [X] or {X}. Thus, if both Trp and Lys may be substituted instead of the amino acid occupying at position #₁ this is indicated as X #₁ {W, K}, X #₁ [W, K] or X #₁ [W/K], where the X indicate the amino acid residue present at the position of the parent GH114 glycosyl hydrolase, e.g., such as a GH114 glycosyl hydrolase shown in SEQ ID NO: 18. In some cases, the variants may be represented as #₁ {W, K} or X #₂P indicating that the amino acids to be substituted vary depending on the parent. For convenience, as SEQ ID NO: 18 is used for numbering the substitutions and the amino acid in the corresponding position in SEQ ID NO: 18 is indicated, e.g., A64S. However, it will be clear to the skilled artisan that a GH114 glycosyl hydrolase variant comprising A64S is not limited to parent GH114 glycosyl hydrolase having alanine at a position corresponding to position 64 of SEQ ID NO: 18. In a parent GH114 glycosyl hydrolase having, e.g., asparagine in position 64, the skilled person would translate the mutation specified as A64S to N64S. In the event the parent GH114 glycosyl hydrolase has serine in position 64, the skilled person would recognize that the parent GH114 glycosyl hydrolase is not changed at this position. The same applies for deletions and insertions described below.

Deletions: For an amino acid deletion, the following nomenclature is used: Original amino acid, position, *. Accordingly, the deletion of valine at position #₁ is designated as “Val #₁*” or “V #₁*”. Multiple deletions are separated by addition marks (“+”) or commas, e.g., “Val #₁*+Pro #₂*” or “V #₁*, P #₂*”.

Insertions: The insertion of an additional amino acid residue such as, e.g., a lysine after Val #₁ may be indicated by: Val #₁ValLys or V #₁VK. Alternatively, insertion of an additional amino acid residue such as lysine after V #₁ may be indicated by: *#₁aK. When more than one amino acid residue is inserted, such as, e.g., a Lys, and Gly after #₁ this may be indicated as: Val #₁ValLysGly or V #₁VKG. In such cases, the inserted amino acid residue(s) may also be numbered by the addition of lower case letters to the position number of the amino acid residue preceding the inserted amino acid residue(s), in this example: *#₁aK *#₁bG.

Multiple alterations: Variants comprising multiple alterations are separated by addition marks (“+”) or by commas (,), e.g., “Val #₁Trp+Pro #₂Gly” or “V #₁W, P #₂G” representing a substitution of valine and proline at positions #₁ and #₂ with tryptophan and glycine, respectively as described above.

Different alterations: Where different alterations can be introduced at a position, the different alterations may be separated by a comma, e.g., “Val #₁Trp, Lys” or V #₁W, K representing a substitution of valine at position #₁ with tryptophan or lysine. Thus, “Val #₁Trp, Lys+Pro #₂Asp” designates the following variants: “Val #₁ Trp+Pro #₂Asp”, “Val #₁Lys+Pro #₂Asp” or V #₁W, K+P #₂D.

### DETAILED DESCRIPTION OF THE INVENTION

Various enzymes are applied in cleaning processes each targeting specific types of soiling such as protein, starch and grease soiling. Enzymes are now standard ingredients in detergents for laundry and dish wash. The effectiveness of these commercial enzymes provides detergents which removes much of the soiling. However, organic matters such as EPS (extracellular polymeric substance) comprised in much biofilm constitute a challenging type of soiling due to the complex nature of such organic matters. None of the commercially available detergents effectively remove or reduce EPS related soiling. Biofilm is produced by a group of microorganisms in which cells stick to each other or stick to a surface, such as a textile, dishware or hard surface or another kind of surface. These adherent cells are frequently embedded within a self-produced matrix of extracellular polymeric substance (EPS), which constitute 50% to 90% of the biofilm's total organic matter. EPS is mostly composed of polysaccharides (exopolysaccharides) and proteins, but include other macro-molecules such as DNA, lipids and human substances. EPS is the construction material of bacterial settlements and either remain attached to the cell's outer surface or is secreted into its growth medium. EPS is required for the development and integrity of biofilms produced by a wide variety of bacteria. The inventors have shown that the GH114 polypeptides comprising the GH114 glycosyl hydrolase domain have hydrolytic activity to EPS components, e.g., PEL and thus having the potential to reduce or remove components of EPS and thus reduce or remove EPS and biofilm related soiling, e.g., pel of, e.g., textiles. It is well known that polypeptides deriving from organisms may share common structural elements, which can be identified by comparing the primary structures, e.g., amino acid sequences and grouping the polypeptides according to sequence homology. However, common structural elements may also be identified by comparing the three-dimensional (3D) structure of various polypeptides. Both approaches have been applied in the present invention.

The polypeptides of the invention comprise a domain termed GH114 as defined in CAZY (GH114, Glycoside Hydrolase Family 114, CAZy database, cazy.org, Lombard et al., 2014, Nucleic Acids Res. 42: D490-D495). The polypeptides of the present invention comprise the GH114 domain and several motifs. One example is [VLI]XE[EDSQ]C (SEQ ID NO: 60) situated in positions 182 to 186 in Amycolatopsis circi (SEQ ID NO: 3). Another motif which may be comprised by the polypeptides of the invention is CY[FLIV][SDN][ATVG] (SEQ ID NO: 61) situated in positions corresponding to positions 55 to 59 in Amycolatopsis circi (SEQ ID NO: 3). The polypeptides in GH114 can be separated into distinct sub-clusters, where we denoted one sub-cluster comprising the motif [VLI]XE[EDSQ]C (SEQ ID NO: 60) as clade or family VAE. Another motif characteristic of this clade is CY[FLIV][SDN][ATVG] (SEQ ID NO: 61).

One embodiment of the invention relates a glycosyl hydrolase, preferably a GH114 glycosyl hydrolase polypeptide, preferably comprising the motif [VLI]XE[EDSQ]C (SEQ ID NO: 60) and/or the motif CY[FLIV][SDN][ATVG] (SEQ ID NO: 61), wherein the polypeptide has hydrolytic activity, and wherein the polypeptide is selected from the group consisting of:
(a) a polypeptide having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54; and
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57.

One preferred embodiment relates to a GH114 glycosyl hydrolase comprising the motif [VLI]XE[EDSQ]C (SEQ ID NO: 60) and/or the motif CY[FLIV][SDN][ATVG] (SEQ ID NO: 61), wherein the GH114 glycosyl hydrolase is selected from the group consisting of:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(h) a polypeptide having, at least 50% at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(i) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(j) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 36;
(k) a polypeptide having at least 99% or 100% sequence identity to the polypeptide shown in SEQ ID NO: 39;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(m) a polypeptide having at least 85%, at least 90%, at least 95%, at least 96%, at least 97%, at least 98%, at least 99% or 100% sequence identity to the polypeptide shown in SEQ ID NO: 45;
(n) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(o) a polypeptide having at least 98%, at least 99% or 100% sequence identity to the polypeptide shown in SEQ ID NO: 51;
(p) a polypeptide having at least 80%, at least 85%, at least 90%, at least 95%, at least 96%, at least 97%, at least 98%, at least 99% or 100% sequence identity to the polypeptide shown in SEQ ID NO: 54; and
(q) a polypeptide having at least 85%, at least 90%, at least 95%, at least 96%, at least 97%, at least 98%, at least 99% or 100% sequence identity to the polypeptide shown in SEQ ID NO: 57.

The VAE clade has been identified as a specific group of GH114 glycosyl hydrolases, which has particularly high substrate activity and which are particularly active, e.g., in deep cleaning, malodor, biofilm, EPS or pel reduction or removal compared to other GH114 glycosyl hydrolases. Preferably the VAE clade GH114 glycosyl hydrolases have superior wash performance compared to other GH114 glycosyl hydrolases not comprised in the clade, i.e., not have the two motifs set forth in SEQ ID NO: 60 and SEQ ID NO: 61. The clade comprises the motif [VLI]XE[EDSQ]C (SEQ ID NO: 60), where the glutamic acid residue (E) at position 184 in SEQ ID NO: 3 is fully conserved in the clade, and one of the two catalytic residues. The clade also comprises motif CY[FLIV][SDN][ATVG] (SEQ ID NO: 61), where the tyrosine (Y) at position 56 in SEQ ID NO: 3 is fully conserved in the clade and involved in substrate binding. The VAE-clade GH114 glycosyl hydrolases have wash performance, i.e., capable of removing, e.g., pel stains under wash conditions, e.g., in a laundry detergent. The VAE-clade GH114 glycosyl hydrolases have wash performance and are stable in cleaning compositions, e.g., in a laundry detergent.

One embodiment of the invention relates a glycosyl hydrolase, preferably a GH114 glycosyl hydrolase, preferably comprising the motif [VLI]XE[EDSQ]C (SEQ ID NO: 60) and/or the motif CY[FLIV][SDN][ATVG] (SEQ ID NO: 61), wherein the GH114 glycosyl hydrolase has hydrolytic activity, and wherein the GH114 glycosyl hydrolase comprises an amino acid sequence selected from the group consisting of amino acid sequences having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146.

One embodiment of the invention relates a glycosyl hydrolase, preferably a GH114 glycosyl hydrolase, preferably comprising the motif [VLI]XE[EDSQ]C (SEQ ID NO: 60) and/or the motif CY[FLIV][SDN][ATVG] (SEQ ID NO: 61), wherein the GH114 glycosyl hydrolase has hydrolytic activity, and wherein the GH114 glycosyl hydrolase comprises an amino acid sequence selected from the group consisting of amino acid sequences having at least 70% sequence identity to the polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146.

One embodiment of the invention relates a glycosyl hydrolase, preferably a GH114 glycosyl hydrolase, preferably comprising the motif [VLI]XE[EDSQ]C (SEQ ID NO: 60) and/or the motif CY[FLIV][SDN][ATVG] (SEQ ID NO: 61), wherein the GH114 glycosyl hydrolase has hydrolytic activity, and wherein the GH114 glycosyl hydrolase comprises an amino acid sequence selected from the group consisting of amino acid sequences having at least 80% sequence identity to the polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146.

One embodiment of the invention relates a glycosyl hydrolase, preferably a GH114 glycosyl hydrolase, preferably comprising the motif [VLI]XE[EDSQ]C (SEQ ID NO: 60) and/or the motif CY[FLIV][SDN][ATVG] (SEQ ID NO: 61), wherein the GH114 glycosyl hydrolase has hydrolytic activity, and wherein the GH114 glycosyl hydrolase comprises an amino acid sequence selected from the group consisting of amino acid sequences having at least 90% sequence identity to the polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146.

The polypeptides of the invention preferably belong to the cluster VAE, which comprises the glycosyl hydrolytic domain GH114 and have hydrolytic activity.

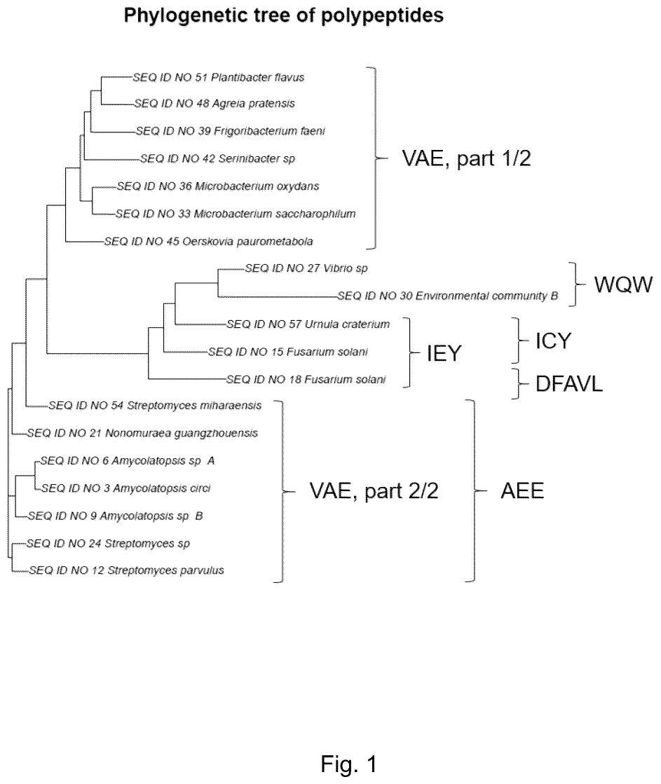

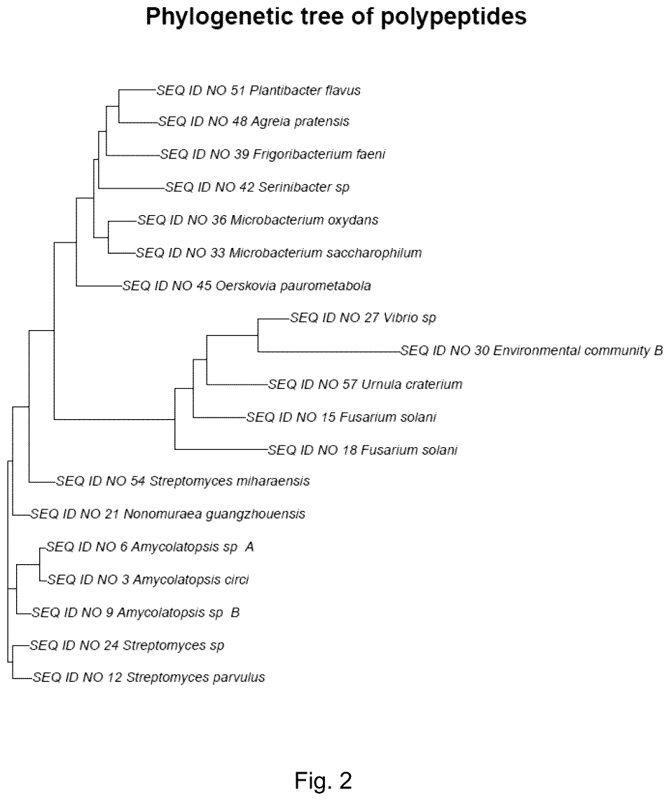

The polypeptides of the VAE clade includes all GH114 glycosyl hydrolases comprising the amino acids sequence shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146. The VAE-clade (or polypeptides of the VAE-clade) may be divided in further clades or sub-clades (see FIG. 1 and FIG. 4), which again may be divided into further sub-clades, see below.

These sub-clades are termed DYQ, AEE, WOW, IEY, ICY and DFAVL clades and comprises polypeptides having hydrolase activity, wherein the polypeptides comprise a GH114 domain and may belong to the VAE clade.

The polypeptides of the DYQ clade comprise the motif DYQ[LI]G (SEQ ID NO: 62), corresponding to amino acids DYQIG at positions 23 to 27 in SEQ ID NO: 3, where D at position 23 is fully conserved in the polypeptides of this clade. An additional motif of the DYQ clade is FQ[TAV]Q[PSD] (SEQ ID NO: 63), corresponding to amino acid 60 to 64 in the reference polypeptide (SEQ ID NO: 3). Examples of polypeptides of the DYQ clade includes SEQ ID NO: 3, SEQ ID NO: 6, SEQ ID NO: 9, SEQ ID NO: 12, SEQ ID NO: 21, SEQ ID NO: 24, SEQ ID NO:

39, SEQ ID NO: 42, SEQ ID NO: 45, SEQ ID NO: 48, SEQ ID NO: 51, and SEQ ID NO: 54 as well as the homologue sequences SEQ ID NO: 79, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 90, SEQ ID NO: 91, SEQ ID NO: 92, SEQ ID NO: 93, SEQ ID NO: 94, SEQ ID NO: 96, SEQ ID NO: 131, SEQ ID NO: 132, SEQ ID NO: 134, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144 and SEQ ID NO: 145.

One embodiment of the invention relates a glycosyl hydrolase preferably a GH114 glycosyl hydrolase comprising the motif(s) DYQ[LI]G (SEQ ID NO: 62) and/or FQ[TAV]Q[PSD] (SEQ ID NO: 63), wherein the glycosyl hydrolase is selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 39;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 42;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 45;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 48;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 51; and
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54.

One preferred embodiment relates to a glycosyl hydrolase, preferably a GH114 glycosyl hydrolase comprising the motif(s) DYQ[LI]G (SEQ ID NO: 62) and/or FQ[TAV]Q[PSD] (SEQ ID NO: 63), wherein the glycosyl hydrolase is selected from the group consisting:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 99% or 100% sequence identity to the polypeptide shown in SEQ ID NO: 39;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(i) a polypeptide having at least 85%, at least 90%, at least 95%, at least 96%, at least 97%, at least 98%, at least 99% or 100% sequence identity to the polypeptide shown in SEQ ID NO: 45;
(j) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(k) a polypeptide having at least 98%, at least 99% or 100% sequence identity to the polypeptide shown in SEQ ID NO: 51; and
(l) a polypeptide having at least 80%, at least 85%, at least 90%, at least 95%, at least 96%, at least 97%, at least 98%, at least 99% or 100% sequence identity to the polypeptide shown in SEQ ID NO: 54.

One embodiment of the invention relates a glycosyl hydrolase preferably a GH114 glycosyl hydrolase comprising the motif(s) DYQ[LI]G (SEQ ID NO: 62) and/or FQ[TAV]Q[PSD] (SEQ ID NO: 63), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 79, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 90, SEQ ID NO: 91, SEQ ID NO: 92, SEQ ID NO: 93, SEQ ID NO: 94, SEQ ID NO: 96, SEQ ID NO: 131, SEQ ID NO: 132, SEQ ID NO: 134, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144 and SEQ ID NO: 145. The glycine G in motif DYQ[LI]G (SEQ ID NO: 62) is involved in substrate binding, and fully conserved in the clade.

One embodiment of the invention relates a glycosyl hydrolase preferably a GH114 glycosyl hydrolase comprising the motif(s) DYQ[LI]G (SEQ ID NO: 62) and/or FQ[TAV]Q[PSD] (SEQ ID NO: 63), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3, SEQ ID NO: 6, SEQ ID NO: 9, SEQ ID NO: 12, SEQ ID NO: 21, SEQ ID NO: 24, SEQ ID NO: 39, SEQ ID NO: 42, SEQ ID NO: 45, SEQ ID NO: 48, SEQ ID NO: 51, SEQ ID NO: 54, SEQ ID NO: 79, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 90, SEQ ID NO: 91, SEQ ID NO: 92, SEQ ID NO: 93, SEQ ID NO: 94, SEQ ID NO: 96, SEQ ID NO: 131, SEQ ID NO: 132, SEQ ID NO: 134, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144 and SEQ ID NO: 145.

The DYQ sub-clade may be further be divided into yet another subgroup, which here is termed AEE clade (se FIG. 1 or 4). The AEE clade comprises polypeptides of bacterial origin having hydrolase activity, wherein the polypeptides comprise a GH114 domain and belong to the VAE part 2 clade (and the DYQ-clade). The polypeptides of the clade comprise the motif example AEECG (SEQ ID NO: 64), corresponding to amino acids AEECG at positions 183 to 187 of SEQ ID NO: 3 where all amino acids are fully conserved in AEE clade. An additional motif of the AEE clade is NAFQ[AT]Q (SEQ ID NO: 65), corresponding to amino acids 58 to 63 in the reference polypeptide (SEQ ID NO: 3). Examples of polypeptides of the AEE clade includes SEQ ID NO: 3, SEQ ID NO: 6, SEQ ID NO: 9, SEQ ID NO: 12, SEQ ID NO: 21, SEQ ID NO: 24, and SEQ ID NO: 54 as well as the homologue sequences SEQ ID NO: 79, SEQ ID NO: 82, SEQ ID NO: 83, SEQ ID NO: 84, SEQ ID NO: 85, SEQ ID NO: 132, SEQ ID NO: 136, SEQ ID NO: 138 and SEQ ID NO: 142.

One embodiment of the invention relates a glycosyl hydrolase, preferably a GH114 glycosyl hydrolase comprising one, two, three or all four motif(s) DYQ[LI]G (SEQ ID NO: 62), FQ[TAV]Q[PSD] (SEQ ID NO: 63), AEECG (SEQ ID NO: 64) and/or NAFQ[AT]Q (SEQ ID NO: 65), wherein the glycosyl hydrolase is selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 21;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 24; and
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 54.

One preferred embodiment of the invention relates a GH114 glycosyl hydrolase, comprising one, two, three or all four motif(s) DYQ[LI]G (SEQ ID NO: 62), FQ[TAV]Q[PSD] (SEQ ID NO: 63), AEECG (SEQ ID NO: 64) and/or NAFQ[AT]Q (SEQ ID NO: 65), wherein the glycosyl hydrolase is selected from the group consisting of:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 80%, at least 85%, at least 90%, at least 95%, at least 96%, at least 97%, at least 98%, at least 99% or 100% sequence identity to the polypeptide shown in SEQ ID NO: 54.

One embodiment of the invention relates a glycosyl hydrolase, preferably a GH114 glycosyl hydrolase comprising one, to, three or all four motif(s) DYQ[LI]G (SEQ ID NO: 62), FQ[TAV]Q[PSD] (SEQ ID NO: 63), AEECG (SEQ ID NO: 64) and/or NAFQ[AT]Q (SEQ ID NO: 65), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 79, SEQ ID NO: 82, SEQ ID NO: 83, SEQ ID NO: 84, SEQ ID NO: 85, SEQ ID NO: 132, SEQ ID NO: 136, SEQ ID NO: 138, and SEQ ID NO: 142.

One embodiment of the invention relates a glycosyl hydrolase, preferably a GH114 glycosyl hydrolase comprising one, to, three or all four motif(s) DYQ[LI]G (SEQ ID NO: 62), FQ[TAV]Q[PSD] (SEQ ID NO: 63), AEECG (SEQ ID NO: 64) and/or NAFQ[AT]Q (SEQ ID NO: 65), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 3, SEQ ID NO: 6, SEQ ID NO: 9, SEQ ID NO: 12, SEQ ID NO: 21, SEQ ID NO: 24, SEQ ID NO: 54, SEQ ID NO: 79, SEQ ID NO: 82, SEQ ID NO: 83, SEQ ID NO: 84, SEQ ID NO: 85, SEQ ID NO: 132, SEQ ID NO: 136, SEQ ID NO: 138, and SEQ ID NO: 142.

Another sub-clade of the VAE clade is the IEY clade. The IEY clade comprises polypeptides of fungal origin, containing a GH114 domain and belong to the VAE clade. The polypeptides of the clade comprise the motif GXXVX[NHQTS]IEY[PG] (SEQ ID NO: 68), corresponding to amino acids “GKPVLQIEYP”, positions 230 to 239 of SEQ ID NO: 18 where E (corresponding to position 237 of SEQ ID NO: 3) is fully conserved in IEY clade, part of the substrate binding pocket, and one of the two putative catalytic site residues. An additional motif of the IEY clade is VICYF (SEQ ID NO: 69), corresponding to amino acids VICYF positions 68 to 72 of (SEQ ID NO: 18), 27 where CYF (corresponding to positions 70 and 72 of SEQ ID NO: 18) is fully conserved in IEY clade.

Examples of polypeptides of the IEY clade includes SEQ ID NO: 15, SEQ ID NO: 18, and, SEQ ID NO: 57 as well as the homologue sequences SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 133 and SEQ ID NO: 135.

One embodiment of the invention relates a glycosyl hydrolase preferably a GH114 glycosyl hydrolase comprising the motif GXXVX[NHQTS]IEY[PG] (SEQ ID NO: 68) and/or the motif VICYF (SEQ ID NO: 69), wherein the glycosyl hydrolase is selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18; and
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57.

One preferred embodiment of the invention relates a GH114 glycosyl hydrolase comprising the motif GXXVX[NHQTS]IEY[PG] (SEQ ID NO: 68) and/or the motif VICYF (SEQ ID NO: 69), wherein the glycosyl hydrolase is a polypeptide having at least 85%, at least 90%, at least 95%, at least 96%, at least 97%, at least 98%, at least 99% or 100% sequence identity to the polypeptide shown in SEQ ID NO: 57.

One embodiment of the invention relates a glycosyl hydrolase preferably a GH114 glycosyl hydrolase comprising the motif GXXVX[NHQTS]IEY[PG] (SEQ ID NO: 68) and/or the motif VICYF (SEQ ID NO: 69), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 133 and SEQ ID NO: 135.

One embodiment of the invention relates a glycosyl hydrolase preferably a GH114 glycosyl hydrolase comprising the motif GXXVX[NHQTS]IEY[PG] (SEQ ID NO: 68) and/or the motif VICYF (SEQ ID NO: 69), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15, SEQ ID NO: 18, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 133 and SEQ ID NO: 135.

The IEY sub-clade may be further be divided into yet another subgroup, which here is termed ICY clade (se FIG. 1 and FIG. 4). The ICY clade comprises polypeptides of fungal origin having hydrolase activity, wherein the polypeptides comprise a GH114 domain and belong to the IEY clade (and the VAE-clade). The polypeptides of the clade comprise the motif example ICYFSA (SEQ ID NO: 70), corresponding to amino acids 81 to 86 in SEQ ID NO: 15. Examples of polypeptides of the ICY clade is SEQ ID NO: 15, and SEQ ID NO: 57 as well as the homologue sequences SEQ ID NO: 98, SEQ ID NO: 101, SEQ ID NO: 104, SEQ ID NO: 107, SEQ ID NO: 114, SEQ ID NO: 115, SEQ ID NO: 120, SEQ ID NO: 124, SEQ ID NO: 125, SEQ ID NO: 127, SEQ ID NO: 129 and SEQ ID NO: 133.

One embodiment of the invention relates a glycosyl hydrolase preferably a GH114 glycosyl hydrolase comprising one, two or all three motif(s) GXXVX[NHQTS]IEY[PG] (SEQ ID NO: 68), VICYF (SEQ ID NO: 69) and/or ICYFSA (SEQ ID NO: 70), wherein the glycosyl hydrolase is selected from the group consisting of:
(a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15; and
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 57.

One preferred embodiment of the invention relates a GH114 glycosyl hydrolase comprising one, two or all three motif(s) GXXVX[NHQTS]IEY[PG] (SEQ ID NO: 68), VICYF (SEQ ID NO: 69) and/or ICYFSA (SEQ ID NO: 70), wherein the glycosyl hydrolase is a polypeptide having at least 85%, at least 90%, at least 95%, at least 96%, at least 97%, at least 98%, at least 99% or 100% sequence identity to the polypeptide shown in SEQ ID NO: 57.

One embodiment of the invention relates a glycosyl hydrolase preferably a GH114 glycosyl hydrolase comprising one, two or all three motif(s) GXXVX[NHQTS]IEY[PG] (SEQ ID NO: 68), VICYF (SEQ ID NO: 69) and/or ICYFSA (SEQ ID NO: 70), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 98, SEQ ID NO: 101, SEQ ID NO: 104, SEQ ID NO: 107, SEQ ID NO: 114, SEQ ID NO: 115, SEQ ID NO: 120, SEQ ID NO: 124, SEQ ID NO: 125, SEQ ID NO: 127, SEQ ID NO: 129 and SEQ ID NO: 133.

One embodiment of the invention relates a glycosyl hydrolase preferably a GH114 glycosyl hydrolase comprising one, two or all three motif(s) GXXVX[NHQTS]IEY[PG] (SEQ ID NO: 68), VICYF (SEQ ID NO: 69) and/or ICYFSA (SEQ ID NO: 70), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 15, SEQ ID NO: 57, SEQ ID NO: 98, SEQ ID NO: 101, SEQ ID NO: 104, SEQ ID NO: 107, SEQ ID NO: 114, SEQ ID NO: 115, SEQ ID NO: 120, SEQ ID NO: 124, SEQ ID NO: 125, SEQ ID NO: 127, SEQ ID NO: 129 and SEQ ID NO: 133.

The IEY sub-clade may be further be divided into yet another subgroup, which here is termed DFAVL clade (see FIG. 1). The DFAVL clade comprises polypeptides of fungal origin having hydrolase activity, wherein the polypeptides comprise a GH114 domain and belong to the IEY clade (and the VAE-clade). The Leucine L in the motif is fully conserved in the clade, and the adjacent glutamic acid E is one of the two catalytic active site residues. The polypeptides of the clade comprise the motif DFAVL (SEQ ID NO: 71), corresponding to amino acids DFAVL at positions 199 to 203 of SEQ ID NO: 18.

An example of a polypeptide of the DFAVL clade is SEQ ID NO: 18 as well as the homologue sequences SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 110, SEQ ID NO: 111, SEQ ID NO: 112, SEQ ID NO: 113, SEQ ID NO: 116, SEQ ID NO: 117, SEQ ID NO: 118, SEQ ID NO: 119, SEQ ID NO: 121, SEQ ID NO: 123, SEQ ID NO: 126, SEQ ID NO: 128, SEQ ID NO: 130 and SEQ ID NO: 135.

One embodiment of the invention relates a glycosyl hydrolase preferably a GH114 glycosyl hydrolase comprising one, two or all three motif(s) GXXVX[NHQTS]IEY[PG] (SEQ ID NO: 68), VICYF (SEQ ID NO: 69) and/or DFAVL (SEQ ID NO: 71), wherein the glycosyl hydrolase is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18.

One embodiment of the invention relates a glycosyl hydrolase preferably a GH114 glycosyl hydrolase comprising one, two or all three motif(s) GXXVX[NHQTS]IEY[PG] (SEQ ID NO: 68), VICYF (SEQ ID NO: 69) and/or DFAVL (SEQ ID NO: 71), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 110, SEQ ID NO: 111, SEQ ID NO: 112, SEQ ID NO: 113, SEQ ID NO: 116, SEQ ID NO: 117, SEQ ID NO: 118, SEQ ID NO: 119, SEQ ID NO: 121, SEQ ID NO: 123, SEQ ID NO: 126, SEQ ID NO: 128, SEQ ID NO: 130 and SEQ ID NO: 135.

One embodiment of the invention relates a glycosyl hydrolase preferably a GH114 glycosyl hydrolase comprising one, two or all three motif(s) GXXVX[NHQTS]IEY[PG] (SEQ ID NO: 68), VICYF (SEQ ID NO: 69) and/or DFAVL (SEQ ID NO: 71), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 18, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 110, SEQ ID NO: 111, SEQ ID NO: 112, SEQ ID NO: 113, SEQ ID NO: 116, SEQ ID NO: 117, SEQ ID NO: 118, SEQ ID NO: 119, SEQ ID NO: 121, SEQ ID NO: 123, SEQ ID NO: 126, SEQ ID NO: 128, SEQ ID NO: 130 and SEQ ID NO: 135.

Another sub-clade of the VAE clade is the WQW clade. The WOW clade comprises polypeptides of bacterial and fungal origin, containing a GH114 domain and belonging to the VAE clade, having hydrolase activity. The polypeptides of the clade comprise the motif example WQWQL (SEQ ID NO: 66), corresponding to amino acids WQWQL positions 30 to 34 of Vibrio sp. SEQ ID NO: 27 where WQW (corresponding to positions 30 and 32 of SEQ ID NO: 27) is fully conserved in WQW clade. An additional motif of the WQW clade is [VLI][GASD]LKN[DGS][VLIP] (SEQ ID NO: 67), corresponding to amino acids IGLKNDL positions 171 to 177 of Vibrio sp. SEQ ID NO: 27 where LKN (corresponding to positions 173 and 175 of SEQ ID NO: 27) is fully conserved in WQW clade. An example of a polypeptide of the WOW clade includes the polypeptide shown in SEQ ID NO: 27. Another example of a polypeptide of the WQW clade is includes the polypeptide shown in SEQ ID NO: 33 and SEQ ID NO: 36 as well as the homologue sequences SEQ ID NO: 80, SEQ ID NO: 81, SEQ ID NO: 89, SEQ ID NO: 95 and SEQ ID NO: 146.

One embodiment of the invention relates a glycosyl hydrolase preferably a GH114 glycosyl hydrolase comprising the motif WQWQL (SEQ ID NO: 66) and/or the motif [VLI][GASD]LKN[DGS][VLIP] (SEQ ID NO: 67), wherein the glycosyl hydrolase is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 27.

One embodiment of the invention relates a glycosyl hydrolase preferably a GH114 glycosyl hydrolase comprising the motif WQWQL (SEQ ID NO: 66) and/or the motif [VLI][GASD]LKN[DGS][VLIP] (SEQ ID NO: 67), wherein the glycosyl hydrolase is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 33.

One embodiment of the invention relates a glycosyl hydrolase preferably a GH114 glycosyl hydrolase comprising the motif WQWQL (SEQ ID NO: 66) and/or the motif [VLI][GASD]LKN[DGS][VLIP] (SEQ ID NO: 67), wherein the glycosyl hydrolase is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 36.

One embodiment of the invention relates a glycosyl hydrolase preferably a GH114 glycosyl hydrolase comprising the motif WQWQL (SEQ ID NO: 66) and/or the motif [VLI][GASD]LKN[DGS][VLIP] (SEQ ID NO: 67), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 80, SEQ ID NO: 81, SEQ ID NO: 89, SEQ ID NO: 95 and SEQ ID NO: 146.

One embodiment of the invention relates a glycosyl hydrolase preferably a GH114 glycosyl hydrolase comprising the motif WQWQL (SEQ ID NO: 66) and/or the motif [VLI][GASD]LKN[DGS][VLIP] (SEQ ID NO: 67), wherein the glycosyl hydrolase is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide of SEQ ID NO: 27, SEQ ID NO: 33, SEQ ID NO: 36, SEQ ID NO: 80, SEQ ID NO: 81, SEQ ID NO: 89, SEQ ID NO: 95 and SEQ ID NO: 146.

One embodiment of the invention relates to a GH114 glycosyl hydrolase, having alpha-1,4-polygalactosaminidase activity, wherein the polypeptides comprises one or more of the motif(s) selected from the group consisting of: [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70) and DFAVL (SEQ ID NO: 71).

The polypeptides of the invention have activity to the exopolysaccharide alpha-1,4-polygalactosamine and preferably to pel, which is a polysaccharide component of, e.g., some biofilm matrix. One embodiment of the invention relates to the use of a GH114 glycosyl hydrolase according to the invention for reduction or removal of pel or alpha-1,4-polygalactosamine stain, e.g., wherein in the pel or alpha-1,4-polygalactosamine is comprised in a biofilm or biofilm EPS (extracellular polymeric substances). One embodiment of the invention relates to the use of a GH114 glycosyl hydrolase according to the invention for reduction or removal of biofilm. In particular, the GH114 glycosyl hydrolase polypeptides of the invention have activity in cleaning compositions such as laundry or dish wash detergents and is useful in cleaning processes such as laundry and/or dish wash, e.g., for cleaning organic stains such as dead cell material, skin debris, sebum, sweat, grease and other stains derived from, e.g., humans (body soils) or microbes but also from the environment, from surfaces such as textiles and hard surfaces. In particular, the GH114 glycosyl hydrolase polypeptides of the invention have activity in cleaning compositions such as laundry or dish wash detergents and is useful in cleaning processes such as laundry and/or dish wash, e.g., for deep cleaning of surfaces such as textiles and hard surfaces. The present invention also provides a method for preventing, reduction or removal of pel or alpha-1,4-polygalactosamine containing stains from an item comprising applying at least one GH114 glycosyl hydrolase polypeptide of the invention, preferably having alpha-1,4-polygalactosaminidase activity, to an item and optionally rinse the item. The item is preferably a textile or a hard surface, e.g., a non-medical hard surface such as dish ware. The present disclosure also provides a method for reduction or removal of dead cell material, skin debris, sebum, sweat and grease stains from an item comprising applying at least one GH114 glycosyl hydrolase polypeptide of the invention to an item and optionally rinse the item. The item is preferably a textile or a hard surface, e.g., a non-medical hard surface such as dish ware.

Organic matters such as biofilm EPS, cell debris and body soil or components hereof may have glue-like properties and the presence of biofilm on, e.g., textiles and may result in items or areas on items which are “sticky”. Soil will in general adhere to the sticky areas and such soil has shown difficult to remove by commercially available detergent compositions. Further, when dirty laundry items are washed together with less dirty laundry items the dirt present in the wash liquor tend to stick to the organic matter and, e.g., EPS. As a result, the laundry item is more “soiled” after wash than before wash. This effect may also be termed re-deposition. One embodiment of the invention relates to the use of a GH114 glycosyl hydrolase polypeptides comprising one or more of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) for reducing or removing re-deposition.

One embodiment of the invention relates to the use of a GH114 glycosyl hydrolase polypeptides comprising one or more of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) for reducing or removing malodor of items, e.g., being washed. The inventors have surprisingly found that the polypeptides comprising one or more of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) as defined above are useful in reducing or removing laundry associated alpha-1,4-polygalactosamine. One embodiment of the invention relates to the use of a GH114 glycosyl hydrolase, having alpha-1,4-polygalactosaminidase activity, wherein the polypeptides comprises one or more of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) for reducing or removing malodor of an item, e.g., a textile.

The polypeptides of the present invention are useful in cleaning compositions and are effective in deep cleaning of surfaces such as fabrics. The polypeptides of the present invention are effective in reducing or removing polysaccharide soiling, e.g., polylactosamine from, e.g., organic matter. One example of organic matter is biofilm, which is produced by various microorganisms. The extracellular polymeric matrix of biofilm, EPS is composed of polysaccharides, such as polylactosamine, e.g., pel, extracellular DNA and proteins. Biofilm EPS may be sticky or gluing, which when present on textile, may give rise to re-deposition or back staining of soil resulting in a greying of the textile. Another drawback of organic matter, e.g., biofilm is the malodor as various malodor related molecules are often associated with organic matter, e.g., biofilm. One aspect of the invention relates to a method for laundering an item comprising the steps of:
a. exposing an item to a wash liquor comprising a polypeptide or a cleaning composition comprising a polypeptide selected from the group consisting of polypeptides comprising the sequences shown in: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto, wherein the polypeptide has hydrolytic activity;
b. completing at least one wash cycle; and
c. optionally rinsing the item,
   wherein the item is a textile.

The polypeptides of the invention are therefore useful for prevention, reduction or removal of malodor and for prevention, reduction of re-deposition and improving whiteness.

One embodiment of the invention relates to the use of polypeptide selected from the group consisting of polypeptides comprising the sequences shown in: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto for deep cleaning of an item, wherein the item is a textile.

One embodiment of the invention relates to the use of polypeptide selected from the group consisting of polypeptides comprising the sequences shown in: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto for reduction or removal of dead cell material, skin debris, sebum, sweat or grease stains from an item, wherein the item is a textile.

One embodiment of the invention relates to the use of polypeptide selected from the group consisting of polypeptides comprising the sequences shown in: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54 SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto;
(i) for preventing, reducing or removing stickiness of the item;
(ii) for pretreating stains on the item;
(iii) for preventing, reducing or removing redeposition of soil during a wash cycle;
(iv) for preventing, reducing or removing adherence of soil to the item;
(v) for maintaining or improving whiteness of the item;
(vi) for preventing, reducing or removal malodor from the item,
   wherein the item is a textile. The textile may, e.g., be cotton or polyester or a mixture hereof.

Further methods and uses are described in the “use” section below.

One embodiment of the invention relates to a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54 SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146.

One embodiment of the invention relates a GH114 glycosyl hydrolase polypeptide, wherein the polypeptide has hydrolytic activity, preferably alpha-1,4-polygalactosaminidase activity and wherein the polypeptide is selected from the group consisting of:
(a) a polypeptide having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 55;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54;
(t) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57;
(u) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 72,
(v) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 73,
(w) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 74,
(x) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 75,
(y) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 76,
(z) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 77,
(aa) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 78,
(bb) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 79,
(cc) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 80,
(dd) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 81,
(ee) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 82,
(ff) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 83,
(gg) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 84,
(hh) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 85,
(ii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 86,
(jj) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 87,
(kk) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 88,
(ll) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 89, a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 90,
(mm) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 91, a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 92,
(nn) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 93,
(oo) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 94,
(pp) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 95,
(qq) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 96,
(rr) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 97,
(ss) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 98,
(tt) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 99,
(uu) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 100,
(vv) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 101,
(ww) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 102,
(xx) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 103,
(yy) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 104,
(zz) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 105,
(aaa) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 106,
(bbb) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 107,
(ccc) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 108,
(ddd) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 109,
(eee) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 110,
(fff) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 111,
(ggg) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 112,
(hhh) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 113,
(iii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 114,
(jjj) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 115,
(kkk) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 116,
(lll) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 117,
(mmm) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 118,
(nnn) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 119,
(ooo) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 120,
(ppp) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 121,
(qqq) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 122,
(rrr) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 123,
(sss) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 124,
(ttt) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 125,
(uuu) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 126,
(vvv) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 127,
(www) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 128,
(xxx) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 129,
(yyy) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 130,
(zzz) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 131,
(aaaa) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 132,
(bbbb) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 133,
(cccc) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 134,
(dddd) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 135,
(eeee) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 136,
(ffff) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 137,
(gggg) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 138,
(hhhh) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 139,
(iiii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 140,
(jjjj) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 141,
(kkkk) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 142,
(llll) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 143,
(mmmm) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 144, a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 145, and
(nnnn) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 146, wherein the polypeptide has glycosyl hydrolase activity, preferably alpha-1,4-polygalactosaminidase activity.

One embodiment of the invention relates a GH114 glycosyl hydrolase polypeptide, wherein the polypeptide has hydrolytic activity, preferably alpha-1,4-polygalactosaminidase activity, and wherein the polypeptide is selected from the group consisting of: a polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146 or a polypeptide having at least 60%, at least 70%, at least 75%, at least 80%, at least 85%, at least 90%, at least 95%, at least 96%, at least 97%, at least 98%, at least 99% or 100% sequence identity thereto.

One preferred embodiment relates to a GH114 glycosyl hydrolase polypeptide, wherein the polypeptide has hydrolytic activity, preferably alpha-1,4-polygalactosaminidase activity, and wherein the polypeptide is selected from the group consisting of:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(h) a polypeptide having, at least 50% at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(i) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(j) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 36;
(k) a polypeptide having at least 99% or 100% sequence identity to the polypeptide shown in SEQ ID NO: 39;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(m) a polypeptide having at least 85%, at least 90%, at least 95%, at least 96%, at least 97%, at least 98%, at least 99% or 100% sequence identity to the polypeptide shown in SEQ ID NO: 45;
(n) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(o) a polypeptide having at least 98%, at least 99% or 100% sequence identity to the polypeptide shown in SEQ ID NO: 51;
(p) a polypeptide having at least 80%, at least 85%, at least 90%, at least 95%, at least 96%, at least 97%, at least 98%, at least 99% or 100% sequence identity to the polypeptide shown in SEQ ID NO: 54; and
(q) a polypeptide having at least 85%, at least 90%, at least 95%, at least 96%, at least 97%, at least 98%, at least 99% or 100% sequence identity to the polypeptide shown in SEQ ID NO: 57.

Another preferred embodiment of the invention relates a GH114 glycosyl hydrolase polypeptide comprising one, two, three, four or five of the motifs [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71), wherein the polypeptide has hydrolytic activity, preferably alpha-1,4-polygalactosaminidase activity, and wherein the polypeptide is selected from the group consisting of:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(h) a polypeptide having, at least 50% at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(i) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(j) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 36;
(k) a polypeptide having at least 99% or 100% sequence identity to the polypeptide shown in SEQ ID NO: 39;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(m) a polypeptide having at least 85%, at least 90%, at least 95%, at least 96%, at least 97%, at least 98%, at least 99% or 100% sequence identity to the polypeptide shown in SEQ ID NO: 45;
(n) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(o) a polypeptide having at least 98%, at least 99% or 100% sequence identity to the polypeptide shown in SEQ ID NO: 51;
(p) a polypeptide having at least 80%, at least 85%, at least 90%, at least 95%, at least 96%, at least 97%, at least 98%, at least 99% or 100% sequence identity to the polypeptide shown in SEQ ID NO: 54; and
(q) a polypeptide having at least 85%, at least 90%, at least 95%, at least 96%, at least 97%, at least 98%, at least 99% or 100% sequence identity to the polypeptide shown in SEQ ID NO: 57.

Another preferred embodiment of the invention relates a GH114 glycosyl hydrolase polypeptide comprising one, two, three, four or five of the motifs [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71), wherein the polypeptide has hydrolytic activity, preferably alpha-1,4-polygalactosaminidase activity, and wherein the polypeptide is selected from the group consisting of: a polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146 or a polypeptide having at least 60%, at least 70%, at least 75%, at least 80%, at least 85%, at least 90%, at least 95%, at least 96%, at least 97%, at least 98%, at least 99% or 100% sequence identity thereto.

In one embodiment, the present invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 2.

In a particular embodiment, the invention relates to polypeptides having a sequence identity to the mature polypeptide of SEQ ID NO: 5 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 5.

In a particular embodiment, the invention relates to polypeptides having a sequence identity to the mature polypeptide of SEQ ID NO: 8 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 8.

In a particular embodiment, the invention relates to polypeptides having a sequence identity to the mature polypeptide of SEQ ID NO: 11 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 11.

In a particular embodiment, the invention relates to polypeptides having a sequence identity to the mature polypeptide of SEQ ID NO: 14 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 14.

In some embodiment, the present invention relates to polypeptides having a sequence identity to the mature polypeptide of SEQ ID NO: 17 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 7.

In a particular embodiment, the invention relates to polypeptides having a sequence identity to the mature polypeptide of SEQ ID NO: 20 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 20.

In a particular embodiment, the invention relates to polypeptides having a sequence identity to the mature polypeptide of SEQ ID NO: 23 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 23.

In a particular embodiment, the invention relates to polypeptides having a sequence identity to the mature polypeptide of SEQ ID NO: 26 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 26.

In a particular embodiment, the invention relates to polypeptides having a sequence identity to the mature polypeptide of SEQ ID NO: 29 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 29.

In a particular embodiment, the invention relates to polypeptides having a sequence identity to the mature polypeptide of SEQ ID NO: 32 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 32.

In a particular embodiment, the invention relates to polypeptides having a sequence identity to the mature polypeptide of SEQ ID NO: 35 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 35.

In a particular embodiment, the invention relates to polypeptides having a sequence identity to the mature polypeptide of SEQ ID NO: 38 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 38.

In a particular embodiment, the invention relates to polypeptides having a sequence identity to the mature polypeptide of SEQ ID NO: 41 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 41.

In a particular embodiment, the invention relates to polypeptides having a sequence identity to the mature polypeptide of SEQ ID NO: 44 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 44.

In a particular embodiment, the invention relates to polypeptides having a sequence identity to the mature polypeptide of SEQ ID NO: 47 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 47.

In a particular embodiment, the invention relates to polypeptides having a sequence identity to the mature polypeptide of SEQ ID NO: 50 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 50.

In a particular embodiment, the invention relates to polypeptides having a sequence identity to the mature polypeptide of SEQ ID NO: 53 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 53.

In a particular embodiment, the invention relates to polypeptides having a sequence identity to the mature polypeptide of SEQ ID NO: 56 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 56.

In some embodiment, the polypeptide has been isolated. A polypeptide of the present invention preferably comprises or consists of the amino acid sequence shown in SEQ ID NO: 3 or an allelic variant thereof; or is a fragment thereof having hydrolytic activity. In another aspect, the polypeptide comprises or consists of the mature polypeptide of SEQ ID NO: 2. In another aspect, the polypeptide comprises or consists of amino acids 1 to 246 of SEQ ID NO: 2.

In one embodiment, the polypeptide preferably comprises or consists of the amino acid sequence shown in SEQ ID NO: 3; comprises the amino acid sequence shown in SEQ ID NO: 3 and a N-terminal and/or C-terminal His-tag and/or HQ-tag; comprises the amino acid sequence of SEQ ID NO: 3 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 3.

In some embodiment, the polypeptide has been isolated. A polypeptide of the present invention preferably comprises or consists of the amino acid sequence shown in SEQ ID NO: 6 or an allelic variant thereof; or is a fragment thereof having hydrolytic activity. In another aspect, the polypeptide comprises or consists of the mature polypeptide of SEQ ID NO: 5. In another aspect, the polypeptide comprises or consists of amino acids 1 to 243 of SEQ ID NO: 5.

In one embodiment, the polypeptide preferably comprises or consists of the amino acid sequence shown in SEQ ID NO: 6; comprises the amino acid sequence shown in SEQ ID NO: 6 and a N-terminal and/or C-terminal His-tag and/or HQ-tag; comprises the amino acid sequence of SEQ ID NO: 6 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 6.

In some embodiment, the polypeptide has been isolated. A polypeptide of the present invention preferably comprises or consists of the amino acid sequence shown in SEQ ID NO: 9 or an allelic variant thereof; or is a fragment thereof having hydrolytic activity. In another aspect, the polypeptide comprises or consists of the mature polypeptide of SEQ ID NO: 8. In another aspect, the polypeptide comprises or consists of amino acids 1 to 237 of SEQ ID NO: 8.

In one embodiment, the polypeptide preferably comprises or consists of the amino acid sequence shown in SEQ ID NO: 9; comprises the amino acid sequence shown in SEQ ID NO: 9 and a N-terminal and/or C-terminal His-tag and/or HQ-tag; comprises the amino acid sequence of SEQ ID NO: 9 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 9.

In some embodiment, the polypeptide has been isolated. A polypeptide of the present invention preferably comprises or consists of the amino acid sequence shown in SEQ ID NO: 12 or an allelic variant thereof; or is a fragment thereof having hydrolytic activity. In another aspect, the polypeptide comprises or consists of the mature polypeptide of SEQ ID NO: 11. In another aspect, the polypeptide comprises or consists of amino acids 1 to 237 of SEQ ID NO: 11.

In one embodiment, the polypeptide preferably comprises or consists of the amino acid sequence shown in SEQ ID NO: 12; comprises the amino acid sequence shown in SEQ ID NO: 12 and a N-terminal and/or C-terminal His-tag and/or HQ-tag; comprises the amino acid sequence of SEQ ID NO: 12 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 12.

In some embodiment, the polypeptide has been isolated. A polypeptide of the present invention preferably comprises or consists of the amino acid sequence shown in SEQ ID NO: 15 or an allelic variant thereof; or is a fragment thereof having hydrolytic activity. In another aspect, the polypeptide comprises or consists of the mature polypeptide of SEQ ID NO: 14. In another aspect, the polypeptide comprises or consists of amino acids 1 to 274 of SEQ ID NO: 14.

In one embodiment, the polypeptide preferably comprises or consists of the amino acid sequence shown in SEQ ID NO: 15; comprises the amino acid sequence shown in SEQ ID NO: 15 and a N-terminal and/or C-terminal His-tag and/or HQ-tag; comprises the amino acid sequence of SEQ ID NO: 15 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 15.

In some embodiment, the polypeptide has been isolated. A polypeptide of the present invention preferably comprises or consists of the amino acid sequence shown in SEQ ID NO: 18 or an allelic variant thereof; or is a fragment thereof having hydrolytic activity. In another aspect, the polypeptide comprises or consists of the mature polypeptide of SEQ ID NO: 17. In another aspect, the polypeptide comprises or consists of amino acids 1 to 296 of SEQ ID NO: 17.

In one embodiment, the polypeptide preferably comprises or consists of the amino acid sequence shown in SEQ ID NO: 18; comprises the amino acid sequence shown in SEQ ID NO: 18 and a N-terminal and/or C-terminal His-tag and/or HQ-tag; comprises the amino acid sequence of SEQ ID NO: 18 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 18.

In some embodiment, the polypeptide has been isolated. A polypeptide of the present invention preferably comprises or consists of the amino acid sequence shown in SEQ ID NO: 21 or an allelic variant thereof; or is a fragment thereof having hydrolytic activity. In another aspect, the polypeptide comprises or consists of the mature polypeptide of SEQ ID NO: 20. In another aspect, the polypeptide comprises or consists of amino acids 1 to 237 of SEQ ID NO: 20.

In one embodiment, the polypeptide preferably comprises or consists of the amino acid sequence shown in SEQ ID NO: 21; comprises the amino acid sequence shown in SEQ ID NO: 21 and a N-terminal and/or C-terminal His-tag and/or HQ-tag; comprises the amino acid sequence of SEQ ID NO: 21 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 21.

In some embodiment, the polypeptide has been isolated. A polypeptide of the present invention preferably comprises or consists of the amino acid sequence shown in SEQ ID NO: 24 or an allelic variant thereof; or is a fragment thereof having hydrolytic activity. In another aspect, the polypeptide comprises or consists of the mature polypeptide of SEQ ID NO: 23. In another aspect, the polypeptide comprises or consists of amino acids 1 to 239 of SEQ ID NO: 23.

In one embodiment, the polypeptide preferably comprises or consists of the amino acid sequence shown in SEQ ID NO: 24; comprises the amino acid sequence shown in SEQ ID NO: 24 and a N-terminal and/or C-terminal His-tag and/or HQ-tag; comprises the amino acid sequence of SEQ ID NO: 24 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 24.

In some embodiment, the polypeptide has been isolated. A polypeptide of the present invention preferably comprises or consists of the amino acid sequence shown in SEQ ID NO: 27 or an allelic variant thereof; or is a fragment thereof having hydrolytic activity. In another aspect, the polypeptide comprises or consists of the mature polypeptide of SEQ ID NO: 26. In another aspect, the polypeptide comprises or consists of amino acids 1 to 234 of SEQ ID NO: 26.

In one embodiment, the polypeptide preferably comprises or consists of the amino acid sequence shown in SEQ ID NO: 27; comprises the amino acid sequence shown in SEQ ID NO: 27 and a N-terminal and/or C-terminal His-tag and/or HQ-tag; comprises the amino acid sequence of SEQ ID NO: 27 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 27.

In some embodiment, the polypeptide has been isolated. A polypeptide of the present invention preferably comprises or consists of the amino acid sequence shown in SEQ ID NO: 30 or an allelic variant thereof; or is a fragment thereof having hydrolytic activity. In another aspect, the polypeptide comprises or consists of the mature polypeptide of SEQ ID NO: 29. In another aspect, the polypeptide comprises or consists of amino acids 1 to 242 of SEQ ID NO: 29.

In one embodiment, the polypeptide preferably comprises or consists of the amino acid sequence shown in SEQ ID NO: 30; comprises the amino acid sequence shown in SEQ ID NO: 30 and a N-terminal and/or C-terminal His-tag and/or HQ-tag; comprises the amino acid sequence of SEQ ID NO: 30 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 30.

In some embodiment, the polypeptide has been isolated. A polypeptide of the present invention preferably comprises or consists of the amino acid sequence shown in SEQ ID NO: 33 or an allelic variant thereof; or is a fragment thereof having hydrolytic activity. In another aspect, the polypeptide comprises or consists of the mature polypeptide of SEQ ID NO: 32. In another aspect, the polypeptide comprises or consists of amino acids 1 to 238 of SEQ ID NO: 32.

In one embodiment, the polypeptide preferably comprises or consists of the amino acid sequence shown in SEQ ID NO: 33; comprises the amino acid sequence shown in SEQ ID NO: 33 and a N-terminal and/or C-terminal His-tag and/or HQ-tag; comprises the amino acid sequence of SEQ ID NO: 33 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 33.

In some embodiment, the polypeptide has been isolated. A polypeptide of the present invention preferably comprises or consists of the amino acid sequence shown in SEQ ID NO: 36 or an allelic variant thereof; or is a fragment thereof having hydrolytic activity. In another aspect, the polypeptide comprises or consists of the mature polypeptide of SEQ ID NO: 35. In another aspect, the polypeptide comprises or consists of amino acids 1 to 261 of SEQ ID NO: 35.

In one embodiment, the polypeptide preferably comprises or consists of the amino acid sequence shown in SEQ ID NO: 36; comprises the amino acid sequence shown in SEQ ID NO: 36 and a N-terminal and/or C-terminal His-tag and/or HQ-tag; comprises the amino acid sequence of SEQ ID NO: 36 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 36.

In some embodiment, the polypeptide has been isolated. A polypeptide of the present invention preferably comprises or consists of the amino acid sequence shown in SEQ ID NO: 39 or an allelic variant thereof; or is a fragment thereof having hydrolytic activity. In another aspect, the polypeptide comprises or consists of the mature polypeptide of SEQ ID NO: 38. In another aspect, the polypeptide comprises or consists of amino acids 1 to 265 of SEQ ID NO: 38.

In one embodiment, the polypeptide preferably comprises or consists of the amino acid sequence shown in SEQ ID NO: 39; comprises the amino acid sequence shown in SEQ ID NO: 39 and a N-terminal and/or C-terminal His-tag and/or HQ-tag; comprises the amino acid sequence of SEQ ID NO: 39 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 39.

In some embodiment, the polypeptide has been isolated. A polypeptide of the present invention preferably comprises or consists of the amino acid sequence shown in SEQ ID NO: 42 or an allelic variant thereof; or is a fragment thereof having hydrolytic activity. In another aspect, the polypeptide comprises or consists of the mature polypeptide of SEQ ID NO: 41. In another aspect, the polypeptide comprises or consists of amino acids 1 to 254 of SEQ ID NO: 41.

In one embodiment, the polypeptide preferably comprises or consists of the amino acid sequence shown in SEQ ID NO: 42; comprises the amino acid sequence shown in SEQ ID NO: 42 and a N-terminal and/or C-terminal His-tag and/or HQ-tag; comprises the amino acid sequence of SEQ ID NO: 42 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 42.

In some embodiment, the polypeptide has been isolated. A polypeptide of the present invention preferably comprises or consists of the amino acid sequence shown in SEQ ID NO: 45 or an allelic variant thereof; or is a fragment thereof having hydrolytic activity. In another aspect, the polypeptide comprises or consists of the mature polypeptide of SEQ ID NO: 44. In another aspect, the polypeptide comprises or consists of amino acids 1 to 268 of SEQ ID NO: 44.

In one embodiment, the polypeptide preferably comprises or consists of the amino acid sequence shown in SEQ ID NO: 45; comprises the amino acid sequence shown in SEQ ID NO: 45 and a N-terminal and/or C-terminal His-tag and/or HQ-tag; comprises the amino acid sequence of SEQ ID NO: 45 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 45.

In some embodiment, the polypeptide has been isolated. A polypeptide of the present invention preferably comprises or consists of the amino acid sequence shown in SEQ ID NO: 48 or an allelic variant thereof; or is a fragment thereof having hydrolytic activity. In another aspect, the polypeptide comprises or consists of the mature polypeptide of SEQ ID NO: 47. In another aspect, the polypeptide comprises or consists of amino acids 1 to 267 of SEQ ID NO: 47.

In one embodiment, the polypeptide preferably comprises or consists of the amino acid sequence shown in SEQ ID NO: 48; comprises the amino acid sequence shown in SEQ ID NO: 48 and a N-terminal and/or C-terminal His-tag and/or HQ-tag; comprises the amino acid sequence of SEQ ID NO: 48 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 48.

In some embodiment, the polypeptide has been isolated. A polypeptide of the present invention preferably comprises or consists of the amino acid sequence shown in SEQ ID NO: 51 or an allelic variant thereof; or is a fragment thereof having hydrolytic activity. In another aspect, the polypeptide comprises or consists of the mature polypeptide of SEQ ID NO: 50. In another aspect, the polypeptide comprises or consists of amino acids 1 to 251 of SEQ ID NO: 50.

In one embodiment, the polypeptide preferably comprises or consists of the amino acid sequence shown in SEQ ID NO: 51; comprises the amino acid sequence shown in SEQ ID NO: 51 and a N-terminal and/or C-terminal His-tag and/or HQ-tag; comprises the amino acid sequence of SEQ ID NO: 51 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 51.

In some embodiment, the polypeptide has been isolated. A polypeptide of the present invention preferably comprises or consists of the amino acid sequence shown in SEQ ID NO: 54 or an allelic variant thereof; or is a fragment thereof having hydrolytic activity. In another aspect, the polypeptide comprises or consists of the mature polypeptide of SEQ ID NO: 53. In another aspect, the polypeptide comprises or consists of amino acids 1 to 239 of SEQ ID NO: 53.

In one embodiment, the polypeptide preferably comprises or consists of the amino acid sequence shown in SEQ ID NO: 54; comprises the amino acid sequence shown in SEQ ID NO: 54 and a N-terminal and/or C-terminal His-tag and/or HQ-tag; comprises the amino acid sequence of SEQ ID NO: 54 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 54.

In some embodiment, the polypeptide has been isolated. A polypeptide of the present invention preferably comprises or consists of the amino acid sequence shown in SEQ ID NO: 57 or an allelic variant thereof; or is a fragment thereof having hydrolytic activity. In another aspect, the polypeptide comprises or consists of the mature polypeptide of SEQ ID NO: 56. In another aspect, the polypeptide comprises or consists of amino acids 1 to 279 of SEQ ID NO: 56.

In one embodiment, the polypeptide preferably comprises or consists of the amino acid sequence shown in SEQ ID NO: 57; comprises the amino acid sequence shown in SEQ ID NO: 57 and a N-terminal and/or C-terminal His-tag and/or HQ-tag; comprises the amino acid sequence of SEQ ID NO: 57 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 57.

In one embodiment, the polypeptide preferably comprises or consists of the amino acid sequence shown in:
a) SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146;
b) comprises the amino acid sequence shown in a) and a N-terminal and/or C-terminal His-tag and/or HQ-tag;
c) comprises the amino acid sequence of a) and a N-terminal and/or C-terminal extension of between 1 and 10 amino acids; or
d) is a fragment thereof having hydrolytic activity, preferably alpha-1,4-polygalactosaminidase activity, and having at least 50% such as at least 60%, at least 70%, at least 75%, at least 80%, at least 85%, at least 90%, at least 95%, at least 96%, at least 97%, at least 98% or at least 99% of the length of any of the polypeptides of a).

In some aspect, the invention relates to a polypeptide which comprises or consists of the amino acid sequence shown in SEQ ID NO: 3.

In some aspect, the invention relates to a polypeptide which comprises or consists of the amino acid sequence shown in SEQ ID NO: 6.

In some aspect, the invention relates to a polypeptide which comprises or consists of the amino acid sequence shown in SEQ ID NO: 9.

In some aspect, the invention relates to a polypeptide which comprises or consists of the amino acid sequence shown in SEQ ID NO: 12.

In some aspect, the invention relates to a polypeptide which comprises or consists of the amino acid sequence shown in SEQ ID NO: 15.

In some aspect, the invention relates to a polypeptide which comprises or consists of the amino acid sequence shown in SEQ ID NO: 18.

In some aspect, the invention relates to a polypeptide which comprises or consists of the amino acid sequence shown in SEQ ID NO: 21.

In some aspect, the invention relates to a polypeptide which comprises or consists of the amino acid sequence shown in SEQ ID NO: 24.

In some aspect, the invention relates to a polypeptide which comprises or consists of the amino acid sequence shown in SEQ ID NO: 27.

In some aspect, the invention relates to a polypeptide which comprises or consists of the amino acid sequence shown in SEQ ID NO: 30.

In some aspect, the invention relates to a polypeptide which comprises or consists of the amino acid sequence shown in SEQ ID NO: 33.

In some aspect, the invention relates to a polypeptide which comprises or consists of the amino acid sequence shown in SEQ ID NO: 36.

In some aspect, the invention relates to a polypeptide which comprises or consists of the amino acid sequence shown in SEQ ID NO: 39.

In some aspect, the invention relates to a polypeptide which comprises or consists of the amino acid sequence shown in SEQ ID NO: 42.

In some aspect, the invention relates to a polypeptide which comprises or consists of the amino acid sequence shown in SEQ ID NO: 45.

In some aspect, the invention relates to a polypeptide which comprises or consists of the amino acid sequence shown in SEQ ID NO: 48.

In some aspect, the invention relates to a polypeptide which comprises or consists of the amino acid sequence shown in SEQ ID NO: 51.

In some aspect, the invention relates to a polypeptide which comprises or consists of the amino acid sequence shown in SEQ ID NO: 54.

In some aspect, the invention relates to a polypeptide which comprises or consists of the amino acid sequence shown in SEQ ID NO: 57.

In some aspect, the invention relates to a polypeptide which comprises or consists of the amino acid sequence shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146.

In some embodiment, the present invention relates to variants of the mature polypeptide shown in SEQ ID NO: 3 comprising a substitution, deletion, and/or insertion at one or more (e.g., several) positions. In some embodiment, the number of amino acid substitutions, deletions and/or insertions introduced into the mature polypeptide shown in SEQ ID NO: 3 is up to 10, e.g., 1, 2, 3, 4, 5, 6, 7, 8, 9, or 10.

In some embodiment, the present invention relates to variants of the mature polypeptide shown in SEQ ID NO: 6 comprising a substitution, deletion, and/or insertion at one or more (e.g., several) positions. In some embodiment, the number of amino acid substitutions, deletions and/or insertions introduced into the mature polypeptide shown in SEQ ID NO: 6 is up to 10, e.g., 1, 2, 3, 4, 5, 6, 7, 8, 9, or 10.

In some embodiment, the present invention relates to variants of the mature polypeptide shown in SEQ ID NO: 9 comprising a substitution, deletion, and/or insertion at one or more (e.g., several) positions. In some embodiment, the number of amino acid substitutions, deletions and/or insertions introduced into the mature polypeptide shown in SEQ ID NO: 9 is up to 10, e.g., 1, 2, 3, 4, 5, 6, 7, 8, 9, or 10.

In some embodiment, the present invention relates to variants of the mature polypeptide shown in SEQ ID NO: 12 comprising a substitution, deletion, and/or insertion at one or more (e.g., several) positions. In some embodiment, the number of amino acid substitutions, deletions and/or insertions introduced into the mature polypeptide shown in SEQ ID NO: 12 is up to 10, e.g., 1, 2, 3, 4, 5, 6, 7, 8, 9, or 10.

In some embodiment, the present invention relates to variants of the mature polypeptide shown in SEQ ID NO: 15 comprising a substitution, deletion, and/or insertion at one or more (e.g., several) positions. In some embodiment, the number of amino acid substitutions, deletions and/or insertions introduced into the mature polypeptide shown in SEQ ID NO: 15 is up to 10, e.g., 1, 2, 3, 4, 5, 6, 7, 8, 9, or 10.

In some embodiment, the present invention relates to variants of the mature polypeptide shown in SEQ ID NO: 18 comprising a substitution, deletion, and/or insertion at one or more (e.g., several) positions. In some embodiment, the number of amino acid substitutions, deletions and/or insertions introduced into the mature polypeptide shown in SEQ ID NO: 18 is up to 10, e.g., 1, 2, 3, 4, 5, 6, 7, 8, 9, or 10.

In some embodiment, the present invention relates to variants of the mature polypeptide shown in SEQ ID NO: 21 comprising a substitution, deletion, and/or insertion at one or more (e.g., several) positions. In some embodiment, the number of amino acid substitutions, deletions and/or insertions introduced into the mature polypeptide shown in SEQ ID NO: 21 is up to 10, e.g., 1, 2, 3, 4, 5, 6, 7, 8, 9, or 10.

In some embodiment, the present invention relates to variants of the mature polypeptide shown in SEQ ID NO: 24 comprising a substitution, deletion, and/or insertion at one or more (e.g., several) positions. In some embodiment, the number of amino acid substitutions, deletions and/or insertions introduced into the mature polypeptide shown in SEQ ID NO: 24 is up to 10, e.g., 1, 2, 3, 4, 5, 6, 7, 8, 9, or 10.

In some embodiment, the present invention relates to variants of the mature polypeptide shown in SEQ ID NO: 27 comprising a substitution, deletion, and/or insertion at one or more (e.g., several) positions. In some embodiment, the number of amino acid substitutions, deletions and/or insertions introduced into the mature polypeptide shown in SEQ ID NO: 27 is up to 10, e.g., 1, 2, 3, 4, 5, 6, 7, 8, 9, or 10.

In some embodiment, the present invention relates to variants of the mature polypeptide shown in SEQ ID NO: 30 comprising a substitution, deletion, and/or insertion at one or more (e.g., several) positions. In some embodiment, the number of amino acid substitutions, deletions and/or insertions introduced into the mature polypeptide shown in SEQ ID NO: 30 is up to 10, e.g., 1, 2, 3, 4, 5, 6, 7, 8, 9, or 10.

In some embodiment, the present invention relates to variants of the mature polypeptide shown in SEQ ID NO: 33 comprising a substitution, deletion, and/or insertion at one or more (e.g., several) positions. In some embodiment, the number of amino acid substitutions, deletions and/or insertions introduced into the mature polypeptide shown in SEQ ID NO: 33 is up to 10, e.g., 1, 2, 3, 4, 5, 6, 7, 8, 9, or 10.

In some embodiment, the present invention relates to variants of the mature polypeptide shown in SEQ ID NO: 36 comprising a substitution, deletion, and/or insertion at one or more (e.g., several) positions. In some embodiment, the number of amino acid substitutions, deletions and/or insertions introduced into the mature polypeptide shown in SEQ ID NO: 36 is up to 10, e.g., 1, 2, 3, 4, 5, 6, 7, 8, 9, or 10.

In some embodiment, the present invention relates to variants of the mature polypeptide shown in SEQ ID NO: 39 comprising a substitution, deletion, and/or insertion at one or more (e.g., several) positions. In some embodiment, the number of amino acid substitutions, deletions and/or insertions introduced into the mature polypeptide shown in SEQ ID NO: 39 is up to 10, e.g., 1, 2, 3, 4, 5, 6, 7, 8, 9, or 10.

In some embodiment, the present invention relates to variants of the mature polypeptide shown in SEQ ID NO: 42 comprising a substitution, deletion, and/or insertion at one or more (e.g., several) positions. In some embodiment, the number of amino acid substitutions, deletions and/or insertions introduced into the mature polypeptide shown in SEQ ID NO: 42 is up to 10, e.g., 1, 2, 3, 4, 5, 6, 7, 8, 9, or 10.

In some embodiment, the present invention relates to variants of the mature polypeptide shown in SEQ ID NO: 45 comprising a substitution, deletion, and/or insertion at one or more (e.g., several) positions. In some embodiment, the number of amino acid substitutions, deletions and/or insertions introduced into the mature polypeptide shown in SEQ ID NO: 45 is up to 10, e.g., 1, 2, 3, 4, 5, 6, 7, 8, 9, or 10.

In some embodiment, the present invention relates to variants of the mature polypeptide shown in SEQ ID NO: 48 comprising a substitution, deletion, and/or insertion at one or more (e.g., several) positions. In some embodiment, the number of amino acid substitutions, deletions and/or insertions introduced into the mature polypeptide shown in SEQ ID NO: 48 is up to 10, e.g., 1, 2, 3, 4, 5, 6, 7, 8, 9, or 10.

In some embodiment, the present invention relates to variants of the mature polypeptide shown in SEQ ID NO: 51 comprising a substitution, deletion, and/or insertion at one or more (e.g., several) positions. In some embodiment, the number of amino acid substitutions, deletions and/or insertions introduced into the mature polypeptide shown in SEQ ID NO: 51 is up to 10, e.g., 1, 2, 3, 4, 5, 6, 7, 8, 9, or 10.

In some embodiment, the present invention relates to variants of the mature polypeptide shown in SEQ ID NO: 54 comprising a substitution, deletion, and/or insertion at one or more (e.g., several) positions. In some embodiment, the number of amino acid substitutions, deletions and/or insertions introduced into the mature polypeptide shown in SEQ ID NO: 54 is up to 10, e.g., 1, 2, 3, 4, 5, 6, 7, 8, 9, or 10.

In some embodiment, the present invention relates to variants of the mature polypeptide shown in SEQ ID NO: 57 comprising a substitution, deletion, and/or insertion at one or more (e.g., several) positions. In some embodiment, the number of amino acid substitutions, deletions and/or insertions introduced into the mature polypeptide shown in SEQ ID NO: 57 is up to 10, e.g., 1, 2, 3, 4, 5, 6, 7, 8, 9, or 10.

In some embodiment, the present invention relates to variants of the mature polypeptide shown SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146 comprising a substitution, deletion, and/or insertion at one or more (e.g., several) positions. In some embodiment, the number of amino acid substitutions, deletions and/or insertions introduced into the mature polypeptide shown above is up to 10, e.g., 1, 2, 3, 4, 5, 6, 7, 8, 9, or 10.

One particular embodiment relates to a GH114 glycosyl hydrolase variant, which compared to a GH114 glycosyl hydrolase with SEQ ID NO: 18, comprises one or more alteration selected from the group consisting of: K1A, L11I, C23V, T39A, A64S, N73Q, N73R, N73K, G75V, A76M, A76R, A76V, L77V, L77A, L77T, L77K, Q78E, Q78M, Q78K, Q78N, Q78R, D79K, D79N, D79P, D79Q, D79V, D79W, D79T, W80M, W80Y, W80E, W80I, W80R, D81V, S85A, S85G, S85W, S85C, K86D, K86E, K89F, K86W, K89D, K89A, K89E, K89P, K89S, K89V, K89W, E90A, E90D, E90N, E90S, V91M, V91I, V91S, V91T, 192E, S97G, Y98W, D101*, S102*, E104*, E116D, E116K, T119K, I124L, I124V, D136E, D144*, D145E, D145G, E146*, K152D, K152G, K154E, K154T, K167D, K167T, K173T, Q178N, N209T, N209S, P212*, D222E, D222W, L234F, V242S, E243A, E243S, E243L, E243P, E243V, K244P, K244L, K244Q, K244R, T245C, T245D, G246Q, K247A, K247C, K247E, K247R, K247S, K247V, K247M, V248M, V248L, A250D, A250E, A250P, S251A, S251C, S251E, S251W, S251G, S251K, S251L, S251Q, S251T, N253C, N253E, N253L, N253M, N253A, N253P, N253W, K254M, K254R, K254A, K254C, K254D, K254E, K254H, K254V, K254T, Y256E, Y256G, Y256M, Y256R, Y256S, Y256W, Y256T, T258A, T258G, T258Q, T258D, T258E, T258S, A259E, A259P, E260G, E260K, E260Q, E260R, E260T, E260W, D261A, D261L, D261R, E262A, E262F, E262G, E262I, E262K, E262L, E262M, E262Q, E262R, E262S, E262T, E262V, E262W, E262C, E262D, K264R, 1270L, G279E, G279K, G279R, G279S, K295N and Y296*, wherein the positions correspond to the positions of SEQ ID NO: 18 (numbering according to SEQ ID NO: 18), wherein the variant has hydrolase activity, preferably alpha-1,4-polygalactosaminidase activity.

One particular embodiment relates to a GH114 glycosyl hydrolase variant, which compared to a GH114 glycosyl hydrolase with SEQ ID NO: 18, comprises one or more alteration selected from the group consisting of: K1A, L11I, C23V, T39A, A64S, N73Q, N73R, N73K, G75V, A76M, A76R, A76V, L77V, L77A, L77T, L77K, Q78E, Q78M, Q78K, Q78N, Q78R, D79K, D79N, D79P, D79Q, D79V, D79W, D79T, W80M, W80Y, W80E, W80I, W80R, D81V, S85A, S85G, S85W, S85C, K86D, K86E, K89F, K86W, K89D, K89A, K89E, K89P, K89S, K89V, K89W, E90A, E90D, E90N, E90S, V91M, V91I, V91S, V91T, 192E, S97G, Y98W, D101*, S102*, E104*, E116D, E116K, T119K, I124L, I124V, D136E, D144*, D145E, D145G, E146*, K152D, K152G, K154E, K154T, K167D, K167T, K173T, Q178N, N209T, N209S, P212*, D222E, D222W, L234F, V242S, E243A, E243S, E243L, E243P, E243V, K244P, K244L, K244Q, K244R, T245C, T245D, G246Q, K247A, K247C, K247E, K247R, K247S, K247V, K247M, V248M, V248L, A250D, A250E, A250P, S251A, S251C, S251E, S251W, S251G, S251K, S251L, S251Q, S251T, N253C, N253E, N253L, N253M, N253A, N253P, N253W, K254M, K254R, K254A, K254C, K254D, K254E, K254H, K254V, K254T, Y256E, Y256G, Y256M, Y256R, Y256S, Y256W, Y256T, T258A, T258G, T258Q, T258D, T258E, T258S, A259E, A259P, E260G, E260K, E260Q, E260R, E260T, E260W, D261A, D261L, D261R, E262A, E262F, E262G, E262I, E262K, E262L, E262M, E262Q, E262R, E262S, E262T, E262V, E262W, E262C, E262D, K264R, 1270L, G279E, G279K, G279R, G279S, K295N and Y296*, wherein the positions correspond to the positions of SEQ ID NO: 18 (numbering according to SEQ ID NO: 18), wherein the variant has a sequence identity to the polypeptide shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54 SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146 of at least 60%, at least 70%, at least 75%, at least 80%, at least 85%, at least 90%, at least 95%, at least 96%, at least 97%, at least 98%, of at least 99% but less than 100%, and wherein the variant has hydrolase activity, preferably alpha-1,4-polygalactosaminidase activity.

One particular embodiment relates to a GH114 glycosyl hydrolase variant, which compared to a GH114 glycosyl hydrolase with SEQ ID NO: 18, comprises one or more alteration selected from the group consisting of: K1A, L11I, C23V, T39A, A64S, N73Q, N73R, N73K, G75V, A76M, A76R, A76V, L77V, L77A, L77T, L77K, Q78E, Q78M, Q78K, Q78N, Q78R, D79K, D79N, D79P, D79Q, D79V, D79W, D79T, W80M, W80Y, W80E, W80I, W80R, D81V, S85A, S85G, S85W, S85C, K86D, K86E, K89F, K86W, K89D, K89A, K89E, K89P, K89S, K89V, K89W, E90A, E90D, E90N, E90S, V91M, V91I, V91S, V91T, 192E, S97G, Y98W, D101*, S102*, E104*, E116D, E116K, T119K, I124L, I124V, D136E, D144*, D145E, D145G, E146*, K152D, K152G, K154E, K154T, K167D, K167T, K173T, Q178N, N209T, N209S, P212*, D222E, D222W, L234F, V242S, E243A, E243S, E243L, E243P, E243V, K244P, K244L, K244Q, K244R, T245C, T245D, G246Q, K247A, K247C, K247E, K247R, K247S, K247V, K247M, V248M, V248L, A250D, A250E, A250P, S251A, S251C, S251E, S251W, S251G, S251K, S251L, S251Q, S251T, N253C, N253E, N253L, N253M, N253A, N253P, N253W, K254M, K254R, K254A, K254C, K254D, K254E, K254H, K254V, K254T, Y256E, Y256G, Y256M, Y256R, Y256S, Y256W, Y256T, T258A, T258G, T258Q, T258D, T258E, T258S, A259E, A259P, E260G, E260K, E260Q, E260R, E260T, E260W, D261A, D261L, D261R, E262A, E262F, E262G, E262I, E262K, E262L, E262M, E262Q, E262R, E262S, E262T, E262V, E262W, E262C, E262D, K264R, 1270L, G279E, G279K, G279R, G279S, K295N and Y296*, wherein the positions correspond to the positions of SEQ ID NO: 18 (numbering according to SEQ ID NO: 18), wherein the variant has a sequence identity to the polypeptide shown in SEQ ID NO: 18 of at least 60%, at least 70%, at least 75%, at least 80%, at least 85%, at least 90%, at least 95%, at least 96%, at least 97%, at least 98%, of at least 99% but less than 100%, and wherein variant has hydrolase activity, preferably alpha-1,4-polygalactosaminidase activity.

One embodiment relates to a GH114 glycosyl hydrolase variant, which compared to a GH114 glycosyl hydrolase with SEQ ID NO: 18, comprises one or more substitution selected from the group consisting of: K1A, L11I, C23V, T39A, A64S, N73Q, N73R, N73K, G75V, A76M, A76R, A76V, L77V, L77A, L77T, L77K, Q78E, Q78M, Q78K, Q78N, Q78R, D79K, D79N, D79P, D79Q, D79V, D79W, D79T, W80M, W80Y, W80E, W80I, W80R, D81V, S85A, S85G, S85W, S85C, K86D, K86E, K89F, K86W, K89D, K89A, K89E, K89P, K89S, K89V, K89W, E90A, E90D, E90N, E90S, V91M, V91I, V91S, V91T, 192E, S97G, Y98W, E116D, E116K, T119K, I124L, I124V, D136E, D145E, D145G, K152D, K152G, K154E, K154T, K167D, K167T, K173T, Q178N, N209T, N209S, D222E, D222W, L234F, V242S, E243A, E243S, E243L, E243P, E243V, K244P, K244L, K244Q, K244R, T245C, T245D, G246Q, K247A, K247C, K247E, K247R, K247S, K247V, K247M, V248M, V248L, A250D, A250E, A250P, S251A, S251C, S251E, S251W, S251G, S251K, S251L, S251Q, S251T, N253C, N253E, N253L, N253M, N253A, N253P, N253W, K254M, K254R, K254A, K254C, K254D, K254E, K254H, K254V, K254T, Y256E, Y256G, Y256M, Y256R, Y256S, Y256W, Y256T, T258A, T258G, T258Q, T258D, T258E, T258S, A259E, A259P, E260G, E260K, E260Q, E260R, E260T, E260W, D261A, D261L, D261R, E262A, E262F, E262G, E262I, E262K, E262L, E262M, E262Q, E262R, E262S, E262T, E262V, E262W, E262C, E262D, K264R, 1270L, G279E, G279K, G279R, G279S and K295N, wherein the positions correspond to the positions of SEQ ID NO: 18 (numbering according to SEQ ID NO: 18), wherein the variant has a sequence identity to the polypeptide shown in SEQ ID NO: 18 of at least 60%, at least 70%, at least 75%, at least 80%, at least 85%, at least 90%, at least 95%, at least 96%, at least 97%, at least 98%, of at least 99% but less than 100%, and wherein the variant has hydrolase activity, preferably alpha-1,4-polygalactosaminidase activity.

One particular embodiment relates to a GH114 glycosyl hydrolase variant, which compared to a GH114 glycosyl hydrolase with SEQ ID NO: 18, comprises one or more deletion selected from the group consisting of: D101*, S102*, E104*, D144*, E146*, P212* and Y296*, wherein the positions correspond to the positions of SEQ ID NO: 18 (numbering according to SEQ ID NO: 18), wherein the variant has a sequence identity to the polypeptide shown in SEQ ID NO: 18 at least 60%, at least 70%, at least 75%, at least 80%, at least 85%, at least 90%, at least 95%, at least 96%, at least 97%, at least 98%, of at least 99% but less than 100%, and wherein the variant has hydrolase activity, preferably having alpha-1,4-polygalactosaminidase activity.

The GH114 glycosyl hydrolase may comprise two or more alterations selected from the group consisting of: K1A, L11I, C23V, T39A, A64S, N73Q, N73R, N73K, G75V, A76M, A76R, A76V, L77V, L77A, L77T, L77K, Q78E, Q78M, Q78K, Q78N, Q78R, D79K, D79N, D79P, D79Q, D79V, D79W, D79T, W80M, W80Y, W80E, W80I, W80R, D81V, S85A, S85G, S85W, S85C, K86D, K86E, K89F, K86W, K89D, K89A, K89E, K89P, K89S, K89V, K89W, E90A, E90D, E90N, E90S, V91M, V91I, V91S, V91T, 192E, S97G, Y98W, D101*, S102*, E104*, E116D, E116K, T119K, I124L, I124V, D136E, D144*, D145E, D145G, E146*, K152D, K152G, K154E, K154T, K167D, K167T, K173T, Q178N, N209T, N209S, P212*, D222E, D222W, L234F, V242S, E243A, E243S, E243L, E243P, E243V, K244P, K244L, K244Q, K244R, T245C, T245D, G246Q, K247A, K247C, K247E, K247R, K247S, K247V, K247M, V248M, V248L, A250D, A250E, A250P, S251A, S251C, S251E, S251W, S251G, S251K, S251L, S251Q, S251T, N253C, N253E, N253L, N253M, N253A, N253P, N253W, K254M, K254R, K254A, K254C, K254D, K254E, K254H, K254V, K254T, Y256E, Y256G, Y256M, Y256R, Y256S, Y256W, Y256T, T258A, T258G, T258Q, T258D, T258E, T258S, A259E, A259P, E260G, E260K, E260Q, E260R, E260T, E260W, D261A, D261L, D261R, E262A, E262F, E262G, E262I, E262K, E262L, E262M, E262Q, E262R, E262S, E262T, E262V, E262W, E262C, E262D, K264R, 1270L, G279E, G279K, G279R, G279S, K295N and Y296*.

One embodiment relates to a GH114 glycosyl hydrolase variant, which compared to a GH114 glycosyl hydrolase with SEQ ID NO: 18, comprises one or more of the alterations selected from the group consisting of: G230D+A259P, 1172V+A250D, D101*+A166V, E103*+E104*, N160D+K254S, T119M+P212*, W80R+D145G, W80R+K154T and Y100*+D101*, wherein the variant has at least 60%, at least 70%, at least 75%, at least 80%, at least 85%, at least 90%, at least 95%, at least 96%, at least 97%, at least 98%, of at least 99% but less than 100% sequence identity to SEQ ID NO: 18.

One embodiment, relates to a method for obtaining a GH114 glycosyl hydrolase variant, comprising:
a) introducing into a parent GH114 glycosyl hydrolase, e.g., SEQ ID NO: 18, one or more of the following mutations: K1A, L11I, C23V, T39A, A64S, N73Q, N73R, N73K, G75V, A76M, A76R, A76V, L77V, L77A, L77T, L77K, Q78E, Q78M, Q78K, Q78N, Q78R, D79K, D79N, D79P, D79Q, D79V, D79W, D79T, W80M, W80Y, W80E, W80I, W80R, D81V, S85A, S85G, S85W, S85C, K86D, K86E, K89F, K86W, K89D, K89A, K89E, K89P, K89S, K89V, K89W, E90A, E90D, E90N, E90S, V91M, V91I, V91S, V91T, 192E, S97G, Y98W, D101*, S102*, E104*, E116D, E116K, T119K, I124L, I124V, D136E, D144*, D145E, D145G, E146*, K152D, K152G, K154E, K154T, K167D, K167T, K173T, Q178N, N209T, N209S, P212*, D222E, D222W, L234F, V242S, E243A, E243S, E243L, E243P, E243V, K244P, K244L, K244Q, K244R, T245C, T245D, G246Q, K247A, K247C, K247E, K247R, K247S, K247V, K247M, V248M, V248L, A250D, A250E, A250P, S251A, S251C, S251E, S251W, S251G, S251K, S251L, S251Q, S251T, N253C, N253E, N253L, N253M, N253A, N253P, N253W, K254M, K254R, K254A, K254C, K254D, K254E, K254H, K254V, K254T, Y256E, Y256G, Y256M, Y256R, Y256S, Y256W, Y256T, T258A, T258G, T258Q, T258D, T258E, T258S, A259E, A259P, E260G, E260K, E260Q, E260R, E260T, E260W, D261A, D261L, D261R, E262A, E262F, E262G, E262I, E262K, E262L, E262M, E262Q, E262R, E262S, E262T, E262V, E262W, E262C, E262D, K264R, 1270L, G279E, G279K, G279R, G279S, K295N and Y296*, wherein the positions correspond to the positions of SEQ ID NO: 18 (numbering according to SEQ ID NO: 18) and wherein the variant has hydrolase activity, preferably having alpha-1,4-polygalactosaminidase activity, and
b) recovering the variant.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for hydrolytic activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide shown in the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

### Sources of Polypeptides Having Hydrolytic Activity

A polypeptide having hydrolytic activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly. In one aspect, the polypeptide is a Amycolatopsis polypeptide, e.g., a polypeptide obtained from Amycolatopsis circi or Amycolatopsis sp. In one aspect, the polypeptide is a Streptomyces polypeptide, e.g., a polypeptide obtained from Streptomyces sp., Streptomyces miharaensis or Streptomyces parvulus. In one aspect, the polypeptide is a Fusarium polypeptide, e.g., a polypeptide obtained from Fusarium solani. In one aspect, the polypeptide is a Nonomuraea polypeptide, e.g., a polypeptide obtained from Nonomuraea guangzhouensis. In one aspect, the polypeptide is a Vibrio polypeptide, e.g., a polypeptide obtained from Vibrio sp. In one aspect, the polypeptide is a Microbacterium polypeptide, e.g., a polypeptide obtained from Microbacterium oxydans or Microbacterium saccharophilum. In one aspect, the polypeptide is a Frigoribacterium polypeptide, e.g., a polypeptide obtained from Frigoribacterium faeni. In one aspect, the polypeptide is a Serinibacter polypeptide, e.g., a polypeptide obtained from Serinibacter sp. In one aspect, the polypeptide is a Oerskovia polypeptide, e.g., a polypeptide obtained from Oerskovia paurometabola. In one aspect, the polypeptide is a Agreia polypeptide, e.g., a polypeptide obtained from Agreia pratensis. In one aspect, the polypeptide is a Plantibacter polypeptide, e.g., a polypeptide obtained from Plantibacter flavus. In one aspect, the polypeptide is a Urnula polypeptide, e.g., a polypeptide obtained from Urnula craterium.

In one aspect, the polypeptide is polypeptide obtained from Chaetomium globosum, Stilbella fimetaria, Volutella ciliata, Clonostachys epichloe, Myrothecium sp., Botryotrichum piluliferum, Myrothecium sp., Amycolatopsis sp., Methylothermaceae bacteria B42, Amycolatopsis niigatensis, Streptomyces sp. AA4, Amycolatopsis alba DSM 44262, Streptomyces griseoaurantiacus, Kutzneria albida, Microbacterium sp, Microbacterium oleivorans O827CG, Nocardiopsis alba, Microbacterium oxydans, Agreia pratensis, Microbacterium lemovicicum, Xylanibacterium sp, Curtobacterium oceanosedimentum, Leucobacter tardus, Salinibacterium amurskyense, Neonectria candida, Fusarium compactum, Fusarium compactum, Fusarium avenaceum, Preussia aemulans, Fusarium proliferatum, Fusarium lateritium, Fusarium proliferatum, Chaetomium ancistrocladum, Fusarium verticillioides, Fusarium lateritium, Chaetomium sp. ZY474, Scytalidium sp. T045-6, Acremonium sp., Chaetomium strumarium, Fusarium oxysporum, Fusarium temperatum, Thermoascus taitungiacus, Plectosphaerella alismatis, Thermothelomyces hinnuleus, Ovatospora brasiliensis, Fusarium acuminatum, Fusarium euwallaceae, Fusarium neocosmosporiellum, Thielavia antarctica, Acremonium thermophilum, Crassicarpon hotsonii, Helicosporium sp, Pyrenochaetopsis sp., Acremonium cf. fusifioides, Rasamsonia byssochlamydoides, Acremonium dichromosporum, Thermomyces dupontii, Ovatospora medusarum, Marasmius oreades, Amycolatopsis niigatensis, Byssochlamys spectabilis, Coprinopsis sp, Fusarium sambucinum, Amycolatopsis orientalis DSM 46075, Xanthan alkaline community D, Pilimelia columellifera subsp. pallida, Cellulomonas cellasea, Gluten B enrichment AX2, Microbacterium sp., Arthrobacter agilis, Amycolatopsis bullii, Microbacterium oxydans, Microbacterium phyllosphaerae or Phycicoccus dokdonensis.

In one embodiment, the GH114 glycosyl hydrolase is obtained from Amycolatopsis, preferably Amycolatopsis circi or Amycolatopsis sp., wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65). In one embodiment, the GH114 glycosyl hydrolase is obtained from Amycolatopsis, preferably Amycolatopsis circi or Amycolatopsis sp, wherein the GH114 glycosyl hydrolase is a polypeptide having;
a) at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 3;
b) at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 6; or
c) at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 9.

In one embodiment, the GH114 glycosyl hydrolase is obtained from Streptomyces, preferably Streptomyces parvulus or Streptomyces miharaensis, wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65). In one embodiment, the GH114 glycosyl hydrolase is obtained from Streptomyces, preferably Streptomyces parvulus or Streptomyces miharaensis, wherein the GH114 glycosyl hydrolase is;
a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 12;
b) is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 24; or
c) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 54.

In one embodiment, the GH114 glycosyl hydrolase is obtained from Fusarium, preferably Fusarium solani, wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), GXXVX[NHQTS]IEY[PG] (SEQ ID NO: 68), VICYF (SEQ ID NO: 69), ICYFSA (SEQ ID NO: 70), DFAVL (SEQ ID NO: 71). In one embodiment, the GH114 glycosyl hydrolase is obtained from Fusarium, preferably Fusarium solani, wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 15 or a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 18.

In one embodiment, the GH114 glycosyl hydrolase is obtained from Nonomuraea, preferably Nonomuraea guangzhouensis, wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65). In one embodiment, the GH114 glycosyl hydrolase is obtained from Nonomuraea, preferably Nonomuraea guangzhouensis, wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 21.

In one embodiment, the GH114 glycosyl hydrolase is obtained from Vibrio, preferably Vibrio sp., wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), WQWQL (SEQ ID NO: 66), [VLI][GASD]LKN[DGS][VLIP] (SEQ ID NO: 67). In one embodiment, the GH114 glycosyl hydrolase is obtained from Vibrio, preferably Vibrio sp., wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 27.

In one embodiment, the GH114 glycosyl hydrolase is obtained from Microbacterium, preferably Microbacterium saccharophilum or Microbacterium oxydans, wherein the GH114 glycosyl hydrolase comprises one or more, or even both of the motif(s) [VLI]XE[EDSQ]C (SEQ ID NO: 60), CY[FLIV][SDN][ATVG] (SEQ ID NO: 61). In one embodiment, the GH114 glycosyl hydrolase is obtained from Microbacterium, preferably Microbacterium saccharophilum or Microbacterium oxydans, wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 33 or a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 36.

In one embodiment, the GH114 glycosyl hydrolase is obtained from Frigoribacterium, preferably Frigoribacterium faeni, wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63). In one embodiment, the GH114 glycosyl hydrolase is obtained from Frigoribacterium, preferably Frigoribacterium faeni, wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 39.

In one embodiment, the GH114 glycosyl hydrolase is obtained from Serinibacter, preferably Serinibacter sp, wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63). In one embodiment, the GH114 glycosyl hydrolase is obtained from Serinibacter, preferably Serinibacter sp., wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 42.

In one embodiment, the GH114 glycosyl hydrolase is obtained from Oerskovia, preferably Oerskovia paurometabola, wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63). In one embodiment, the GH114 glycosyl hydrolase is obtained from Oerskovia, preferably Oerskovia paurometabola, wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 45.

In one embodiment, the GH114 glycosyl hydrolase is obtained from Agreia, preferably Agreia pratensis, wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63). In one embodiment, the GH114 glycosyl hydrolase is obtained from Agreia, preferably Agreia pratensis, wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 48.

In one embodiment, the GH114 glycosyl hydrolase is obtained from Plantibacter, preferably Plantibacter flavus, wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63). In one embodiment, the GH114 glycosyl hydrolase is obtained from Plantibacter, preferably Plantibacter flavus, wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 51.

In one embodiment, the GH114 glycosyl hydrolase is obtained from Urnula, preferably Urnula craterium, wherein the GH114 glycosyl hydrolase comprises one or more, or even all of the motif(s) [VLI]XE[EDSQ]C (SEQ ID NO: 60), CY[FLIV][SDN][ATVG] (SEQ ID NO: 61), GXXVX[NHQTS]IEY[PG] (SEQ ID NO: 68), VICYF (SEQ ID NO: 69), ICYFSA (SEQ ID NO: 70). In one embodiment, the GH114 glycosyl hydrolase is obtained from Urnula, preferably Urnula craterium, wherein the GH114 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 57.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptide of the present invention, as described herein. In some embodiment, the polynucleotide encoding the polypeptide shown in the present invention has been isolated.

In some embodiment, the present invention relates to a polynucleotide encoding a polypeptide having hydrolytic activity, wherein the polynucleotide having a sequence identity to the mature polypeptide coding sequence of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 4 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 7 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 10 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 13 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 16 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 19 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 22 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 25 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 28 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 31 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 34 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 37 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 40 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 43 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 46 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 49 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 52 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 55 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

One embodiment, relates to an isolated nucleic acid sequence selected from the group consisting of:
a) a nucleic acid sequence encoding an enzyme comprising an amino acid sequence selected from the amino acid sequences of: SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146;
b) a nucleic acid sequence encoding a fragment of an GH114 glycosyl hydrolase enzyme selected from (a), wherein the fragment has alpha-1,4-polygalactosaminidase activity; and
c) a nucleic acid sequence encoding a polypeptide having an amino acid sequence having at least 60%, at least 70%, at least 75%, at least 80%, at least 85%, at least 90%, at least 95% at least 98% or 100% sequence identity to an GH114 glycosyl hydrolase enzyme selected from (a) and has alpha-1,4-polygalactosaminidase activity.

One embodiment, relates to an isolated nucleic acid sequence selected from the group consisting of:
a) a nucleic acid sequence encoding an enzyme comprising an amino acid sequence selected from the amino acid sequences of: SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146;
b) a nucleic acid sequence encoding a fragment of an GH114 glycosyl hydrolase enzyme selected from (a), wherein the fragment has alpha-1,4-polygalactosaminidase activity; and
c) a nucleic acid sequence encoding a polypeptide having an amino acid sequence having at least 70% sequence identity to an GH114 glycosyl hydrolase enzyme selected from (a) and has alpha-1,4-polygalactosaminidase activity.

One embodiment, relates to an isolated nucleic acid sequence selected from the group consisting of:
a) a nucleic acid sequence encoding an enzyme comprising an amino acid sequence selected from the amino acid sequences of: SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146;
b) a nucleic acid sequence encoding a fragment of an GH114 glycosyl hydrolase enzyme selected from (a), wherein the fragment has alpha-1,4-polygalactosaminidase activity; and
c) a nucleic acid sequence encoding a polypeptide having an amino acid sequence having at least 80% sequence identity to an GH114 glycosyl hydrolase enzyme selected from (a) and has alpha-1,4-polygalactosaminidase activity.

One embodiment, relates to an isolated nucleic acid sequence selected from the group consisting of:
a) a nucleic acid sequence encoding an enzyme comprising an amino acid sequence selected from the amino acid sequences of: SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146;
b) a nucleic acid sequence encoding a fragment of an GH114 glycosyl hydrolase enzyme selected from (a), wherein the fragment has alpha-1,4-polygalactosaminidase activity; and
c) a nucleic acid sequence encoding a polypeptide having an amino acid sequence having at least 90% sequence identity to an GH114 glycosyl hydrolase enzyme selected from (a) and has alpha-1,4-polygalactosaminidase activity.

The techniques used to isolate or clone a polynucleotide are known in the art and include isolation from genomic DNA or cDNA, or a combination thereof. The cloning of the polynucleotides from genomic DNA can be affected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xyIB genes, Bacillus thuringiensis crylllA gene (Agaisse and Lereclus, 1994, Molecular Microbiology 13:97-107), E. coli lac operon, E. coli trc promoter (Egon et al., 1988, Gene 69:301-315), Streptomyces coelicolor agarase gene (dagA), and prokaryotic beta-lactamase gene (Villa-Kamaroff et al., 1978, Proc. Natl. Acad. Sci. USA 75:3727-3731), as well as the tac promoter (DeBoer et al., 1983, Proc. Natl. Acad. Sci. USA 80:21-25). Further promoters are described in “Useful proteins from recombinant bacteria” in Gilbert et al., 1980, Scientific American 242:74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8:423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis crylllA gene (WO 94/25612) and a Bacillus subtilis SP82 gene (Hue et al., 1995, Journal of Bacteriology 177:3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57:109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMB1 permitting replication in Bacillus.

Examples of origins of replication for use in a yeast host cell are the 2-micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98:61-67; Cullen et al., 1987, Nucleic Acids Res. 15:9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be affected by protoplast transformation (see, e.g., Chang and Cohen, 1979, Mol. Gen. Genet. 168:111-115), competent cell transformation (see, e.g., Young and Spizizen, 1961, J. Bacteriol. 81:823-829, or Dubnau and Davidoff-Abelson, 1971, J. Mol. Biol. 56:209-221), electroporation (see, e.g., Shigekawa and Dower, 1988, Biotechniques 6:742-751), or conjugation (see, e.g., Koehler and Thorne, 1987, J. Bacteriol. 169:5271-5278). The introduction of DNA into an E. coli cell may be affected by protoplast transformation (see, e.g., Hanahan, 1983, J. Mol. Biol. 166:557-580) or electroporation (see, e.g., Dower et al., 1988, Nucleic Acids Res. 16:6127-6145). The introduction of DNA into a Streptomyces cell may be affected by protoplast transformation, electroporation (see, e.g., Gong et al., 2004, Folia Microbiol. (Praha) 49:399-405), conjugation (see, e.g., Mazodier et al., 1989, J. Bacteriol. 171:3583-3585), or transduction (see, e.g., Burke et al., 2001, Proc. Natl. Acad. Sci. USA 98:6289-6294). The introduction of DNA into a Pseudomonas cell may be affected by electroporation (see, e.g., Choi et al., 2006, J. Microbiol. Methods 64:391-397) or conjugation (see, e.g., Pinedo and Smets, 2005, Appl. Environ. Microbiol. 71:51-57). The introduction of DNA into a Streptococcus cell may be affected by natural competence (see, e.g., Perry and Kuramitsu, 1981, Infect. Immun. 32:1295-1297), protoplast transformation (see, e.g., Catt and Jollick, 1991, Microbios 68:189-207), electroporation (see, e.g., Buckley et al., 1999, Appl. Environ. Microbiol. 65:3800-3804), or conjugation (see, e.g., Clewell, 1981, Microbiol. Rev. 45:409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolismmay be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81:1470-1474, and Christensen et al., 1988, Bio/Technology 6:1419-1422. Suitable methods for transforming Fusarium species are described by Malardier et al., 1989, Gene 78:147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153:163; and Hinnen et al., 1978, Proc. Natl. Acad. Sci. USA 75:1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide.

The present invention also relates to methods of producing a polypeptide, wherein the polypeptide is selected from the group consisting of:
(a) a polypeptide having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54; and
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57, comprising (a) cultivating a cell, which in its wild-type form produces the polypeptide, under conditions conducive for production of the polypeptide; and optionally, (b) recovering the polypeptide.

The present invention also relates to methods of producing a polypeptide, wherein the polypeptide is selected from the group consisting of:
(a) a polypeptide having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54; and
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermenters in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides having hydrolytic activity. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide shown in the present invention which are used to produce the polypeptide shown in interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The invention relates to compositions comprising a GH114 glycosyl hydrolase polypeptide of the present invention in combination with one or more additional component(s). The choice of additional components is within the skill of the artisan and includes conventional ingredients, including the exemplary non-limiting components set forth below.

Some embodiments of the invention relate to a composition comprising:
a) at least 0.001 ppm of at least one polypeptide having hydrolytic activity, preferably alpha-1,4-polygalactosaminidase activity, wherein the polypeptide is selected from the group consisting of polypeptides comprising the amino acid sequence shown in: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146 and polypeptides having at least 60%, at least 65%, at least 70%, at least 75%, at least 80%, at least 85%, at least 90%, at least 95%, at least 96%, at least 97%, at least 98%, at least 99% or 100% sequence identity thereto;
b) one or more adjunct ingredient.

Some embodiments of the invention relate to a cleaning composition comprising:
a) at least 0.001 ppm of at least one polypeptide having hydrolytic activity, preferably alpha-1,4-polygalactosaminidase activity, wherein the polypeptide is selected from the group consisting of polypeptides comprising the amino acid sequence shown in: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146 and polypeptides having at least 60%, at least 65%, at least 70%, at least 75%, at least 80%, at least 85%, at least 90%, at least 95%, at least 96%, at least 97%, at least 98%, at least 99% or 100% sequence identity thereto;
b) one or more cleaning composition component, preferably selected from surfactants, builders, bleach components, polymers, dispersing agents and additional enzymes.

Some embodiments of the invention relate to a cleaning composition comprising:
a) at least 0.001 ppm of at least one polypeptide having hydrolytic activity, preferably alpha-1,4-polygalactosaminidase activity, wherein the polypeptide comprises one or more motif selected from the group consisting of: [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71); and
b) one or more cleaning composition component, preferably selected from surfactants, builders, bleach components, polymers, dispersing agents and additional enzymes.

One embodiment relates to a cleaning composition comprising:
a) at least 0.001 ppm of at least one polypeptide having hydrolytic activity, preferably alpha-1,4-polygalactosaminidase activity, e.g., a GH114 glycosyl hydrolase, wherein the polypeptide is selected from the group consisting of:
   i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
   ii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
   iii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
   iv) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
   v) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
   vi) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
   vii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
   viii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
   ix) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
   x) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
   xi) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
   xii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
   xiii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
   xiv) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
   xv) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
   xvi) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO:
   xvii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
   xviii) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54;
   xix) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57; and
b) one or more cleaning composition component, preferably selected from surfactants, builders, bleach components, polymers, dispersing agents and additional enzymes.

The polypeptides to be formulated in the cleaning compositions of the invention are preferably belonging to the VAE clade as shown in FIG. 1. The polypeptides of this clade comprise one or two distinct conservative motifs [VLI]XE[EDSQ]C (SEQ ID NO: 60) and/or CY[FLIV][SDN][ATVG] (SEQ ID NO: 61). Polypeptides comprising these motifs will share these common conserved regions and will have similar properties, e.g., in respect of stability and substrate binding.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) [VLI]XE[EDSQ]C (SEQ ID NO: 60) or CY[FLIV][SDN][ATVG] (SEQ ID NO: 61); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES).

The cleaning composition according to claim 1, further comprising at least one non-ionic surfactant, preferably an alcohol ethoxylates (AE or AEO), preferably the weight ratio of anionic to non-ionic surfactant is from 10:1 to 1:10. More surfactants suitable for a composition of the invention are described in the surfactant section below.

The polypeptides of the invention and disclosed herein all belong to the VAE clade as also could be visualized in FIG. 1.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) [VLI]XE[EDSQ]C (SEQ ID NO: 60) or CY[FLIV][SDN][ATVG] (SEQ ID NO: 61); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES), wherein the GH114 glycosyl hydrolase enzyme is selected from the list consisting of:
i. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
ii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
iii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
iv. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
v. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
vi. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
vii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
viii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
ix. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
x. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
xi. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
xii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
xiii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
xiv. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
xv. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
xvi. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
xvii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
xviii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54; and
xix. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) [VLI]XE[EDSQ]C (SEQ ID NO: 60) or CY[FLIV][SDN][ATVG] (SEQ ID NO: 61); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES),
   wherein the GH114 glycosyl hydrolase enzyme is selected from the list consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146.

Another interesting clade derived from the VAE clade is the DYQ-clade, which comprises many of the polypeptides of the VAE-clade. The polypeptides of this clade comprise one or two distinct conservative motifs DYQ[LI]G (SEQ ID NO: 62) or FQ[TAV]Q[PSD] (SEQ ID NO: 63), which are conserved within the clade.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) DYQ[LI]G (SEQ ID NO: 62) or FQ[TAV]Q[PSD] (SEQ ID NO: 63); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES).

Many of the polypeptides of the invention are also comprised in the DYQ clade, these polypeptides will comprise at least one or both motifs [VLI]XE[EDSQ]C (SEQ ID NO: 60) and/or CY[FLIV][SDN][ATVG] (SEQ ID NO: 61) or one or both motifs DYQ[LI]G (SEQ ID NO: 62) and/or FQ[TAV]Q[PSD] (SEQ ID NO: 63).

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) DYQ[LI]G (SEQ ID NO: 62) or FQ[TAV]Q[PSD] (SEQ ID NO: 63); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES), wherein the GH114 glycosyl hydrolase enzyme is selected from the list consisting of:
   i. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
   ii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
   iii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
   iv. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
   v. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
   vi. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
   vii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
   viii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
   ix. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
   x. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
   xi. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51; and
   xii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) DYQ[LI]G (SEQ ID NO: 62) or FQ[TAV]Q[PSD] (SEQ ID NO: 63); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES),
   wherein the GH114 glycosyl hydrolase enzyme is selected from the list consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 79, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 90, SEQ ID NO: 91, SEQ ID NO: 92, SEQ ID NO: 93, SEQ ID NO: 94, SEQ ID NO: 96, SEQ ID NO: 131, SEQ ID NO: 132, SEQ ID NO: 134, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144 and SEQ ID NO: 145.

Another interesting clade derived from the VAE clade (and the DYQ clade) is the AEE clade, which comprises some of the polypeptides of the VAE clade. The polypeptides of this clade comprise one or two distinct conservative motifs AEECG (SEQ ID NO: 64) or NAFQ[AT]Q (SEQ ID NO: 65), which are conserved within the clade.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) AEECG (SEQ ID NO: 64) or NAFQ[AT]Q (SEQ ID NO: 65); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES).

The polypeptides of the AEE clade are also comprised in the VAE and DYQ clade, these polypeptides will comprise at least one or both motifs [VLI]XE[EDSQ]C (SEQ ID NO: 60) and/or CY[FLIV][SDN][ATVG] (SEQ ID NO: 61), one or both motifs DYQ[LI]G (SEQ ID NO: 62) and/or FQ[TAV]Q[PSD] (SEQ ID NO: 63) and one or both motifs AEECG (SEQ ID NO: 64) and/or NAFQ[AT]Q (SEQ ID NO: 65).

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) AEECG (SEQ ID NO: 64) or NAFQ[AT]Q (SEQ ID NO: 65); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES), wherein the GH114 glycosyl hydrolase enzyme is selected from the list consisting of:
   i. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
   ii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
   iii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
   iv. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
   v. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
   vi. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24; and
   vii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) AEECG (SEQ ID NO: 64) or NAFQ[AT]Q (SEQ ID NO: 65); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES),
   wherein the GH114 glycosyl hydrolase enzyme is selected from the list consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 79, SEQ ID NO: 82, SEQ ID NO: 83, SEQ ID NO: 84, SEQ ID NO: 85, SEQ ID NO: 132, SEQ ID NO: 136, SEQ ID NO: 138, and SEQ ID NO: 142.

Another interesting clade derived from the VAE clade is the IEY clade, which comprises some of the polypeptides of the VAE-clade. The polypeptides of this clade comprise one or two distinct conservative motifs GXXVX[NHQTS]IEY[PG] (SEQ ID NO: 68), VICYF (SEQ ID NO: 69), which are conserved within the clade.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) GXXVX[NHQTS]IEY[PG] (SEQ ID NO: 68) or VICYF (SEQ ID NO: 69); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES).

The polypeptides of the IEY clade is also comprised in the VAE, these polypeptides will comprise at least one or both motifs [VLI]XE[EDSQ]C (SEQ ID NO: 60) and/or CY[FLIV][SDN][ATVG] (SEQ ID NO: 61), and one or both motifs GXXVX[NHQTS]IEY[PG] (SEQ ID NO: 68) and/or VICYF (SEQ ID NO: 69).

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) GXXVX[NHQTS]IEY[PG] (SEQ ID NO: 68) or VICYF (SEQ ID NO: 69); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES), wherein the GH114 glycosyl hydrolase enzyme is selected from the list consisting of:
   i. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
   ii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18; and
   iii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) GXXVX[NHQTS]IEY[PG] (SEQ ID NO: 68) or VICYF (SEQ ID NO: 69); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES),
   wherein the GH114 glycosyl hydrolase enzyme is selected from the list consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 133 and SEQ ID NO: 135.

Another interesting clade derived from the VAE and the IEY clade is the ICY clade, which comprises some of the polypeptides of the VAE-clade and the IEY clade. The polypeptides of this clade comprise the distinct conservative motif ICYFSA (SEQ ID NO: 70), which is conserved within the clade.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises the motif ICYFSA (SEQ ID NO: 70); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES).

The polypeptides of the ICY clade is also comprised in the VAE and the IEY clade, these polypeptides will comprise at least one or both motifs [VLI]XE[EDSQ]C (SEQ ID NO: 60) and/or CY[FLIV][SDN][ATVG] (SEQ ID NO: 61), one or both motifs GXXVX[NHQTS]IEY[PG] (SEQ ID NO: 68) and/or VICYF (SEQ ID NO: 69) and the motif ICYFSA (SEQ ID NO: 70).

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises the motif ICYFSA (SEQ ID NO: 70); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES), wherein the GH114 glycosyl hydrolase enzyme is selected from the list consisting of:
   i. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15; and
   ii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57;

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises the motif ICYFSA (SEQ ID NO: 70); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES),
   wherein the GH114 glycosyl hydrolase enzyme is selected from the list consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 98, SEQ ID NO: 101, SEQ ID NO: 104, SEQ ID NO: 107, SEQ ID NO: 114, SEQ ID NO: 115, SEQ ID NO: 120, SEQ ID NO: 124, SEQ ID NO: 125, SEQ ID NO: 127, SEQ ID NO: 129 and SEQ ID NO: 133.

Another interesting clade derived from the VAE and the IEY clade is the DFAVL clade, which comprises some of the polypeptides of the VAE clade and the IEY clade. The polypeptides of this clade comprise the distinct conservative motif DFAVL (SEQ ID NO: 71), which is conserved within the clade.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises the motif DFAVL (SEQ ID NO: 71); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES).

The polypeptides of the DFAVL clade are also comprised in the VAE and the IEY clade, these polypeptides will comprise at least one or both motifs [VLI]XE[EDSQ]C (SEQ ID NO: 60) and/or CY[FLIV][SDN][ATVG] (SEQ ID NO: 61), one or both motifs GXXVX[NHQTS]IEY[PG] (SEQ ID NO: 68) and/or VICYF (SEQ ID NO: 69) and the motif DFAVL (SEQ ID NO: 71).

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises the motif DFAVL (SEQ ID NO: 71); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES), wherein the GH114 glycosyl hydrolase enzyme is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises the motif DFAVL (SEQ ID NO: 71); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES),
   wherein the GH114 glycosyl hydrolase enzyme is a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 110, SEQ ID NO: 111, SEQ ID NO: 112, SEQ ID NO: 113, SEQ ID NO: 116, SEQ ID NO: 117, SEQ ID NO: 118, SEQ ID NO: 119, SEQ ID NO: 121, SEQ ID NO: 123, SEQ ID NO: 126, SEQ ID NO: 128, SEQ ID NO: 130 and SEQ ID NO: 135.

Another interesting clade derived from the VAE clade is the WQW clade, which comprises some of the polypeptides of the VAE-clade. The polypeptides of this clade comprise one or both distinct conservative motif(s) WQWQL (SEQ ID NO: 66) or [VLI][GASD]LKN[DGS][VLIP] (SEQ ID NO: 67), which are conserved within the clade.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl one or both motif(s) WQWQL (SEQ ID NO: 66) or [VLI][GASD]LKN[DGS][VLIP] (SEQ ID NO: 67); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES).

The polypeptides of the WQW clade is also comprised in the VAE, these polypeptides will comprise at least one or both motifs [VLI]XE[EDSQ]C (SEQ ID NO: 60) and/or CY[FLIV][SDN][ATVG] (SEQ ID NO: 61) and/or one or both motif(s) WQWQL (SEQ ID NO: 66) or [VLI][GASD]LKN[DGS][VLIP] (SEQ ID NO: 67).

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) WQWQL (SEQ ID NO: 66) or [VLI][GASD]LKN[DGS][VLIP] (SEQ ID NO: 67); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES), wherein the GH114 glycosyl hydrolase enzyme is selected from the list consisting of:
   i. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
   ii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33; and
   iii. a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36.

One embodiment of the invention relates to a cleaning composition comprising:
(a) at least 0.01 ppm GH114 glycosyl hydrolase enzyme, preferably having alpha-1,4-polygalactosaminidase activity, wherein the GH114 glycosyl comprises one or both motif(s) WQWQL (SEQ ID NO: 66) or [VLI][GASD]LKN[DGS][VLIP] (SEQ ID NO: 67); and
(b) one or more cleaning composition components, preferably at least one surfactant, such as an anionic surfactant, wherein the anionic surfactant preferably include sulfates and sulfonates, such as linear alkylbenzene sulfonates (LAS), alpha-olefin sulfonates (AOS) and alkyl alcohol ether sulfates (AES or AEOS or FES), wherein the GH114 glycosyl hydrolase enzyme is selected from the list consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 80, SEQ ID NO: 81, SEQ ID NO: 89, SEQ ID NO: 95 and SEQ ID NO: 146.

The compositions according to the invention preferably comprises a GH114 glycosyl hydrolase, wherein the enzyme is capable of “deep cleaning” of an item.

Furthermore, the GH114 to be formulated into the composition of the invention preferably has improved wash performance or cleaning performance and the GH114 enzyme to be formulated into a composition according to the invention preferably has “enzyme detergency benefit” which is defined herein as the advantageous effect an enzyme may add to a detergent compared to the same detergent without the enzyme. The terms wash performance and detergency is explained further in the examples.

In one embodiment, the composition according to invention comprising a GH114 glycosyl hydrolase, wherein the GH114 glycosyl hydrolase has wash performance (WP) in liquid Model detergent A, measured as a delta L (ΔL)>1, preferably a delta L (ΔL) above 5, preferably a delta L (ΔL) above 10, preferably a delta L (ΔL) above 15, preferably a delta L (ΔL) above 20, when delta L (ΔL) is calculated (L(swatch washed with enzyme)−L(swatch washed without enzyme)) as described in examples 5 and 7.

The GH114 of the invention preferably has enzyme detergency benefit properties and one embodiment of the invention relates to a composition, comprising a GH114 glycosyl hydrolase enzyme, wherein the GH114 glycosyl hydrolase enzyme has biofilm removal activity, wherein in the % remaining biofilm is less than 80%, such as less than 70%, such as less than 60%, such as less than 50%, such as less than 40%, such as less than 30%, such as less than 20%, such as less than 10%, such as less than 1% or even no biofilm remaining on the item, e.g., textile, preferably when measured as described in Example 8.

The choice of cleaning components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

### Surfactants

The cleaning composition may comprise one or more surfactants, which may be anionic and/or cationic and/or non-ionic and/or semi-polar and/or zwitterionic, or a mixture thereof. In a particular embodiment, the detergent composition includes a surfactant system (comprising more than one surfactant), e.g., a mixture of one or more non-ionic surfactants and one or more anionic surfactants. In one embodiment the detergent comprises at least one anionic surfactant than at least one non-ionic surfactant, the weight ratio of anionic to non-ionic surfactant may be from 10:1 to 1:10. In one embodiment the amount of anionic surfactant is higher than the amount of non-ionic surfactant, e.g., the weight ratio of anionic to non-ionic surfactant may be from 10:1 to 1.1:1 or from 5:1 to 1.5:1. The amount of anionic to non-ionic surfactant may also be equal and the weight ratios 1:1. In one embodiment the amount of non-ionic surfactant is higher than the amount of anionic surfactant and the weight ratio may be 1:10 to 1:1.1. Preferably the weight ratio of anionic to non-ionic surfactant is from 10:1 to 1:10, such as from 5:1 to 1:5, or from 5:1 to 1:1.2. Preferably, the weight fraction of non-ionic surfactant to anionic surfactant is from 0 to 0.5 or 0 to 0.2 thus non-ionic surfactant can be present or absent if the weight fraction is 0, but if non-ionic surfactant is present, then the weight fraction of the non-ionic surfactant is preferably at most 50% or at most 20% of the total weight of anionic surfactant and non-ionic surfactant. Light duty detergent usually comprises more non-ionic than anionic surfactant and there the fraction of non-ionic surfactant to anionic surfactant is preferably from 0.5 to 0.9. The total weight of surfactant(s) is typically present at a level of from about 0.1% to about 60% by weight, such as about 1% to about 40%, or about 3% to about 20%, or about 3% to about 10%. The surfactant(s) is chosen based on the desired cleaning application, and may include any conventional surfactant(s) known in the art. When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, typically available as sodium or potassium salts or salts of monoethanolamine (MEA, 2-aminoethan-1-ol) or triethanolamine (TEA, 2,2′,2″-nitrilotriethan-1-ol); in particular, linear alkylbenzenesulfonates (LAS), isomers of LAS such as branched alkylbenzenesulfonates (BABS) and phenylalkanesulfonates; olefin sulfonates, in particular alpha-olefinsulfonates (AOS); alkyl sulfates (AS), in particular fatty alcohol sulfates (FAS), i.e., primary alcohol sulfates (PAS) such as dodecyl sulfate; alcohol ethersulfates (AES or AEOS or FES, also known as alcohol ethoxysulfates or fatty alcohol ether sulfates); paraffin sulfonates (PS) including alkane-1-sulfonates and secondary alkanesulfonates (SAS); ester sulfonates, including sulfonated fatty acid glycerol esters and alpha-sulfo fatty acid methyl esters (alpha-SFMe or SES or MES); alkyl- or alkenylsuccinic acids such as dodecenyl/tetradecenyl succinic acid (DTSA); diesters and monoesters of sulfosuccinic acid; fatty acid derivatives of amino acids. Furthermore, salts of fatty acids (soaps) may be included.

When included therein the detergent will usually contain from about 1% to about 40% by weight of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of non-ionic surfactants include alcohol ethoxylates (AE or AEO), alcohol propoxylates, in particular propoxylated fatty alcohols (PFA), ethoxylated and propoxylated alcohols, alkoxylated fatty acid alkyl esters, such as ethoxylated and/or propoxylated fatty acid alkyl esters (in particular methyl ester ethoxylates, MEE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01 to about 10% by weight of a semipolar surfactant. Non-limiting examples of semipolar surfactants include amine oxides (AO) such as alkyldimethylamine oxides, in particular N-(coco alkyl)-N,N-dimethylamine oxide and N-(tallow-alkyl)-N,N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

Additional bio-based surfactants may be used, e.g., wherein the surfactant is a sugar-based non-ionic surfactant which may be a hexyl-β-D-maltopyranoside, thiomaltopyranoside or a cyclic-maltopyranoside, such as described in EP 2516606 B1.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl) inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 2009/102854, U.S. Pat. No. 5,977,053.

### Bleaching Systems

The detergent may contain 0-30% by weight, such as about 1% to about 20%, of a bleaching system. Any bleaching system comprising components known in the art for use in cleaning detergents may be utilized. Suitable bleaching system components include sources of hydrogen peroxide; sources of peracids; and bleach catalysts or boosters.

### Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide—urea (1/1).

### Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.
a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.
b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO 98/17767. A particular family of bleach activators of interest was disclosed in EP 624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster. Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese (III). The bleach catalysts may also be other metal compounds; such as iron or cobalt complexes.

In some embodiments, where a source of a peracid is included, an organic bleach catalyst or bleach booster may be used having one of the following formulae:

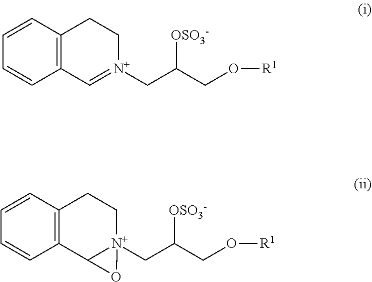

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g., in WO 2007/087258, WO 2007/087244, WO 2007/087259, EP 1867708 (Vitamin K) and WO 2007/087242. Suitable photobleaches may for example be sulfonated zinc or aluminium phthalocyanines.

### Metal Care Agents

Metal care agents may prevent or reduce the tarnishing, corrosion or oxidation of metals, including aluminium, stainless steel and non-ferrous metals, such as silver and copper. Suitable examples include one or more of the following:

(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain Ci-C20-alkyl groups (e.g., C1-C20-alkyl groups) and hydroxyl, thio, phenyl or halogen such as fluorine, chlorine, bromine and iodine.

(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one aspect, suitable metal salts and/or metal complexes may be chosen from the group consisting of Mn(II) sulphate, Mn(II) citrate, Mn(II) stearate, Mn(II) acetylacetonate, K\hat{ }TiF6 (e.g., K2TiF6), K\hat{ }ZrF6 (e.g., K2ZrF6), CoSO4, Co(NOs)2 and Ce(NOs)3, zinc salts, for example zinc sulphate, hydrozincite or zinc acetate.;

(c) silicates, including sodium or potassium silicate, sodium disilicate, sodium metasilicate, crystalline phyllosilicate and mixtures thereof.

Further suitable organic and inorganic redox-active substances that act as silver/copper corrosion inhibitors are disclosed in WO 94/26860 and WO 94/26859. Preferably the composition of the invention comprises from 0.1 to 5% by weight of the composition of a metal care agent, preferably the metal care agent is a zinc salt.

### Hydrotropes

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fibre protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl) cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Particularly preferred polymer is ethoxylated homopolymer Sokalan® HP 20 from BASF, which helps to prevent redeposition of soil in the wash liquor.

Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/003274, WO 2005/003275, WO 2005/003276 and EP 1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g., WO 2007/087257 and WO 2007/087243.

### Enzymes

The detergent additive as well as the detergent composition may comprise one or more additional enzymes such as one or more lipase, cutinase, an amylase, carbohydrase, cellulase, pectinase, mannanase, arabinase, galactanase, xylanase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include mono-component and mixtures of enzymes of bacterial or fungal origin. Chemically modified or protein engineered mutants are also contemplated. The cellulase may for example be a mono-component or a mixture of mono-component endo-1,4-beta-glucanase also referred to as endoglucanase. Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g., T. reesei or T. viride. Other suitable cellulases are from Thielavia, e.g., Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307.

Other cellulases are endo-beta-1,4-glucanase enzyme having a sequence of at least 97% identity to the amino acid sequence of position 1 to position 773 of SEQ ID NO: 2 of WO 2002/099091 or a family 44 xyloglucanase, which a xyloglucanase enzyme having a sequence of at least 60% identity to positions 40-559 of SEQ ID NO: 2 of WO 01/62903.

Commercially available cellulases include Carezyme®, Carezyme® Premium, Celluzyme®, Celluclean®, Celluclast®, Endolase®, Renozyme®; Whitezyme® Celluclean® Classic, Cellusoft® (Novozymes A/S), Puradax®, Puradax HA, and Puradax EG (available from Genencor International Inc.) and KAC-500 (B)™ (Kao Corporation).

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 99/64619. A commercially available mannanase is Mannaway (Novozymes A/S).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

### Lipases and Cutinases

Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Humicola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 2007/87508 and WO 2009/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g., acyltransferases with homology to Candida antarctica lipase A (WO 2010/111143), acyltransferase from Mycobacterium smegmatis (WO 2005/56782), perhydrolases from the CE 7 family (WO 2009/67279), and variants of the M. smegmatis perhydrolase in particular the S54V variant used in the commercial product Gentle Power Bleach from Huntsman Textile Effects Pte Ltd (WO 2010/100028).

### Amylases

Suitable amylases include alpha-amylases and/or a glucoamylases and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/19467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

### M197T;

### H156Y+A181T+N190F+A209V+Q264S; or

### G48A+T491+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/23873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/23873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 2008/153815, SEQ ID NO: 10 in WO 01/66712 or variants thereof having 90% sequence identity to SEQ ID NO: 2 of WO 2008/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 2009/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T1311, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

### N128C+K178L+T182G+Y305R+G475K;

### N128C+K178L+T182G+F202Y+Y305R+D319T+G475K;

### S125A+N128C+K178L+T182G+Y305R+G475K; or

S125A+N128C+T1311+T165|+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO 2013/184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

### E187P+1203Y+G476K

### E187P+1203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO 2010/104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

### N21D+D97N+V128I

wherein the variants optionally further comprise a substitution at position 200 and/or a deletion at position 180 and/or position 181.

Other suitable amylases are the alpha-amylase having SEQ ID NO: 12 in WO 01/66712 or a variant having at least 90% sequence identity to SEQ ID NO: 12. Preferred amylase variants are those having a substitution, a deletion or an insertion in one of more of the following positions of SEQ ID NO: 12 in WO 01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particularly preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO 2013/001078 and WO 2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

### Proteases

Suitable proteases include those of bacterial, fungal, plant, viral or animal origin, e.g., vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from, e.g., family M4 or other metalloproteases such as those from M5, M7 or M8 families.

The term “subtilases” refers to a sub-group of serine protease according to Siezen et al., 1991, Protein Engng. 4:719-737 and Siezen et al., 1997, Protein Science 6:501-523. Serine proteases are a subgroup of proteases characterized by having a serine in the active site, which forms a covalent adduct with the substrate. The subtilases may be divided into 6 sub-divisions, i.e., the Subtilisin family, the Thermitase family, the Proteinase K family, the Lantibiotic peptidase family, the Kexin family and the Pyrolysin family. Examples of subtilases are those derived from Bacillus such as Bacillus lentus, Bacillus alkalophilus, Bacillus subtilis, Bacillus amyloliquefaciens, Bacillus pumilus and Bacillus gibsonii described in; U.S. Pat. No. 7,262,042 and WO 2009/021867, and Subtilisin lentus, Subtilisin Novo, subtilisin Carlsberg, Bacillus licheniformis, subtilisin BPN′, subtilisin 309, subtilisin 147 and subtilisin 168 and, e.g., protease PD138 described in (WO 93/18140). Other useful proteases may be those described in WO 01/16285 and WO 02/016547. Examples of trypsin-like proteases are trypsin (e.g., of porcine or bovine origin) and the Fusarium protease described in WO 94/25583 and WO 2005/040372, and the chymotrypsin proteases derived from Cellumonas described in WO 2005/052161 and WO 2005/052146. A further preferred protease is the alkaline protease from Bacillus lentus DSM 5483, as described for example in WO 95/23221, and variants thereof which are described in WO 92/21760, WO 95/23221, EP 1921147 and EP 1921148. Examples of metalloproteases are the neutral metalloprotease as described in WO 2007/044993 (Proctor & Gamble/Genencor Int.) such as those derived from Bacillus amyloliquefaciens. Examples of useful proteases are the variants described in: WO 89/06279 WO 92/19729, WO 96/34946, WO 98/20115, WO 98/20116, WO 99/11768, WO 01/44452, WO 2003/006602, WO 2004/03186, WO 2004/041979, WO 2007/006305, WO 2011/036263, WO 2011/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus lentus protease shown in SEQ ID NO: 1 of WO 2016/001449. More preferred the protease variants may comprise one or more of the mutations selected from the group consisting of: S3T, V41, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Y203W, S206G, L211Q, L211D, N212D, N212S, M216S, A226V, K229L, Q230H, Q239R, N246K, N255W, N255D, N255E, L256E, L256D T268A and R269H. The protease variants are preferably variants of the Bacillus lentus protease (Savinase®) shown in SEQ ID NO: 1 of WO 2016/001449, the Bacillus amyloliquefaciens protease (BPN′) shown in SEQ ID NO: 2 of WO 2016/001449. The protease variants preferably have at least 80% sequence identity to SEQ ID NO: 1 or SEQ ID NO: 2 of WO 2016/001449.

A protease variant comprising a substitution at one or more positions corresponding to positions 171, 173, 175, 179, or 180 of SEQ ID NO: 1 of WO 2004/067737, wherein said protease variant has a sequence identity of at least 75% but less than 100% to SEQ ID NO: 1 of WO 2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes A/S), those sold under the tradename Maxatase®, Maxacal®, Maxapem®, Purafect Ox®, Purafect OxPR, Puramax®, FN2®, FN3®, FN4®, Excellase®, Excellenz P1000™, Excellenz P1250™, Eraser®, Preferenz P100™, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Peroxidases/Oxidases

A suitable peroxidase is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A suitable peroxidase includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. Preferably, the haloperoxidase is a vanadium haloperoxidase, i.e., a vanadate-containing haloperoxidase. Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

A suitable oxidase includes in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5). Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular, a laccase derived from Coprinopsis cinerea, as disclosed in WO 97/08325; or from Myceliophthora thermophila, as disclosed in WO 95/33836.

### Nucleases

Suitable nucleases include deoxyribonucleases (DNases) and ribonucleases (RNases), which are any enzyme that catalyzes the hydrolytic cleavage of phosphodiester linkages in the DNA or RNA backbone respectively, thus degrading DNA and RNA. There are two primary classifications based on the locus of activity. Exonucleases digest nucleic acids from the ends. Endonucleases act on regions in the middle of target molecules. The nuclease is preferably a DNase, which is preferable is obtainable from a microorganism, preferably a bacterium; in particular a DNase which is obtainable from a species of Bacillus is preferred; in particular a DNase which is obtainable from Bacillus cibi, Bacillus subtilis or Bacillus licheniformis is preferred. Examples of suitable DNases are described in WO 2011/098579, WO 2014/087011 and WO 2017/060475.

### Dispersants

The cleaning, e.g., detergent compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The cleaning, e.g., detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The cleaning, e.g., detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl) stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be non-ionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C 6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose-based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040. Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The cleaning, e.g., detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g., without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water-soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water-soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids: US 2009/0011970.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Granular Detergent Formulations

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The glycosyl hydrolase, e.g., GH114 glycosyl hydrolase may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulate for the detergent industry is disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. The multi-enzyme co-granule may comprise an enzyme of the invention and one or more enzymes selected from the group consisting of proteases, lipases, cellulases, xyloglucanases, perhydrolases, peroxidases, lipoxygenases, laccases, hemicellulases, proteases, cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof. WO 2013/188331 also relates to a method of treating and/or cleaning a surface, preferably a fabric surface comprising the steps of (i) contacting said surface with the detergent composition as claimed and described herein in aqueous wash liquor, (ii) rinsing and/or drying the surface.

An embodiment of the invention relates to an enzyme granule/particle comprising the glycosyl hydrolase, e.g., GH114 glycosyl hydrolase. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm. The core may include additional materials such as fillers, fibre materials (cellulose or synthetic fibres), stabilizing agents, solubilising agents, suspension agents, viscosity regulating agents, light spheres, plasticizers, salts, lubricants and fragrances. The core may include binders, such as synthetic polymer, wax, fat, or carbohydrate. The core may comprise a salt of a multivalent cation, a reducing agent, an antioxidant, a peroxide decomposing catalyst and/or an acidic buffer component, typically as a homogenous blend. The core may consist of an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606. The coating may be applied in an amount of at least 0.1% by weight of the core, e.g., at least 0.5%, 1% or 5%. The amount may be at most 100%, 70%, 50%, 40% or 30%. The coating is preferably at least 0.1 μm thick, particularly at least 0.5 μm, at least 1 μm or at least 5 μm. In a one embodiment, the thickness of the coating is below 100 μm. In another embodiment, the thickness of the coating is below 60 μm. In an even more particular embodiment, the total thickness of the coating is below 40 μm. The coating should encapsulate the core unit by forming a substantially continuous layer. A substantially continuous layer is to be understood as a coating having few or no holes, so that the core unit it is encapsulating/enclosing has few or no uncoated areas. The layer or coating should be homogeneous in thickness. The coating can further contain other materials as known in the art, e.g., fillers, antisticking agents, pigments, dyes, plasticizers and/or binders, such as titanium dioxide, kaolin, calcium carbonate or talc. A salt coating may comprise at least 60% by weight w/w of a salt, e.g., at least 65%, at least 70%, at least 75%, at least 80%, at least 85%, at least 90%, at least 95% or at least 99% by weight w/w. The salt may be added from a salt solution where the salt is completely dissolved or from a salt suspension wherein the fine particles is less than 50 μm, such as less than 10 μm or less than 5 μm. The salt coating may comprise a single salt or a mixture of two or more salts. The salt may be water soluble and may have a solubility at least 0.1 grams in 100 g of water at 20° C., preferably at least 0.5 g per 100 g water, e.g., at least 1 g per 100 g water, e.g., at least 5 g per 100 g water. The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used. The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710. Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate. The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate. Preferably the salt is applied as a solution of the salt, e.g., using a fluid bed.

One embodiment of the present invention provides a granule, which comprises:
(a) a core comprising a GH114 glycosyl hydrolase according to the invention, and
(b) optionally a coating consisting of one or more layer(s) surrounding the core.

One embodiment of the invention relates to a granule, which comprises:
(a) a core comprising a GH114 glycosyl hydrolase, preferably having alpha-1,4-polygalactosaminidase activity, having at least 60%, at least 65%, at least 70%, at least 75%, at least 80%, at least 85%, at least 90%, at least 95%, at least 98% or 100% sequence identity to the amino acid sequence shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146 and
(b) optionally a coating consisting of one or more layer(s) surrounding the core.

### Medical Cleaning

The present invention further relates to methods of cleaning a medical device and to the use of a composition comprising a glycosyl hydrolase, preferably a GH114 glycosyl hydrolase and at least one adjunct ingredient for cleaning of a medical device. The invention further relates to a method of preventing biofilm formation on a medical device, e.g., an indwelling medical device or implant comprising coating the device with at least one GH114 glycosyl hydrolase.

One embodiment of the invention relates to a method of preventing biofilm formation on a medical device, e.g., an indwelling medical device or implant comprising coating the device with at least one GH114 glycosyl hydrolase.

The polypeptides suitable for use in medical cleaning and in compositions for medical cleaning are described above and include polypeptides which comprises one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) and/or polypeptide selected from the group consisting of polypeptides having the amino acid sequence of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity thereto.

One aspect of the invention relates to a method of cleaning a medical device, wherein the method comprises
a) contacting the medical device with the composition comprising a glycosyl hydrolase, preferably having alpha-1,4-polygalactosaminidase activity, preferably a GH114 glycosyl hydrolase, for a period effective to clean the medical device;
b) cleaning, the medical device; and
c) optionally disinfect the medical device.

One aspect of the invention relates to a method of cleaning a medical device, wherein the method comprises
a) contacting the medical device with the composition comprising a GH114 glycosyl hydrolase, preferably having alpha-1,4-polygalactosaminidase activity, which comprises one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71)
b) and/or is selected from the group consisting of GH114 glycosyl hydrolases having the amino acid sequence shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity thereto, for a period effective to clean the medical device;
c) cleaning, the medical device; and
d) optionally disinfect the medical device.

One embodiment relates to a composition comprising a glycosyl hydrolase, preferably a GH114 glycosyl hydrolase, which comprises one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) and/or is selected from the group consisting of glycosyl hydrolases having the amino acid sequence of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity thereto and preferably an adjunct ingredient. The composition may be an anti-biofouling composition and the composition may be a cleaning or pharmaceutical composition. The adjunct ingredient may be any excipient suitable for, e.g., cleaning or pharmaceutical compositions. The adjuncts/excipients are within the choice of the skilled artisan. The adjunct ingredient may be selected from the group consisting of surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxidoreductases, bluing agents and fluorescent dyes, antioxidants, and solubilizers. The compositions may be used for detaching biofilm or preventing biofilm formation on surfaces such as medical devices.

One embodiment of the invention relates to the use of a composition comprising a glycosyl hydrolase, preferably having alpha-1,4-polygalactosaminidase activity, preferably a GH114 glycosyl hydrolase, which comprises one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) and/or is selected from the group consisting glycosyl hydrolases having the amino acid sequence of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity thereto and preferably an adjunct ingredient for cleaning a medical device or an implant.

Medical devices are products which are used to diagnose, prevent, relieve or treat a disease, disability, injury, etc. There term includes wheelchairs, glasses to pacemakers, mobile phone apps and state-of-the-art surgical equipment. Medical devices are grouped into four classes known as I, Ia, IIb and III, with Class I being the lowest risk and Class Ill being the highest risk. The medical device may be characterized in that at least a portion of a patient-contactable surface of said device is coated with composition comprising a GH114 glycosyl hydrolase of the invention. The medical device or implant may be any device or implant that is susceptible to biofilm formation. The medical device may be selected from the group consisting of a catheter such as a central venous catheter, intravascular catheter, urinary catheter, Hickman catheter, peritoneal dialysis catheter, endrotracheal catheter, endoscope, or wherein the device is a mechanical heart valve, a cardiac pacemaker, an arteriovenous shunt, a scleral buckle, a prosthetic joint, a tympanostomy tube, a tracheostomy tube, a voice prosthetic, a penile prosthetic, an artificial urinary sphincter, a synthetic pubovaginal sling, a surgical suture, a bone anchor, a bone screw, an intraocular lens, a contact lens, an intrauterine device, an aortofemoral graft, a vascular graft, a needle, a Luer-Lok connector, a needleless connector and a surgical instrument.

### Uses

The polypeptides of the invention having hydrolytic activity may be used for cleaning, e.g., deep cleaning of an item, such as a textile. One embodiment of the invention relates to the use of a glycosyl hydrolase, preferably a GH114 glycosyl hydrolase in a cleaning process, such as laundry and/or dish wash.

In a preferred embodiment, the GH114 glycosyl hydrolase polypeptides of the invention comprise one or more of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71). In a preferred embodiment, the GH114 glycosyl hydrolase comprising one or more of the motif(s) selected from the group consisting of: [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71).

In some embodiments of the invention relate to the use of glycosyl hydrolase, preferably having alpha-1,4-polygalactosaminidase activity, preferably a GH114 glycosyl hydrolase according to the invention for prevention reduction or removal of malodor. Some embodiment of the invention relates to the use of a polypeptide of the invention for prevention or reduction of anti-redeposition and improvement of whiteness of a textile subjected to multiple washes. One embodiment of the invention relates to the use of a glycosyl hydrolase, preferably having alpha-1,4-polygalactosaminidase activity, preferably a GH114 glycosyl hydrolase according to the invention for deep cleaning of an item, wherein item is a textile. One embodiment of the invention relates to the use of a glycosyl hydrolase, preferably having alpha-1,4-polygalactosaminidase activity, preferably a GH114 glycosyl hydrolase polypeptide according to the invention
(i) for preventing, reducing or removing stickiness of the item;
(ii) for pretreating stains on the item;
(iii) for preventing, reducing or removing redeposition of soil during a wash cycle;
(iv) for preventing, reducing or removing adherence of soil to the item;
(v) for maintaining or improving whiteness of the item;
(vi) for preventing, reducing or removal malodor from the item,
   wherein the item is a textile.

One embodiment of the invention relates to the use of a glycosyl hydrolase, preferably having alpha-1,4-polygalactosaminidase activity, preferably a GH114 glycosyl hydrolase polypeptide according to the invention for deep cleaning of an item, wherein item is a textile. One embodiment of the invention relates to the use of a glycosyl hydrolase, preferably having alpha-1,4-polygalactosaminidase activity, preferably a GH114 glycosyl hydrolase polypeptide,
(i) for preventing, reducing or removing stickiness of the item;
(ii) for pretreating stains on the item;
(iii) for preventing, reducing or removing redeposition of soil during a wash cycle;
(iv) for preventing, reducing or removing adherence of soil to the item;
(v) for maintaining or improving whiteness of the item;
(vi) for preventing, reducing or removal malodor from the item,
   optionally wherein the item is a textile, wherein the glycosyl hydrolase polypeptide is selected from the group consisting of:
(a) a polypeptide having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54;
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57.

One preferred embodiment relates to the use of a GH114 glycosyl hydrolase,
i. for preventing, reducing or removing stickiness of the item;
ii. for preventing, reducing or removing biofilm or biofilm components
iii. for reducing or removing pel stains on the item;
iv. for preventing, reducing or removing redeposition of soil during a wash cycle;
V. for preventing, reducing or removing adherence of soil to the item;
vi. for maintaining or improving whiteness of the item;
vii. for preventing, reducing or removal malodor from the item,
   wherein the item is a textile.

One preferred embodiment relates to the use of a GH114 glycosyl hydrolase,
i. for preventing, reducing or removing stickiness of the item;
ii. for preventing, reducing or removing biofilm or biofilm components
iii. for reducing or removing pel stains on the item;
iv. for preventing, reducing or removing redeposition of soil during a wash cycle;
V. for preventing, reducing or removing adherence of soil to the item;
vi. for maintaining or improving whiteness of the item;
vii. for preventing, reducing or removal malodor from the item,
   wherein the item is a textile and wherein the polypeptide is selected from the group consisting of:
(a) a polypeptide having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
(b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
(c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
(d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
(e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
(f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
(g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
(h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
(i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
(m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
(n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
(p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
(r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54;
(s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57.

One preferred embodiment relates to the use of a GH114 glycosyl hydrolase,
i. for preventing, reducing or removing stickiness of the item;
ii. for preventing, reducing or removing biofilm or biofilm components
iii. for reducing or removing pel stains on the item;
iv. for preventing, reducing or removing redeposition of soil during a wash cycle;
V. for preventing, reducing or removing adherence of soil to the item;
vi. for maintaining or improving whiteness of the item;
vii. for preventing, reducing or removal malodor from the item,
   wherein the item is a textile and wherein the polypeptide is selected from the group consisting of:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(h) a polypeptide having, at least 50% at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(i) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(j) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 36;
(k) a polypeptide having at least 99% or 100% sequence identity to the polypeptide shown in SEQ ID NO: 39;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(m) a polypeptide having at least 85%, at least 90%, at least 95%, at least 96%, at least 97%, at least 98%, at least 99% or 100% sequence identity to the polypeptide shown in SEQ ID NO: 45;
(n) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(o) a polypeptide having at least 98%, at least 99% or 100% sequence identity to the polypeptide shown in SEQ ID NO: 51;
(p) a polypeptide having at least 80%, at least 85%, at least 90%, at least 95%, at least 96%, at least 97%, at least 98%, at least 99% or 100% sequence identity to the polypeptide shown in SEQ ID NO: 54; and
(q) a polypeptide having at least 85%, at least 90%, at least 95%, at least 96%, at least 97%, at least 98%, at least 99% or 100% sequence identity to the polypeptide shown in SEQ ID NO: 57.

One preferred embodiment relates to the use of a GH114 glycosyl hydrolase,
i. for preventing, reducing or removing stickiness of the item;
ii. for preventing, reducing or removing biofilm or biofilm components
iii. for reducing or removing pel stains on the item;
iv. for preventing, reducing or removing redeposition of soil during a wash cycle;
V. for preventing, reducing or removing adherence of soil to the item;
vi. for maintaining or improving whiteness of the item;
vii. for preventing, reducing or removal malodor from the item,
   wherein the item is a textile and wherein the polypeptide is selected from the group consisting of: a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146.

The polypeptides of the invention are particularly useful in cleaning processes such as laundry, where the polypeptide effectively reduces biofilm components such as alpha-1,4-polygalactosamine comprising biofilm as shown in the examples below. One embodiment of the invention relates to a method for laundering an item comprising the steps of:
a. exposing an item to a wash liquor comprising the GH114 glycosyl hydrolase, preferably having alpha-1,4-polygalactosaminidase activity, or a composition comprising a GH114 glycosyl hydrolase, preferably having alpha-1,4-polygalactosaminidase activity;
b. completing at least one wash cycle; and
c. optionally rinsing the item,
   wherein the item is a textile.

One preferred embodiment of the invention relates to a method for laundering an item comprising the steps of:
a. exposing an item to a wash liquor comprising a glycosyl hydrolase, preferably having alpha-1,4-polygalactosaminidase activity, preferably a GH114 glycosyl hydrolase or a composition comprising a glycosyl hydrolase, preferably having alpha-1,4-polygalactosaminidase activity, wherein glycosyl hydrolase is selected from the group consisting of:
   (a) a polypeptide having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 3;
   (b) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 6;
   (c) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 9;
   (d) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 12;
   (e) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 15;
   (f) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 18;
   (g) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 21;
   (h) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 24;
   (i) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
   (j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
   (k) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
   (l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 36;
   (m) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 39;
   (n) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO:
   (o) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 45;
   (p) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
   (q) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 51;
   (r) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 54;
   (s) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 57;
b. completing at least one wash cycle; and
c. optionally rinsing the item,
   wherein the item is a textile.

A preferred embodiment relates to a method for laundering an item comprising the steps of:
a. exposing an item to a wash liquor comprising a GH114 glycosyl hydrolase, preferably having alpha-1,4-polygalactosaminidase activity, or a composition comprising a GH114 glycosyl hydrolase, preferably having alpha-1,4-polygalactosaminidase activity, wherein the GH114 glycosyl hydrolase, is selected from the group consisting of:
   (a) a polypeptide having at least 90%, at least 95%, at least 96%, at least 97%, at least 98%, at least 99% or 100% sequence identity to the polypeptide shown in SEQ ID NO: 3;
   (b) a polypeptide having at least 90%, at least 95%, at least 96%, at least 97%, at least 98%, at least 99% or 100% sequence identity to the polypeptide shown in SEQ ID NO: 6;
   (c) a polypeptide having at least 85%, at least 90%, at least 95%, at least 96%, at least 97%, at least 98%, at least 99% or 100% sequence identity to the polypeptide shown in SEQ ID NO: 9;
   (d) a polypeptide having at least 85%, at least 90%, at least 95%, at least 96%, at least 97%, at least 98%, at least 99% or 100% sequence identity to the polypeptide shown in SEQ ID NO: 12;
   (e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
   (f) a polypeptide having at least 90%, at least 95%, at least 96%, at least 97%, at least 98%, at least 99% or 100% sequence identity to the polypeptide shown in SEQ ID NO: 24;
   (g) a polypeptide having at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
   (h) a polypeptide having, at least 50% at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
   (i) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
   (j) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 36;
   (k) a polypeptide having at least 99% or 100% sequence identity to the polypeptide shown in SEQ ID NO: 39;
   (l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
   (m) a polypeptide having at least 85%, at least 90%, at least 95%, at least 96%, at least 97%, at least 98%, at least 99% or 100% sequence identity to the polypeptide shown in SEQ ID NO: 45;
   (n) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
   (o) a polypeptide having at least 98%, at least 99% or 100% sequence identity to the polypeptide shown in SEQ ID NO: 51;
   (p) a polypeptide having at least 80%, at least 85%, at least 90%, at least 95%, at least 96%, at least 97%, at least 98%, at least 99% or 100% sequence identity to the polypeptide shown in SEQ ID NO: 54; and
   (q) a polypeptide having at least 85%, at least 90%, at least 95%, at least 96%, at least 97%, at least 98%, at least 99% or 100% sequence identity to the polypeptide shown in SEQ ID NO: 57
b. completing at least one wash cycle; and
c. optionally rinsing the item,
   wherein the item is a textile.

One preferred embodiment of the invention relates to a method for laundering an item comprising the steps of:
a. exposing an item to a wash liquor comprising a glycosyl hydrolase, preferably having alpha-1,4-polygalactosaminidase activity, preferably a GH114 glycosyl hydrolase or a composition comprising a glycosyl hydrolase, preferably having alpha-1,4-polygalactosaminidase activity, wherein glycosyl hydrolase is selected from the group consisting of: a polypeptide having at least 60%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ED NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 and SEQ ID NO: 146,
b. completing at least one wash cycle; and
c. optionally rinsing the item,
   wherein the item is a textile.

The invention is further summarized in the following paragraphs:

1. Use of a polypeptide comprising one or more of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) for deep cleaning of an item, wherein the item is a textile.
2. Use according to paragraph 1 for preventing, reducing or removing stickiness of the item.
3. Use according to paragraph 1 or 2 for pre-treating stains on the item.
4. Use according to any of paragraphs 1-3 for preventing, reducing or removing re-deposition of soil during a wash cycle.
5. Use according to any of paragraphs 1-4 for preventing, reducing or removing adherence of soil to the item.
6. Use according to any of paragraphs 1-5 for maintaining or improving the whiteness of the item.
7. Use according to any of paragraphs 1-6, wherein a malodor is reduced or removed from the item.
8. Use according to any of paragraphs 1-7, wherein the surface is a textile surface.
9. Use according to any of paragraphs 1-8, wherein the textile is made of cotton, Cotton/Polyester, Polyester, Polyamide, Polyacryl and/or silk.
10. Use according to any of paragraphs 1-9, wherein the polypeptide is a polypeptide of paragraphs 68-108.
11. A composition comprising a polypeptide comprising one or more of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) and a cleaning or an adjunct ingredient.
12. Composition according to paragraph 11, wherein the polypeptide is the polypeptide shown in paragraphs 55-79.
13. Composition according to paragraph 11 or 12, wherein the detergent adjunct ingredient is selected from the group consisting of surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.
14. Composition according to any of paragraphs 11-13, wherein the composition comprises from about 5 wt % to about 50 wt %, from about 5 wt % to about 40 wt %, from about 5 wt % to about 30 wt %, from about 5 wt % to about 20 wt %, from about 5 wt % to about 10 wt % anionic surfactant, preferably selected from linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.
15. Composition according to any of paragraphs 11-14, wherein the composition comprises from about 10 wt % to about 50 wt % of at least one builder, preferably selected from citric acid, methylglycine-N,N-diacetic acid (MGDA) and/or glutamic acid-N,N-diacetic acid (GLDA) and mixtures thereof.
16. Composition according to any of paragraphs 11-15 comprising from about 5 wt % to about 40 wt % nonionic surfactants, and from about 0 wt % to about 5 wt % anionic surfactants.
17. Composition according to paragraph 16, wherein the nonionic surfactant is selected from alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA) and combinations thereof.
18. Composition according to any of paragraphs 11-17, wherein the composition further comprises one or more enzymes selected from the group consisting of proteases, lipases, cutinases, amylases, carbohydrases, cellulases, pectinases, mannanases, arabinases, galactanases, xylanases and oxidases.
19. Composition according to any of paragraphs 11-18, wherein the composition is a bar, a homogenous tablet, a tablet having two or more layers, a pouch having one or more compartments, a regular or compact powder, a granule, a paste, a gel, or a regular, compact or concentrated liquid.
20. Composition according to any of paragraphs 11-19, wherein the composition is a cleaning composition selected from liquid detergent, powder detergent and granule detergent compositions.
21. Composition according to any of paragraphs 11-20, wherein the polypeptide comprises one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71) and wherein the polypeptide is selected from the group consisting of polypeptides having the amino acid sequence of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 39, SEQ ID NO: 42, SEQ ID NO: 45, SEQ ID NO: 48, SEQ ID NO: 51, SEQ ID NO: 54, SEQ ID NO: 57 SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146 and polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
22. Composition according to any of paragraphs 11-21, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65) and comprises the amino acid sequence shown SEQ ID NO: 3 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
23. Composition according to any of paragraphs 11-22, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65) and comprises the amino acid sequence shown SEQ ID NO: 6 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
24. Composition according to any of paragraphs 11-23, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65) and comprises the amino acid sequence shown SEQ ID NO: 9 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
25. Composition according to any of paragraphs 11-24, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65) and comprises the amino acid sequence shown SEQ ID NO: 12 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
26. Composition according to any of paragraphs 11-25, wherein the polypeptide comprising one or more motif(s) [VLI]XE[EDSQ]C (SEQ ID NO: 60), CY[FLIV][SDN][ATVG] (SEQ ID NO: 61), GXXVX[NHQTS]IEY[PG] (SEQ ID NO: 68), VICYF (SEQ ID NO: 69), ICYFSA (SEQ ID NO: 70), and comprises the amino acid sequence shown SEQ ID NO: 15 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
27. Composition according to any of paragraphs 11-26, wherein the polypeptide comprising one or more motif(s) [VLI]XE[EDSQ]C (SEQ ID NO: 60), CY[FLIV][SDN][ATVG] (SEQ ID NO: 61), GXXVX[NHQTS]IEY[PG] (SEQ ID NO: 68), VICYF (SEQ ID NO: 69), DFAVL (SEQ ID NO: 71) and comprises the amino acid sequence shown SEQ ID NO: 18 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
28. Composition according to any of paragraphs 11-27, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65) and comprises the amino acid sequence shown SEQ ID NO: 21 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
29. Composition according to any of paragraphs 11-28, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65) and comprises the amino acid sequence shown SEQ ID NO: 24 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
30. Composition according to any of paragraphs 11-29, wherein the polypeptide comprising one or more motif(s) [VLI]XE[EDSQ]C (SEQ ID NO: 60), CY[FLIV][SDN][ATVG] (SEQ ID NO: 61), WQWQL (SEQ ID NO: 66), [VLI][GASD]LKN[DGS][VLIP] (SEQ ID NO: 67) and comprises the amino acid sequence shown SEQ ID NO: 27 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
31. Composition according to any of paragraphs 11-30, wherein the polypeptide comprising one or more motif(s) [VLI]XE[EDSQ]C (SEQ ID NO: 60), CY[FLIV][SDN][ATVG] (SEQ ID NO: 61), WQWQL (SEQ ID NO: 66), [VLI][GASD]LKN[DGS][VLIP] (SEQ ID NO: 67) and comprises the amino acid sequence shown SEQ ID NO: 33 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
32. Composition according to any of paragraphs 11-31, wherein the polypeptide comprising one or more motif(s) [VLI]XE[EDSQ]C (SEQ ID NO: 60), CY[FLIV][SDN][ATVG] (SEQ ID NO: 61), WQWQL (SEQ ID NO: 66), [VLI][GASD]LKN[DGS][VLIP] (SEQ ID NO: 67) and comprises the amino acid sequence shown SEQ ID NO: 36 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
33. Composition according to any of paragraphs 11-32, wherein the polypeptide comprising one or more motif(s) [VLI]XE[EDSQ]C (SEQ ID NO: 60), CY[FLIV][SDN][ATVG] (SEQ ID NO: 61) and comprises the amino acid sequence shown SEQ ID NO: 30 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
34. Composition according to any of paragraphs 11-33, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63) and comprises the amino acid sequence shown SEQ ID NO: 39 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
35. Composition according to any of paragraphs 11-34, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63) and comprises the amino acid sequence shown SEQ ID NO: 42 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
36. Composition according to any of paragraphs 11-35, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63) and comprises the amino acid sequence shown SEQ ID NO: 45 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
37. Composition according to any of paragraphs 11-36, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63) and comprises the amino acid sequence shown SEQ ID NO: 48 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
38. Composition according to any of paragraphs 11-37, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63) and comprises the amino acid sequence shown SEQ ID NO: 51 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
39. Composition according to any of paragraphs 11-38, wherein the polypeptide comprising one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65) and comprises the amino acid sequence shown SEQ ID NO: 54 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
40. Composition according to any of paragraphs 11-39, wherein the polypeptide comprising one or more motif(s) [VLI]XE[EDSQ]C (SEQ ID NO: 60), CY[FLIV][SDN][ATVG] (SEQ ID NO: 61), GXXVX[NHQTS]IEY[PG] (SEQ ID NO: 68), VICYF (SEQ ID NO: 69), ICYFSA (SEQ ID NO: 70) and comprises the amino acid sequence shown SEQ ID NO: 57 or polypeptides having at least 60%, at least 65%, at least 70%, at least 75%, at least 80%, at least 85%, at least 90%, at least 95%, at least 96%, at least 97%, at least 98%, at least 99% or 100% sequence identity thereto.
41. A method for laundering an item comprising the steps of:
a) Exposing an item to a wash liquor comprising a polypeptide of paragraphs 55-79 or a composition according to any of paragraphs 11-40;
b) Completing at least one wash cycle; and
c) Optionally rinsing the item, wherein the item is a textile.
   42. A method of treating an item, wherein the item is preferably a textile, said method comprising the steps of:
a. Exposing an item to a polypeptide selected from the group consisting of a polypeptide having at least 60%, at least 65%, at least 70%, at least 75%, at least 80%, at least 85%, at least 90%, at least 95%, at least 96%, at least 97%, at least 98%, at least 99% or 100% sequence identity to the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56 or the polypeptide comprising the amino acid sequence shown in: SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146; contacting the item with a wash liquor comprising the polypeptide or a composition according to any proceeding paragraphs.
   43. The method according to paragraph 41 or 42, wherein the pH of the wash liquor is in the range of 1 to 11.
   44. The method according to any of paragraphs 41-43, wherein the pH of the wash liquor is in the range 5.5 to 11, such as in the range of 7 to 9, in the range of 7 to 8 or in the range of 7 to 8.5
   45. The method according to any of paragraphs 41-44, wherein the temperature of the wash liquor is in the range of 5° C. to 95° C., or in the range of 10° C. to 80° C., in the range of 10° C. to 70°, in the range of 10° C. to 60° C., in the range of 10° C. to 50° C., in the range of 15° C. to 40° C., in the range of 20° C. to 40° C., in the range of 15° C. to 30° C. or in the range of 20° C. to 30° C.
   46. The method according to any of paragraphs 41-45, wherein the temperature of the wash liquor is from about 20° C. to about 40° C.
   47. The method according to any of paragraphs 41-46, wherein the temperature of the wash liquor is from about 15° C. to about 30° C.
   48. The method according to any of paragraphs 41-47, wherein stains present on the item is pre-treated with a polypeptide of paragraphs 55-79 or a detergent composition according to any of paragraphs 11-40.
   49. The method according to any of paragraphs 41-48, wherein stickiness of the item is reduced.
   50. The method according to any of paragraphs 41-49, wherein redeposition of soil is reduced.
   51. The method according to any of paragraphs 41-50, wherein adherence of soil to the item is reduced or removed.
   52. The method according to any of paragraphs 41-51, wherein whiteness of the item is maintained or improved.
   53. The method according to any of paragraphs 41-52, wherein malodor is reduced or removed from the item.
   54. The method according to any of paragraphs 41-53, wherein the concentration of the 54. polypeptide having hydrolytic activity in the wash liquor is in the range 0.002 mg/L to 2 mg/L, such as 0.02 mg/L to 2 mg/L, such as 0.2 mg/L to 2 mg/L or in the range of 0.0001 mg/L to 10 mg/L or in the range of in the range of 0.001 mg/L to 10 mg/L, or in the range of 0.01 mg/L to 10 mg/L, or in in the range of 0.1 mg/L to 10 mg/L per litre of wash liquor, optionally the concentration of the polypeptide shown in the invention is 0.0001% to 2 wt %, such as 0.001 to 0.1 wt %, such as 0.005 to 0.1 wt %, such as 0.01 to 0.1 wt %, such as 0.01 to 0.5 wt % or most preferred 0.002 to 0.09 wt % in the total detergent concentration.
   55. A polypeptide having hydrolytic activity, preferably having alpha-1,4-polygalactosaminidase activity, selected from the group consisting of:
a) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56, a polypeptide having at least 60%, at least 65%, at least 70%, at least 75%, at least 80%, at least 85%, at least 90%, at least 95%, at least 96%, at least 97%, at least 98%, at least 99% or 100% sequence identity to the mature polypeptide shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57 or a polypeptide having at least 60%, at least 65%, at least 70%, at least 75%, at least 80%, at least 85%, at least 90%, at least 95%, at least 96%, at least 97%, at least 98%, at least 99% or 100% sequence identity to the mature polypeptide shown in SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146;
b) a polypeptide encoded by a polynucleotide that hybridizes under low stringency conditions with:
   i. the mature polypeptide coding sequence shown in SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55;
   ii. the cDNA sequence thereof, or
   iii. the full-length complement of (i) or (ii);
c) a polypeptide encoded by a polynucleotide having at least 60% sequence identity to the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55 or the cDNA sequence thereof;
d) a variant of the mature polypeptide shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146 comprising a substitution, deletion, and/or insertion at one or more positions; and
e) a fragment of the polypeptide shown in (a), (b), (c), or (d) that comprises one or more motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71).
   56. The polypeptide of paragraph 55, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56 or to the mature polypeptide shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146.
   57. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2 or to the mature polypeptide shown in SEQ ID NO: 3.
   58. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 5 or to the mature polypeptide shown in SEQ ID NO: 6.
   59. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 8 or to the mature polypeptide shown in SEQ ID NO: 9.
   60. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 11 or to the mature polypeptide shown in SEQ ID NO: 12.
   61. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 14 or to the mature polypeptide shown in SEQ ID NO: 15.
   62. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 17 or to the mature polypeptide shown in SEQ ID NO: 18.
   63. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 20 or to the mature polypeptide shown in SEQ ID NO: 21.
   64. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 23 or to the mature polypeptide shown in SEQ ID NO: 24.
   65. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 26 or to the mature polypeptide shown in SEQ ID NO: 27.
   66. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 29 or to the mature polypeptide shown in SEQ ID NO: 30.
   67. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 32 or to the mature polypeptide shown in SEQ ID NO: 33.
   68. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 35 or to the mature polypeptide shown in SEQ ID NO: 36.
   69. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 38 or to the mature polypeptide shown in SEQ ID NO: 39.
   70. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 41 or to the mature polypeptide shown in SEQ ID NO: 42.
   71. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 44 or to the mature polypeptide shown in SEQ ID NO: 45.
   72. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 47 or to the mature polypeptide shown in SEQ ID NO: 48.
   73. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 50 or to the mature polypeptide shown in SEQ ID NO: 51.
   74. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 53 or to the mature polypeptide shown in SEQ ID NO: 54.
   75. The polypeptide of paragraph 55 or 56, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 56 or to the mature polypeptide shown in SEQ ID NO: 57.
   76. The polypeptide according to any of paragraphs 55 to 75, which is encoded by a polynucleotide that hybridizes under low stringency conditions, low-medium stringency conditions, medium stringency conditions, medium-high stringency conditions, high stringency conditions, or very high stringency conditions with
i. the mature polypeptide coding sequence shown in SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55;
ii. the cDNA sequence thereof, or
iii. the full-length complement of (i) or (ii).
   77 The polypeptide according to any of paragraphs 55 to 76, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55 or the cDNA sequence thereof.
   78. The polypeptide according to any of paragraphs 55 to 77, comprising or consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57 or the mature polypeptide of SEQ ID NO: 2, SEQ ID NO: 5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50, SEQ ID NO: 53, SEQ ID NO: 56.
   79. The polypeptide according to any of paragraphs 55 to 78, which is a variant of the any of the polypeptides with SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145 or SEQ ID NO: 146 comprising a substitution, deletion, and/or insertion at one or more positions.
   80. The polypeptide according to any of paragraphs 55 to 79 for use as a medicament.
   81. The polypeptide according to any of paragraphs 55 to 79 for use in treatment or prevention of a bacterial infection, preferably said bacterial infection is an infection caused by Gram-positive or Gram-negative bacteria, further preferably said bacterial infection is selected from a group consisting of: Staphylococcus spp. (e.g., Staphylococcus epidermidis, S. aureus), Enterococcus spp. (e.g., Enterococcus faecalis), Escherichia spp. (e.g., Escherichia coli), Listeria spp. (e.g., Listeria monocytogenes), Pseudomonas spp. (e.g., Pseudomonas aeruginosa), Bacillus spp., Salmonella spp., Coagulase-negative Staphylococci, Klebsiella spp. (e.g., Klebsiella pneumoniae) infections.
   82. The polypeptide according to any of paragraphs 55 to 79 for use in treatment or prevention of a disease selected from the group consisting of: Cystic fibrosis pneumonia (e.g., caused by Pseudomonas aeruginosa and/or Burkholderia cepacia), Meloidosis (e.g., caused by Pseudomonas pseudomallei), Necrotizing fasciitis (e.g., caused by Group A streptococci), Musculoskeletal infections (e.g., caused by Staphylococci and other Gram-positive cocci), Otitis media (e.g., caused by Haemophilus influenzae), Biliary tract infection (e.g., caused by E. coli and other enteric bacteria), Urinary catheter cystitis (e.g., caused by E. coli and other Gram-negative rods), Bacterial prostatitis (e.g., E. coli and other Gram-negative bacteria), Periodontitis (e.g., caused by Gram negative anaerobic oral bacteria), Dental caries (e.g., caused by Streptococcus spp. and other acidogenic Gram positive cocci).
   83. A polynucleotide encoding the polypeptide according to any of paragraphs 55-79.
   84. A nucleic acid construct or expression vector comprising the polynucleotide of paragraph 83 operably linked to one or more control sequences that direct the production of the polypeptide in an expression host.
   85. A recombinant host cell comprising the polynucleotide of paragraph 83 operably linked to one or more control sequences that direct the production of the polypeptide.
   86. A method of producing the polypeptide shown in any of paragraphs 55-79, comprising cultivating a cell, which in its wild-type form produces the polypeptide, under conditions conducive for production of the polypeptide.
   87. The method of paragraph 86, further comprising recovering the polypeptide.
   88. A method of producing a polypeptide according to any of paragraphs 55-79, comprising cultivating the host cell of paragraph 85 under conditions conducive for production of the polypeptide.
   89. The method of paragraph 88, further comprising recovering the polypeptide.
   90. A nucleic acid construct or expression vector comprising a gene encoding a protein operably linked to the polynucleotide of paragraph 83, wherein the gene is foreign to the polynucleotide encoding the signal peptide.
   91. A recombinant host cell comprising a gene encoding a protein operably linked to the polynucleotide of paragraph 83, wherein the gene is foreign to the polynucleotide encoding the signal peptide.
   92. A method of producing a protein, comprising cultivating a recombinant host cell comprising a gene encoding a protein operably linked to the polynucleotide of paragraph 83, wherein the gene is foreign to the polynucleotide encoding the signal peptide, under conditions conducive for production of the protein.
   93. The method of paragraph 92, further comprising recovering the protein.
   94. An item laundered according to the method of any of paragraphs 41-54.

The invention is further described in the following paragraphs:

Paragraph 1. A GH114 glycosyl hydrolase comprising the motif [VLI]XE[EDSQ]C (SEQ ID NO: 60) and/or the motif CY[FLIV][SDN][ATVG] (SEQ ID NO: 61), wherein the GH114 glycosyl hydrolase has hydrolytic activity, and wherein the GH114 glycosyl hydrolase comprises or consist of a polypeptide selected from the group consisting of:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27;
(h) a polypeptide having, at least 50% at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 30;
(i) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(j) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 36;
(k) a polypeptide having at least 99% or 100% sequence identity to the polypeptide shown in SEQ ID NO: 39;
(l) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(m) a polypeptide having at least 85%, at least 90%, at least 95%, at least 96%, at least 97%, at least 98%, at least 99% or 100% sequence identity to the polypeptide shown in SEQ ID NO: 45;
(n) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(o) a polypeptide having at least 98%, at least 99% or 100% sequence identity to the polypeptide shown in SEQ ID NO: 51;
(p) a polypeptide having at least 80%, at least 85%, at least 90%, at least 95%, at least 96%, at least 97%, at least 98%, at least 99% or 100% sequence identity to the polypeptide shown in SEQ ID NO: 54; and
(q) a polypeptide having at least 85%, at least 90%, at least 95%, at least 96%, at least 97%, at least 98%, at least 99% or 100% sequence identity to the polypeptide shown in SEQ ID NO: 57.
   Paragraph 2. A GH114 glycosyl hydrolase according to paragraph 1, comprising the motif DYQ[LI]G (SEQ ID NO: 62) and/or the motif FQ[TAV]Q[PSD] (SEQ ID NO: 63), wherein the GH114 glycosyl hydrolase comprises or consist of a polypeptide selected from the group consisting of:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 33;
(h) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 36;
(i) a polypeptide having at least 99% or 100% sequence identity to the polypeptide shown in SEQ ID NO: 39;
(j) a polypeptide having at least 60%, at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 42;
(k) a polypeptide having at least 85%, at least 90%, at least 95%, at least 96%, at least 97%, at least 98%, at least 99% or 100% sequence identity to the polypeptide shown in SEQ ID NO: 45;
(l) a polypeptide having at least 70%, at least 75%, at least 80%, at least 85%, at least 90%, at least 95%, at least 96%, at least 97%, at least 98%, at least 99% or 100% sequence identity to the polypeptide shown in SEQ ID NO: 48;
(m) a polypeptide having at least 98%, at least 99% or 100% sequence identity to the polypeptide shown in SEQ ID NO: 51; and
(n) a polypeptide having at least 80%, at least 85%, at least 90%, at least 95%, at least 96%, at least 97%, at least 98%, at least 99% or 100% sequence identity to the polypeptide shown in SEQ ID NO: 54.
   Paragraph 3. A GH114 glycosyl hydrolase according to paragraph 1 or 2, comprising the motif AEECG (SEQ ID NO: 64) and/or the motif NAFQ[AT]Q (SEQ ID NO: 65), wherein the GH114 glycosyl hydrolase comprises or consist of a polypeptide selected from the group consisting of:
(a) a polypeptide having at least 90%, at least 95%, at least 96%, at least 97%, at least 98%, at least 99% or 100% sequence identity to the polypeptide shown in SEQ ID NO: 3;
(b) a polypeptide having at least 90%, at least 95%, at least 96%, at least 97%, at least 98%, at least 99% or 100% sequence identity to the polypeptide shown in SEQ ID NO: 6;
(c) a polypeptide having at least 85%, at least 90%, at least 95%, at least 96%, at least 97%, at least 98%, at least 99% or 100% sequence identity to the polypeptide shown in SEQ ID NO: 9;
(d) a polypeptide having at least 85%, at least 90%, at least 95%, at least 96%, at least 97%, at least 98%, at least 99% or 100% sequence identity to the polypeptide shown in SEQ ID NO: 12;
(e) a polypeptide having at least 75%, at least 80%, at least 85%, at least 90%, at least 95%, at least 96%, at least 97%, at least 98%, at least 99% or 100% sequence identity to the polypeptide shown in SEQ ID NO: 21;
(f) a polypeptide having at least 90%, at least 95%, at least 96%, at least 97%, at least 98%, at least 99% or 100% sequence identity to the polypeptide shown in SEQ ID NO: 24;
(g) a polypeptide having at least 80%, at least 85%, at least 90%, at least 95%, at least 96%, at least 97%, at least 98%, at least 99% or 100% sequence identity to the polypeptide shown in SEQ ID NO: 54.
   Paragraph 4. A GH114 glycosyl hydrolase according to paragraph 1, comprising one, two or all three motif(s) GXXVX[NHQTS]IEY[PG] (SEQ ID NO: 68), VICYF (SEQ ID NO: 69) or ICYFSA (SEQ ID NO: 70), wherein the GH114 glycosyl hydrolase is a polypeptide having at least 85%, at least 90%, at least 95%, at least 96%, at least 97%, at least 98%, at least 99% or 100% sequence identity to the polypeptide shown in SEQ ID NO: 57.
   Paragraph 5. A GH114 glycosyl hydrolase according to paragraph 1, comprising the motif WQWQL (SEQ ID NO: 66) and/or the motif [VLI][GASD]LKN[DGS][VLIP] (SEQ ID NO: 67), wherein the GH114 glycosyl hydrolase is a polypeptide having at least 65%, at least 70%, at least 75%, at least 80%, at least 85%, at least 90%, at least 95%, at least 96%, at least 97%, at least 98%, at least 99% or 100% sequence identity to the polypeptide shown in SEQ ID NO: 27.
   Paragraph 6. A GH114 glycosyl hydrolase according to any of paragraphs 1 to 3, wherein the GH114 glycosyl hydrolase is obtained from Amycolatopsis, preferably Amycolatopsis circi or Amycolatopsis sp. and wherein the GH114 glycosyl hydrolase comprises or consist of a polypeptide selected from the group consisting of:
a) at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 3;
b) at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 6; or
c) at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 9.
   Paragraph 7. A GH114 glycosyl hydrolase according to any of paragraphs 1 to 3, wherein the GH114 glycosyl hydrolase is obtained from Streptomyces, preferably Streptomyces parvulus or Streptomyces miharaensis, and wherein the GH114 glycosyl hydrolase comprises or consist of a polypeptide selected from the group consisting of:
a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 12;
b) is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 24; or
c) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 54.
   Paragraph 8. The GH114 glycosyl hydrolase according to paragraph 6 or 7, wherein the GH114 glycosyl comprises one or more, or even all of the motif(s) [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65).
   Paragraph 9. A granule comprising:
(a) a core comprising a GH114 glycosyl hydrolase according to any of paragraphs 1 to 8, and
(b) optionally a coating consisting of one or more layer(s) surrounding the core.
   Paragraph 10. A cleaning composition comprising:
(a) at least 0.001 ppm of at least one GH114 glycosyl hydrolase according to any of paragraphs 1 to 8;
(b) one or more cleaning composition components, preferably selected from surfactants, builders, bleach components, polymers, dispersing agents and additional enzymes.
   Paragraph 11. A method for laundering an item comprising the steps of:
(a) exposing an item to a wash liquor comprising the GH114 glycosyl hydrolase according to any of paragraphs 1 to 8 or the composition according to paragraph 10;
(b) completing at least one wash cycle; and
(c) optionally rinsing the item, wherein the item is a textile.
   Paragraph 12. Use of a GH114 glycosyl hydrolase in a cleaning process, such as laundry and/or dish wash.
   Paragraph 13. Use of a GH114 glycosyl hydrolase,
i. for preventing, reducing or removing stickiness of the item;
ii. for preventing, reducing or removing biofilm or biofilm components
iii. for reducing or removing pel stains on the item;
iv. for preventing, reducing or removing redeposition of soil during a wash cycle;
v. for preventing, reducing or removing adherence of soil to the item;
vi. for maintaining or improving whiteness of the item;
vii. for preventing, reducing or removing malodor from the item,
   wherein the item is a textile.
   Paragraph 14. Use according to paragraph 12 or 13, wherein GH114 glycosyl hydrolase comprising one or more of the motif(s) selected from the group consisting of: [VLI]XE[EDSQ]C (SEQ ID NO: 60), CY[FLIV][SDN][ATVG] (SEQ ID NO: 61), DYQ[LI]G (SEQ ID NO: 62), FQ[TAV]Q[PSD] (SEQ ID NO: 63), AEECG (SEQ ID NO: 64), NAFQ[AT]Q (SEQ ID NO: 65), WQWQL (SEQ ID NO: 66), [VLI][GASD]LKN[DGS][VLIP] (SEQ ID NO: 67), GXXVX[NHQTS]IEY[PG] (SEQ ID NO: 68), VICYF (SEQ ID NO: 69), ICYFSA (SEQ ID NO: 70), DFAVL (SEQ ID NO: 71).
   Paragraph 15. Use according to any of paragraphs 12 to 14, wherein the polypeptide is selected from any of the GH114 glycosyl hydrolases of paragraphs 1 to 8.

### EXAMPLES

### Model Detergents

Model detergent A wash liquor (100%) was prepared by dissolving 3.33 g/l of model detergent A containing 12% LAS, 11% AEO Biosoft N25-7 (NI), 5% AEOS (SLES), 6% MPG (monopropylene glycol), 3% ethanol, 3% TEA, 2.75% coco soap, 2.75% soya soap, 2% glycerol, 2% sodium hydroxide, 2% sodium citrate, 1% sodium formate, 0.2% DTMPA and 0.2% PCA (all percentages are w/w) in water with hardness 15 dH.

Triple-20 Nonionic Model Detergent (Model NI) (60% surfactant) was prepared by dissolving 3.33 g/l non-ionic detergent containing NaOH 0.87%, MPG (Monopropylenglycol) 6%, Glycerol 2%, Soap-soy 2.75%, Soap-coco 2.75%, PCA (Sokalon CP-5) 0.2%, AEO Biosoft N25-7 (NI) 16%, Sodium formate 1%, Sodium Citrate 2%, DTMPA 0.2%, Ethanol (96%) 3% (all percentages are w/w) in water with hardness 15 dH.

Model Detergent MC: A medical cleaning model detergent (model detergent MC) was prepared containing 5% MPG (propylene glycol), 5% Pluronic PE 4300 (PO/EO block polymer; 70%/30%, approx. 1750 g/mol), 2% Plurafac LF 305 (fatty alcohol alkoxylate; C6-10+EO/PO), 1% MGDA (methyl glycine diacetic acid, 1% TEA (triethanolamine) (all percentages are w/w). The pH was adjusted to 8.7 with phosphoric acid.

### Wash Assay

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM is basically a large temperature-controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full-scale experiments in front loader washing machines. In miniLOM, washes are performed in 50 ml test tubes placed in Stuart rotator.

### Activity Towards Poly-Galactosamine Substrate

Enzyme activity has been determined using reducing ends assay. 90 μl poly-galactosamine, 2 mg/ml in 20 mM acetic buffer pH 6, was incubated with 10 μl 50 nM enzyme in PCR-MTP wells for 30 min at 30° C. The number of reducing ends formed was determined by reaction with 75 μl p-Hydroxybenzoic acid hydrazide (Sigma, H-9882, 15 mg/ml) for 10 min at 95° C., followed by absorbance measurement at 405 nm.

TABLE 1
activity towards poly-galactosamine
substrate

| | Absorbance at |
|---|---|
| SEQ ID NO: | 405 nm-blank |
| 3 | 1.47 |
| 12 | 0.36 |
| 18 | 0.60 |
| 21 | 0.72 |
| 24 | 0.28 |
| 27 | 0.52 |
| 33 | 1.33 |
| 36 | 1.24 |
| 39 | 1.16 |
| 42 | 0.29 |
| 45 | 0.95 |
| 48 | 1.36 |
| 51 | 1.19 |
| 57 | 0.70 |
| 72 | 0.36 |
| 73 | 0.13 |
| 74 | 0.35 |
| 75 | 0.25 |
| 76 | 0.23 |
| 77 | 0.12 |
| 78 | 0.15 |
| 81 | 2.04 |
| 82 | 2.05 |
| 83 | 1.77 |
| 84 | 0.18 |
| 85 | 1.89 |
| 86 | 1.42 |
| 87 | 1.67 |
| 88 | 0.66 |
| 89 | 1.54 |
| 90 | 1.47 |
| 91 | 1.54 |
| 92 | 1.41 |
| 93 | 1.27 |
| 94 | 1.30 |
| 95 | 1.01 |
| 96 | 1.46 |
| 97 | 0.11 |
| 99 | 0.18 |
| 101 | 0.93 |
| 102 | 0.80 |
| 103 | 1.20 |
| 104 | 0.12 |
| 106 | 0.62 |
| 107 | 0.23 |
| 136 | 1.93 |
| 137 | 1.61 |
| 138 | 0.87 |
| 139 | 1.58 |
| 140 | 1.32 |
| 141 | 1.43 |
| 142 | 1.11 |
| 143 | 0.62 |
| 144 | 1.48 |
| 145 | 1.40 |
| 146 | 0.81 |

### Production of Poly-Galactosamine

A crude poly-galactosamine substrate has been prepared as described in Takagi and Kadowaki, 1985, Agricultural and Biological Chemistry 49 (11): 3151-3157, using the same Simplicillum strain (previously designated Paecilomyces sp.), obtained from the Japanese depository in September 2018. Briefly, the strain was cultured in GPC medium (3% glucose, 0.3% peptone, 0.5% CaCl2, pH7) for 5 days at 26° C. under shaking conditions (100 rpm). Following cultivation, the supernatant was isolated (Miracloth filtration) and used directly for wash experiments or precipitated with ethanol as described in Takagi & Kadowaki, 1985, Agricultural and Biological Chemistry 49 (11): 3159-3164. The resulting precipitate was dissolved in 1 M acetic acid overnight, dialyzed with 20 mM acetic buffer pH 6 and used for enzyme activity measurements.

### Example 1. Cloning and Expression of Polypeptides

The DNA encoding the gene of SEQ ID NO: 1, SEQ ID NO: 4, SEQ ID NO: 7, SEQ ID NO: 10, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, were isolated from bacterial strains and environmental bacterial communities isolated from soil samples collected in different countries (see table 2). Chromosomal DNA from the different strains and bacterial communities was subjected to full genome sequencing using Illumina technology. The genome sequence was analyzed for protein sequences that contained glycosyl hydrolase domains, as defined in the CAZy database (cazy.org, Lombard et al., 2014, Nucleic Acids Res. 42: D490-D495). 16 sequences containing a Glycoside Hydrolase Family 114 domain (GH114, CAZy database, cazy.org, Lombard et al., 2014, Nucleic Acids Res. 42: D490-D495) were identified in the genomes”.

TABLE 2
DNA sequences and mature protein

| | Mature | | |
|---|---|---|---|
| DNA | protein | | |
| SEQ | SEQ | | |
| ID NO: | ID NO: | Donor | country of origin |
| 1 | 3 | Amycolatopsis circi | Spain |
| 4 | 6 | Amycolatopsis sp-63060 | Spain |
| 7 | 9 | Amycolatopsis sp-63067 | Spain |
| 10 | 12 | Streptomyces parvulus | Jamaica |
| 19 | 21 | Nonomuraea guangzhouensis | United Kingdom |
| 22 | 24 | Streptomyces sp-63031 | Denmark |
| 25 | 27 | Vibrio sp-62464 | United States |
| 28 | 30 | Environmental bacterial | United States |
| | | community B | |
| 31 | 33 | Microbacterium saccharophilum | Denmark |
| 34 | 36 | Microbacterium oxydans | Denmark |
| 37 | 39 | Frigoribacterium faeni | Denmark |
| 40 | 42 | Serinibacter sp-64503 | Denmark |
| 43 | 45 | Oerskovia paurometabola | Denmark |
| 46 | 48 | Agreia pratensis | Denmark |
| 49 | 51 | Plantibacter flavus | Denmark |
| 52 | 54 | Streptomyces miharaensis | Korea obtained |
| | | | in 1988 |

The DNA encoding the mature peptide of GH114 genes SEQ ID NO: 1, SEQ ID NO: 4, SEQ ID NO: 7, SEQ ID NO: 10, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, were amplified from the genomic DNA of the corresponding bacterial strains by standard PCR techniques using specific primers containing an overhang to cloning vector. The amplified PCR fragments were inserted into a Bacillus expression vector as described in WO 2012/025577. Briefly, the DNA encoding the mature peptide of the gene was cloned in frame to a Bacillus clausii secretion signal (BcSP; with the following amino acid sequence: MKKPLGKIVASTALLISVAFSSSIASA (SEQ ID NO: 58). BcSP replaced the native secretion signal in the gene. Downstream of the BcSP sequence, an affinity tag sequence was introduced to ease the purification process (His-tag; with the following amino acid sequence: HHHHHHPR (SEQ ID NO: 59) The gene that was expressed therefore comprised the BcSP sequence followed by the His-tag sequence followed by the mature wild type GH114 gene sequence. The final expression plasmid (BcSP-His-tag-GH114) was transformed into a Bacillus subtilis expression host. The GH114 BcSP-fusion gene was integrated by homologous recombination into the Bacillus subtilis host cell genome upon transformation. The gene construct was expressed under the control of a triple promoter system (as described in WO 99/43835). The gene coding for chloramphenicol acetyltransferase was used as maker (as described in (Diderichsen et al., 1993, Plasmid 30:312-315)). Transformants were selected on LB media agar supplemented with 6 micrograms of chloramphenicol per ml. One recombinant Bacillus subtilis clone containing the GH114 expression construct was selected and was cultivated on a rotary shaking table in 500 ml baffled Erlenmeyer flasks each containing 100 ml yeast extract-based media. After 3-5 days' cultivation time at 30° C. to 37° C., the enzyme containing supernatant was harvested by centrifugation and the enzymes was purified by His-tag purification.

The codon optimized synthetic DNA encoding the mature peptide sequence of the putative endo-α-1,4-polygalactosaminidases belonging to family GH114 glycosyl hydrolases listed below in Table 3, were purchased from TWIST Bioscience. The cloning and expression of the synthetic genes in the Bacillus subtilis expression host was carried out as described above.

TABLE 3

| enzyme | donor | country of origin |
|---|---|---|
| SEQ ID NO: 79 | Amycolatopsis sp. | Spain |
| SEQ ID NO: 80 | Methylothermaceae bacteria B42 | Pacific Ocean |
| | | floor |
| SEQ ID NO: 81 | Amycolatopsis niigatensis | Japan |
| SEQ ID NO: 82 | Streptomyces sp. AA4 | unknown |
| SEQ ID NO: 83 | Amycolatopsis alba DSM 44262 | unknown before |
| | | 1996 |
| SEQ ID NO: 84 | Streptomyces griseoaurantiacus | United States |
| SEQ ID NO: 85 | Kutzneria albida | Japan |
| SEQ ID NO: 86 | Microbacterium sp. | Denmark |
| SEQ ID NO: 87 | Microbacterium oleivorans | United States |
| SEQ ID NO: 88 | Nocardiopsis alba | Denmark |
| SEQ ID NO: 89 | Microbacterium oxydans | United States |
| SEQ ID NO: 90 | Agreia pratensis | United States |
| SEQ ID NO: 91 | Microbacterium lemovicicum | United States |
| SEQ ID NO: 92 | Xylanibacterium sp. | United Kingdom |
| SEQ ID NO: 93 | Curtobacterium oceanosedimentum | United States |
| SEQ ID NO: 94 | Leucobacter tardus | Denmark |
| SEQ ID NO: 95 | Salinibacterium amurskyense | Denmark |
| SEQ ID NO: 96 | Environmental sample | Denmark |
| SEQ ID NO: 136 | Amycolatopsis orientalis DSM 46075 | unknown before |
| | | 1979 |
| SEQ ID NO: 137 | Environmental sample | Spain |
| SEQ ID NO: 138 | Pilimelia columellifera subsp. | United States |
| | pallida Spain | |
| SEQ ID NO: 139 | Cellulomonas cellasea | United States |
| SEQ ID NO: 140 | Environmental sample | Denmark |
| SEQ ID NO: 141 | Microbacterium sp. | United States |
| SEQ ID NO: 142 | Amycolatopsis bullii | United States |
| SEQ ID NO: 143 | Microbacterium oxydans | United States |
| SEQ ID NO: 144 | Microbacterium phyllosphaerae | United States |
| SEQ ID NO: 145 | Phycicoccus dokdonensis | United States |
| SEQ ID NO: 146 | Environmental sample | Denmark |

### Example 2. His Tag Purification Method

The His-tagged GH114 polypeptides were purified by immobilized metal chromatography (IMAC) using Ni²⁺ as the metal ion on 5 mL HisTrap Excel columns (GE Healthcare Life Sciences). The purification took place at pH 7 and the bound protein was eluted with imidazole. The purity of the purified enzymes was checked by SDS-PAGE and the concentration of the enzyme determined by Absorbance 280 nm after a buffer exchange in 50 mM HEPES, 100 mM NaCl pH 7.0.

### Example 3. Cloning, Expression and Fermentation of Two GH114 Polypeptides from Fusarium solani and One from Urnula Craterium

Genes (SEQ ID NO: 13, SEQ ID NO: 16, SEQ ID NO: 55) encoding three putative genes belonging to family GH114 as defined by CAZy (cazy.org, Lombard et al., 2014, Nucleic Acids Res. 42: D490-D495) were cloned from a strain of Fusarium solani that was isolated from an environmental sample collected in Denmark and a strain of Urnula criterium isolated from an environmental sample collected in Sweden. For Fusarium solani, chromosomal DNA was isolated from the strain, and whole genome sequencing was purchased from Exiqon A/S, Vedbaek, Denmark. The genome sequence was assembled with the SPAdes Genome Assembler, v3.5.0 (Bankevich et al., 2012, J. Comput. Biol. 19 (5): 455-477) and annotated with the GeneMark v2.3c gene prediction software (Ter-Hovhannisyan et al., 2008, Genome Res. 18 (12): 1979-1990). For Urnula criterium, chromosomal DNA was isolated from the strain, and whole genome sequencing was determined using Illumina technology. The genome sequence was assembled with the SPAdes Genome Assembler, v3.9.0, and annotated with the Augustus v2.4 gene prediction software (Stanke et al., 2008, Bioinformatics 24 (5): 637-644) trained on a gene set from Coprinus cinereus. Peptides predicted from the annotated genome were searched for similarity to the GH114 domain, and the three peptides with SEQ ID NO: 14, 17 and 56 were identified. The corresponding DNA sequences (SEQ ID NOs: 13, 16 and 55) were PCR amplified from genomic DNA isolated from Fusarium solani with gene-specific primers that also append a Kozak translation initiation sequence “TCACC” immediately 5′ of the start codon and cloned into the Aspergillus expression vector pMStr57 (WO 2004/032648) that had been digested with BamHI and XhoI.

The cloned GH114 encoding genes were sequenced and confirmed to be identical to the corresponding genes found in the genome sequences and transformed into the Aspergillus oryzae strain MT3568 (WO 2011/057140) by the methods described in Christensen et al., 1988, Biotechnology 6:1419-1422 and WO 2004/032648. Transformants were selected during regeneration from protoplasts based on the ability, conferred by a selectable marker in the expression vector, to utilize acetamide as a nitrogen source, and were subsequently re-isolated under selection.

Production of the recombinant GH114 peptides was evaluated by culturing the transformants in 96-well deep-well microtiter plates for 4 days at 30° C. in 0.25 ml of both YPG medium (WO 2005/066338) and DAP-4C-1 medium (WO 2012/103350) and monitoring peptide expression by SDS-PAGE. For larger-scale production of the recombinant putative GH114 genes, a single Aspergillus transformant was selected for each GH114 peptide and the transformants were cultured in 500 ml baffled flasks containing 150 ml of DAP-4C-1 medium. The cultures were shaken on a rotary table at 150 RPM at for 4 days. The culture broth was subsequently separated from cellular material by passage through a 0.22 μm filtration unit.

### Example 4. Chromatographic Purification of Two GH114 Polypeptides from Fusarium solani and One from Urnula Craterium

The pH of the filtered sample was adjusted to around pH 7.5 and 1.8 M ammonium sulfate was added. The sample was applied to a 5 ml HiTrap™ Phenyl (HS) column on an Äkta Explorer. Prior to loading, the column had been equilibrated in 5 column volumes (CV) of 50 mM HEPES+1.8 M AMS pH 7. In order to remove unbound material, the column was washed with 5 CV of 50 mM HEPES+1.8 M AMS pH 7. The target protein was eluted from the column into a 10 ml loop using 50 mM HEPES+20% isopropanol pH 7. From the loop, the sample was loaded onto a desalting column (HiPrep™ 26/10 Desalting), which had been equilibrated with 3CV of 50 mM HEPES+100 mM NaCl pH 7.0. The target protein was eluted with 50 mM HEPES+100 mM NaCl pH 7.0 and relevant fractions were selected and pooled based on the chromatogram. The flow rate was 5 ml/min. For the two GH114 peptides from Fusarium solani, the sample collected from HIC purification (described above), was applied to a 1 mL HiTrap™ Blue HP column on an Äkta PURE system. Prior to loading the column had been equilibrated in 10 CV of 50 mM HEPES+100 mM NaCl pH 7.0. During loading of the sample to the column, the flow through was collected. In order to remove any unbound material, the column was washed with 7 CV 50 mM HEPES+100 mM NaCl pH 7.0. The target protein was eluted with 11.2 CV of 50 mM Tris, 1 M NaCl, pH 8. Based on the chromatogram, relevant fractions were collected. The protein was either in the flow through or in the eluate. Protein concentration in the final sample was estimated by measuring absorption at 280 nm.

### Example 5. Cleaning in Liquid Model Detergent a on Pel Swatches

A crude extract of biofilm EPS was prepared from Pseudomonas aeruginosa PA14 (DSM 19882) as follows; The strain was restreaked on Tryptone Soya Agar (TSA) (pH 7.3) (CM0131; Oxoid Ltd, Basingstoke, UK) and incubated for 3 days at 30° C. Flasks with T-broth (10 g/L Bacto™ Tryptone (211705, BD), 5 g/L sodium chloride (31434, Sigma-Aldrich)) were then inoculated with single colonies and incubated statically for 6 days at 20° C. The biofilm pellicles were carefully removed from the flasks, and pelleted by centrifugation (5 min, 16000 g, 25° C.). The pellets were resuspended in 3 M NaCl, vortexed vigorously and incubated for 15 min at ambient temperature to extract the surface-associated polymer. The cells were then re-pelleted (5 min, 10000 g, 25° C.) and the EPS-containing supernatant was retrieved and pooled. The extract was stored at −20° C. until further use (termed EPS extract).

The wash performance was determined as follows; 50 μl aliquots of the crude Pel extract were spotted on sterile textile swatches (WFK20A) and incubated for 15 min at ambient temperature. Control swatches were spotted with 3 M NaCl. The swatches (sterile or with the extract) were placed in 50 mL test tubes and 10 mL of wash liquor (15° dH water with 0.2 g/L iron (III) oxide nano-powder (544884; Sigma-Aldrich) with 3.33 g/L liquid model A detergent) and the 2 μg/ml enzyme was added to each tube. Washes without enzyme were included as controls. The test tubes were placed in a Stuart rotator and incubated for 1 hour at 30° C. and 20 rpm. The wash liquor was then removed, and the swatches were rinsed twice with 15° dH water and dried on filter paper over night. The color difference (L) values were measured using a Handheld Minolta CR-300 and are displayed in table 4. Wash performance (WP) values (L_{(swatch washed with enzyme)}−L_{(swatch washed without enzyme)}) are also indicated.

TABLE 4
Cleaning effects of the GH114 homologues in model A detergent

| | | Enzyme | | |
|---|---|---|---|---|
| | | conc. | Average | WP |
| Swatch/Enzyme | SEQ ID NO: | (μg/ml) | L values | (ΔL) |
| wfk20A, no EPS | | 0.0 | 88.1 | |
| Wfk20A, EPS, no enzyme | | 2 | 82.2 | |
| Wfk20A, EPS, | SEQ ID NO: 3 | 2 | 91.4 | 9.3 |
| Wfk20A, EPS, | SEQ ID NO: 6 | 2 | 88.4 | 6.3 |
| Wfk20A, EPS, | SEQ ID NO: 9 | 2 | 90.3 | 8.2 |
| Wfk20A, EPS, | SEQ ID NO: 12 | 2 | 89.7 | 7.5 |
| Wfk20A, EPS, | SEQ ID NO: 21 | 2 | 90.6 | 8.4 |
| Wfk20A, EPS, | SEQ ID NO: 24 | 2 | 90.3 | 8.1 |
| Wfk20A, EPS, | SEQ ID NO: 27 | 2 | 89.0 | 6.8 |
| Wfk20A, EPS, | SEQ ID NO: 33 | 2 | 90.3 | 8.1 |
| Wfk20A, EPS, | SEQ ID NO: 36 | 2 | 87.6 | 5.4 |
| Wfk20A, EPS, | SEQ ID NO: 39 | 2 | 90.7 | 8.5 |
| Wfk20A, EPS, | SEQ ID NO: 45 | 2 | 85.9 | 3.7 |
| Wfk20A, EPS, | SEQ ID NO: 48 | 2 | 89.0 | 6.8 |
| Wfk20A, EPS, | SEQ ID NO: 54 | 2 | 91.1 | 8.9 |

### Example 6. Cleaning in Liquid Model Detergent NI on Pel Swatches

A crude extract of biofilm EPS was prepared from Pseudomonas aeruginosa PA14 (DSM 19882) as follows; The strain was restreaked on LB Agar (pH 7.3) and incubated for 3 days at 30° C. 500 mL of T-broth (10 g/L Bacto™ Tryptone (211705, BD), 5 g/L sodium chloride (31434, Sigma-Aldrich)) was then inoculated and incubated statically for 6 days at 20° C. The biofilm pellicle was carefully removed from the flask, and pelleted by centrifugation (5 min, 10000 g, 25° C.). The pellet was then resuspended in 3 M NaCl, vortexed vigorously and incubated for 15 min at ambient temperature to extract the surface-associated polymer. The cells were then re-pelleted (5 min, 10000 g, 25° C.) and the EPS-containing supernatant was retrieved. The extract was stored at −20° C. until further use (termed EPS extract). Wash performance was determined as follows; 50 μl aliquots of the crude Pel extract were spotted on sterile textile swatches (WFK20A) and incubated for 15 min at ambient temperature. Control swatches without EPS were used as controls. The swatches (sterile or with the extract) were placed in 50 mL test tubes and 10 ml of wash liquor (15° dH water with 0.2 g/L iron (III) oxide nano-powder (544884; Sigma-Aldrich) with 3.33 g/L liquid model NI detergent) and the 10 μg/ml enzyme was added to each tube. Washes without enzyme were included as controls. The test tubes were placed in a Stuart rotator and incubated for 1 hour at 30° C. and 20 rpm. The wash liquor was then removed, and the swatches were rinsed twice with 15° dH water and dried on filter paper over night. The tristimulus light intensity (Y) values were measured using a Handheld Minolta CR-300 and are displayed in Table 5. Wash performance, WP (ΔY=Y_{(swatches washed with enzyme)}−Y_{(swatches washed without enzyme)}) are also indicated.

TABLE 5
Cleaning effects of the GH114 glycosyl hydrolases in model NI detergent

| | | Enzyme | | |
|---|---|---|---|---|
| | | concentration | Average | WP |
| Swatch | Enzyme | (μg/ml) | Y values | (ΔY) |
| Wfk20A, no EPS | No enzyme | 0 | 74.9 | |
| Wfk20A, Pel EPS swatch | No enzyme | 0 | 64.1 | |
| Wfk20A, Pel EPS swatch | SEQ ID NO: 54 | 10 | 68.8 | 4.7 |
| Wfk20A, Pel EPS swatch | SEQ ID NO: 30 | 10 | 68.4 | 4.3 |
| Wfk20A, Pel EPS swatch | SEQ ID NO: 42 | 10 | 68.7 | 4.6 |

### Example 7. Cleaning in Liquid Model Detergent a on Pel Swatches

A crude extract of the biofilm extracellular polymer Pel was prepared from Pseudomonas aeruginosa PA14 (DSM 19882) as described above. The wash performance was determined as follows; 50 μl aliquots of the crude Pel extract were spotted on sterile textile swatches (WFK20A) and incubated for 15 min at ambient temperature. Control swatches were spotted with 3 M NaCl. The swatches (sterile or with the extract) were placed in 50 mL test tubes and 10 mL of wash liquor (15° dH water with 0.2 g/L iron (III) oxide nano-powder (544884; Sigma-Aldrich) with 3.33 g/L liquid model A detergent) and the enzyme was added to each tube. Washes without enzyme were included as controls. The test tubes were placed in a Stuart rotator and incubated for 1 hour at 30° C. and 20 rpm. The wash liquor was then removed, and the swatches were rinsed twice with 15° dH water and dried on filter paper over night. The color difference (L) values were measured using a Handheld Minolta CR-300 and are displayed in table 6. Wash performance (WP) values (L_{(swatch washed with enzyme)}−L_{(swatch washed without enzyme)}) are also indicated.

TABLE 6
Cleaning effects of the GH114 glycosyl hydrolases in model A detergent

| | | Enzyme | | |
|---|---|---|---|---|
| | | conc. | Average | WP |
| Swatch | Enzyme | (μg/ml) | L values | (ΔL) |
| Wfk20A, no EPS | no enzyme | 0 | 84.3 | |
| Wfk20A, Pel EPS | no enzyme | 0 | 63.6 | |
| Wfk20A, Pel EPS | SEQ ID NO: 18 | 0.2 | 75.8 | 12.2 |
| Wfk20A, Pel EPS | SEQ ID NO: 18 | 2 | 78.8 | 15.2 |
| Wfk20A, Pel EPS | SEQ ID NO: 15 | 0.2 | 72.9 | 9.3 |
| Wfk20A, Pel EPS | SEQ ID NO: 15 | 2 | 76.4 | 12.8 |
| Wfk20A, Pel EPS | SEQ ID NO: 57 | 0.2 | 66.8 | 3.2 |
| Wfk20A, Pel EPS | SEQ ID NO: 57 | 2 | 78.6 | 15.0 |

### Example 8. Biofilm Removal Activity

The Pel-producing Pseudomonas aeruginosa strain DSM19882 was used as a model microorganism in the present example. The strain was restreaked on LB agar and incubated at 30° C. An overnight culture was inoculated in 10 mL LB and the culture was incubated for 16 hours at 37° C. under shaking conditions. The culture was subsequently diluted (1:100) in LBNS, added to 96-well microtiter plates (150 μL per well, Thermo Scientific, cat #₁₆₇₀₀₈) and Peg lids were inserted (NUNC-TSP, Thermo Scientific, cat #₄₄₅₄₉₇). The microtiter plates were incubated for 24 hours at 26° C. under static conditions. After incubation, the peg lids were rinsed in MTP plates with 5° dH water hardness, and transferred to treatment plates with LBNS containing no enzyme (control) or 20 μg/mL enzyme for 1 hour at 26° C. The lids were subsequently rinsed in water hardness and stained with 0.095% crystal violet (Sigma-Aldrich, cat #V5265) for 15 min. Following the staining, the peg lids were rinsed twice, moved to clean microtiter plates and the remaining dye was dissolved with 30% acetic acid. The absorbance was measured at 595 nm. The results are displayed in Tables 7 and 8.

TABLE 7
Biofilm removal properties

| | | % |
|---|---|---|
| | | remaining |
| Enzyme | Conc | biofilm |
| no enzyme | 0 | 100.0 |
| SEQ ID NO: 3 | 20 | 30.0 |
| SEQ ID NO: 6 | 20 | 26.3 |
| SEQ ID NO: 9 | 20 | 27.1 |
| SEQ ID NO: 12 | 20 | 24.6 |
| SEQ ID NO: 21 | 20 | 19.9 |
| SEQ ID NO: 24 | 20 | 20.6 |
| SEQ ID NO: 27 | 20 | 34.8 |
| SEQ ID NO: 33 | 20 | 3.8 |
| SEQ ID NO: 36 | 20 | 10.5 |
| SEQ ID NO: 39 | 20 | 35.4 |
| SEQ ID NO: 45 | 20 | 73.0 |
| SEQ ID NO: 48 | 20 | 49.4 |
| SEQ ID NO: 51 | 20 | 14.9 |
| SEQ ID NO: 18 | 20 | 3.7 |
| SEQ ID NO: 15 | 20 | 3.7 |
| SEQ ID NO: 57 | 20 | 3.2 |

The following batch of GH114 glycosyl hydrolase enzymes with the corresponding SEQ ID NO: listed in Table 8 were tested at a concentration of 40 g/mL for biofilm removal activity as described above.

TABLE 8
Biofilm removal properties

| Enzyme | % Remaining biofilm |
|---|---|
| no enzyme | 100 |
| SEQ ID NO: 72 | 35.73 |
| SEQ ID NO: 73 | 35.48 |
| SEQ ID NO: 74 | 19.15 |
| SEQ ID NO: 75 | 15.01 |
| SEQ ID NO: 76 | 25.27 |
| SEQ ID NO: 77 | 56.65 |
| SEQ ID NO: 78 | 18.84 |
| SEQ ID NO: 81 | 74.27 |
| SEQ ID NO: 82 | 95.78 |
| SEQ ID NO: 83 | 46.26 |
| SEQ ID NO: 84 | 49.36 |
| SEQ ID NO: 86 | 47.41 |
| SEQ ID NO: 87 | 39.26 |
| SEQ ID NO: 88 | 99.42 |
| SEQ ID NO: 89 | 22.13 |
| SEQ ID NO: 90 | 33.58 |
| SEQ ID NO: 91 | 70.30 |
| SEQ ID NO: 92 | 66.34 |
| SEQ ID NO: 93 | 30.79 |
| SEQ ID NO: 94 | 93.82 |
| SEQ ID NO: 95 | 21.01 |
| SEQ ID NO: 96 | 15.88 |
| SEQ ID NO: 101 | 22.75 |
| SEQ ID NO: 102 | 16.55 |
| SEQ ID NO: 103 | 7.54 |
| SEQ ID NO: 104 | 10.68 |
| SEQ ID NO: 105 | 26.45 |
| SEQ ID NO: 106 | 18.63 |
| SEQ ID NO: 107 | 11.80 |
| SEQ ID NO: 108 | 18.83 |
| SEQ ID NO: 136 | 27.02 |
| SEQ ID NO: 137 | 24.68 |
| SEQ ID NO: 138 | 25.78 |
| SEQ ID NO: 139 | 22.56 |
| SEQ ID NO: 140 | 28.82 |
| SEQ ID NO: 141 | 32.21 |
| SEQ ID NO: 142 | 41.56 |
| SEQ ID NO: 143 | 9.27 |
| SEQ ID NO: 144 | 12.13 |
| SEQ ID NO: 145 | 21.16 |
| SEQ ID NO: 146 | 35.32 |

Tables 7 and 8 show that the tested GH114 glycosyl hydrolases have biofilm removal properties and that the remaining biofilm is less than 100%, which means that some biofilm has been reduced.

### Example 9. Effect of GH114 Glycosyl Hydrolases on P. aeruginosa Biofilms Washed in Model Detergent

Two different clinical isolates of P. aeruginosa were for formation of medical biofilms in the example. One biofilm was produced by P. aeruginosa PA14 (DSM19882) and another one by P. aeruginosa PA01 (DSM22644). The bacteria were re-streaked on TSA plates and incubated for three days at 30° C. After three days of incubation, 8 mL of Tryptic Soy Broth (TSB) was inoculated with one colony of P. aeruginosa PA14 (DSM19882), and 8 mL of TSB was inoculated with P. aeruginosa PA01 (DSM22644). The inoculated TSB tubes were all incubated overnight at 30° C., 200 rpm, and diluted in TSB media to a specific optical density (OD). 150 μl of diluted overnight culture was added to each well in Thermo Scientific™ Nunc™ MicroWell™ 96-Well Microplates (sterile, non-treated). Two plates with P. aeruginosa PA14 (DSM19882) were prepared. One plate with P. aeruginosa PA01 (DSM22644) was prepared. The plates were incubated at 30° C. for 24 hours. After 24 hours of incubation, the microtiter plates containing biofilm were removed from the incubator and emptied for media using Vacusafe™ Vacuum Aspiration System (INTEGRA Biosciences). Each well was rinsed twice with 200 μl 0.9% NaCl solution. To each well, 200 μl of model detergent liquor with 20 g/ml enzyme was added. Treatment without enzyme was included as controls. Each treatment was tested in quadruplicates. After addition of detergent liquor+/−enzyme, the microtiter plates were incubated static for 60 minutes at 30° C. After 60 minutes of incubation, the treatment liquor was removed using the vacuum system. Each well was rinsed twice with 200 μl 0.9% NaCl solution, and 200 μl of 0.095% crystal violet solution was added to each well. The plates were incubated for 15 minutes at ambient temperature. The crystal violet solution was removed using the vacuum system, and each well was rinsed twice with 200 μl 0.9% NaCl solution. 150 μl of 30% acetic acid was added to each well. The plates were incubated for 10 minutes at ambient temperature, where after the absorbance at 595 nm was measured using a spectrophotometer (SpectraMax M3, Molecular Devices). The plates were shaked for 10 seconds before absorbance measurements were performed.

The % remaining biofilm after enzymatic treatment was calculated as ABS_{595(biofilm treated with model detergent+enzyme)}/ABS_{595(biofilm treated with model detergent)}×100%.

For P. aeruginosa PA14 (DSM19882) and average of two plates was calculated. The results are displayed in Table 9.

TABLE 9
% remaining biofilm after treatment with
GH114 glycosyl hydrolases in model detergent

| | | % remaining biofilm | |
|---|---|---|---|
| | | PA14 | PA01 |
| Enzyme | Concentration (μg/mL) | (DSM19882) | (DSM22644) |
| No enzyme | 0 | 100.0 | 100.0 |
| SEQ ID NO: 18 | 20 | 43.4 | 55.7 |
| SEQ ID NO: 33 | 20 | 57.9 | 55.4 |
| SEQ ID NO: 18 | 20 | 37.9 | 59.4 |
| SEQ ID NO: 15 | 20 | 22.6 | 55.2 |
| SEQ ID NO: 36 | 20 | 52.6 | 62.0 |
| SEQ ID NO: 51 | 20 | 54.3 | 61.3 |
| SEQ ID NO: 21 | 20 | 51.6 | 58.9 |
| SEQ ID NO: 24 | 20 | 49.9 | 48.0 |
| SEQ ID NO: 12 | 20 | 55.1 | 54.7 |
| SEQ ID NO: 6 | 20 | 75.4 | 70.3 |
| SEQ ID NO: 27 | 20 | 73.8 | 74.8 |

### Example 9. Endoscope Cleaning in Liquid Model Detergent

Endoscope biofilms were established using P. aeruginosa DSM19882: The strain was inoculated into 10 mL LB and incubated at 37° C. for 16 hours with shaking (200 rpm). After propagation, the culture was diluted (1:100) in LBNS and the bacterial suspension was added to 96-well microtiter plates (Thermo Scientific, cat #₁₆₇₀₀₈) containing sterile pieces (1 cm) of endoscope tubing (4.7 mm diameter, Fluoroelastomer/Viton®, USP Class VI, Endoscopy Development Company, LLC). Sterile medium was added to control wells. After 24 hours at 26° C. (static incubation), the endoscope tubes were treated with a model cleaning solution (5 g/L Model detergent MC in 5° dH water hardness) containing no enzyme (control) or 20 g/mL enzyme for 1 hour at 26° C. The endoscope pieces were subsequently rinsed with 5° dH water and stained with 0.095% crystal violet (SIGMA V5265) for 15 min. After additional rinses, the endoscope pieces were blotted on absorbent paper and the remaining dye was dissolved using 30% acetic acid. 200 μl aliquots of the suspensions were transferred to a 96-well microtiter plate and the absorbance was measured at 595 nm. The results are displayed in table 10 as percentages of remaining biofilm after enzymatic treatment as compared to the control (endoscope biofilm treated without enzyme).

TABLE 10
Endoscope cleaning properties in medical cleaning model detergent MC

| | Enzyme dosage | Remaining biofilm (% of |
|---|---|---|
| Enzyme | (μg/ml) | untreated control) |
| No enzyme | 0 | 100.0 |
| SEQ ID NO: 21 | 20 | 43.3 |
| SEQ ID NO: 27 | 20 | 58.9 |
| SEQ ID NO: 33 | 20 | 58.4 |
| SEQ ID NO: 18 | 20 | 30.1 |

The results show that the polypeptides of the invention have endoscope cleaning properties, i.e., disrupt and/or remove the biofilm or components of the biofilm tested when compared to samples comprising no enzyme.

### Example 10. Construction of Clades and Phylogenetic Trees

The polypeptides of the invention having hydrolase activity and comprises a GH114 domain as well as clusters such as the clades. A phylogenetic tree was constructed, of polypeptide sequences containing a GH114 domain, as defined in CAZY (GH114, Glycoside Hydrolase Family 114, CAZy database, cazy.org, Lombard et al., 2014, Nucleic Acids Res. 42: D490-D495). The phylogenetic tree was constructed from a multiple alignment of mature polypeptide sequences containing at least one GH114 domain. The sequences were aligned using the MUSCLE algorithm version 3.8.31 (Edgar, 2004, Nucleic Acids Research 32 (5): 1792-1797), and the trees were constructed using FastTree version 2.1.8 (Price et al., 2010, PloS one 5(3)) and visualized using iTOL (Letunic & Bork, 2007, Bioinformatics 23 (1): 127-128). The polypeptide comprises of the GH114 domain comprises several motifs. One example is [VLI]XE[EDSQ]C (SEQ ID NO: 60) situated in positions 182 to 186 in Amycolatopsis circi (SEQ ID NO: 3), where E at position 184 is one of the two catalytic site residues and fully conserved in the polypeptides of the invention. Another motif which may be comprised by the polypeptides of the invention is CY[FLIV][SDN][ATVG] (SEQ ID NO: 61) situated in positions corresponding to positions 55 to 59 in Amycolatopsis circi (SEQ ID NO: 3). The tyrosine at position 56 in SEQ ID NO: 3 is involved in substrate-binding.

The polypeptides containing a GH114 domain can be separated into distinct sub-clusters. The sub-clusters are defined by one or more short sequence motifs, as well as containing a GH114 domain as defined in CAZY (GH114, CAZy database, cazy.org, Lombard et al., 2014, Nucleic Acids Res. 42: D490-D495). We denoted one sub-cluster comprising the motif [VLI]XE[EDSQ]C (SEQ ID NO: 60) as the VAE clade. All polypeptide sequences containing a GH114 domain as well as the motif will be denoted as belonging to the VAE clade.

The polypeptides in the VAE clade can be further separated into multiple distinct sub-clusters, or clades, where we denoted the clades listed below. The relationship between the described clades are shown in FIG. 4.

### Generation of DYQ Clade

A phylogenetic tree was constructed, of polypeptide sequences containing a GH114 domain and a VAE motif, as defined above. The sequences were aligned using the MUSCLE algorithm version 3.8.31 (Edgar, 2004. Nucleic Acids Research 32 (5): 1792-1797), and the tree was constructed using FastTree version 2.1.8 (Price et al., 2010, PloS one 5 (3)) and visualized using iTOL (Letunic & Bork, 2007. Bioinformatics 23 (1): 127-128). Using the phylogenetic tree, the polypeptides containing a GH114 domain and a VAE clade motif can be separated into additional distinct sub-clusters, one which we denoted DYQ.

A characteristic motif for this sub-cluster is the motif DYQ[LI]G (SEQ ID NO: 62), corresponding to amino acids DYQIG at positions 23 to 27 in SEQ ID NO: 3, where G at position 27 is fully conserved in the polypeptides and involved in substrate binding. An additional motif of the DYQ clade is FQ[TAV]Q[PSD] (SEQ ID NO: 63), corresponding to amino acid 60 to 64 in the reference polypeptide (SEQ ID NO: 3). Examples of polypeptides of the DYQ clade include SEQ ID NO: 3, SEQ ID NO: 6, SEQ ID NO: 9, SEQ ID NO: 12, SEQ ID NO: 21, SEQ ID NO: 24, SEQ ID NO: 39, SEQ ID NO: 42, SEQ ID NO: 45, SEQ ID NO: 48, SEQ ID NO: 51, SEQ ID NO: 54, SEQ ID NO: 79, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 90, SEQ ID NO: 91, SEQ ID NO: 92, SEQ ID NO: 93, SEQ ID NO: 94, SEQ ID NO: 96, SEQ ID NO: 131, SEQ ID NO: 132, SEQ ID NO: 134, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144 and SEQ ID NO: 145.

### Generation of AEE Clade

The AEE clade comprises polypeptides of bacterial origin, containing a GH114 domain and belonging to the DYQ clade, having hydrolase activity. The polypeptides of the clade comprise the motif example AEECG (SEQ ID NO: 64), corresponding to amino acids AEECG at positions 183 to 187 of SEQ ID NO: 3 where all amino acids are fully conserved in AEE clade. An additional motif of the AEE clade is NAFQ[AT]Q (SEQ ID NO: 65), corresponding to amino acids 58 to 63 in the reference polypeptide (SEQ ID NO: 3). Examples of polypeptides of the AEE clade includes SEQ ID NO: 3, SEQ ID NO: 6, SEQ ID NO: 9, SEQ ID NO: 12, SEQ ID NO: 21, SEQ ID NO: 24, SEQ ID NO: 54, SEQ ID NO: 79, SEQ ID NO: 82, SEQ ID NO: 83, SEQ ID NO: 84, SEQ ID NO: 85, SEQ ID NO: 132, SEQ ID NO: 136, SEQ ID NO: 138, and SEQ ID NO: 142.

### Generation of WQW Clade

The WQW clade comprises polypeptides of bacterial and fungal origin, containing a GH114 domain and belonging to the VAE clade, having hydrolase activity. The polypeptides of the clade comprise the motif example WQWQL (SEQ ID NO: 66), corresponding to amino acids WQWQL positions 30 to 34 of Vibrio sp. SEQ ID NO: 27 where L (corresponding to position 34 of SEQ ID NO: 27) is fully conserved in WOW clade and involved in substrate binding. An additional motif of the WOW clade is [VLI][GASD]LKN[DGS][VLIP] (SEQ ID NO: 67), corresponding to amino acids IGLKNDL positions 171 to 177 of Vibrio sp. SEQ ID NO: 27 where LKN (corresponding to positions 173 and 175 of SEQ ID NO: 27) is fully conserved in WQW clade. Examples of polypeptides of the WQW clade include SEQ ID NO: 27, SEQ ID NO: 33, SEQ ID NO: 36, SEQ ID NO: 80, SEQ ID NO: 81, SEQ ID NO: 89, SEQ ID NO: 95 and SEQ ID NO: 146.

### Generation of IEY Clade

The IEY clade comprises polypeptides of fungal origin, containing a GH114 domain and belonging to the VAE clade, having hydrolase activity. The polypeptides of the clade comprise the motif example GXXVX[NHQTS]IEY[PG] (SEQ ID NO: 68), corresponding to amino acids “GKPVLQIEYP” positions 230 to 239 of SEQ ID NO: 18 where E (corresponding to position 237 of SEQ ID NO: 3) is fully conserved in GVFLD clade, part of the substrate binding pocket, and one of the two putative catalytic site residues.

An additional motif of the IEY clade is VICYF (SEQ ID NO: 69), corresponding to amino acids VICYF positions 68 to 72 of Fusarium solani (SEQ ID NO: 18), 27 where CYF (corresponding to positions 70 and 72 of SEQ ID NO: 18) is fully conserved in IEY clade. Examples of polypeptides of the IEY clade includes SEQ ID NO: 15, SEQ ID NO: 18, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 133 and SEQ ID NO: 135.

### Generation of ICY Clade

The ICY clade comprises polypeptides of fungal origin, containing a GH114 domain and belonging to the IEY clade, having hydrolase activity. The polypeptides of the clade comprise the motif example ICYFSA (SEQ ID NO: 70), corresponding to amino acids ICYFSA in SEQ ID NO: 15. Examples of polypeptides of the ICY clade is SEQ ID NO: 15, SEQ ID NO: 57, SEQ ID NO: 98, SEQ ID NO: 101, SEQ ID NO: 104, SEQ ID NO: 107, SEQ ID NO: 114, SEQ ID NO: 115, SEQ ID NO: 120, SEQ ID NO: 124, SEQ ID NO: 125, SEQ ID NO: 127, SEQ ID NO: 129 and SEQ ID NO: 133.

### Generation of DFAVL Clade

The DFAVL_clade comprises polypeptides of fungal origin, containing a GH114 domain and belonging to the IEY clade, having hydrolase activity. The polypeptides of the clade comprise the motif example DFAVL (SEQ ID NO: 71), corresponding to amino acids DFAVL at positions 199 to 203 of SEQ ID NO: 18, where L at position 203 in SEQ ID NO: 71 is fully conserved in the clade, and positioned next to one of the catalytic residues E at position 204. Examples of polypeptides of the DFAVL clade are SEQ ID NO: 18, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 110, SEQ ID NO: 111, SEQ ID NO: 112, SEQ ID NO: 113, SEQ ID NO: 116, SEQ ID NO: 117, SEQ ID NO: 118, SEQ ID NO: 119, SEQ ID NO: 121, SEQ ID NO: 123, SEQ ID NO: 126, SEQ ID NO: 128, SEQ ID NO: 130 and SEQ ID NO: 135.

A phylogenetic tree of the polypeptides in the clades is shown in FIG. 1 and FIG. 4.

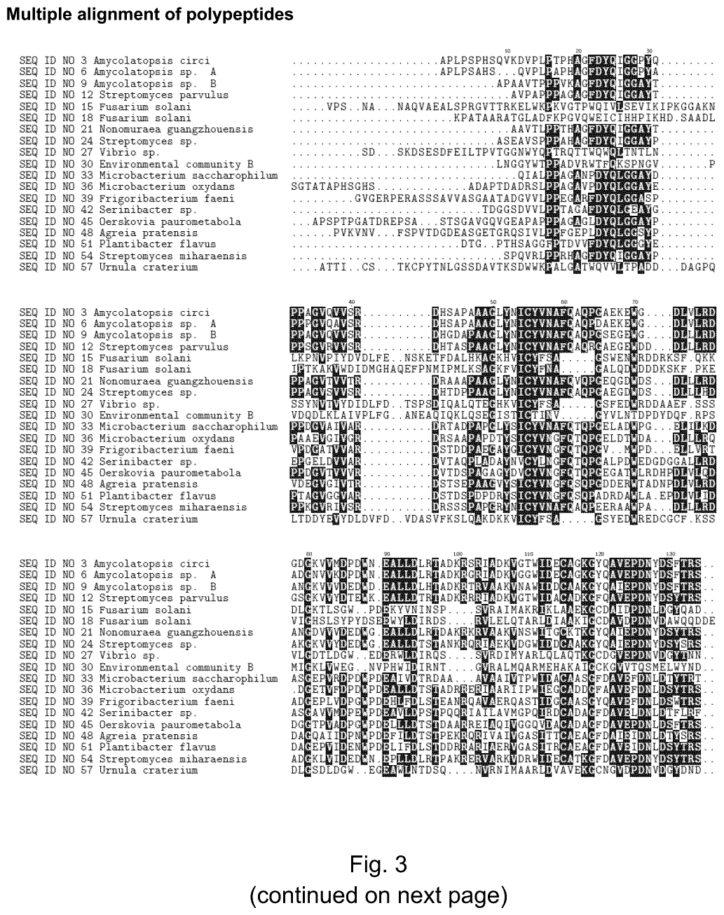

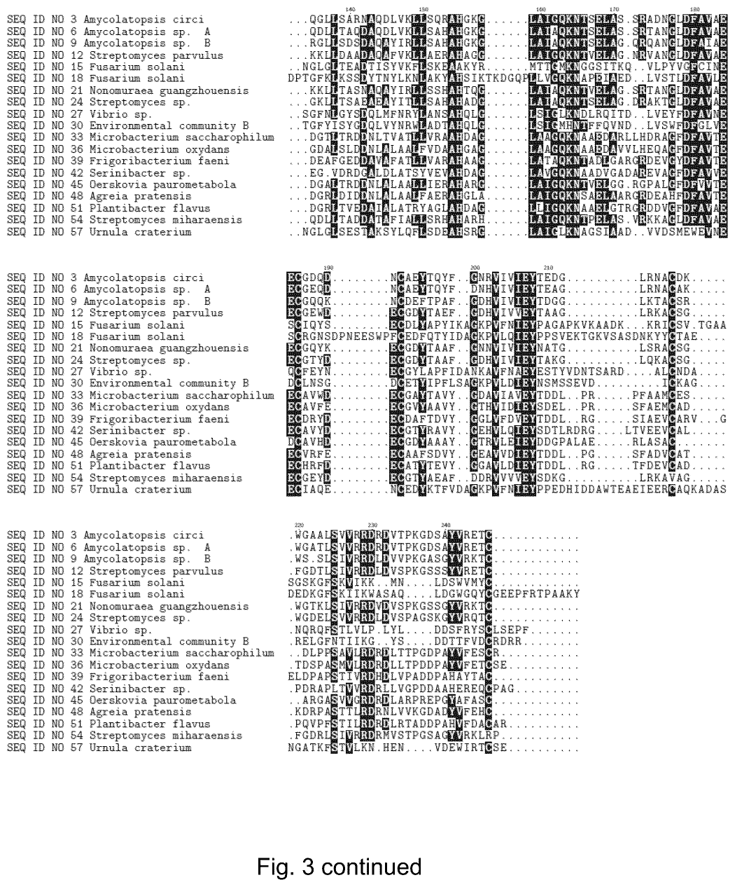

An alignment of the polypeptides of the invention comprised in the clade is shown in FIG. 3.

### Example 11. Cloning, Expression and Fermentation of GH114 Glycosyl Hydrolase Enzyme Polypeptides from Fusarium compactum, Neonectria candida, Fusarium avenaceum and Preussia aemulans

Genes encoding eight putative endo-α-1,4-polygalactosaminidases belonging to family GH114 glycosyl hydrolases, were cloned from the strains listed in Table 11 below. The strains were isolated from environmental samples collected in the countries indicated. For all strains, chromosomal DNA was isolated and a whole genome sequence was determined using Illumina technology. For Neonectria candida, whole genome sequencing was purchased from Exiqon A/S, Vedbaek, Denmark.

TABLE 11

| | | Genome | Gene | SEQ ID |
|---|---|---|---|---|
| Strain | Source country | Assembler | Annotation | NO |
| Neonectria candida | Sweden | Spades v3.5.0 | GeneMark | 97 |
| | | | 2.3c | |
| Fusarium | Australia | Spades v3.9.0 | GeneMark | 98, 99 |
| compactum | | | ES 4.28 | |
| Fusarium | Denmark | Spades v3.9.0 | GeneMark | 100 |
| avenaceum | | | ES 4.28 | |
| Preussia aemulans | Denmark | ABySS 1.2.7 | GeneMark | 101 |
| | | | 2.3c | |

### References for Analysis Programs Listed Above

Spades genome assemblers: Bankevich et al., 2012, J. Comput. Biol. 19 (5): 455-477

ABySS genome assembler: Simpson et al., 2009, Genome Res. 19 (6): 1117-1123.

GeneMark gene annotation: Ter-Hovhannisyan et al., 2008, Genome Res. 18 (12): 1979-1990.

The whole genome sequences were assembled and annotated with the software packages indicated in Table 11. The gene model coding for the polypeptide in SEQ ID NO: 101 was adjusted after analysis with the GenWise gene prediction software (Li et al., 2015, Nucleic Acids Res. 43 (W1): W580-W584).

Peptides predicted from the annotated genome were searched for similarity to the GH114 domain as defined by CAZy (cazy.org, Lombard et al., 2014, Nucleic Acids Res. 42: D490-D495), and the eight polypeptides were identified. Corresponding DNA sequences were PCR amplified from genomic DNA with gene-specific primers that also append a Kozak translation initiation sequence “TCACC” immediately 5′ of the start codon, and the amplified DNA fragments were cloned into the Aspergillus expression vector pMStr57 (WO 2004/032648) that had been digested with BamHI and XhoI.

The cloned GH114 encoding genes were transformed into the Aspergillus oryzae strain MT3568 (WO 2011/057140) by the methods described in Christensen et al., 1988, Biotechnology 6:1419-1422 and WO 2004/032648. Transformants were selected during regeneration from protoplasts based on the ability, conferred by a selectable marker in the expression vector, to utilize acetamide as a nitrogen source, and were subsequently re-isolated under selection.

Production of the recombinant GH114 glycosyl hydrolase enzymes was evaluated by culturing the transformants in 96-well deep-well microtiter plates for 4 days at 30° C. in 0.25 ml of both YPG medium (WO 2005/066338) and DAP-4C-1 medium (WO 2012/103350) and monitoring peptide expression by SDS-PAGE. For larger-scale production of the recombinant putative GH114 glycosyl hydrolase enzymes, a single Aspergillus transformant was selected for each GH114 glycosyl hydrolase enzyme and the transformants were cultured in 500 ml baffled flasks containing 150 ml of DAP-4C-1 medium. The cultures were shaken on a rotary table at 150 RPM at for 4 days. The culture broth was subsequently separated from cellular material by passage through a 0.22 μm filtration unit.

### Example 12. Chromatographic Purification of GH114 Peptides from Fusarium compactum, Neonectria candida, Fusarium avenaceum and Preussia aemulans

The pH of the filtered sample was adjusted to around pH 7.5 and 1.8 M ammonium sulfate was added. The sample was applied to a 5 ml HiTrap™ Phenyl (HS) column on an Äkta Explorer. Prior to loading, the column had been equilibrated in 5 column volumes (CV) of 50 mM HEPES+1.8 M AMS pH 7. In order to remove unbound material, the column was washed with 5 CV of 50 mM HEPES+1.8 M AMS pH 7. The target protein was eluted from the column into a 10 ml loop using 50 mM HEPES+20% isopropanol pH 7. From the loop, the sample was loaded onto a desalting column (HiPrep™ 26/10 Desalting), which had been equilibrated with 3CV of 50 mM HEPES+100 mM NaCl pH 7.0. The target protein was eluted with 50 mM HEPES+100 mM NaCl pH 7.0 and relevant fractions were selected and pooled based on the chromatogram. The flow rate was 5 ml/min.

For the polypeptides shown in SEQ ID NOs: 98 and 99, the sample collected from the purification process described above, was applied to a 1 mL HiTrap™ Blue HP column on an Äkta PURE system. Prior to loading the column had been equilibrated in 10 CV of 50 mM HEPES+100 mM NaCl pH 7.0. During loading of the sample to the column, the flow through was collected. In order to remove any unbound material, the column was washed with 7 CV 50 mM HEPES+100 mM NaCl pH 7.0. The target protein was eluted with 11.2 CV of 50 mM Tris, 1 M NaCl, pH 8. Based on the chromatogram, relevant fractions were collected. The protein was either in the flow through or in the eluate. Protein concentration in the final samples was estimated by measuring absorption at 280 nm.

### Example 13. Cloning and Expression of Two GH114 Glycosyl Hydrolase Polypeptides from Fusarium proliferatum, and Fusarium lateritium

Two putative endo-α-1,4-polygalactosaminidases belonging to family GH114 were derived from fungal strains isolated from environmental samples by standard microbiological isolation techniques. Strains were identified, and taxonomy was assigned based on DNA sequencing of the ITS (Table 12).

TABLE 12

| SEQ ID NO | Organism_name | Source country |
|---|---|---|
| 102 | Fusarium proliferatum | China |
| 103 | Fusarium lateritium | China |

Chromosomal DNA from individual strains (Table 12) was isolated by DNeasy Plant Mini Kit (Qiagen, Hilden, Germany). 5 μg of chromosomal DNA were sent for full genome sequencing using Illumina technology. Genome sequencing, the subsequent assembly of reads and the gene discovery (i.e., annotation of gene functions) is known to the person skilled in the art and the service can be purchased commercially.

Peptides predicted from the annotated genome were searched for similarity to the GH114 domain as defined by CAZy (cazy.org, Lombard et al., 2014, Nucleic Acids Res. 42: D490-D495), and the three peptides were identified. Corresponding DNA sequences were PCR amplified from genomic DNA with gene-specific primers, and the amplified DNA fragments were cloned into the expression vector pCaHj505 (WO 2013/029496).

The final expression plasmid was transformed into the Aspergillus oryzae MT3568 expression host. A. oryzae MT3568 is a derivative of A. oryzae JaL355 (WO 02/40694) in which pyrG auxotrophy was restored by disrupting the A. oryzae acetamidase (amdS) gene with the pyrG gene. The GH114 gene was integrated by homologous recombination into the A. oryzae MT3568 host cell genome upon transformation.

The gene coding for amdS was used as marker. Transformants were selected on pyrG media agar supplemented with 10 mM acetamide. One recombinant A. oryzae clone containing the GH114 expression construct was selected and cultivated in 2400 ml of Dap4C medium in shake flasks for 3 days at 30° C. under 80 rpm agitation. Enzyme containing supernatants were harvested by filtration using a 0.22 μm 1-liter bottle top vacuum filter (Thermo Fisher Scientific Inc., Waltham, MA, USA).

### Example 14. Cloning, Expression and Fermentation of Fungal GH114 Glycosyl Hydrolases

### Strains

Escherichia coli Top-10 strain purchased from TIANGEN (TIANGEN Biotech Co. Ltd., Beijing, China) was used to propagate our expression vector.

Aspergillus oryzae strain DAU785 (described in WO 2018/113745, page 293) was used for heterologous expression of the genes described in the Table 1.

### Media

Dap4C medium was composed of 11 g MgSO₄·7H₂O, 1 g KH₂PO₄, 2.2 g Citric acid·H₂O, 20 g glucose, 10 g maltose, 5.2 g K₃PO₄·H₂O, 0.5 g yeast extract, 1.25 g CaCO₃, 0.5 ml AMG Trace element solution and deionized water to 1 litre. After autoclave, 3.3 ml of 20% Lactic Acid (autoclaved) and 9.3 ml of 50% (NH₄)₂HPO₄ (sterile filtered) were added to every 400 ml above medium.

AMG Trace element solution was composed of 6.8 g ZnCl₂, 2.5 g CuSO₄·5H₂O, 0.24 g NiCl₂·5H₂O, 13.9 g FeSO₄·7H2O, 13.6 g MnSO₄·5H₂O, 3 g Citric acid·H₂O, and deionised water to 1000 ml.

LB plates were composed of 10 g of Bacto-tryptone, 5 g of yeast extract, 10 g of sodium chloride, 15 g of Bacto-agar, and deionised water to 1000 ml.

LB medium was composed of 1 g of Bacto-tryptone, 5 g of yeast extract, and 10 g of sodium chloride, and deionised water to 1000 ml.

COVE sucrose plates were composed of 342 g of sucrose, 20 g of agar powder, 20 ml of COVE salt solution, and deionized water to 1 litre. The medium was sterilized by autoclaving at 15 psi for 15 minutes. The medium was cooled to 60° C. and 10 mM acetamide, 15 mM CsCl, Triton X-100 (50 μl/500 ml) were added.

COVE-2 plate/tube for isolation: 30 g/L sucrose, 20 ml/L COVE salt solution, 10 mM acetamide, 30 g/L noble agar (Difco, Cat #₂₁₄₂₂₀).

COVE salt solution was composed of 26 g of MgSO₄·7H₂O, 26 g of KCL, 26 g of KH₂PO₄, 50 ml of COVE trace metal solution, and deionised water to 1000 ml.

COVE trace metal solution was composed of 0.04 g of Na₂B₄O₇·10H₂O, 0.4 g of CuSO₄·5H₂O, 1.2 g of FeSO₄·7H₂O, 0.7 g of MnSO₄·H₂O, 0.8 g of Na₂MoO₄·2H₂O, 10 g of ZnSO₄·7H₂O, and deionised water to 1000 ml.

The GH114 glycosyl hydrolase genes derived from fungal strains isolated from environmental samples by standard microbiological isolation techniques. Strains were identified, and taxonomy was assigned based on DNA sequencing of the ITS (Table 13).

TABLE 13

| Protein SEQ ID | | source |
|---|---|---|
| NO: | Donor Organism name | country |
| 72 | Chaetomium globosum | China |
| 73 | Stilbella fimetaria | China |
| 74 | Volutella ciliata | China |
| 75 | Clonostachys epichloe | China |
| 76 | Myrothecium sp | China |
| 77 | Botryotrichum piluliferum | China |
| 78 | Myrothecium sp. | China |
| 104 | Fusarium proliferatum | China |
| 105 | Chaetomium ancistrocladum | China |
| 106 | Fusarium verficillioides | China |
| 107 | Fusarium lateritium | China |
| 108 | Chaetomium sp. ZY474 | China |

Chromosomal DNA from individual strains (Table 13) was isolated by QIAamp Dneasy Kit (Qiagen, Hilden, Germany). 5 μg of chromosomal DNA were sent for full genome sequencing using Illumina technology. Genome sequencing, the subsequent assembly of reads and the gene discovery (i.e., annotation of gene functions) is known to the person skilled in the art and the service can be purchased commercially.

The genome sequences were analyzed for putative GH114 glycosyl hydrolases from the PFAM database families PF03537. This analysis identified genes encoding putative GH114, which were subsequently cloned and recombinantly expressed in Aspergillus oryzae. Using the SignalP program v.3 (Nielsen et al., 1997, Protein Engineering 10:1-6), the signal peptide and accordingly the mature peptide (SEQ ID NOs: 72-78, 104-108) were predicted.

The GH114 glycosyl hydrolase genes were amplified by PCR respectively from above isolated genomic DNA. The purified PCR product was cloned into the previously digested expression vector pDAU724 (described in WO 2018/113745, page 293) by ligation with an IN-FUSION™ CF Dry-down Cloning Kit (Clontech Laboratories, Inc., Mountain View, CA, USA) according to the manufacturer's instructions. The ligation mixture was used to transform E. coli TOP10 chemically competent cells (described in Strains). Correct colonies containing the corresponding GH114 gene was selected and verified by DNA sequencing (by SinoGenoMax Company Limited, Beijing, China). The correct GH114 containing colony was cultivated overnight in 3 ml of LB medium supplemented with 100 μg of ampicillin per ml. Plasmid DNA was purified using a Qiagen Spin Miniprep kit (Cat. 27106) (QIAGEN GmbH, Hilden, Germany) according to the manufacturer's instructions. Protoplasts of Aspergillus oryzae DAU785 were prepared according to WO 95/02043. 100 μl of protoplasts were mixed with 2.5-10 μg of the Aspergillus expression vector (above extracted plasmid) comprising the GH114 glycosyl hydrolase gene and 250 μl of 60% PEG 4000, 10 mM CaCl₂), and 10 mM Tris-HCl PH 7.5 and gently mixed. The mixture was incubated at 37° C. for 30 minutes and the protoplasts were spread onto COVE sucrose plates for selection. After incubation for 4-7 days at 37° C. spores of 4 transformants were inoculated into 3 ml of DAP4C medium. After 3 days cultivation at 30° C., the culture broths were analyzed by SDS-PAGE using Novex® 4-20% Tris-Glycine Gel (Invitrogen Corporation, Carlsbad, CA, USA) to identify the transformants producing the largest amount of recombinant GH114 with respective estimated mature peptide size. Spores of the best expressed transformant were spread on COVE-2 plates for re-isolation in order to isolate single colonies. Then a single colony was spread on a COVE-2 tube until sporulation. Spores from the best expressed transformant were cultivated in 2400 ml of Dap4C medium in shake flasks during 3 days at a temperature of 30° C. under 80 rpm agitation. Culture broth was harvested by filtration using a 0.22 μm filter device. The filtered fermentation broth was used for enzyme characterization.

### Example 15. Cloning and Expression for GH114 Glycosyl Hydrolases Having SEQ ID NO: 109 to SEQ ID NO: 135

### Sequence Trimming and Codon Optimization

Selected protein sequences were submitted to subcellular localization predictors (DeepLoc-1.0: Bioinformatics, volume 33, Issue 24, 15 Dec. 2017, Page 4049; SignalP-5.0: Nature Biotechnology, volume 37, 2019, page 420) to evaluate extracellular secretion probability. SEQ ID NO: 114, SEQ ID NO: 115, SEQ ID NO: 121, SEQ ID NO: 122, SEQ ID NO: 127, SEQ ID NO: 129, and SEQ ID NO: 133 had lysosomal predicted targeting and Nt lysosomal targeting signal sequence were truncated.

All protein sequences were reverse-translated with a method preferentially utilizing frequently used Aspergillus oryzae codons and algorithms designed to identify and remove sequence features that might hinder cloning or expression were used. A single DNA sequence was selected for each GH114 protein in this process as described in patent US 2015/0152452.

### Cloning, Transformation and Expression

Codon-optimized DNA sequences were purchased from TwistBioscience. A PCR amplification step consisting of one 3 min. denaturation step at 98° C. followed by 30 cycles of 20 sec. denaturation, 2 min. annealing/amplification at 72° C. and one final amplification step of 10 min. at 72° C. was carried out on 20 μL mixes consisting of 4 μL KAPA polymerase buffer, 0.4 μL 10 mM KAPA dNTP, 0.4 μL 10 UM forward primer (SEQ ID NO: 147); 0.4 μL 10 UM reverse primer (SEQ ID NO: 148), 2 ng synthetic genes resuspended in 10 mM TE buffer pH 8, 0.4 μL KAPA polymerase (KK2502) and 14 μL PCR grade water.

SEQ ID NO 147:
gacagccgcatccgtggtctccgca
SEQ ID NO 148:
ctaccgccagcagtgtctgcgattta

Cloning strategy was carried out by in vivo assembly of three overlapping DNA fragments in Aspergillus oryzae strain ColS1300 protoplasts following a procedure described in WO 2018/050666.

To ensure extracellular secretion of the heterologous GH114 proteins, all synthetic sequences were fused to a DNA sequence encoding for the extracellular secretion signal of sequence MKLSWLVAAALTAASVVSA (SEQ ID NO: 149). Wild-type secretion signal peptides were also replaced by the synthetic extracellular secretion signal of sequence MKLSWLVAAALTAASVVSA.

ColS1300 strain transformation libraries were screened for protein expression after a first selection on NaNo3 plates following a procedure described in WO 2018/050666. Protein expression analyses were carried out on a Perkin Elmer (Waltham, Massachusetts, U.S) LAbChip GXII.

### Example 16. Purification of GH114 Glycosyl Hydrolases

### Purification

Some of the recombinant GH114 glycosyl hydrolase enzymes (SEQ ID NOs: 72-79 and SEQ ID NOs: 106-116) were purified by hydrophobic interaction chromatography process, with a little difference on pH of buffer and concentration of ammonium sulfate for different molecules. The culture supernatant of recombinant was firstly added by ammonium sulfate with a final concentration of 1.5 M and loaded into Phenyl Sepharose 6 Fast Flow column or Phenyl High Performance column (GE Healthcare) equilibrated with 20 mM PBS at pH 6.0 with 1.5 M ammonium sulfate added. A gradient decrease of ammonium sulfate concentration from 1.5 M to 0 was set up as elution condition. The elution fractions and flow-through faction were assayed by SDS-PAGE. The fractions with target protein were pooled together and then diafiltrated with 20 mM PBS at pH6.0. The protein concentration was determined by Qubit® Protein Assay Kit (Invitrogen, cat Q33212). Enzyme activity assay was carried out as described below.

### Substrate Preparation

Based on the article (Takada et al., 1981, J. Biochem. 89:1265-1274), fungal strain Aspergillus parasiticus was cultured in the medium suggested from literature at 30° C. for 5 days. Then the culture broth was filtrated, and the culture supernatant was kept. The culture supernatant was added with same volume of ethanol, mixed for several minutes and centrifuged. The precipitant was washed three times with ethanol. Finally, the precipitant was washed with pure water and stored as substrate.

### Enzyme Assay

The precipitant was dissolved in 20 mM PBS at pH 6.0 with the final concentration of 0.2 mg/ml as substrate solution. 10 ul enzyme solution at 0.1 mg/mL was added into 90 ul substrate solution, incubated at 40° C. for 30 minutes. 50 ul of reaction mixture was added into 150 μl PHBAH solution (4-hydroxybenzhydtazide (CAS: 5351-23-5) diluted in 0.5 M NAOH to a final concentration of 1.5%), incubated at 100° C. for 5 minutes. Then 70 ul of the incubation solution was picked up and tested for absorbance at 410 nm. 10 ul water added into substrate solution was set as blank control.

### Example 17. Measuring Thermal Stability of GH114 Glycosyl Hydrolases

Thermal stability measurements were performed using a capillary based nano differential scanning fluorescence instrument (nanoDSF); Prometheus NT.Plex (NanoTemper Technologies GmbH, München, Germany). Standard nanoDSF grade capillary chips were used (Cat #: PR-AC002) from NanoTemper Technologies. The protein samples were mixed 1:10 in 50 mM NaAcetate, 50 mM MES, 50 mM Glycine pH 8 or in 3.3 g/L Model A and loaded into the capillaries (each sample in triplicate) by capillary action. The emission intensities at 330 and 350 nm were optimized by altering the LED power on the instrument to ensure sufficient signal. The fluorescence signals at 330 and 350 nm were monitored continuously as a function of temperature (heating rate used for thermal unfolding was 3.3° C. per minute from 20 to 95° C.). The data was analyzed using the PR. ThermControl_2.1.2.6031 software provided by the manufacturer. The analysis is model independent and simply takes the peak maximum of the first derivative which corresponds to the approximate thermal unfolding transition midpoint, defined as Td.

TABLE 14

| | | nDSF | nDSF |
|---|---|---|---|
| SEQ ID NO | Organism name | pH 8 | Model A |
| 6 | Amycolatopsis sp. A | 63.43 | 58.50 |
| 18 | Fusarium solani | 48.35 | 35.15 |
| 72 | Chaetomium globosum | 56.32 | 52.49 |
| 73 | Stilbella fimetaria | 51.95 | 41.94 |
| 74 | Volutella ciliata | 41.95 | 29.54 |
| 75 | Clonostachys epichloe | 45.59 | 41.73 |
| 76 | Myrothecium sp-75362 | 57.79 | 48.90 |
| 77 | Botryotrichum piluliferum | 43.92 | 36.05 |
| 78 | Myrothecium sp. | 51.14 | 40.04 |
| 79 | Amycolatopsis sp-63067 | 57.00 | 40.65 |
| 81 | Amycolatopsis niigatensis | 56.63 | 51.34 |
| 82 | Streptomyces sp. AA4 | 63.77 | 52.16 |
| 83 | Amycolatopsis alba DSM 44262 | 58.30 | 50.74 |
| 87 | Microbacterium oleivorans | 48.43 | 40.54 |
| | O827CG | | |
| 89 | Microbacterium oxydans | 53.87 | 48.17 |
| 90 | Agreia pratensis | 56.60 | 53.64 |
| 91 | Microbacterium lemovicicum | 42.43 | 31.98 |
| 93 | Curtobacterium oceanosedimentum | 43.10 | 30.51 |
| 96 | Enviromental sample | 51.77 | 44.12 |

### Example 18. Deep-Cleaning in Liquid Model Detergent A on Fungal EPS Swatches

The wash performance was determined as follows; 50 μl aliquots of crude fungal EPS extract (see example “production of poly-galactosamine” above) were spotted on sterile textile swatches (WFK20A) and incubated for 15 min at ambient temperature. Control swatches were spotted with 3 M NaCl. The swatches (sterile or with the extract) were placed in 50 mL test tubes and 10 mL of wash liquor (15° dH water with 0.2 g/L iron (III) oxide nano-powder (544884; Sigma-Aldrich) and 3.33 g/L liquid model A detergent) and the enzyme(s) was added to each tube. Washes without enzyme were included as controls. The test tubes were placed in a Stuart rotator and incubated for 1 hour at 30° C. and 20 rpm. The wash liquor was then removed, and the swatches were rinsed twice with 15° dH water and dried on filter paper over night. The remission values (Rem460 nm) were measured using a Datacolor 800V and are displayed in Tables 9 and 10. Wash performance (WP) values (Rem460 nm_{(swatch washed with enzyme)}−Rem460 nm_{(swatch washed without enzyme})) are also indicated. Alternatively, swatches were scanned using an Epson Expression 10000XL flatbed scanner. R (red), G (green) and B (blue) values in the RGB color space were determined using Color Analyzer software and Intensity was calculated as \sqrt{R²+G²+B²} for each swatch (data is shown in Table 12). Wash performance (WP) values (ΔIntensity=Intensity_{(swatch washed with enzyme)}−Intensity_{(swatch washed without enzyme)} are also indicated.

TABLE 15
Deep-cleaning effects of the GH114 glycosyl hydrolase
homologues in model A detergent

| | | Enzyme | | WP |
|---|---|---|---|---|
| | SEQ | conc. | Average | (ΔRem460 |
| Swatch | ID NO: | (μg/ml) | Rem460 nm | nm) |
| wfk20A, clean textile | | 0 | 70.7 | |
| Wfk20A, EPS, no enzyme | | 0 | 43.9 | |
| Wfk20A, EPS | 80 | 0.1 | 73.4 | 29.5 |
| Wfk20A, EPS | 82 | 0.1 | 52.2 | 8.3 |
| Wfk20A, EPS | 83 | 0.1 | 56.5 | 12.6 |
| Wfk20A, EPS | 84 | 0.1 | 52.7 | 8.8 |
| Wfk20A, EPS | 85 | 0.1 | 51.5 | 7.6 |
| Wfk20A, EPS | 86 | 0.1 | 49.8 | 5.9 |
| Wfk20A, EPS | 87 | 0.1 | 54.0 | 10.1 |
| Wfk20A, EPS | 88 | 0.1 | 51.0 | 7.1 |
| Wfk20A, EPS | 89 | 0.1 | 54.4 | 10.5 |
| Wfk20A, EPS | 90 | 0.1 | 50.9 | 7.0 |
| Wfk20A, EPS | 91 | 0.1 | 56.0 | 12.1 |
| Wfk20A, EPS | 92 | 0.1 | 51.4 | 7.5 |
| Wfk20A, EPS | 94 | 0.1 | 56.9 | 13.0 |
| Wfk20A, EPS | 95 | 0.1 | 45.6 | 1.7 |
| Wfk20A, EPS | 96 | 0.1 | 54.6 | 10.7 |
| Wfk20A, EPS | 97 | 0.1 | 50.1 | 6.2 |

TABLE 16
Deep-cleaning effects of the GH114 glycosyl hydrolase
homologues in model A detergent

| | | Enzyme | | WP |
|---|---|---|---|---|
| | SEQ | conc. | Average | (ΔRem460 |
| Swatch | ID NO: | (μg/ml) | Rem460 nm | nm) |
| wfk20A, clean textile | | 0 | 71.2 | |
| Wfk20A, EPS, no enzyme | | 0 | 40.3 | |
| Wfk20A, EPS | 72 | 0.1 | 42.5 | 2.2 |
| Wfk20A, EPS | 73 | 0.1 | 43.4 | 3.1 |
| Wfk20A, EPS | 74 | 0.1 | 53.3 | 13.0 |
| Wfk20A, EPS | 75 | 0.1 | 47.4 | 7.1 |
| Wfk20A, EPS | 76 | 0.1 | 53.1 | 12.8 |
| Wfk20A, EPS | 77 | 0.1 | 54.4 | 14.1 |
| Wfk20A, EPS | 78 | 0.1 | 49.7 | 9.4 |
| Wfk20A, EPS | 79 | 0.1 | 59.1 | 18.8 |

TABLE 17
Deep-cleaning effects of the GH114 homologues in model A detergent.

| | SEQ | Enzyme | | WP |
|---|---|---|---|---|
| Swatch | ID NO: | conc. (μg/ml) | Intensity | (ΔIntensity) |
| Wfk20A, EPS, no enzyme | | 0 | 345.0 | |
| Wfk20A, EPS | 103 | 0.003 | 358.4 | 13.4 |
| Wfk20A, EPS | 104 | 0.003 | 385.0 | 40.0 |
| Wfk20A, EPS | 107 | 0.003 | 373.7 | 28.6 |
| Wfk20A, EPS | 136 | 0.003 | 373.6 | 28.6 |
| Wfk20A, EPS | 137 | 0.003 | 363.7 | 18.7 |
| Wfk20A, EPS | 138 | 0.003 | 378.5 | 33.5 |
| Wfk20A, EPS | 139 | 0.003 | 367.7 | 22.7 |
| Wfk20A, EPS | 142 | 0.003 | 390.9 | 45.9 |
| Wfk20A, EPS | 143 | 0.003 | 352.0 | 7.0 |
| Wfk20A, EPS | 146 | 0.003 | 367.1 | 22.1 |

### Example 19. Deep-Cleaning in Liquid Model Detergent A on Pel Swatches

A crude extract of the biofilm extracellular polymer Pel was prepared from Pseudomonas aeruginosa PA14 (DSM 19882) as described above. The wash performance was determined as follows; 50 μl aliquots of the crude Pel extract were spotted on sterile textile swatches (WFK20A) and incubated for 15 min at ambient temperature. Control swatches were spotted with 3 M NaCl. The swatches (sterile or with the extract) were placed in 50 mL test tubes and 10 ml of wash liquor (15° dH water with 0.2 g/L iron (III) oxide nano-powder (544884; Sigma-Aldrich) with 3.33 g/L liquid model A detergent) and the enzyme was added to each tube. Washes without enzyme were included as controls. The test tubes were placed in a Stuart rotator and incubated for 1 hour at 30° C. and 20 rpm. The wash liquor was then removed, and the swatches were rinsed twice with 15° dH water and dried on filter paper over night. The tristimulus Y values were measured using a Handheld Minolta CR-300 and are displayed in Table 18. Wash performance (WP) values (Y_{(swatch washed with enzyme)}−Y_{(swatch washed without enzyme)}) are also indicated.

TABLE 18
Deep-cleaning effects of the GH114 homologues in model A detergent

| | SEQ | Enzyme conc. | Average | WP |
|---|---|---|---|---|
| Swatch | ID NO: | (μg/ml) | Y | (ΔY) |
| WFK20, P. aeruginosa Pel, no | | 0 | 66.0 | |
| enzyme | | | | |
| WFK20, P. aeruginosa Pel | 98 | 2 | 73.4 | 7.4 |
| WFK20, P. aeruginosa Pel | 98 | 0.2 | 72.4 | 6.4 |
| WFK20, P. aeruginosa Pel | 97 | 2 | 75.3 | 9.3 |
| WFK20, P. aeruginosa Pel | 97 | 0.2 | 67.5 | 1.5 |
| WFK20, P. aeruginosa Pel | 99 | 2 | 77.6 | 11.6 |
| WFK20, P. aeruginosa Pel | 99 | 0.2 | 71.0 | 5.0 |
| WFK20, P. aeruginosa Pel | 100 | 2 | 71.3 | 5.3 |
| WFK20, P. aeruginosa Pel | 100 | 0.2 | 66.5 | 0.5 |

### Example 20. Protein Thermal Unfolding Analysis (TSA, Thermal Shift Assay)

Protein thermal stability were tested for homologue or variants of the GH114 glycosyl hydrolase having SEQ ID NO: 18. The thermal unfolding was monitored with Sypro Orange (Invitrogen, S-6650) using a real-time PCR instrument (Applied Biosystems; Step-One-Plus). In a 96-well white PCR-plate, 15 μl sample (supernatant diluted in 100 mM EPPS pH8,0) was mixed (1:1) with Sypro Orange (Conc.=10×; stock solution from supplier=5000×) in water.

The plate was sealed with an optical PCR seal. The PCR instrument was set at a scan-rate of 76° C. per hour, starting at 25° C. and finishing at 96° C. Fluorescence was monitored every 20 seconds using in-built LED blue light for excitation and ROX-filter (610 nm, emission). Tm-values were calculated as: The temperature causing the maximum value of the first derivative (dF/dk) (Gregory et al., 2009, J. Biomol. Screen. 14:700).

TSA data for the SEQ ID NO: 18 variants using two criteria, either delta (Tm)>1 degree or between 0 and 1 degrees.

Tables 19-22 shows thermostability of the GH114 glycosyl hydrolase variants. Values above 0 means that the variant is more stable than the parent or starting GH114 glycosyl hydrolase, here SEQ ID NO: 18.

TABLE 19

| Mutation(s) | Tm_C | dTm_C |
|---|---|---|
| A250D | 44.67 | 1.93 |
| A259P | 44.85 | 1.96 |
| D222W | 46.38 | 3.79 |
| G230D + A259P | 45.64 | 2.75 |
| I172V + A250D | 45.15 | 2.73 |
| K86D | 43.61 | 1.17 |
| L234F | 44.83 | 1.94 |
| N209S | 44.77 | 1.89 |
| T39A | 43.69 | 1.25 |
| V248L | 44.75 | 2.02 |

TABLE 20

| Mutation(s) | Tm_C | dTm_C |
|---|---|---|
| C23V | 42.91 | 0.03 |
| D101* + A166V | 43.24 | 0.36 |
| D101* | 43.51 | 0.63 |
| D136E | 44.49 | 0.74 |
| D144* | 43.01 | 0.14 |
| D145E | 42.92 | 0.49 |
| D145G | 43.71 | 0.83 |
| D222E | 42.73 | 0.13 |
| E103* + E104* | 42.97 | 0.56 |
| E104* | 42.60 | 0.19 |
| E116D | 42.41 | 0.01 |
| E116K | 42.68 | 0.25 |
| E146* | 42.62 | 0.18 |
| I124L | 42.73 | 0.33 |
| I124V | 43.49 | 0.62 |
| K152D | 44.01 | 0.26 |
| K152G | 43.35 | 0.47 |
| K154E | 42.66 | 0.07 |
| K154T | 43.37 | 0.49 |
| K167D | 42.93 | 0.05 |
| K167T | 43.00 | 0.13 |
| K173T | 43.40 | 0.53 |
| K1A | 43.03 | 0.60 |
| K254T | 43.38 | 0.50 |
| K295N | 42.67 | 0.08 |
| K86E | 42.56 | 0.12 |
| K89D | 43.10 | 0.06 |
| L11I | 42.66 | 0.26 |
| L77V | 42.71 | 0.29 |
| N160D + K254S | 43.00 | 0.11 |
| N209T | 43.26 | 0.37 |
| P212* | 43.00 | 0.11 |
| Q178N | 43.51 | 0.64 |
| S102* | 42.98 | 0.55 |
| S97G | 42.46 | 0.04 |
| T119K | 43.50 | 0.63 |
| T119M + P212* | 43.28 | 0.39 |
| W80R + D145G | 43.44 | 0.57 |
| W80R + K154T | 42.93 | 0.05 |
| Y100* + D101* | 42.58 | 0.17 |
| Y296* | 42.61 | 0.02 |
| Y98W | 43.01 | 0.14 |

TABLE 21

| Mutation | Tm_C | dTm_C |
|---|---|---|
| N73Q | 44.99 | 1.14 |
| N73R | 45.11 | 1.26 |
| G75V | 44.81 | 1.25 |
| L77A | 44.70 | 1.14 |
| L77T | 45.04 | 1.48 |
| Q78M | 44.58 | 1.02 |
| D79T | 44.72 | 1.16 |
| W80M | 45.02 | 1.17 |
| W80Y | 44.98 | 1.13 |
| S85C | 45.27 | 1.42 |
| K86W | 44.74 | 1.40 |
| K89F | 44.88 | 1.32 |
| K89W | 44.62 | 1.06 |
| E90A | 44.37 | 1.03 |
| V91M | 44.59 | 1.03 |
| V242S | 45.06 | 1.19 |
| E243A | 45.33 | 2.27 |
| E243S | 45.37 | 2.32 |
| K244P | 44.54 | 1.49 |
| K247M | 45.12 | 1.25 |
| V248M | 45.99 | 2.40 |
| A250E | 45.15 | 2.05 |
| A250P | 44.30 | 1.20 |
| S251A | 44.37 | 1.43 |
| S251C | 44.53 | 1.55 |
| S251E | 46.19 | 3.21 |
| S251W | 44.32 | 1.30 |
| N253C | 44.33 | 1.26 |
| N253E | 44.79 | 1.72 |
| N253L | 44.61 | 1.55 |
| N253M | 44.10 | 1.03 |
| K254A | 44.44 | 1.42 |
| K254C | 44.11 | 1.13 |
| K254D | 46.18 | 3.32 |
| K254E | 45.87 | 2.81 |
| K254H | 44.98 | 2.00 |
| K254V | 44.13 | 1.11 |
| Y256E | 46.01 | 2.91 |
| Y256G | 44.28 | 1.18 |
| Y256M | 45.98 | 2.88 |
| Y256R | 46.59 | 3.49 |
| Y256S | 44.83 | 1.73 |
| Y256W | 45.74 | 2.64 |
| T258D | 45.05 | 1.59 |
| T258E | 44.56 | 1.10 |
| T258S | 45.21 | 1.34 |
| A259E | 45.64 | 2.41 |
| E260G | 45.17 | 1.94 |
| E260K | 48.09 | 4.86 |
| E260Q | 46.39 | 3.16 |
| E260R | 48.43 | 5.20 |
| E260T | 45.70 | 2.47 |
| E260W | 47.02 | 3.79 |
| D261A | 44.62 | 1.04 |
| E262A | 46.62 | 3.48 |
| E262F | 47.19 | 3.96 |
| E262G | 47.11 | 3.94 |
| E262I | 45.65 | 2.60 |
| E262K | 45.82 | 2.50 |
| E262L | 44.97 | 1.65 |
| E262M | 47.41 | 4.27 |
| E262Q | 47.45 | 4.40 |
| E262R | 45.47 | 2.29 |
| E262S | 47.17 | 4.12 |
| E262T | 45.33 | 2.19 |
| E262V | 45.23 | 2.12 |
| E262W | 44.93 | 1.61 |
| I270L | 47.87 | 4.81 |

TABLE 22

| Mutation | Tm_C | dTm_C |
|---|---|---|
| A64S | 44.70 | 0.85 |
| N73K | 44.81 | 0.96 |
| A76M | 43.69 | 0.13 |
| A76R | 43.96 | 0.40 |
| A76V | 43.83 | 0.27 |
| L77K | 43.87 | 0.31 |
| Q78E | 43.95 | 0.39 |
| Q78K | 43.63 | 0.29 |
| Q78N | 43.50 | 0.16 |
| Q78R | 43.97 | 0.63 |
| D79K | 44.11 | 0.55 |
| D79N | 44.09 | 0.53 |
| D79P | 44.19 | 0.63 |
| D79Q | 43.69 | 0.13 |
| D79V | 43.79 | 0.23 |
| D79W | 43.75 | 0.19 |
| W80E | 43.92 | 0.07 |
| W80I | 44.11 | 0.26 |
| W80R | 44.17 | 0.32 |
| D81V | 44.27 | 0.93 |
| S85A | 43.86 | 0.01 |
| S85G | 43.92 | 0.07 |
| S85W | 43.86 | 0.01 |
| K89A | 43.72 | 0.16 |
| K89D | 43.67 | 0.11 |
| K89E | 43.57 | 0.01 |
| K89P | 43.71 | 0.15 |
| K89S | 43.81 | 0.25 |
| K89V | 44.03 | 0.47 |
| E90D | 43.89 | 0.55 |
| E90N | 43.76 | 0.42 |
| E90S | 43.86 | 0.52 |
| V91I | 44.29 | 0.73 |
| V91S | 43.86 | 0.30 |
| V91T | 43.81 | 0.25 |
| I92E | 43.59 | 0.06 |
| E243L | 43.96 | 0.09 |
| E243P | 43.81 | 0.76 |
| E243V | 44.16 | 0.69 |
| K244L | 43.41 | 0.33 |
| K244Q | 43.09 | 0.01 |
| K244R | 43.67 | 0.60 |
| T245C | 44.50 | 0.78 |
| T245D | 43.95 | 0.09 |
| G246Q | 44.39 | 0.53 |
| K247A | 44.42 | 0.69 |
| K247C | 44.14 | 0.41 |
| K247E | 44.27 | 0.54 |
| K247R | 44.05 | 0.28 |
| K247S | 44.58 | 0.81 |
| K247V | 44.43 | 0.75 |
| V248L | 44.42 | 0.83 |
| S251G | 43.46 | 0.48 |
| S251K | 42.88 | 0.02 |
| S251L | 43.81 | 0.83 |
| S251Q | 43.98 | 0.88 |
| S251T | 43.50 | 0.52 |
| N253A | 43.38 | 0.32 |
| N253P | 43.99 | 0.92 |
| N253W | 43.09 | 0.03 |
| K254M | 43.72 | 0.86 |
| K254R | 43.25 | 0.19 |
| Y256T | 43.28 | 0.18 |
| T258A | 43.73 | 0.13 |
| T258G | 43.65 | 0.18 |
| T258Q | 44.24 | 0.77 |
| D261L | 43.38 | 0.16 |
| D261R | 43.67 | 0.09 |
| E262C | 43.54 | 0.40 |
| E262D | 43.47 | 0.33 |
| K264R | 43.76 | 0.76 |
| G279E | 43.29 | 0.24 |
| G279K | 42.99 | 0.73 |
| G279R | 43.01 | 0.75 |
| G279S | 43.09 | 0.82 |

1. A cleaning composition comprising:
(a) at least 0.01 ppm GH114 glycosyl hydrolase enzyme, wherein the GH114 glycosyl comprises one or both motif(s) [VLI]XE[EDSQ]C (SEQ ID NO: 60) or CY[FLIV][SDN][ATVG] (SEQ ID NO: 61); and
(b) one or more cleaning composition components.

2. The cleaning composition of claim 1, further comprising at least one nonionic surfactant.

3. The cleaning composition of claim 1, wherein the GH114 glycosyl hydrolase comprises one or both motifs DYQ[LI]G (SEQ ID NO: 62) or FQ[TAV]Q[PSD] (SEQ ID NO: 63).

4. The cleaning composition of claim 3, wherein the glycosyl hydrolase comprises one or both motifs AEECG (SEQ ID NO: 64) or NAFQ[AT]Q (SEQ ID NO: 65).

5. The cleaning composition of claim 1, wherein the GH114 glycosyl hydrolase comprises one or both motifs GXXVX[NHQTS]IEY[PG] (SEQ ID NO: 68) or VICYF (SEQ ID NO: 69).

6. The cleaning composition of claim 5, wherein the GH114 glycosyl hydrolase comprises the motif ICYFSA (SEQ ID NO: 70).

7. The cleaning composition of claim 5, wherein the GH114 glycosyl hydrolase comprises the motif DFAVL (SEQ ID NO: 71).

8. The cleaning composition of claim 1, wherein the GH114 glycosyl hydrolase comprises one or both motifs WQWQL (SEQ ID NO: 66) or [VLI][GASD]LKN[DGS][VLIP] (SEQ ID NO: 67).

9. The cleaning composition of claim 3, wherein the GH114 glycosyl hydrolase is selected from the group consisting of: a polypeptide having at least 60% sequence identity to the polypeptide of SEQ ID NO: 3, SEQ ID NO: 6, SEQ ID NO: 9, SEQ ID NO: 12, SEQ ID NO: 21, SEQ ID NO: 24, SEQ ID NO: 39, SEQ ID NO: 42, SEQ ID NO: 45, SEQ ID NO: 48, SEQ ID NO: 51, SEQ ID NO: 54, SEQ ID NO: 79, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 90, SEQ ID NO: 91, SEQ ID NO: 92, SEQ ID NO: 93, SEQ ID NO: 94, SEQ ID NO: 96, SEQ ID NO: 131, SEQ ID NO: 132, SEQ ID NO: 134, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144 and SEQ ID NO: 145.

10. The cleaning composition of claim 4, wherein the GH114 glycosyl hydrolase is selected from the group consisting of: a polypeptide having at least 60% sequence identity to the polypeptide of SEQ ID NO: 3, SEQ ID NO: 6, SEQ ID NO: 9, SEQ ID NO: 12, SEQ ID NO: 21, SEQ ID NO: 24, SEQ ID NO: 54, SEQ ID NO: 79, SEQ ID NO: 82, SEQ ID NO: 83, SEQ ID NO: 84, SEQ ID NO: 85, SEQ ID NO: 132, SEQ ID NO: 136, SEQ ID NO: 138, and SEQ ID NO: 142.

11. The cleaning composition of claim 5, wherein the GH114 glycosyl hydrolase is selected from the group consisting of: a polypeptide having at least 60%, sequence identity to the polypeptide of SEQ ID NO: 15, SEQ ID NO: 18, SEQ ID NO: 57, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 133 and SEQ ID NO: 135.

12. The cleaning composition of claim 6, wherein the GH114 glycosyl hydrolase is selected from the group consisting of: a polypeptide having at least 60%, sequence identity to the polypeptide of SEQ ID NO: 15, SEQ ID NO: 57, SEQ ID NO: 98, SEQ ID NO: 101, SEQ ID NO: 104, SEQ ID NO: 107, SEQ ID NO: 114, SEQ ID NO: 115, SEQ ID NO: 120, SEQ ID NO: 124, SEQ ID NO: 125, SEQ ID NO: 127, SEQ ID NO: 129 and SEQ ID NO: 133.

13. The cleaning composition of claim 7, wherein the GH114 glycosyl hydrolase is selected from the group consisting of: a polypeptide having at least 60%, sequence identity to the polypeptide of SEQ ID NO: 18, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 110, SEQ ID NO: 111, SEQ ID NO: 112, SEQ ID NO: 113, SEQ ID NO: 116, SEQ ID NO: 117, SEQ ID NO: 118, SEQ ID NO: 119, SEQ ID NO: 121, SEQ ID NO: 123, SEQ ID NO: 126, SEQ ID NO: 128, SEQ ID NO: 130 and SEQ ID NO: 135.

14. The cleaning composition of claim 8, wherein the GH114 glycosyl hydrolase is selected from the group consisting of: a polypeptide having at least 60%, sequence identity to the polypeptide of SEQ ID NO: 27, SEQ ID NO: 33, SEQ ID NO: 36, SEQ ID NO: 80, SEQ ID NO: 81, SEQ ID NO: 89, SEQ ID NO: 95 and SEQ ID NO: 146.

15. (canceled)

16. The composition of claim 1, wherein the GH114 glycosyl hydrolase has wash performance (WP) in liquid Model detergent A, measured as a delta L (ΔL)>1, when delta L (ΔL) is calculated (L(swatch washed with enzyme)−L(swatch washed without enzyme)).

17. The composition of claim 1, wherein the GH114 glycosyl hydrolase enzyme has biofilm removal activity, wherein the % remaining biofilm is less than 80% biofilm remaining on the item.

18. A method for laundering an item comprising:
(a) exposing an item to a wash liquor comprising a composition of claim 1;
(b) completing at least one wash cycle; and
(c) optionally rinsing the item, wherein the item is a textile.

19-20. (canceled)
